SEQUENCE LISTING (322 sequences)

SEQ ID NO: 1 - protein, 217 aa, Homo sapiens
  REGION 1..217: note = MISC_FEATURE - Human Wild-Type IgG CH2-CH3 Domain
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 440 aa, Homo sapiens [sequence omitted]

SEQ ID NO: 3 - protein, 111 aa, Mus musculus
  REGION 1..111: note = MISC_FEATURE - Murine Anti-Human DR5 Antibody ("DR5 mAb 1") Light Chain Variable Domain
DIVLTQSPASLAVSLGQRATISCRASKSVSSSGYSYMHWYQQKPGQPPKVLIFLSSNLDSGVPARFSGSGSGTDFTLNIHPVEDGDAATYYCQHSRDLPPTFGGGTKLEIK

SEQ ID NO: 4 - protein, 15 aa, Mus musculus
  REGION 1..15: note = MISC_FEATURE - Murine Anti-Human DR5 Antibody ("DR5 mAb 1") Light Chain Variable Domain CDRL1
RASKSVSSSGYSYMH

SEQ ID NO: 5 - protein, 7 aa, Mus musculus
  REGION 1..7: note = MISC_FEATURE - Murine Anti-Human DR5 Antibody ("DR5 mAb 1") Light Chain Variable Domain CDRL2
LSSNLDS

SEQ ID NO: 6 - protein, 9 aa, Mus musculus
  REGION 1..9: note = MISC_FEATURE - Murine Anti-Human DR5 Antibody ("DR5 mAb 1") Light Chain Variable Domain CDRL3
QHSRDLPPT

SEQ ID NO: 7 - DNA, 333 bp, Mus musculus
  misc_feature 1..333: note = Polynucleotide Encoding Murine Anti-Human DR5 Antibody ("DR5 mAb 1") Light Chain Variable Domain
  source 1..333: mol_type = genomic DNA
gacattgtgctgacacagtctcctgcttccttagctgtatctctcgggcagagggccaccatctcatgcagggccagcaaaagtgtcagttcctctggctatagttatatgcactggtaccaacagaaaccaggacagccacccaaagtcctcatctttctttcatccaacctagattctggggtccctgccaggttcagtggcagtgggtctgggacagacttcaccctcaacatccatcctgtggaggatggggatgctgcaacctattactgtcagcacagtagggatcttcctccgacgttcggtggaggcaccaagctggaaatcaaa

SEQ ID NO: 8 - protein, 121 aa, Mus musculus
  REGION 1..121: note = MISC_FEATURE - Murine Anti-Human DR5 Antibody ("DR5 mAb 1") Heavy Chain Variable Domain
EVKFLESGGGLVQPGGSLKLSCVASGFDFSRYWMSWVRQAPGKGLEWIGEINPDSNTINYTPSLKDKFIISRDNAKNTLYLQMTKVRSEDTALYYCTRRAYYGNPAWFAYWGQGTLVTVSS

SEQ ID NO: 9 - protein, 10 aa, Mus musculus
  REGION 1..10: note = MISC_FEATURE - Murine Anti-Human DR5 Antibody ("DR5 mAb 1") Heavy Chain Variable Domain CDRH1
GFDFSRYWMS

SEQ ID NO: 10 - protein, 17 aa, Mus musculus
  REGION 1..17: note = MISC_FEATURE - Murine Anti-Human DR5 Antibody ("DR5 mAb 1") Heavy Chain Variable Domain CDRH2
EINPDSNTINYTPSLKD

SEQ ID NO: 11 - protein, 12 aa, Mus musculus
  REGION 1..12: note = MISC_FEATURE - Murine Anti-Human DR5 Antibody ("DR5 mAb 1") Heavy Chain Variable Domain CDRH3
RAYYGNPAWFAY

SEQ ID NO: 12 - DNA, 363 bp, Mus musculus
  misc_feature 1..363: note = Polynucleotide Encoding Murine Anti-Human DR5 Antibody ("DR5 mAb 1") Heavy Chain Variable Domain
  source 1..363: mol_type = genomic DNA
gaggtgaagtttctcgagtctggaggtggcctggtgcagcctggaggatccctgaaactctcctgtgtagcctcaggattcgattttagtagatactggatgagttgggtccggcaggctccagggaaagggctagaatggattggagaaattaatccagatagcaatacgataaactatacgccatctctaaaggataaattcatcatctccagagacaacgccaaaaatacgctgtatctgcaaatgaccaaagtgagatctgaggacacagccctttattattgtacaagaagggcctactatggtaacccggcctggtttgcttactggggccaagggactctggtcactgtctcttcc

SEQ ID NO: 13 - protein, 107 aa, Mus musculus
  REGION 1..107: note = MISC_FEATURE - Murine Anti-Human DR5 Antibody ("DR5 mAb 2") Light Chain Variable Domain
DIVMTQSHKFMSTSVGDRVSITCKASQDVNTAVAWYQQKPGQSPKLLIYWASTRHTGVPDRFTGSGSGTDYTLTIKSVQAEDLTLYYCQQHYITPWTFGGGTKLEIK

SEQ ID NO: 14 - protein, 11 aa, Mus musculus
  REGION 1..11: note = MISC_FEATURE - Murine Anti-Human DR5 Antibody ("DR5 mAb 2") Light Chain Variable Domain CDRL1
KASQDVNTAVA

SEQ ID NO: 15 - protein, 7 aa, Mus musculus
  REGION 1..7: note = MISC_FEATURE - Murine Anti-Human DR5 Antibody ("DR5 mAb 2") Light Chain Variable Domain CDRL2
WASTRHT

SEQ ID NO: 16 - protein, 9 aa, Mus musculus
  REGION 1..9: note = MISC_FEATURE - Murine Anti-Human DR5 Antibody ("DR5 mAb 2") Light Chain Variable Domain CDRL3
QQHYITPWT

SEQ ID NO: 17 - DNA, 321 bp, Mus musculus
  misc_feature 1..321: note = Polynucleotide Encoding Murine Anti-Human DR5 Antibody ("DR5 mAb 2") Light Chain Variable Domain
  source 1..321: mol_type = genomic DNA
gacattgtgatgacccagtctcacaaattcatgtccacttcagtaggagacagggtcagcatcacctgcaaggccagtcaggatgtgaatactgctgtagcctggtatcaacaaaaaccagggcaatctcctaaactactgatttactgggcatccacccggcacactggagtccctgatcgcttcacaggcagtggatctgggacagattatacactcaccatcaaaagtgtgcaggctgaagacctgacactttattactgtcagcaacactatatcactccgtggacgttcggtggaggcaccaagctggaaatcaaa

SEQ ID NO: 18 - protein, 119 aa, Mus musculus
  REGION 1..119: note = MISC_FEATURE - Murine Anti-Human DR5 Antibody ("DR5 mAb 2") Heavy Chain Variable Domain
KVQLQQSGAELVKPGASVKLSCKASGYTFTEYILHWVKQKSGQGLEWIGWFYPGNNNIKYNEKFKDKATLTADKSSSTVYMELSRLTSEDSAVYFCARHEQGPGYFDYWGQGTTLTVSS

SEQ ID NO: 19 - protein, 10 aa, Mus musculus
  REGION 1..10: note = MISC_FEATURE - Murine Anti-Human DR5 Antibody ("DR5 mAb 2") Heavy Chain Variable Domain CDRH1
GYTFTEYILH

SEQ ID NO: 20 - protein, 17 aa, Mus musculus
  REGION 1..17: note = MISC_FEATURE - Murine Anti-Human DR5 Antibody ("DR5 mAb 2") Heavy Chain Variable Domain CDRH2
WFYPGNNNIKYNEKFKD

SEQ ID NO: 21 - protein, 10 aa, Mus musculus
  REGION 1..10: note = MISC_FEATURE - Murine Anti-Human DR5 Antibody ("DR5 mAb 2") Heavy Chain Variable Domain CDRH3
HEQGPGYFDY

SEQ ID NO: 22 - DNA, 357 bp, Mus musculus
  misc_feature 1..357: note = Polynucleotide Encoding Murine Anti-Human DR5 Antibody ("DR5 mAb 2") Heavy Chain Variable Domain
  source 1..357: mol_type = genomic DNA
aaggtccagctgcagcagtctggagctgaactggtgaaacccggggcatcagtgaagctgtcctgcaaggcttctgggtacaccttcactgagtatattttacactgggtaaagcagaagtctggacagggtcttgagtggattgggtggttttatcctggaaataataatataaagtacaatgagaaattcaaggacaaggccacactgactgcggacaaatcctccagcacagtctatatggaacttagtagattgacatctgaagactctgcggtctatttctgtgcaagacacgaacaaggaccaggttactttgactactggggccaaggcaccactctcacagtctcctcc

SEQ ID NO: 23 - protein, 107 aa, synthetic construct
  REGION 1..107: note = Humanized Anti-Human DR5 Antibody ("hDR5 mAb 2 VL-2") Light Chain Variable Domain
DIQMTQSPSFLSASVGDRVTITCKASQDVNTAVAWYQQKPGKAPKLLIYWASTRHTGVPSRFSGSGSGTDFTLTISSLQPEDVATYYCQQHYITPWTFGGGTKLEIK

SEQ ID NO: 24 - DNA, 321 bp, synthetic construct
  misc_feature 1..321: note = Polynucleotide Encoding Humanized Anti-Human DR5 Antibody ("hDR5 mAb 2 VL-2") Light Chain Variable Domain
  source 1..321: mol_type = other DNA
gatattcagatgacccagagtccctcatttctgtccgcctccgtcggtgaccgcgtgactattacttgtaaagcttctcaggatgtcaacaccgccgtggcttggtaccagcagaagcccggtaaagcacctaagctgctgatctattgggccagcactcggcacaccggagtcccatctaggttctctggcagtggatcagggacagactttaccctgacaattagctccctgcagcccgaggatgtggctacttactattgtcagcagcactacatcactccttggaccttcggcgggggcacaaaactggaaatcaaa

SEQ ID NO: 25 - protein, 107 aa, synthetic construct
  REGION 1..107: note = Humanized Anti-Human DR5 Antibody ("hDR5 mAb 2 VL-3") Light Chain Variable Domain
DIQMTQSPSFLSASVGDRVTITCRASQDVNTAVAWYQQKPGKAPKLLIYWASTRHTGVPDRFSGSGSGTDFTLTISSLQPEDVATYYCQQHYITPWTFGGGTKLEIK

SEQ ID NO: 26 - DNA, 321 bp, synthetic construct
  misc_feature 1..321: note = Polynucleotide Encoding Humanized Anti-Human DR5 Antibody ("hDR5 mAb 2 VL-3") Light Chain Variable Domain
  source 1..321: mol_type = other DNA
gatattcagatgacccagagtccctcatttctgtccgcctccgtcggtgaccgcgtgactattacttgtcgggcttctcaggatgtcaacaccgccgtggcttggtaccagcagaagcccggtaaagcacctaagctgctgatctattgggccagcactcggcacaccggagtcccagataggttctctggcagtggatcagggacagactttaccctgacaattagctccctgcagcccgaggatgtggctacttactattgtcagcagcactacatcactccttggaccttcggcgggggcacaaaactggaaatcaaa

SEQ ID NO: 27 - protein, 107 aa, synthetic construct
  REGION 1..107: note = Murine Anti-Human DR5 Antibody ("DR5 mAb 4") Light Chain Variable Domain
DIQMTQSPSFLSASVGDRVTITCRASQDVNTAVAWYQQKPGKAPKLLIYWASTRHTGVPSRFSGSGSGTDFTLTISSLQPEDIATYYCQQHYITPWTFGGGTKLEIK

SEQ ID NO: 28 - DNA, 321 bp, synthetic construct
  misc_feature 1..321: note = Polynucleotide Encoding Humanized Anti-Human DR5 Antibody ("hDR5 mAb 2 VL-4") Light Chain Variable Domain
  source 1..321: mol_type = other DNA
gatattcagatgacccagagtccctcatttctgtccgcctccgtcggtgaccgcgtgactattacttgtcgggcttctcaggatgtcaacaccgccgtggcttggtaccagcagaagcccggtaaagcacctaagctgctgatctattgggccagcactcggcacaccggagtcccatctaggttctctggcagtggatcagggacagactttaccctgacaattagctccctgcagccagaggatatcgctacatactattgtcagcagcactacatcactccttggaccttcggcgggggcacaaaactggaaatcaaa

SEQ ID NO: 29 - protein, 107 aa, synthetic construct
  REGION 1..107: note = Humanized Anti-Human DR5 Antibody ("hDR5 mAb 2 VL-5") Light Chain Variable Domain
DIQMTQSPSFLSASVGDRVTITCRASQDVNTAVAWYQQKPGKAPKLLIYWASTRHTGVPDRFSGSGSGTDFTLTISSLQPEDIATYYCQQHYITPWTFGGGTKLEIK

SEQ ID NO: 30 - DNA, 321 bp, synthetic construct
  misc_feature 1..321: note = Polynucleotide Encoding Humanized Anti-Human DR5 Antibody ("hDR5 mAb 2 VL-5") Light Chain Variable Domain
  source 1..321: mol_type = other DNA
gatattcagatgacccagagtccctcatttctgtccgcctccgtcggtgaccgcgtgactattacttgtcgggcttctcaggatgtcaacaccgccgtggcttggtaccagcagaagcccggtaaagcacctaagctgctgatctattgggccagcactcggcacaccggagtcccagataggttctctggcagtggatcagggacagactttaccctgacaattagctccctgcagcccgaggatatcgctacttactattgtcagcagcactacatcactccttggaccttcggcgggggcacaaaactggaaatcaaa

SEQ ID NO: 31 - protein, 119 aa, synthetic construct
  REGION 1..119: note = Humanized Anti-Human DR5 Antibody ("hDR5 mAb 2 VH-2") Heavy Chain Variable Domain
QVQLVQSGAEVKKPGASVKVSCKASGYTFTEYILHWVRQAPGQGLEWMGWFYPGNNNIKYNEKFKDRVTITADKSTSTVYMELSSLRSEDTAVYYCARHEQGPGYFDYWGQGTLVTVSS

SEQ ID NO: 32 - DNA, 357 bp, synthetic construct
  misc_feature 1..357: note = Polynucleotide Encoding Humanized Anti-Human DR5 Antibody ("hDR5 mAb 2 VH-2") Heavy Chain Variable Domain
  source 1..357: mol_type = other DNA
caggtccagctggtgcagagtggggcagaggtgaaaaagccaggggcatcagtgaaagtgtcttgtaaagcatcaggttatacatttactgagtacatcctgcactgggtgcgacaggcaccaggacagggactggaatggatggggtggttctaccctggcaacaacaacattaagtacaacgagaagtttaaagaccgggtgaccatcacagcggataagtctaccagtacagtctatatggagctgagctccctgagaagcgaagacaccgccgtctactattgcgctcgccacgaacagggtccaggttactttgattattgggggcagggaactctggtcacagtcagctcc

SEQ ID NO: 33 - protein, 8 aa, synthetic construct
  REGION 1..8: note = Linker 1
GGGSGGGG

SEQ ID NO: 34 - protein, 6 aa, synthetic construct
  REGION 1..6: note = Cysteine-Containing Spacer Peptide ("Linker 2")
GGCGGG

SEQ ID NO: 35 - protein, 7 aa, synthetic construct
  REGION 1..7: note = Cysteine-Containing Peptide Variant of Human IgG Hinge Domain
GVEPKSC

SEQ ID NO: 36 - protein, 6 aa, synthetic construct
  REGION 1..6: note = Cysteine-Containing Peptide Variant of Human IgG Hinge Domain
VEPKSC

SEQ ID NO: 37 - protein, 7 aa, synthetic construct
  REGION 1..7: note = Cysteine-Containing Peptide Variant of Human IgG CL Domain
GFNRGEC

SEQ ID NO: 38 - protein, 6 aa, synthetic construct
  REGION 1..6: note = Cysteine-Containing Peptide Variant of Human IgG CL Domain
FNRGEC

SEQ ID NO: 39 - protein, 28 aa, synthetic construct
  REGION 1..28: note = Heterodimer-Promoting "E-coil" Domain
EVAALEKEVAALEKEVAALEKEVAALEK

SEQ ID NO: 40 - protein, 28 aa, synthetic construct
  REGION 1..28: note = Heterodimer-Promoting "K-coil" Domain
KVAALKEKVAALKEKVAALKEKVAALKE

SEQ ID NO: 41 - protein, 28 aa, synthetic construct
  REGION 1..28: note = Cysteine-Containing Heterodimer-Promoting "E-coil" Domain
EVAACEKEVAALEKEVAALEKEVAALEK

SEQ ID NO: 42 - protein, 28 aa, synthetic construct
  REGION 1..28: note = Cysteine-Containing Heterodimer-Promoting "K-coil" Domain
KVAACKEKVAALKEKVAALKEKVAALKE

SEQ ID NO: 43 - protein, 46 aa, Streptococcus dysgalactiae
  REGION 1..46: note = MISC_FEATURE - Albumin-Binding Domain 3 (ABD3) of Protein G of Streptococcus Strain G148
LAEAKVLANRELDKYGVSDYYKNLIDNAKSAEGVKALIDEILAALP

SEQ ID NO: 44 - protein, 46 aa, synthetic construct
  REGION 1..46: note = Variant Deimmunized Albumin-Binding Domain 3 (ABD3) of Protein G of Streptococcus Strain G148
LAEAKVLANRELDKYGVSDYYKNAANNAKTVEGVKALIAEILAALP

SEQ ID NO: 45 - protein, 46 aa, synthetic construct
  REGION 1..46: note = Variant Deimmunized Albumin-Binding Domain 3 (ABD3) of Protein G of Streptococcus Strain G148
LAEAKVLANRELDKYGVSDYYKNLISNAKSVEGVKALIAEILAALP

SEQ ID NO: 46 - protein, 4 aa, synthetic construct
  REGION 1..4: note = Peptide Linker ("Linker 3")
GGGS

SEQ ID NO: 47 - protein, 5 aa, synthetic construct
  REGION 1..5: note = Peptide Linker
ASTKG

SEQ ID NO: 48 - protein, 10 aa, synthetic construct
  REGION 1..10: note = Peptide Linker
DKTHTCPPCP

SEQ ID NO: 49 - protein, 6 aa, synthetic construct
  REGION 1..6: note = Peptide Linker
LEPKSS

SEQ ID NO: 50 - protein, 8 aa, synthetic construct
  REGION 1..8: note = Peptide Linker
APSSSPME

SEQ ID NO: 51 - protein, 16 aa, synthetic construct
  REGION 1..16: note = Peptide Linker
LEPKSSDKTHTCPPCP

SEQ ID NO: 52 - protein, 217 aa, synthetic construct
  REGION 1..217: note = "Knob-Bearing" Variant Human IgG Fc Domain
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 217 aa, synthetic construct
  REGION 1..217: note = "Hole-Bearing" Variant Human IgG Fc Domain
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 54 - protein, 108 aa, Homo sapiens
  REGION 1..108: note = MISC_FEATURE - human Anti-Human DR5 Antibody (Drozitumab; "DR5 mAb 3") Light Chain Variable Domain
SELTQDPAVSVALGQTVRITCSGDSLRSYYASWYQQKPGQAPVLVIYGANNRPSGIPDRFSGSSSGNTASLTITGAQAEDEADYYCNSADSSGNHVVFGGGTKLTVLG

SEQ ID NO: 55 - protein, 11 aa, Homo sapiens
  REGION 1..11: note = MISC_FEATURE - Human Anti-Human DR5 Antibody (Drozitumab; "DR5 mAb 3") Light Chain Variable Domain CDRL1
SGDSLRSYYAS

SEQ ID NO: 56 - protein, 7 aa, Homo sapiens
  REGION 1..7: note = MISC_FEATURE - Human Anti-Human DR5 Antibody (Drozitumab; "DR5 mAb 3") Light Chain Variable Domain CDRL2
GANNRPS

SEQ ID NO: 57 - protein, 11 aa, Homo sapiens
  REGION 1..11: note = MISC_FEATURE - Human Anti-Human DR5 Antibody (Drozitumab; "DR5 mAb 3") Light Chain Variable Domain CDRL3
NSADSSGNHVV

SEQ ID NO: 58 - protein, 121 aa, Homo sapiens [sequence omitted]

SEQ ID NO: 59 - protein, 10 aa, Homo sapiens
  REGION 1..10: note = MISC_FEATURE - Human Anti-Human DR5 Antibody (Drozitumab; "DR5 mAb 3") Heavy Chain Variable Domain CDRH1
GFTFDDYAMS

SEQ ID NO: 60 - protein, 16 aa, Homo sapiens
  REGION 1..16: note = MISC_FEATURE - Human Anti-Human DR5 Antibody (Drozitumab; "DR5 mAb 3") Heavy Chain Variable Domain CDRH2
INWQGGSTGYADSVKG

SEQ ID NO: 61 - protein, 12 aa, Homo sapiens
  REGION 1..12: note = MISC_FEATURE - Human Anti-Human DR5 Antibody (Drozitumab; "DR5 mAb 3") Heavy Chain Variable Domain CDRH2
ILGAGRGWYFDY

SEQ ID NO: 62 - protein, 108 aa, Homo sapiens
  REGION 1..108: note = MISC_FEATURE - Human Anti-Human DR5 Antibody (Conatumumab; "DR5 mAb 4") Light Chain Variable Domain
EIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIK

SEQ ID NO: 63 - protein, 12 aa, Homo sapiens
  REGION 1..12: note = MISC_FEATURE - Human Anti-Human DR5 Antibody (Conatumumab; "DR5 mAb 4") Light Chain Variable Domain CDRL1
RASQGISRSYLA

SEQ ID NO: 64 - protein, 7 aa, Homo sapiens
  REGION 1..7: note = MISC_FEATURE - Huamn Anti-Human DR5 Antibody (Conatumumab; "DR5 mAb 4") Light Chain Variable Domain CDRL2
GASSRAT

SEQ ID NO: 65 - protein, 9 aa, Homo sapiens
  REGION 1..9: note = MISC_FEATURE - Human Anti-Human DR5 Antibody (Conatumumab; "DR5 mAb 4") Light Chain Variable Domain CDRL3
QQFGSSPWT

SEQ ID NO: 66 - protein, 122 aa, Homo sapiens
  REGION 1..122: note = MISC_FEATURE - Human Anti-Human DR5 Antibody (Conatumumab; "DR5 mAb 4") Heavy Chain Variable Domain
QVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSS

SEQ ID NO: 67 - protein, 12 aa, Homo sapiens
  REGION 1..12: note = MISC_FEATURE - Human Anti-Human DR5 Antibody (Conatumumab; "DR5 mAb 4") Heavy Chain Variable Domain CDRH1
GGSISSGDYFWS

SEQ ID NO: 68 - protein, 16 aa, Homo sapiens
  REGION 1..16: note = MISC_FEATURE - Human Anti-Human DR5 Antibody (Conatumumab; "DR5 mAb 4") Heavy Chain Variable Domain CDRH2
HIHNSGTTYYNPSLKS

SEQ ID NO: 69 - protein, 12 aa, Homo sapiens
  REGION 1..12: note = MISC_FEATURE - Human Anti-Human DR5 Antibody (Conatumumab; "DR5 mAb 4") Heavy Chain Variable Domain CDRH3
DRGGDYYYGMDV

SEQ ID NO: 70 - protein, 106 aa, synthetic construct [sequence omitted]

SEQ ID NO: 71 - protein, 11 aa, synthetic construct
  REGION 1..11: note = Humanized Anti-Human DR5 Antibody (Tigatuzumab; "DR5 mAb 5") Light Chain Variable Domain CDRL1
KASQDVGTAVA

SEQ ID NO: 72 - protein, 7 aa, synthetic construct
  REGION 1..7: note = Humanized Anti-Human DR5 Antibody (Tigatuzumab; "DR5 mAb 5") Light Chain Variable Domain CDRL2
WASTRHT

SEQ ID NO: 73 - protein, 8 aa, synthetic construct
  REGION 1..8: note = Humanized Anti-Human DR5 Antibody (Tigatuzumab; "DR5 mAb 5") Light Chain Variable Domain CDRL3
QQYSSYRT

SEQ ID NO: 74 - protein, 119 aa, synthetic construct [sequence omitted]

SEQ ID NO: 75 - protein, 10 aa, synthetic construct
  REGION 1..10: note = Humanized Anti-Human DR5 Antibody (Tigatuzumab; "DR5 mAb 5") Heavy Chain Variable Domain CDRH1
GFTFSSYVMS

SEQ ID NO: 76 - protein, 17 aa, synthetic construct
  REGION 1..17: note = Humanized Anti-Human DR5 Antibody (Tigatuzumab; "DR5 mAb 5") Heavy Chain Variable Domain CDRH2
TISSGGSYTYYPDSVKG

SEQ ID NO: 77 - protein, 10 aa, synthetic construct
  REGION 1..10: note = Humanized Anti-Human DR5 Antibody (Tigatuzumab; "DR5 mAb 5") Heavy Chain Variable Domain CDRH3
RGDSMITTDY

SEQ ID NO: 78 - protein, 109 aa, Mus musculus
  REGION 1..109: note = MISC_FEATURE - Murine Anti-Human DR5 Antibody (LBY135-1; "DR5 mAb 6") Light Chain Variable Domain
DIAMTQSHKFMSTLVGDRVSITCKASQDVNTAIAWYQQKPGQSPKLLIYWASTRHTGVPDRFYGSGSGTDYTLTISSMEAEDAATYYCQQWSSNPLTFGAGTKLELKRA

SEQ ID NO: 79 - protein, 8 aa, Mus musculus
  REGION 1..8: note = MISC_FEATURE - Murine Anti-Human DR5 Antibody (LBY135-1; "DR5 mAb 6") Light Chain Variable Domain CDRL1
QDVNTAIA

SEQ ID NO: 80 - protein, 7 aa, Mus musculus
  REGION 1..7: note = MISC_FEATURE - Murine Anti-Human DR5 Antibody (LBY135-1; "DR5 mAb 6") Light Chain Variable Domain CDRL2
WASTRHT

SEQ ID NO: 81 - protein, 9 aa, Mus musculus
  REGION 1..9: note = MISC_FEATURE - Murine Anti-Human DR5 Antibody (LBY135-1; "DR5 mAb 6") Light Chain Variable Domain CDRL3
QQWSSNPLT

SEQ ID NO: 82 - protein, 118 aa, Mus musculus
  REGION 1..118: note = MISC_FEATURE - Murine Anti-Human DR5 Antibody (LBY135-1; "DR5 mAb 6") Heavy Chain Variable Domain
KVQLQQSGAELVKPGASVKLSCKASGYTFTDYTIHWVKQRSGQGLEWIGWFYPGGGYIKYNEKFKDRATLTADKSSNTVYMELSRLTSEGSAVYFCARHEEGIYFDYWGQGTTLTVSS

SEQ ID NO: 83 - protein, 10 aa, Mus musculus
  REGION 1..10: note = MISC_FEATURE - Murine Anti-Human DR5 Antibody (LBY135-1; "DR5 mAb 6") Heavy Chain Variable Domain CDRH1
GYTFTDYTIH

SEQ ID NO: 84 - protein, 17 aa, Mus musculus
  REGION 1..17: note = MISC_FEATURE - Murine Anti-Human DR5 Antibody (LBY135-1; "DR5 mAb 6") Heavy Chain Variable Domain CDRH2
WFYPGGGYIKYNEKFKD

SEQ ID NO: 85 - protein, 9 aa, Mus musculus
  REGION 1..9: note = MISC_FEATURE - Murine Anti-Human DR5 Antibody (LBY135-1; "DR5 mAb 6") Heavy Chain Variable Domain CDRH3
HEEGIYFDY

SEQ ID NO: 86 - protein, 104 aa, Mus musculus
  REGION 1..104: note = MISC_FEATURE - Murine Anti-Human DR5 Antibody (LBY135-2; "DR5 mAb 7") Light Chain Variable Domain
DIVMTQSHKFMSTSVGDRVSITCKASQDVNTAIAWYQQKPGQSPKLLIYWASTRHTGVPDRFTGSGSGTDYTLTISSVQAEDLALYYCQQHYTTPFTFGSGTKL

SEQ ID NO: 87 - protein, 11 aa, Mus musculus
  REGION 1..11: note = MISC_FEATURE - Murine Anti-Human DR5 Antibody (LBY135-2; "DR5 mAb 7") Light Chain Variable Domain CDRL1
KASQDVNTAIA

SEQ ID NO: 88 - protein, 7 aa, Mus musculus
  REGION 1..7: note = MISC_FEATURE - Murine Anti-Human DR5 Antibody (LBY135-2; "DR5 mAb 7") Light Chain Variable Domain CDRL2
WASTRHT

SEQ ID NO: 89 - protein, 9 aa, Mus musculus
  REGION 1..9: note = MISC_FEATURE - Murine Anti-Human DR5 Antibody (LBY135-2; "DR5 mAb 7") Light Chain Variable Domain CDRL3
QQHYTTPFT

SEQ ID NO: 90 - protein, 118 aa, Mus musculus
  REGION 1..118: note = MISC_FEATURE - Murine Anti-Human DR5 Antibody (LBY135-2; "DR5 mAb 7") Heavy Chain Variable Domain
KVQLQQSGAELVKPGASVKLSCKASGYTFTDYTIHWVKQRSGQGLEWIGWFYPGGGYIKYNEKFKDRATLTADKSSNTVYMELSRLTSEDSAVYFCARHEEGIYFDYWGQGTTLTVSS

SEQ ID NO: 91 - protein, 10 aa, Mus musculus
  REGION 1..10: note = MISC_FEATURE - Murine Anti-Human DR5 Antibody (LBY135-2; "DR5 mAb 7") Heavy Chain Variable Domain CDRH1
GYTFTDYTIH

SEQ ID NO: 92 - protein, 17 aa, Mus musculus
  REGION 1..17: note = MISC_FEATURE - Murine Anti-Human DR5 Antibody (LBY135-2; "DR5 mAb 7") Heavy Chain Variable Domain CDRH2
WFYPGGGYIKYNEKFKD

SEQ ID NO: 93 - protein, 9 aa, Mus musculus
  REGION 1..9: note = MISC_FEATURE - Murine Anti-Human DR5 Antibody (LBY135-2; "DR5 mAb 7") Heavy Chain Variable Domain CDRH3
HEEGIYFDY

SEQ ID NO: 94 - protein, 108 aa, Mus musculus
  REGION 1..108: note = MISC_FEATURE - Murine Anti-Human DR5 Antibody (KMTR2; "DR5 mAb 8") Light Chain Variable Domain
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIKR

SEQ ID NO: 95 - protein, 11 aa, Mus musculus
  REGION 1..11: note = MISC_FEATURE - Murine Anti-Human DR5 Antibody (KMTR2; "DR5 mAb 8") Light Chain Variable Domain CDRL1
RASQSVSSYLA

SEQ ID NO: 96 - protein, 7 aa, Mus musculus
  REGION 1..7: note = MISC_FEATURE - Murine Anti-Human DR5 Antibody (KMTR2; "DR5 mAb 8") Light Chain Variable Domain CDRL2
DASNRAT

SEQ ID NO: 97 - protein, 9 aa, Mus musculus
  REGION 1..9: note = MISC_FEATURE - Murine Anti-Human DR5 Antibody (KMTR2; "DR5 mAb 8") Light Chain Variable Domain CDRL3
QQRSNWPLT

SEQ ID NO: 98 - protein, 126 aa, Mus musculus
  REGION 1..126: note = MISC_FEATURE - Murine Anti-Human DR5 Antibody (KMTR2; "DR5 mAb 8") Heavy Chain Variable Domain
QVQLVQSGAEMKKPGASVKVSCKTSGYTFTNYKINWVRQAPGQGLEWMGWMNPDTDSTGYPQKFQGRVTMTRNTSISTAYMELSSLRSEDTAVYYCARSYGSGSYYRDYYYGMDVWGQGTTVTVSS

SEQ ID NO: 99 - protein, 10 aa, Mus musculus
  REGION 1..10: note = MISC_FEATURE - Murine Anti-Human DR5 Antibody (KMTR2; "DR5 mAb 8") Heavy Chain Variable Domain CDRH1
GYTFTNYKIN

SEQ ID NO: 100 - protein, 17 aa, Mus musculus
  REGION 1..17: note = MISC_FEATURE - Murine Anti-Human DR5 Antibody (KMTR2; "DR5 mAb 8") Heavy Chain Variable Domain CDRH2
WMNPDTDSTGYPQKFQG

SEQ ID NO: 101 - protein, 17 aa, Mus musculus
  REGION 1..17: note = MISC_FEATURE - Murine Anti-Human DR5 Antibody (KMTR2; "DR5 mAb 8") Heavy Chain Variable Domain CDRH3
SYGSGSYYRDYYYGMDV

SEQ ID NO: 102 - protein, 112 aa, Mus musculus
  REGION 1..112: note = MISC_FEATURE - Murine Anti-Human CD2 Antibody (Lo-CD2A mAb ) Light Chain Variable Domain
DVVLTQTPPTLLATIGQSVSISCRSSQSLLHSSGNTYLNWLLQRTGQSPQPLIYLVSKLESGVPNRFSGSGSGTDFTLKISGVEAEDLGVYYCMQFTHYPYTFGAGTKLELK

SEQ ID NO: 103 - protein, 118 aa, Mus musculus
  REGION 1..118: note = MISC_FEATURE - Murine Anti-Human CD2 Antibody (Lo-CD2A mAb ) Heavy Chain Variable Domain
EVQLQQSGPELQRPGASVKLSCKASGYIFTEYYMYWVKQRPKQGLELVGRIDPEDGSIDYVEKFKKKATLTADTSSNTAYMQLSSLTSEDTATYFCARGKFNYRFAYWGQGTLVTVSS

SEQ ID NO: 104 - protein, 110 aa, synthetic construct
  REGION 1..110: note = Humanized Anti-Human CD3 Antibody ("CD3 mAb 2") Light Chain Variable Domain
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLG

SEQ ID NO: 105 - protein, 14 aa, synthetic construct
  REGION 1..14: note = Humanized Anti-Human CD3 Antibody ("CD3 mAb 2") Light Chain Variable Domain CDRL1
RSSTGAVTTSNYAN

SEQ ID NO: 106 - protein, 7 aa, synthetic construct
  REGION 1..7: note = Humanized Anti-Human CD3 Antibody ("CD3 mAb 2") Light Chain Variable Domain CDRL2
GTNKRAP

SEQ ID NO: 107 - protein, 9 aa, synthetic construct
  REGION 1..9: note = Humanized Anti-Human CD3 Antibody ("CD3 mAb 2") Light Chain Variable Domain CDRL3
ALWYSNLWV

SEQ ID NO: 108 - protein, 125 aa, synthetic construct
  REGION 1..125: note = Humanized Anti-Human CD3 Antibody ("CD3 mAb 2") Heavy Chain Variable Domain
EVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSS

SEQ ID NO: 109 - protein, 5 aa, synthetic construct
  REGION 1..5: note = Humanized Anti-Human CD3 Antibody ("CD3 mAb 2") Heavy Chain Variable Domain CDRH1
TYAMN

SEQ ID NO: 110 - protein, 19 aa, synthetic construct
  REGION 1..19: note = Humanized Anti-Human CD3 Antibody ("CD3 mAb 2") Heavy Chain Variable Domain CDRH2
RIRSKYNNYATYYADSVKD

SEQ ID NO: 111 - protein, 14 aa, synthetic construct
  REGION 1..14: note = Humanized Anti-Human CD3 Antibody ("CD3 mAb 2") Heavy Chain Variable Domain CDRH3
HGNFGNSYVSWFAY

SEQ ID NO: 112 - protein, 125 aa, synthetic construct
  REGION 1..125: note = Humanize Anti-Human CD3 Antibody ("CD3 mAb 2") Variant Heavy Chain Variable Domain Containing a D65G (Kabat Numbering; D68G in Sequence Relative to SEQ ID NO:108)
EVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSS

SEQ ID NO: 113 - protein, 19 aa, synthetic construct
  REGION 1..19: note = CDRH2 of Humanized Anti-Human CD3 Antibody ("CD3 mAb 2") Variant Heavy Chain Variable Domain Containing a D65G (Kabat Numbering; D68G in Sequence Relative to SEQ ID NO:108)
RIRSKYNNYATYYADSVKG

SEQ ID NO: 114 - protein, 107 aa, Mus musculus
  REGION 1..107: note = MISC_FEATURE - Murine Anti-Human CD3 Antibody (OKT3) Light Chain Variable Domain
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 115 - protein, 119 aa, Mus musculus [sequence omitted]

SEQ ID NO: 116 - protein, 111 aa, Mus musculus
  REGION 1..111: note = MISC_FEATURE - Murine Anti-Human CD16 Antibody (3G8) Light Chain Variable Domain
DTVLTQSPASLAVSLGQRATISCKASQSVDFDGDSFMNWYQQKPGQPPKLLIYTTSNLESGIPARFSASGSGTDFTLNIHPVEEEDTATYYCQQSNEDPYTFGGGTKLEIK

SEQ ID NO: 117 - protein, 118 aa, Mus musculus
  REGION 1..118: note = MISC_FEATURE - Murine Anti-Human CD16 Antibody (3G8) Heavy Chain Variable Domain
QVTLKESGPGILQPSQTLSLTCSFSGFSLRTSGMGVGWIRQPSGKGLEWLAHIWWDDDKRYNPALKSRLTISKDTSSNQVFLKIASVDTADTATYYCAQINPAWFAYWGQGTLVTVSA

SEQ ID NO: 118 - protein, 111 aa, Mus musculus
  REGION 1..111: note = MISC_FEATURE - Murine Anti-Human CD16 Antibody (A9) Light Chain Variable Domain
DIQAVVTQESALTTSPGETVTLTCRSNTGTVTTSNYANWVQEKPDHLFTGLIGHTNNRAPGVPARFSGSLIGDKAALTITGAQTEDEAIYFCALWYNNHWVFGGGTKLTVL

SEQ ID NO: 119 - protein, 117 aa, Mus musculus
  REGION 1..117: note = MISC_FEATURE - Murine Anti-Human CD16 Antibody (A9) Heavy Chain Variable Domain
QVQLQQSGAELVRPGTSVKISCKASGYTFTNYWLGWVKQRPGHGLEWIGDIYPGGGYTNYNEKFKGKATVTADTSSRTAYVQVRSLTSEDSAVYFCARSASWYFDVWGARTTVTVSS

SEQ ID NO: 120 - protein, 108 aa, Mus musculus
  REGION 1..108: note = MISC_FEATURE - Murine Anti-Human CD19 Antibody (HD37) Light Chain Variable Domain
DILITQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIKR

SEQ ID NO: 121 - protein, 124 aa, Mus musculus
  REGION 1..124: note = MISC_FEATURE - Murine Anti-Human CD19 Antibody (HD37) Heavy Chain Variable Domain
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTSVTVSS

SEQ ID NO: 122 - protein, 107 aa, synthetic construct
  REGION 1..107: note = Chimeric Anti-Human CD20 Antibody (Rituximab) Light Chain Variable Domain
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKR

SEQ ID NO: 123 - protein, 121 aa, synthetic construct [sequence omitted]

SEQ ID NO: 124 - protein, 113 aa, synthetic construct
  REGION 1..113: note = Humanized Anti-Human CD22 Antibody (Epratuzumab) Light Chain Variable Domain
DIQLTQSPSSLSASVGDRVTMSCKSSQSVLYSANHKNYLAWYQQKPGKAPKLLIYWASTRESGVPSRFSGSGSGTDFTFTISSLQPEDIATYYCHQYLSSWTFGGGTKVQIKR

SEQ ID NO: 125 - protein, 116 aa, synthetic construct [sequence omitted]

SEQ ID NO: 126 - protein, 107 aa, synthetic construct
  REGION 1..107: note = Murine Anti-Human CD32B Antibody ("CD32B mAb 1") Light Chain Variable Domain
DIQMTQSPSSLLAALGERVSLTCRASQEISGYLSWLQQKPDGTIKRLIYAASTLDSGVPKRFSGSESGSDYSLTISSLESEDFADYYCLQYFSYPLTFGAGTKLELK

SEQ ID NO: 127 - protein, 116 aa, synthetic construct
  REGION 1..116: note = Murine Anti-Human CD32B Antibody ("CD32B mAb 1") Heavy Chain Variable Domain
EVKLEESGGGLVQPGGSMKLSCEASGFTFSDAWMDWVRQSPEKGLEWVAEIRNKAKNHATYYAESVIGRFTISRDDSKSSVYLQMNSLRAEDTGIYYCGALGLDYWGQGTTLTVSS

SEQ ID NO: 128 - protein, 109 aa, Mus musculus
  REGION 1..109: note = MISC_FEATURE - Murine Anti-Human CD64 Antibody ("CD64 mAb 1") Light Chain Variable Domain
EIVLTQSPATLSLSPGERATLSCRASQSVSSYLAWYQQKPGQAPRLLIYDASSRATGIPARFGGSGSGGTDFTLTISSLEPEDFAVYYCQLRSNWPPYTFGQGTKLEIK

SEQ ID NO: 129 - protein, 118 aa, Mus musculus
  REGION 1..118: note = MISC_FEATURE - Murine Anti-Human CD64 Antibody ("CD64 mAb 1") Heavy Chain Variable Domain
QVQLVESGGGVVQPGRSLRLSCAASGFIFSGYGMHWVRQAPGKGLEWVTVIWYDGSNKYYADSVKGRFTISRDNSKNTLYLQMNSLRAEDTAVYYCARDTGDRFFDYWGQGTLVTVSS

SEQ ID NO: 130 - protein, 112 aa, synthetic construct
  REGION 1..112: note = Chimeric Anti-Human CD79 Antibody ("CD79 mAb 1") Light Chain Variable Domain
DVVMTQTPLTLSVNIGQPASISCKSSQSLLDTDGKTYLNWLLQRPQGSPNRLIYLVSKLDSGVPDRFTGSGSGTDFTLKISRVEAEDLGIYYCWQGTHFPLTFGAGTKLELK

SEQ ID NO: 131 - protein, 113 aa, synthetic construct
  REGION 1..113: note = Chimeric Anti-Human CD79 Antibody ("CD79 mAb 1") Heavy Chain Variable Domain
QVQLQQPGAELVRPGASVKLSCKASGYTFTSYWMNWVKQRPGQGLEWIGMVDPSDSETHYNQMFKDKATLTVDKSSSTAYMQLSSLTSEDSAVYYCARAMGYWGQGTSVTVSS

SEQ ID NO: 132 - protein, 106 aa, Mus musculus
  REGION 1..106: note = MISC_FEATURE - Murine Anti-Human TCR Antibody (BMA 031) Light Chain Variable Domain
EIVLTQSPATLSLSPGERATLSCSATSSVSYMHWYQQKPGKAPKRWIYDTSKLASGVPSRFSGSGSGTEFTLTISSLQPEDFATYYCQQWSSNPLTFGQGTKLEIK

SEQ ID NO: 133 - protein, 120 aa, Mus musculus
  REGION 1..120: note = MISC_FEATURE - Murine Anti-Human TCR Antibody (BMA 031) Heavy Chain Variable Domain
QVQLVQSGAEVKKPGASVKVSCKASGYKFTSYVMHWVRQAPGQGLEWIGYINPYNDVTKYNEKFKGRVTITADKSTSTAYLQMNSLRSEDTAVHYCARGSYYDYDGFVYWGQGTLVTVSS

SEQ ID NO: 134 - protein, 108 aa, Mus musculus
  REGION 1..108: note = MISC_FEATURE - Murine Anti-Human NKG2D Antibody (KYK-1.0) Light Chain Variable Domain
QPVLTQPSSVSVAPGETARIPCGGDDIETKSVHWYQQKPGQAPVLVIYDDDDRPSGIPERFFGSNSGNTATLSISRVEAGDEADYYCQVWDDNNDEWVFGGGTQLTVL

SEQ ID NO: 135 - protein, 118 aa, Mus musculus
  REGION 1..118: note = MISC_FEATURE - Murine Anti-Human NKG2D Antibody (KYK-1.0) Heavy Chain Variable Domain
EVQLVESGGGVVQPGGSLRLSCAASGFTFSSYGMHWVRQAPGKGLEWVAFIRYDGSNKYYADSVKGRFTISRDNSKNTKYLQMNSLRAEDTAVYYCAKDRFGYYLDYWGQGTLVTVSS

SEQ ID NO: 136 - protein, 110 aa, Mus musculus
  REGION 1..110: note = MISC_FEATURE - Murine Anti-Human NKG2D Antibody (KYK-2.0) Light Chain Variable Domain
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVL

SEQ ID NO: 137 - protein, 121 aa, Mus musculus
  REGION 1..121: note = MISC_FEATURE - Murine Anti-Human NKG2D Antibody (KYK-2.0) Heavy Chain Variable Domain
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSS

SEQ ID NO: 138 - protein, 112 aa, Mus musculus
  REGION 1..112: note = MISC_FEATURE - Murine Anti-Fluorescein Antibody 4-4-20 Light Chain Variable Domain
DVVMTQTPFSLPVSLGDQASISCRSSQSLVHSNGNTYLRWYLQKPGQSPKVLIYKVSNRFSGVPDRFSGSGSGTDFTLKISRVEAEDLGVYFCSQSTHVPWTFGGGTKLEIK

SEQ ID NO: 139 - protein, 118 aa, Mus musculus
  REGION 1..118: note = MISC_FEATURE - Murine Anti-Fluorescein Antibody 4-4-20 Heavy Chain Variable Domain
EVKLDETGGGLVQPGRPMKLSCVASGFTFSDYWMNWVRQSPEKGLEWVAQIRNKPYNYETYYSDSVKGRFTISRDDSKSSVYLQMNNLRVEDMGIYYCTGSYYGMDYWGQGTSVTVSS

SEQ ID NO: 140 - protein, 277 aa, synthetic construct
  REGION 1..277: note = First Polypeptide Chain of "DR5 mAb 1 x CD3 mAb 2 Diabody"
DIVLTQSPASLAVSLGQRATISCRASKSVSSSGYSYMHWYQQKPGQPPKVLIFLSSNLDSGVPARFSGSGSGTDFTLNIHPVEDGDAATYYCQHSRDLPPTFGGGTKLEIKGGGSGGGGEVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSSASTKGEVAACEKEVAALEKEVAALEKEVAALEK

SEQ ID NO: 141 - DNA, 831 bp, synthetic construct
  misc_feature 1..831: note = Polynucleotide Encoding First Polypeptide Chain of "DR5 mAb 1 x CD3 mAb 2 Diabody"
  source 1..831: mol_type = other DNA
gacattgtgctgacacagtctcctgcttccttagctgtatctctcgggcagagggccaccatctcatgcagggccagcaaaagtgtcagttcctctggctatagttatatgcactggtaccaacagaaaccaggacagccacccaaagtcctcatctttctttcatccaacctagattctggggtccctgccaggttcagtggcagtgggtctgggacagacttcaccctcaacatccatcctgtggaggatggggatgctgcaacctattactgtcagcacagtagggatcttcctccgacgttcggtggaggcaccaagctggaaatcaaaggaggcggatccggcggcggaggcgaggtgcagctggtggagtctgggggaggcttggtccagcctggagggtccctgagactctcctgtgcagcctctggattcaccttcagcacatacgctatgaattgggtccgccaggctccagggaaggggctggagtgggttggaaggatcaggtccaagtacaacaattatgcaacctactatgccgactctgtgaagggtagattcaccatctcaagagatgattcaaagaactcactgtatctgcaaatgaacagcctgaaaaccgaggacacggccgtgtattactgtgtgagacacggtaacttcggcaattcttacgtgtcttggtttgcttattggggacaggggacactggtgactgtgtcttccgcctccaccaagggcgaagtggccgcatgtgagaaagaggttgctgctttggagaaggaggtcgctgcacttgaaaaggaggtcgcagccctggagaaa

SEQ ID NO: 142 - protein, 272 aa, synthetic construct
  REGION 1..272: note = Second Polypeptide Chain of "DR5 mAb 1 x CD3 mAb 2 Diabody"
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGEVKFLESGGGLVQPGGSLKLSCVASGFDFSRYWMSWVRQAPGKGLEWIGEINPDSNTINYTPSLKDKFIISRDNAKNTLYLQMTKVRSEDTALYYCTRRAYYGNPAWFAYWGQGTLVTVSAASTKGKVAACKEKVAALKEKVAALKEKVAALKE

SEQ ID NO: 143 - DNA, 816 bp, synthetic construct
  misc_feature 1..816: note = Polynucleotide Encoding Second Polypeptide Chain of "DR5 mAb 1 x CD3 mAb 2 Diabody"
  source 1..816: mol_type = other DNA
caggctgtggtgactcaggagccttcactgaccgtgtccccaggcggaactgtgaccctgacatgcagatccagcacaggcgcagtgaccacatctaactacgccaattgggtgcagcagaagccaggacaggcaccaaggggcctgatcgggggtacaaacaaaagggctccctggacccctgcacggttttctggaagtctgctgggcggaaaggccgctctgactattaccggggcacaggccgaggacgaagccgattactattgtgctctgtggtatagcaatctgtgggtgttcgggggtggcacaaaactgactgtgctgggaggtggtggatccggcggcggaggcgaggtgaagtttctcgagtctggaggtggcctggtgcagcctggaggatccctgaaactctcctgtgtagcctcaggattcgattttagtagatactggatgagttgggtccggcaggctccagggaaagggctagaatggattggagaaattaatccagatagcaatacgataaactatacgccatctctaaaggataaattcatcatctccagagacaacgccaaaaatacgctgtatctgcaaatgaccaaagtgagatctgaggacacagccctttattattgtacaagaagggcctactatggtaacccggcctggtttgcttactggggccaagggactctggtcactgtctctgcagcctccaccaagggcaaagtggccgcatgtaaggagaaagttgctgctttgaaagagaaggtcgccgcacttaaggaaaaggtcgcagccctgaaagag

SEQ ID NO: 144 - protein, 273 aa, synthetic construct
  REGION 1..273: note = First Polypeptide Chain of "DR5 mAb 2 x CD3 mAb 2 Diabody"
DIVMTQSHKFMSTSVGDRVSITCKASQDVNTAVAWYQQKPGQSPKLLIYWASTRHTGVPDRFTGSGSGTDYTLTIKSVQAEDLTLYYCQQHYITPWTFGGGTKLEIKGGGSGGGGEVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSSASTKGEVAACEKEVAALEKEVAALEKEVAALEK

SEQ ID NO: 145 - DNA, 819 bp, synthetic construct
  misc_feature 1..819: note = Polynucleotide Encoding First Polypeptide Chain of "DR5 mAb 2 x CD3 mAb 2 Diabody"
  source 1..819: mol_type = other DNA
gacattgtgatgacccagtctcacaaattcatgtccacttcagtaggagacagggtcagcatcacctgcaaggccagtcaggatgtgaatactgctgtagcctggtatcaacaaaaaccagggcaatctcctaaactactgatttactgggcatccacccggcacactggagtccctgatcgcttcacaggcagtggatctgggacagattatacactcaccatcaaaagtgtgcaggctgaagacctgacactttattactgtcagcaacactatatcactccgtggacgttcggtggaggcaccaagctggaaatcaaaggaggcggatccggcggcggaggcgaggtgcagctggtggagtctgggggaggcttggtccagcctggagggtccctgagactctcctgtgcagcctctggattcaccttcagcacatacgctatgaattgggtccgccaggctccagggaaggggctggagtgggttggaaggatcaggtccaagtacaacaattatgcaacctactatgccgactctgtgaagggtagattcaccatctcaagagatgattcaaagaactcactgtatctgcaaatgaacagcctgaaaaccgaggacacggccgtgtattactgtgtgagacacggtaacttcggcaattcttacgtgtcttggtttgcttattggggacaggggacactggtgactgtgtcttccgcctccaccaagggcgaagtggccgcatgtgagaaagaggttgctgctttggagaaggaggtcgctgcacttgaaaaggaggtcgcagccctggagaaa

SEQ ID NO: 146 - protein, 270 aa, synthetic construct
  REGION 1..270: note = Second Polypeptide Chain of "DR5 mAb 2 x CD3 mAb 2 Diabody"
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGKVQLQQSGAELVKPGASVKLSCKASGYTFTEYILHWVKQKSGQGLEWIGWFYPGNNNIKYNEKFKDKATLTADKSSSTVYMELSRLTSEDSAVYFCARHEQGPGYFDYWGQGTTLTVSSASTKGKVAACKEKVAALKEKVAALKEKVAALKE

SEQ ID NO: 147 - DNA, 810 bp, synthetic construct
  misc_feature 1..810: note = Polynucleotide Encoding Second Polypeptide Chain of "DR5 mAb 2 x CD3 mAb 2 Diabody"
  source 1..810: mol_type = other DNA
caggctgtggtgactcaggagccttcactgaccgtgtccccaggcggaactgtgaccctgacatgcagatccagcacaggcgcagtgaccacatctaactacgccaattgggtgcagcagaagccaggacaggcaccaaggggcctgatcgggggtacaaacaaaagggctccctggacccctgcacggttttctggaagtctgctgggcggaaaggccgctctgactattaccggggcacaggccgaggacgaagccgattactattgtgctctgtggtatagcaatctgtgggtgttcgggggtggcacaaaactgactgtgctgggagggggtggatccggcggcggaggcaaggtccagctgcagcagtctggagctgaactggtgaaacccggggcatcagtgaagctgtcctgcaaggcttctgggtacaccttcactgagtatattttacactgggtaaagcagaagtctggacagggtcttgagtggattgggtggttttatcctggaaataataatataaagtacaatgagaaattcaaggacaaggccacactgactgcggacaaatcctccagcacagtctatatggaacttagtagattgacatctgaagactctgcggtctatttctgtgcaagacacgaacaaggaccaggttactttgactactggggccaaggcaccactctcacagtctcctccgcctccaccaagggcaaagtggccgcatgtaaggagaaagttgctgctttgaaagagaaggtcgccgcacttaaggaaaaggtcgcagccctgaaagag

SEQ ID NO: 148 - protein, 274 aa, synthetic construct
  REGION 1..274: note = First Polypeptide Chain of "DR5 mAb 3 x CD3 mAb 2 Diabody"
SELTQDPAVSVALGQTVRITCSGDSLRSYYASWYQQKPGQAPVLVIYGANNRPSGIPDRFSGSSSGNTASLTITGAQAEDEADYYCNSADSSGNHVVFGGGTKLTVLGGGGSGGGGEVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSSASTKGEVAACEKEVAALEKEVAALEKEVAALEK

SEQ ID NO: 149 - protein, 272 aa, synthetic construct
  REGION 1..272: note = Second Polypeptide Chain of "DR5 mAb 3 x CD3 mAb 2 Diabody"
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGEVQLVQSGGGVERPGGSLRLSCAASGFTFDDYAMSWVRQAPGKGLEWVSGINWQGGSTGYADSVKGRVTISRDNAKNSLYLQMNSLRAEDTAVYYCAKILGAGRGWYFDYWGKGTTVTVSSASTKGKVAACKEKVAALKEKVAALKEKVAALKE

SEQ ID NO: 150 - protein, 274 aa, synthetic construct
  REGION 1..274: note = First Polypeptide Chain of "DR5 mAb 4 x CD3 mAb 2 Diabody"
EIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIKGGGSGGGGEVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSSASTKGEVAACEKEVAALEKEVAALEKEVAALEK

SEQ ID NO: 151 - protein, 273 aa, synthetic construct
  REGION 1..273: note = Second Polypeptide Chain of "DR5 mAb 4 x CD3 mAb 2 Diabody"
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGQVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSSASTKGKVAACKEKVAALKEKVAALKEKVAALKE

SEQ ID NO: 152 - protein, 5 aa, synthetic construct
  REGION 1..5: note = Linker
APSSS

SEQ ID NO: 153 - protein, 106 aa, Mus musculus
  REGION 1..106: note = MISC_FEATURE - Murine Anti-Human EphA2 Antibody EphA2 mAb 1") Light Chain Variable Domain
DIQMTQTTSSLSASLGDRITISCRASQDISNYLNWYQQKPDGTVKLLIYYTSRLHSGVPSRFSGSGSGTDYSLTISNLEQEDIATYFCQQGYTLYTFGGGTKLEIK

SEQ ID NO: 154 - protein, 11 aa, Mus musculus
  REGION 1..11: note = MISC_FEATURE - Murine Anti-Human EphA2 Antibody ("EphA2 mAb 1") Light Chain Variable Domain CDRL1
RASQDISNYLN

SEQ ID NO: 155 - protein, 7 aa, Mus musculus
  REGION 1..7: note = MISC_FEATURE - Murine Anti-Human EphA2 Antibody ("EphA2 mAb 1") Light Chain Variable Domain CDRL2
YTSRLHS

SEQ ID NO: 156 - protein, 8 aa, Mus musculus
  REGION 1..8: note = MISC_FEATURE - Murine Anti-Human EphA2 Antibody ("EphA2 mAb 1") Light Chain Variable Domain CDRL3
QQGYTLYT

SEQ ID NO: 157 - DNA, 318 bp, Mus musculus
  misc_feature 1..318: note = Polynucleotide Encoding Murine Anti-Human EphA2 Antibody ("EphA2 mAb 1") Light Chain Variable Domain CDRL1
  source 1..318: mol_type = genomic DNA
gatatccagatgacacagactacatcctccctgtctgcctctctgggagacagaatcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatattgccacttacttttgccaacagggttatacgctgtacacgttcggaggggggaccaagctggaaataaaa

SEQ ID NO: 158 - protein, 118 aa, Mus musculus
  REGION 1..118: note = MISC_FEATURE - Murine Anti-Human EphA2 Antibody ("EphA2 mAb 1") Heavy Chain Variable Domain
QVQLKESGPGLVAPSQSLSITCTVSGFSLSRYSVHWVRQPPGKGLEWLGMIWGGGSTDYNSALKSRLSISKDNSKSQVFLKMNSLQTDDTAMYYCARKHGNYYTMDYWGQGTSVTVSS

SEQ ID NO: 159 - protein, 10 aa, Mus musculus
  REGION 1..10: note = MISC_FEATURE - Murine Anti-Human EphA2 Antibody ("EphA2 mAb 1") Heavy Chain Variable Domain CDRH1
GFSLSRYSVH

SEQ ID NO: 160 - protein, 16 aa, Mus musculus
  REGION 1..16: note = MISC_FEATURE - Murine Anti-Human EphA2 Antibody ("EphA2 mAb 1") Heavy Chain Variable Domain CDRH2
MIWGGGSTDYNSALKS

SEQ ID NO: 161 - protein, 10 aa, Mus musculus
  REGION 1..10: note = MISC_FEATURE - Murine Anti-Human EphA2 Antibody ("EphA2 mAb 1") Heavy Chain Variable Domain CDRH3
KHGNYYTMDY

SEQ ID NO: 162 - DNA, 354 bp, Mus musculus
  misc_feature 1..354: note = Polynucleotide Encoding Murine Anti-Human EphA2 Antibody ("EphA2 mAb 1") Heavy Chain Variable Domain
  source 1..354: mol_type = genomic DNA
caggtgcagctgaaggagtcaggacctggcctggtggcaccctcacagagcctgtccatcacatgcactgtctctgggttctcattatccagatatagtgtacactgggttcgccagcctccaggaaagggtctggagtggctgggaatgatatggggtggtggaagcacagactataattcagctctcaaatccagactgagtatcagcaaggacaactccaagagccaagttttcttaaaaatgaacagtctgcaaactgatgacacagccatgtactactgtgccagaaaacatggtaactactatactatggactactggggtcaaggaacctcagtcaccgtctcctcc

SEQ ID NO: 163 - protein, 111 aa, Mus musculus
  REGION 1..111: note = MISC_FEATURE - Murine Anti-Human EphA2 Antibody ("EphA2 mAb 2") Light Chain Variable Domain
DVVMTQTPLSLPVSLGDQASISCRSSQSLVHSSGNTYLHWYLQKPGQSPKLLIYKVSNRFSGVPDRFSGSGSGTDFTLKISRVEAEDLGVYFCSQSTHVPTFGSGTKLEIK

SEQ ID NO: 164 - protein, 16 aa, Mus musculus
  REGION 1..16: note = MISC_FEATURE - Murine Anti-Human EphA2 Antibody ("EphA2 mAb 2") Light Chain Variable Domain CDRL1
RSSQSLVHSSGNTYLH

SEQ ID NO: 165 - protein, 7 aa, Mus musculus
  REGION 1..7: note = MISC_FEATURE - Murine Anti-Human EphA2 Antibody ("EphA2 mAb 2") Light Chain Variable Domain CDRL2
KVSNRFS

SEQ ID NO: 166 - protein, 8 aa, Mus musculus
  REGION 1..8: note = MISC_FEATURE - Murine Anti-Human EphA2 Antibody ("EphA2 mAb 2") Light Chain Variable Domain CDRL3
SQSTHVPT

SEQ ID NO: 167 - protein, 118 aa, Mus musculus
  REGION 1..118: note = MISC_FEATURE - Murine Anti-Human EphA2 Antibody ("EphA2 mAb 2") Heavy Chain Variable Domain
QIQLVQSGPELKKPGETVKISCKASGFTFTNYGMNWVKQAPGKGLKWMGWINTYIGEPTYADDFKGRFVFSLETSASTAYLQINNLKNEDMATYFCARELGPYYFDYWGQGTTLTVSS

SEQ ID NO: 168 - protein, 10 aa, Mus musculus
  REGION 1..10: note = MISC_FEATURE - Murine Anti-Human EphA2 Antibody ("EphA2 mAb 2") Heavy Chain Variable Domain CDRH1
GFTFTNYGMN

SEQ ID NO: 169 - protein, 17 aa, Mus musculus
  REGION 1..17: note = MISC_FEATURE - Murine Anti-Human EphA2 Antibody ("EphA2 mAb 2") Heavy Chain Variable Domain CDRH2
WINTYIGEPTYADDFKG

SEQ ID NO: 170 - protein, 9 aa, Mus musculus
  REGION 1..9: note = MISC_FEATURE - Murine Anti-Human EphA2 Antibody ("EphA2 mAb 2") Heavy Chain Variable Domain CDRH3
ELGPYYFDY

SEQ ID NO: 171 - DNA, 354 bp, Mus musculus
  misc_feature 1..354: note = Polynucleotide Encoding Murine Anti-Human EphA2 Antibody ("EphA2 mAb 2") Heavy Chain Variable Domain
  source 1..354: mol_type = genomic DNA
cagatccagttggtgcagtctggacctgagctgaagaagcctggagagacagtcaagatctcctgcaaggcttctgggtttaccttcacaaactatggaatgaactgggtgaagcaggctccaggaaagggtttaaagtggatgggctggataaacacctatattggagagccgacatatgctgatgacttcaagggacggtttgtcttctctttggaaacctctgccagcactgcctatttgcagatcaacaacctcaaaaatgaggacatggccacatatttctgtgcaagagaactgggaccatactactttgactactggggccaaggcaccactctcacagtctcctcc

SEQ ID NO: 172 - protein, 107 aa, Mus musculus
  REGION 1..107: note = MISC_FEATURE - Murine Anti-Human EphA2 Antibody ("EphA2 mAb 3") Light Chain Variable Domain
DIVLTQSHRSMSTSVGDRVNITCKASQDVTTAVAWYQQKPGQSPKLLIFWASTRHAGVPDRFTGSGSGTDFTLTISSVQAGDLALYYCQQHYSTPYTFGGGTKLEIK

SEQ ID NO: 173 - protein, 11 aa, Mus musculus
  REGION 1..11: note = MISC_FEATURE - Murine Anti-Human EphA2 Antibody ("EphA2 mAb 3") Light Chain Variable Domain CDRL1
KASQDVTTAVA

SEQ ID NO: 174 - protein, 7 aa, Mus musculus
  REGION 1..7: note = MISC_FEATURE - Murine Anti-Human EphA2 Antibody ("EphA2 mAb 3") Light Chain Variable Domain CDRL2
WASTRHA

SEQ ID NO: 175 - protein, 9 aa, Mus musculus
  REGION 1..9: note = MISC_FEATURE - Murine Anti-Human EphA2 Antibody ("EphA2 mAb 3") Light Chain Variable Domain CDRL3
QQHYSTPYT

SEQ ID NO: 176 - DNA, 321 bp, Mus musculus
  misc_feature 1..321: note = Polynucleotide Encoding Murine Anti-Human EphA2 Antibody ("EphA2 mAb 3") Light Chain Variable Domain
  source 1..321: mol_type = genomic DNA
gacattgtgctgacccagtctcacagatccatgtccacatcagtaggagacagggtcaacatcacctgcaaggccagtcaggatgtgactactgctgtagcctggtatcaacaaaaaccagggcaatctcctaaattactgattttctgggcatccacccggcacgctggagtccctgatcgcttcacaggcagtggatctgggacagattttactctcaccatcagcagtgtgcaggctggagacctggcactttattactgtcaacaacattatagcacaccgtacacattcggaggggggaccaagctggaaataaaa

SEQ ID NO: 177 - protein, 118 aa, Mus musculus
  REGION 1..118: note = MISC_FEATURE - Murine Anti-Human EphA2 Antibody ("EphA2 mAb 3") Heavy Chain Variable Domain
EVQLVESGGGSVKPGGSLKLSCAASGFTFTDHYMYWVRQTPEKRLEWVATISDGGSFTSYPDSVKGRFTISRDIAKNNLYLQMSSLKSEDTAMYYCTRDESDRPFPYWGQGTLVTVSS

SEQ ID NO: 178 - protein, 10 aa, Mus musculus
  REGION 1..10: note = MISC_FEATURE - Murine Anti-Human EphA2 Antibody ("EphA2 mAb 3") Heavy Chain Variable Domain CDRH1
GFTFTDHYMY

SEQ ID NO: 179 - protein, 17 aa, Mus musculus
  REGION 1..17: note = MISC_FEATURE - Murine Anti-Human EphA2 Antibody ("EphA2 mAb 3") Heavy Chain Variable Domain CDRH2
TISDGGSFTSYPDSVKG

SEQ ID NO: 180 - protein, 9 aa, Mus musculus
  REGION 1..9: note = MISC_FEATURE - Murine Anti-Human EphA2 Antibody ("EphA2 mAb 3") Heavy Chain Variable Domain CDRH3
DESDRPFPY

SEQ ID NO: 181 - protein, 106 aa, synthetic construct
  REGION 1..106: note = Humanized Anti-Human gpA33 Antibody ("gpA33 mAb 1") Light Chain Variable Domain
DIQLTQSPSFLSASVGDRVTITCSARSSISFMYWYQQKPGKAPKLLIYDTSNLASGVPSRFSGSGSGTEFTLTISSLEAEDAATYYCQQWSSYPLTFGQGTKLEIK

SEQ ID NO: 182 - protein, 10 aa, synthetic construct
  REGION 1..10: note = Humanized Anti-Human gpA33 Antibody ("gpA33 mAb 1") Light Chain Variable Domain CDRL1
SARSSISFMY

SEQ ID NO: 183 - protein, 7 aa, synthetic construct
  REGION 1..7: note = Humanized Anti-Human gpA33 Antibody ("gpA33 mAb 1") Light Chain Variable Domain CDRL2
DTSNLAS

SEQ ID NO: 184 - protein, 9 aa, synthetic construct
  REGION 1..9: note = Humanized Anti-Human gpA33 Antibody ("gpA33 mAb 1") Light Chain Variable Domain CDRL3
QQWSSYPLT

SEQ ID NO: 185 - DNA, 318 bp, synthetic construct
  misc_feature 1..318: note = Polynucleotide Encoding Humanized Anti-Human gpA33 Antibody ("gpA33 mAb 1") Light Chain Variable Domain
  source 1..318: mol_type = other DNA
gacattcagctgactcagtccccctcttttctgtccgcatccgtcggagatcgagtgactattacttgctctgctaggtcctcaatcagcttcatgtactggtatcagcagaagcccggcaaagcacctaagctgctgatctacgacacaagcaacctggcctccggggtgccatctcggttctctggcagtgggtcaggaactgagtttaccctgacaattagctccctggaggctgaagatgccgctacctactattgccagcagtggagcagctatcctctgaccttcggacaggggactaaactggaaatcaag

SEQ ID NO: 186 - protein, 119 aa, synthetic construct
  REGION 1..119: note = Humanized Anti-Human gpA33 Antibody ("gpA33 mAb 1") Heavy Chain Variable Domain
QVQLVQSGAEVKKPGASVKVSCKASGYTFTGSWMNWVRQAPGQGLEWIGRIYPGDGETNYNGKFKDRVTITADKSTSTAYMELSSLRSEDTAVYYCARIYGNNVYFDVWGQGTTVTVSS

SEQ ID NO: 187 - protein, 10 aa, synthetic construct
  REGION 1..10: note = Humanized Anti-Human gpA33 Antibody ("gpA33 mAb 1") Heavy Chain Variable Domain CDRH1
GYTFTGSWMN

SEQ ID NO: 188 - protein, 17 aa, synthetic construct
  REGION 1..17: note = Humanized Anti-Human gpA33 Antibody ("gpA33 mAb 1") Heavy Chain Variable Domain CDRH2
RIYPGDGETNYNGKFKD

SEQ ID NO: 189 - protein, 10 aa, synthetic construct
  REGION 1..10: note = Humanized Anti-Human gpA33 Antibody ("gpA33 mAb 1") Heavy Chain Variable Domain CDRH3
IYGNNVYFDV

SEQ ID NO: 190 - DNA, 357 bp, synthetic construct
  misc_feature 1..357: note = Polynucelotide Encoding Humanized Anti-Human gpA33 Antibody ("gpA33 mAb 1") Heavy Chain Variable Domain
  source 1..357: mol_type = other DNA
caggtccagctggtccagagcggggccgaagtcaaaaaacccggagcaagcgtgaaggtctcctgcaaagcatcaggctatacatttacaggcagctggatgaactgggtgaggcaggctccaggacagggactggagtggatcgggcgcatctaccctggagacggcgaaactaactataatggaaagttcaaagaccgagtgaccatcacagccgataagtctactagtaccgcctacatggagctgagctccctgcggtctgaagataccgccgtctactattgcgctagaatttacggaaacaatgtctattttgacgtgtgggggcagggaacaactgtgactgtctcctcc

SEQ ID NO: 191 - protein, 109 aa, synthetic construct
  REGION 1..109: note = Humanized Anti-Human Her2 Antibody ("Her2 mAb 1") Light Chain Variable Domain
DIQMTQSPSSLSASVGDRVTITCRASQDVNTAVAWYQQKPGKAPKLLIYSASFLESGVPSRFSGSRSGTDFTLTISSLQPEDFATYYCQQHYTTPPTFGQGTKVEIKRT

SEQ ID NO: 192 - protein, 120 aa, synthetic construct
  REGION 1..120: note = Chimeric Anti-Human Her2 Antibody ("Her2 mAb 1") Heavy Chain Variable Domain
QVQLQQSGPELVKPGASLKLSCTASGFNIKDTYIHWVKQRPEQGLEWIGRIYPTNGYTRYDPKFQDKATITADTSSNTAYLQVSRLTSEDTAVYYCSRWGGDGFYAMDYWGQGASVTVSS

SEQ ID NO: 193 - protein, 108 aa, synthetic construct
  REGION 1..108: note = Humanized Anti-Human Her2 Antibody (Trastuzumab) Light Chain Variable Domain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKR

SEQ ID NO: 194 - protein, 120 aa, synthetic construct [sequence omitted]

SEQ ID NO: 195 - protein, 107 aa, Mus musculus
  REGION 1..107: note = MISC_FEATURE - Murine Anti-Human B7-H3 Antibody ("B7-H3 mAb 1") Light Chain Variable Domain
DIAMTQSQKFMSTSVGDRVSVTCKASQNVDTNVAWYQQKPGQSPKALIYSASYRYSGVPDRFTGSGSGTDFTLTINNVQSEDLAEYFCQQYNNYPFTFGSGTKLEIK

SEQ ID NO: 196 - protein, 122 aa, Mus musculus
  REGION 1..122: note = MISC_FEATURE - Murine Anti-Human B7-H3 Antibody ("B7-H3 mAb 1") Heavy Chain Variable Domain
DVQLVESGGGLVQPGGSRKLSCAASGFTFSSFGMHWVRQAPEKGLEWVAYISSDSSAIYYADTVKGRFTISRDNPKNTLFLQMTSLRSEDTAMYYCGRGRENIYYGSRLDYWGQGTTLTVSS

SEQ ID NO: 197 - protein, 107 aa, Mus musculus
  REGION 1..107: note = MISC_FEATURE - Murine Anti-Human B7-H3 Antibody ("B7-H3 mAb 2") Light Chain Variable Domain
DIQMTQTTSSLSASLGDRVTISCRASQDISNYLNWYQQKPDGTVKLLIYYTSRLHSGVPSRFSGSGSGTDYSLTIDNLEQEDIATYFCQQGNTLPPTFGGGTKLEIK

SEQ ID NO: 198 - protein, 120 aa, Mus musculus
  REGION 1..120: note = MISC_FEATURE - Murine Anti-Human B7-H3 Antibody ("B7-H3 mAb 2") Heavy Chain Variable Domain
QVQLQQSGAELARPGASVKLSCKASGYTFTSYWMQWVKQRPGQGLEWIGTIYPGDGDTRYTQKFKGKATLTADKSSSTAYMQLSSLASEDSAVYYCARRGIPRLWYFDVWGAGTTVTVSS

SEQ ID NO: 199 - protein, 108 aa, Mus musculus
  REGION 1..108: note = MISC_FEATURE - Murine Anti-Human B7-H3 Antibody ("B7-H3 mAb 3") Light Chain Variable Domain
DIQMTQSPASLSVSVGETVTITCRASESIYSYLAWYQQKQGKSPQLLVYNTKTLPEGVPSRFSGSGSGTQFSLKINSLQPEDFGRYYCQHHYGTPPWTFGGGTNLEIK

SEQ ID NO: 200 - protein, 117 aa, Mus musculus
  REGION 1..117: note = MISC_FEATURE - Murine Anti-Human B7-H3 Antibody ("B7-H3 mAb 3") Heavy Chain Variable Domain
EVQQVESGGDLVKPGGSLKLSCAASGFTFSSYGMSWVRQTPDKRLEWVATINSGGSNTYYPDSLKGRFTISRDNAKNTLYLQMRSLKSEDTAMYYCARHDGGAMDYWGQGTSVTVSS

SEQ ID NO: 201 - protein, 108 aa, synthetic construct
  REGION 1..108: note = Chimeric Anti-Human EGFR Antibody (Cetuximab) Light Chain Variable Domain
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKR

SEQ ID NO: 202 - protein, 119 aa, synthetic construct
  REGION 1..119: note = Chimeric Anti-Human EGFR Antibody (Cetuximab) Heavy Chain Variable Domain
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 203 - protein, 108 aa, synthetic construct
  REGION 1..108: note = Humanized Anti-Human VEGF Antibody (Bevacizumab) Light Chain Variable Domain
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKR

SEQ ID NO: 204 - protein, 123 aa, synthetic construct [sequence omitted]

SEQ ID NO: 205 - protein, 108 aa, synthetic construct
  REGION 1..108: note = Humanized Anti-CD52 Antibody (Alemtuzumab) Light Chain Variable Domain
DIQMTQSPSSLSASVGDRVTITCKASQNIDKYLNWYQQKPGKAPKLLIYNTNNLQTGVPSRFSGSGSGTDFTFTISSLQPEDIATYYCLQHISRPRTFGQGTKVEIKR

SEQ ID NO: 206 - protein, 121 aa, synthetic construct
  REGION 1..121: note = Humanized Anti-CD52 Antibody (Alemtuzumab) Heavy Chain Variable Domain
QVQLQESGPGLVRPSQTLSLTCTVSGFTFTDFYMNWVRQPPGRGLEWIGFIRDKAKGYTTEYNPSVKGRVTMLVDTSKNQFSLRLSSVTAADTAVYYCAREGHTAAPFDYWGQGSLVTVSS

SEQ ID NO: 207 - protein, 98 aa, Homo sapiens
  REGION 1..98: note = MISC_FEATURE - Human IgG1 CH1 Domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 208 - protein, 98 aa, synthetic construct
  REGION 1..98: note = Variant Human IgG1 CH1 Domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 209 - protein, 15 aa, Homo sapiens
  REGION 1..15: note = MISC_FEATURE - Human IgG1 Hinge Domain
EPKSCDKTHTCPPCP

SEQ ID NO: 210 - protein, 107 aa, Homo sapiens [sequence omitted]

SEQ ID NO: 211 - protein, 104 aa, Homo sapiens
  REGION 1..104: note = MISC_FEATURE - human IgG2 CL Lambda2 Domain
QPKAAPSVTLFPPSSEELQANKATLVCLISDFYPGAVTVAWKADSSPVKAGVETTPSKQSNNKYAASSYLSLTPEQWKSHRSYSCQVTHEGSTVEKTVAPTECS

SEQ ID NO: 212 - protein, 503 aa, synthetic construct
  REGION 1..503: note = First Polypeptide Chain of "gpA33 mAb 1 x CD3 mAb 2 x DR5 mAb 1" Tri-Specific Binding Molecule
DIQLTQSPSFLSASVGDRVTITCSARSSISFMYWYQQKPGKAPKLLIYDTSNLASGVPSRFSGSGSGTEFTLTISSLEAEDAATYYCQQWSSYPLTFGQGTKLEIKGGGSGGGGEVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSSGGCGGGEVAALEKEVAALEKEVAALEKEVAALEKGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 213 - DNA, 1509 bp, synthetic construct
  misc_feature 1..1509: note = Polynucleotide Encoding First Polypeptide Chain of "gpA33 mAb 1 x CD3 mAb 2 x DR5 mAb 1" Tri-Specific Binding Molecule
  source 1..1509: mol_type = other DNA
gacattcagctgactcagtccccctcttttctgtccgcatccgtcggagatcgagtgactattacttgctctgctaggtcctcaatcagcttcatgtactggtatcagcagaagcccggcaaagcacctaagctgctgatctacgacacaagcaacctggcctccggggtgccatctcggttctctggcagtgggtcaggaactgagtttaccctgacaattagctccctggaggctgaagatgccgctacctactattgccagcagtggagcagctatcctctgaccttcggacaggggactaaactggaaatcaagggtggaggatccggcggcggaggcgaggtgcagctggtggagtctgggggaggcttggtccagcctggagggtccctgagactctcctgtgcagcctctggattcaccttcagcacatacgctatgaattgggtccgccaggctccagggaaggggctggagtgggttggaaggatcaggtccaagtacaacaattatgcaacctactatgccgactctgtgaagggtagattcaccatctcaagagatgattcaaagaactcactgtatctgcaaatgaacagcctgaaaaccgaggacacggccgtgtattactgtgtgagacacggtaacttcggcaattcttacgtgtcttggtttgcttattggggacaggggacactggtgactgtgtcttccggaggatgtggcggtggagaagtggccgcactggagaaagaggttgctgctttggagaaggaggtcgctgcacttgaaaaggaggtcgcagccctggagaaaggcggcggggacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 214 - protein, 271 aa, synthetic construct
  REGION 1..271: note = Second Polypeptide Chain of "gpA33 mAb 1 x CD3 mAb 2 x DR5 mAb 1" Tri-Specific Binding Molecule
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGQVQLVQSGAEVKKPGASVKVSCKASGYTFTGSWMNWVRQAPGQGLEWIGRIYPGDGETNYNGKFKDRVTITADKSTSTAYMELSSLRSEDTAVYYCARIYGNNVYFDVWGQGTTVTVSSGGCGGGKVAALKEKVAALKEKVAALKEKVAALKE

SEQ ID NO: 215 - DNA, 813 bp, synthetic construct
  misc_feature 1..813: note = Polynucleotide Encoding Second Polypeptide Chain of "gpA33 mAb 1 x CD3 mAb 2 x DR5 mAb 1" Tri-Specific Binding Molecule
  source 1..813: mol_type = other DNA
caggctgtggtgactcaggagccttcactgaccgtgtccccaggcggaactgtgaccctgacatgcagatccagcacaggcgcagtgaccacatctaactacgccaattgggtgcagcagaagccaggacaggcaccaaggggcctgatcgggggtacaaacaaaagggctccctggacccctgcacggttttctggaagtctgctgggcggaaaggccgctctgactattaccggggcacaggccgaggacgaagccgattactattgtgctctgtggtatagcaatctgtgggtgttcgggggtggcacaaaactgactgtgctgggagggggtggatccggcggaggtggacaggtccagctggtccagagcggggccgaagtcaaaaaacccggagcaagcgtgaaggtctcctgcaaagcatcaggctatacatttacaggcagctggatgaactgggtgaggcaggctccaggacagggactggagtggatcgggcgcatctaccctggagacggcgaaactaactataatggaaagttcaaagaccgagtgaccatcacagccgataagtctactagtaccgcctacatggagctgagctccctgcggtctgaagataccgccgtctactattgcgctagaatttacggaaacaatgtctattttgacgtgtgggggcagggaacaactgtgactgtctcctccggaggatgtggcggtggaaaagtggccgcactgaaggagaaagttgctgctttgaaagagaaggtcgccgcacttaaggaaaaggtcgcagccctgaaagag

SEQ ID NO: 216 - protein, 451 aa, synthetic construct
  REGION 1..451: note = Third Polypeptide Chain of "gpA33 mAb 1 x CD3 mAb 2 x DR5 mAb 1" Tri-Specific Binding Molecule
EVKFLESGGGLVQPGGSLKLSCVASGFDFSRYWMSWVRQAPGKGLEWIGEINPDSNTINYTPSLKDKFIISRDNAKNTLYLQMTKVRSEDTALYYCTRRAYYGNPAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 217 - DNA, 1353 bp, synthetic construct
  misc_feature 1..1353: note = Polynucleotide Encoding Third Polypeptide Chain of "gpA33 mAb 1 x CD3 mAb 2 x DR5 mAb 1" Tri-Specific Binding Molecule
  source 1..1353: mol_type = other DNA
gaggtgaagtttctcgagtctggaggtggcctggtgcagcctggaggatccctgaaactctcctgtgtagcctcaggattcgattttagtagatactggatgagttgggtccggcaggctccagggaaagggctagaatggattggagaaattaatccagatagcaatacgataaactatacgccatctctaaaggataaattcatcatctccagagacaacgccaaaaatacgctgtatctgcaaatgaccaaagtgagatctgaggacacagccctttattattgtacaagaagggcctactatggtaacccggcctggtttgcttactggggccaagggactctggtcactgtctcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgagttgcgcagtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccgctacacgcagaagagcctctccctgtctccgggtaaa

SEQ ID NO: 218 - protein, 218 aa, synthetic construct
  REGION 1..218: note = Fourth Polypeptide Chain of "gpA33 mAb 1 x CD3 mAb 2 x DR5 mAb 1" Tri-Specific Binding Molecule
DIVLTQSPASLAVSLGQRATISCRASKSVSSSGYSYMHWYQQKPGQPPKVLIFLSSNLDSGVPARFSGSGSGTDFTLNIHPVEDGDAATYYCQHSRDLPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 219 - DNA, 654 bp, synthetic construct
  misc_feature 1..654: note = Polynucleotide Encoding Fourth Polypeptide Chain of "gpA33 mAb 1 x CD3 mAb 2 x DR5 mAb 1" Tri-Specific Binding Molecule
  source 1..654: mol_type = other DNA
gacattgtgctgacacagtctcctgcttccttagctgtatctctcgggcagagggccaccatctcatgcagggccagcaaaagtgtcagttcctctggctatagttatatgcactggtaccaacagaaaccaggacagccacccaaagtcctcatctttctttcatccaacctagattctggggtccctgccaggttcagtggcagtgggtctgggacagacttcaccctcaacatccatcctgtggaggatggggatgctgcaacctattactgtcagcacagtagggatcttcctccgacgttcggtggaggcaccaagctggaaatcaaacgtacggtggctgcaccatcggtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 220 - protein, 503 aa, synthetic construct; identical to SEQ ID NO: 212
  REGION 1..503: note = First Polypeptide Chain of "gpA33 mAb 1 x CD3 mAb 2 x DR5 mAb 2" Tri-Specific Binding Molecule

SEQ ID NO: 221 - DNA, 1509 bp, synthetic construct; identical to SEQ ID NO: 213
  misc_feature 1..1509: note = Polynucleotide Encoding First Polypeptide Chain of "gpA33 mAb 1 x CD3 mAb 2 x DR5 mAb 2" Tri-Specific Binding Molecule
  source 1..1509: mol_type = other DNA

SEQ ID NO: 222 - protein, 271 aa, synthetic construct; identical to SEQ ID NO: 214
  REGION 1..271: note = Second Polypeptide Chain of "gpA33 mAb 1 x CD3 mAb 2 x DR5 mAb 2" Tri-Specific Binding Molecule

SEQ ID NO: 223 - DNA, 813 bp, synthetic construct; identical to SEQ ID NO: 215
  misc_feature 1..813: note = Polynucleotide Encoding Second Polypeptide Chain of "gpA33 mAb 1 x CD3 mAb 2 x DR5 mAb 2" Tri-Specific Binding Molecule
  source 1..813: mol_type = other DNA

SEQ ID NO: 224 - protein, 449 aa, synthetic construct
  REGION 1..449: note = Third Polypeptide Chain of "gpA33 mAb 1 x CD3 mAb 2 x DR5 mAb 2" Tri-Specific Binding Molecule
KVQLQQSGAELVKPGASVKLSCKASGYTFTEYILHWVKQKSGQGLEWIGWFYPGNNNIKYNEKFKDKATLTADKSSSTVYMELSRLTSEDSAVYFCARHEQGPGYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 225 - DNA, 1347 bp, synthetic construct
  misc_feature 1..1347: note = Polynucleotide Encoding Third Polypeptide Chain of "gpA33 mAb 1 x CD3 mAb 2 x DR5 mAb 2" Tri-Specific Binding Molecule
  source 1..1347: mol_type = other DNA
aaggtccagctgcagcagtctggagctgaactggtgaaacccggggcatcagtgaagctgtcctgcaaggcttctgggtacaccttcactgagtatattttacactgggtaaagcagaagtctggacagggtcttgagtggattgggtggttttatcctggaaataataatataaagtacaatgagaaattcaaggacaaggccacactgactgcggacaaatcctccagcacagtctatatggaacttagtagattgacatctgaagactctgcggtctatttctgtgcaagacacgaacaaggaccaggttactttgactactggggccaaggcaccactctcacagtctcctccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgagttgcgcagtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccgctacacgcagaagagcctctccctgtctccgggtaaa

SEQ ID NO: 226 - protein, 214 aa, synthetic construct
  REGION 1..214: note = Fourth Polypeptide Chain of "gpA33 mAb 1 x CD3 mAb 2 x DR5 mAb 2" Tri-Specific Binding Molecule
DIVMTQSHKFMSTSVGDRVSITCKASQDVNTAVAWYQQKPGQSPKLLIYWASTRHTGVPDRFTGSGSGTDYTLTIKSVQAEDLTLYYCQQHYITPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 227 - DNA, 642 bp, synthetic construct
  misc_feature 1..642: note = Polynucleotide Encoding Fourth Polypeptide Chain of "gpA33 mAb 1 x CD3 mAb 2 x DR5 mAb 2" Tri-Specific Binding Molecule
  source 1..642: mol_type = other DNA
gacattgtgatgacccagtctcacaaattcatgtccacttcagtaggagacagggtcagcatcacctgcaaggccagtcaggatgtgaatactgctgtagcctggtatcaacaaaaaccagggcaatctcctaaactactgatttactgggcatccacccggcacactggagtccctgatcgcttcacaggcagtggatctgggacagattatacactcaccatcaaaagtgtgcaggctgaagacctgacactttattactgtcagcaacactatatcactccgtggacgttcggtggaggcaccaagctggaaatcaaacgtacggtggctgcaccatcggtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 228 - protein, 503 aa, synthetic construct
  REGION 1..503: note = First Polypeptide Chain of "EphA2 mAb 1 x CD3 mAb 2 x DR5 mAb 1" Tri-Specific Binding Molecule
DIQMTQTTSSLSASLGDRITISCRASQDISNYLNWYQQKPDGTVKLLIYYTSRLHSGVPSRFSGSGSGTDYSLTISNLEQEDIATYFCQQGYTLYTFGGGTKLEIKGGGSGGGGEVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSSGGCGGGEVAALEKEVAALEKEVAALEKEVAALEKGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 229 - DNA, 1508 bp, synthetic construct
  misc_feature 1..1508: note = Polynucleotide Encoding First Polypeptide Chain of "EphA2 mAb 1 x CD3 mAb 2 x DR5 mAb 1" Tri-Specific Binding Molecule
  source 1..1508: mol_type = other DNA
gatatccagatgacacagactacatcctccctgtctgcctctctgggagacagaatcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatattgccacttacttttgccaacagggttatacgctgtacacgttcggaggggggaccaagctggaaataaaaggtggaggatccggcggcggaggcgaggtgcagctggtggagtctgggggaggcttggtccagcctggagggtccctgagactctcctgtgcagcctctggattcaccttcagcacatacgctatgaattgggtccgccaggctccagggaaggggctggagtgggttggaaggatcaggtccaagtacaacaattatgcaacctactatgccgactctgtgaagggtagattcaccatctcaagagatgattcaaagaactcactgtatctgcaaatgaacagcctgaaaaccgaggacacggccgtgtattactgtgtgagacacggtaacttcggcaattcttacgtgtcttggtttgcttattggggacaggggacactggtgactgtgtcttccggaggatgtggcggtggagaagtggccgcactggagaaagaggttgctgctttggagaaggaggtcgctgcacttgaaaaggaggtcgcagccctggagaaaggcggcggggacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaa

SEQ ID NO: 230 - protein, 270 aa, synthetic construct
  REGION 1..270: note = Second Polypeptide Chain of "EphA2 mAb 1 x CD3 mAb 2 x DR5 mAb 1" Tri-Specific Binding Molecule
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGQVQLKESGPGLVAPSQSLSITCTVSGFSLSRYSVHWVRQPPGKGLEWLGMIWGGGSTDYNSALKSRLSISKDNSKSQVFLKMNSLQTDDTAMYYCARKHGNYYTMDYWGQGTSVTVSSGGCGGGKVAALKEKVAALKEKVAALKEKVAALKE

SEQ ID NO: 231 - DNA, 810 bp, synthetic construct
  misc_feature 1..810: note = Polynucleotide Encoding Second Polypeptide Chain of "EphA2 mAb 1 x CD3 mAb 2 x DR5 mAb 1" Tri-Specific Binding Molecule
  source 1..810: mol_type = other DNA
caggctgtggtgactcaggagccttcactgaccgtgtccccaggcggaactgtgaccctgacatgcagatccagcacaggcgcagtgaccacatctaactacgccaattgggtgcagcagaagccaggacaggcaccaaggggcctgatcgggggtacaaacaaaagggctccctggacccctgcacggttttctggaagtctgctgggcggaaaggccgctctgactattaccggggcacaggccgaggacgaagccgattactattgtgctctgtggtatagcaatctgtgggtgttcgggggtggcacaaaactgactgtgctgggagggggtggatccggcggaggtggacaggtgcagctgaaggagtcaggacctggcctggtggcaccctcacagagcctgtccatcacatgcactgtctctgggttctcattatccagatatagtgtacactgggttcgccagcctccaggaaagggtctggagtggctgggaatgatatggggtggtggaagcacagactataattcagctctcaaatccagactgagtatcagcaaggacaactccaagagccaagttttcttaaaaatgaacagtctgcaaactgatgacacagccatgtactactgtgccagaaaacatggtaactactatactatggactactggggtcaaggaacctcagtcaccgtctcctccggaggatgtggcggtggaaaagtggccgcactgaaggagaaagttgctgctttgaaagagaaggtcgccgcacttaaggaaaaggtcgcagccctgaaagag

SEQ ID NO: 232 - protein, 451 aa, synthetic construct; identical to SEQ ID NO: 216
  REGION 1..451: note = Third Polypeptide Chain of "EphA2 mAb 1 x CD3 mAb 2 x DR5 mAb 1" Tri-Specific Binding Molecule

SEQ ID NO: 233 - DNA, 1353 bp, synthetic construct; identical to SEQ ID NO: 217
  misc_feature 1..1353: note = Polynucleotide Encoding Third Polypeptide Chain of "EphA2 mAb 1 x CD3 mAb 2 x DR5 mAb 1" Tri-Specific Binding Molecule
  source 1..1353: mol_type = other DNA

SEQ ID NO: 234 - protein, 218 aa, synthetic construct; identical to SEQ ID NO: 218
  REGION 1..218: note = Fourth Polypeptide Chain of "EphA2 mAb 1 x CD3 mAb 2 x DR5 mAb 1" Tri-Specific Binding Molecule

SEQ ID NO: 235 - DNA, 654 bp, synthetic construct; identical to SEQ ID NO: 219
  misc_feature 1..654: note = Polynucleotide Encoding Fourth Polypeptide Chain of "EphA2 mAb 1 x CD3 mAb 2 x DR5 mAb 1" Tri-Specific Binding Molecule
  source 1..654: mol_type = other DNA

SEQ ID NO: 236 - protein, 508 aa, synthetic construct
  REGION 1..508: note = First Polypeptide Chain of "EphA2 mAb 2 x CD3 mAb 2 x DR5 mAb 1" Tri-Specific Binding Molecule
DVVMTQTPLSLPVSLGDQASISCRSSQSLVHSSGNTYLHWYLQKPGQSPKLLIYKVSNRFSGVPDRFSGSGSGTDFTLKISRVEAEDLGVYFCSQSTHVPTFGSGTKLEIKGGGSGGGGEVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSSGGCGGGEVAALEKEVAALEKEVAALEKEVAALEKGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 237 - DNA, 1524 bp, synthetic construct
  misc_feature 1..1524: note = Polynucleotide Encoding First Polypeptide Chain of "EphA2 mAb 2 x CD3 mAb 2 x DR5 mAb 1" Tri-Specific Binding Molecule
  source 1..1524: mol_type = other DNA
gatgttgtgatgacccaaactccactctccctgcctgtcagtcttggagatcaagcctccatctcttgcagatctagtcagagccttgtacacagtagtggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttcccacgttcggctcggggacaaagttggaaataaaaggtggaggatccggcggcggaggcgaggtgcagctggtggagtctgggggaggcttggtccagcctggagggtccctgagactctcctgtgcagcctctggattcaccttcagcacatacgctatgaattgggtccgccaggctccagggaaggggctggagtgggttggaaggatcaggtccaagtacaacaattatgcaacctactatgccgactctgtgaagggtagattcaccatctcaagagatgattcaaagaactcactgtatctgcaaatgaacagcctgaaaaccgaggacacggccgtgtattactgtgtgagacacggtaacttcggcaattcttacgtgtcttggtttgcttattggggacaggggacactggtgactgtgtcttccggaggatgtggcggtggagaagtggccgcactggagaaagaggttgctgctttggagaaggaggtcgctgcacttgaaaaggaggtcgcagccctggagaaaggcggcggggacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 238 - protein, 270 aa, synthetic construct
  REGION 1..270: note = Second Polypeptide Chain of "EphA2 mAb 2 x CD3 mAb 2 x DR5 mAb 1" Tri-Specific Binding Molecule
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGQIQLVQSGPELKKPGETVKISCKASGFTFTNYGMNWVKQAPGKGLKWMGWINTYIGEPTYADDFKGRFVFSLETSASTAYLQINNLKNEDMATYFCARELGPYYFDYWGQGTTLTVSSGGCGGGKVAALKEKVAALKEKVAALKEKVAALKE

SEQ ID NO: 239 - DNA, 810 bp, synthetic construct
  misc_feature 1..810: note = Polynucleotide Encoding Second Polypeptide Chain of "EphA2 mAb 2 x CD3 mAb 2 x DR5 mAb 1" Tri-Specific Binding Molecule
  source 1..810: mol_type = other DNA
caggctgtggtgactcaggagccttcactgaccgtgtccccaggcggaactgtgaccctgacatgcagatccagcacaggcgcagtgaccacatctaactacgccaattgggtgcagcagaagccaggacaggcaccaaggggcctgatcgggggtacaaacaaaagggctccctggacccctgcacggttttctggaagtctgctgggcggaaaggccgctctgactattaccggggcacaggccgaggacgaagccgattactattgtgctctgtggtatagcaatctgtgggtgttcgggggtggcacaaaactgactgtgctgggagggggtggatccggcggaggtggacagatccagttggtgcagtctggacctgagctgaagaagcctggagagacagtcaagatctcctgcaaggcttctgggtttaccttcacaaactatggaatgaactgggtgaagcaggctccaggaaagggtttaaagtggatgggctggataaacacctatattggagagccgacatatgctgatgacttcaagggacggtttgtcttctctttggaaacctctgccagcactgcctatttgcagatcaacaacctcaaaaatgaggacatggccacatatttctgtgcaagagaactgggaccatactactttgactactggggccaaggcaccactctcacagtctcctccggaggatgtggcggtggaaaagtggccgcactgaaggagaaagttgctgctttgaaagagaaggtcgccgcacttaaggaaaaggtcgcagccctgaaagag

SEQ ID NO: 240 - protein, 451 aa, synthetic construct; identical to SEQ ID NO: 216
  REGION 1..451: note = Third Polypeptide Chain of "EphA2 mAb 2 x CD3 mAb 2 x DR5 mAb 1" Tri-Specific Binding Molecule

SEQ ID NO: 241 - DNA, 1353 bp, synthetic construct; identical to SEQ ID NO: 217
  misc_feature 1..1353: note = Polynucleotide Encoding Third Polypeptide Chain of "EphA2 mAb 2 x CD3 mAb 2 x DR5 mAb 1" Tri-Specific Binding Molecule
  source 1..1353: mol_type = other DNA

SEQ ID NO: 242 - protein, 218 aa, synthetic construct; identical to SEQ ID NO: 218
  REGION 1..218: note = Fourth Polypeptide Chain of "EphA2 mAb 2 x CD3 mAb 2 x DR5 mAb 1" Tri-Specific Binding Molecule

SEQ ID NO: 243 - DNA, 654 bp, synthetic construct; identical to SEQ ID NO: 219
  misc_feature 1..654: note = Polynucleotide Encoding Fourth Polypeptide Chain of "EphA2 mAb 2 x CD3 mAb 2 x DR5 mAb 1" Tri-Specific Binding Molecule
  source 1..654: mol_type = other DNA

SEQ ID NO: 244 - protein, 504 aa, synthetic construct
  REGION 1..504: note = First Polypeptide Chain of "EphA2 mAb 3 x CD3 mAb 2 x DR5 mAb 1" Tri-Specific Binding Molecule
DIVLTQSHRSMSTSVGDRVNITCKASQDVTTAVAWYQQKPGQSPKLLIFWASTRHAGVPDRFTGSGSGTDFTLTISSVQAGDLALYYCQQHYSTPYTFGGGTKLEIKGGGSGGGGEVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSSGGCGGGEVAALEKEVAALEKEVAALEKEVAALEKGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 245 - DNA, 1512 bp, synthetic construct
  misc_feature 1..1512: note = Polynucleotide Encoding First Polypeptide Chain of "EphA2 mAb 3 x CD3 mAb 2 x DR5 mAb 1" Tri-Specific Binding Molecule
  source 1..1512: mol_type = other DNA
gacattgtgctgacccagtctcacagatccatgtccacatcagtaggagacagggtcaacatcacctgcaaggccagtcaggatgtgactactgctgtagcctggtatcaacaaaaaccagggcaatctcctaaattactgattttctgggcatccacccggcacgctggagtccctgatcgcttcacaggcagtggatctgggacagattttactctcaccatcagcagtgtgcaggctggagacctggcactttattactgtcaacaacattatagcacaccgtacacattcggaggggggaccaagctggaaataaaaggtggaggatccggcggcggaggcgaggtgcagctggtggagtctgggggaggcttggtccagcctggagggtccctgagactctcctgtgcagcctctggattcaccttcagcacatacgctatgaattgggtccgccaggctccagggaaggggctggagtgggttggaaggatcaggtccaagtacaacaattatgcaacctactatgccgactctgtgaagggtagattcaccatctcaagagatgattcaaagaactcactgtatctgcaaatgaacagcctgaaaaccgaggacacggccgtgtattactgtgtgagacacggtaacttcggcaattcttacgtgtcttggtttgcttattggggacaggggacactggtgactgtgtcttccggaggatgtggcggtggagaagtggccgcactggagaaagaggttgctgctttggagaaggaggtcgctgcacttgaaaaggaggtcgcagccctggagaaaggcggcggggacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 246 - protein, 270 aa, synthetic construct
  REGION 1..270: note = Second Polypeptide Chain of "EphA2 mAb 3 x CD3 mAb 2 x DR5 mAb 1" Tri-Specific Binding Molecule
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGEVQLVESGGGSVKPGGSLKLSCAASGFTFTDHYMYWVRQTPEKRLEWVATISDGGSFTSYPDSVKGRFTISRDIAKNNLYLQMSSLKSEDTAMYYCTRDESDRPFPYWGQGTLVTVSSGGCGGGKVAALKEKVAALKEKVAALKEKVAALKE

SEQ ID NO: 247 - DNA, 810 bp, synthetic construct
  misc_feature 1..810: note = Polynucleotide Encoding Second Polypeptide Chain of "EphA2 mAb 3 x CD3 mAb 2 x DR5 mAb 1" Tri-Specific Binding Molecule
  source 1..810: mol_type = other DNA
caggctgtggtgactcaggagccttcactgaccgtgtccccaggcggaactgtgaccctgacatgcagatccagcacaggcgcagtgaccacatctaactacgccaattgggtgcagcagaagccaggacaggcaccaaggggcctgatcgggggtacaaacaaaagggctccctggacccctgcacggttttctggaagtctgctgggcggaaaggccgctctgactattaccggggcacaggccgaggacgaagccgattactattgtgctctgtggtatagcaatctgtgggtgttcgggggtggcacaaaactgactgtgctgggagggggtggatccggcggaggtggagaagtgcagctggtggagtctgggggaggctcagtgaagcctggagggtccctgaaactctcctgtgcagcctctggattcactttcactgaccattacatgtattgggttcgccagactccggaaaagaggctggagtgggtcgcaaccattagtgatggcggtagtttcacctcctatccagacagtgtgaaggggcgattcaccatctccagagacattgccaagaacaacctgtacctccaaatgagcagtctgaagtctgaggacacagccatgtattactgtacaagagatgagagcgataggccgtttccttactggggccaagggactctggtcactgtctcctccggaggatgtggcggtggaaaagtggccgcactgaaggagaaagttgctgctttgaaagagaaggtcgccgcacttaaggaaaaggtcgcagccctgaaagag

SEQ ID NO: 248 - protein, 451 aa, synthetic construct; identical to SEQ ID NO: 216
  REGION 1..451: note = Third Polypeptide Chain of "EphA2 mAb 3 x CD3 mAb 2 x DR5 mAb 1" Tri-Specific Binding Molecule

SEQ ID NO: 249 - DNA, 1353 bp, synthetic construct; identical to SEQ ID NO: 217
  misc_feature 1..1353: note = Polynucleotide Encoding Third Polypeptide Chain of "EphA2 mAb 3 x CD3 mAb 2 x DR5 mAb 1" Tri-Specific Binding Molecule
  source 1..1353: mol_type = other DNA

SEQ ID NO: 250 - protein, 218 aa, synthetic construct; identical to SEQ ID NO: 218
  REGION 1..218: note = Fourth Polypeptide Chain of "EphA2 mAb 3 x CD3 mAb 2 x DR5 mAb 1" Tri-Specific Binding Molecule

SEQ ID NO: 251 - DNA, 654 bp, synthetic construct; identical to SEQ ID NO: 219
  misc_feature 1..654: note = Polynucleotide Encoding Fourth Polypeptide Chain of "EphA2 mAb 3 x CD3 mAb 2 x DR5 mAb 1" Tri-Specific Binding Molecule
  source 1..654: mol_type = other DNA

SEQ ID NO: 252 - protein, 503 aa, synthetic construct; identical to SEQ ID NO: 212
  REGION 1..503: note = First Polypeptide Chain of "gpA33 mAb 1 x CD3 mAb 2 x EphA2 mAb 1" Tri-Specific Binding Molecule

SEQ ID NO: 253 - DNA, 1509 bp, synthetic construct; identical to SEQ ID NO: 213
  misc_feature 1..1509: note = Polynucleotide Encoding First Polypeptide Chain of "gpA33 mAb 1 x CD3 mAb 2 x EphA2 mAb 1" Tri-Specific Binding Molecule
  source 1..1509: mol_type = other DNA

SEQ ID NO: 254 - protein, 271 aa, synthetic construct; identical to SEQ ID NO: 214
  REGION 1..271: note = Second Polypeptide Chain of "gpA33 mAb 1 x CD3 mAb 2 x EphA2 mAb 1" Tri-Specific Binding Molecule

SEQ ID NO: 255 - DNA, 813 bp, synthetic construct; identical to SEQ ID NO: 215
  misc_feature 1..813: note = Polynucleotide Encoding Second Polypeptide Chain of "gpA33 mAb 1 x CD3 mAb 2 x EphA2 mAb 1" Tri-Specific Binding Molecule
  source 1..813: mol_type = other DNA

SEQ ID NO: 256 - protein, 448 aa, synthetic construct
  REGION 1..448: note = Third Polypeptide Chain of "gpA33 mAb 1 x CD3 mAb 2 x EphA2 mAb 1" Tri-Specific Binding Molecule
QVQLKESGPGLVAPSQSLSITCTVSGFSLSRYSVHWVRQPPGKGLEWLGMIWGGGSTDYNSALKSRLSISKDNSKSQVFLKMNSLQTDDTAMYYCARKHGNYYTMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 257 - DNA, 1344 bp, synthetic construct
  misc_feature 1..1344: note = Polynucleotide Encoding Third Polypeptide Chain of "gpA33 mAb 1 x CD3 mAb 2 x EphA2 mAb 1" Tri-Specific Binding Molecule
  source 1..1344: mol_type = other DNA
caggtgcagctgaaggagtcaggacctggcctggtggcaccctcacagagcctgtccatcacatgcactgtctctgggttctcattatccagatatagtgtacactgggttcgccagcctccaggaaagggtctggagtggctgggaatgatatggggtggtggaagcacagactataattcagctctcaaatccagactgagtatcagcaaggacaactccaagagccaagttttcttaaaaatgaacagtctgcaaactgatgacacagccatgtactactgtgccagaaaacatggtaactactatactatggactactggggtcaaggaacctcagtcaccgtctcctccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgagttgcgcagtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccgctacacgcagaagagcctctccctgtctccgggtaaa

SEQ ID NO: 258 - protein, 213 aa, synthetic construct
  REGION 1..213: note = Fourth Polypeptide Chain of "gpA33 mAb 1 x CD3 mAb 2 x EphA2 mAb 1" Tri-Specific Binding Molecule
DIQMTQTTSSLSASLGDRITISCRASQDISNYLNWYQQKPDGTVKLLIYYTSRLHSGVPSRFSGSGSGTDYSLTISNLEQEDIATYFCQQGYTLYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 259 - DNA, 639 bp, synthetic construct
  misc_feature 1..639: note = Polynucleotide Encoding Fourth Polypeptide Chain of "gpA33 mAb 1 x CD3 mAb 2 x EphA2 mAb 1" Tri-Specific Binding Molecule
  source 1..639: mol_type = other DNA
gatatccagatgacacagactacatcctccctgtctgcctctctgggagacagaatcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatattgccacttacttttgccaacagggttatacgctgtacacgttcggaggggggaccaagctggaaataaaacgtacggtggctgcaccatcggtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 260 - protein, 503 aa, synthetic construct; identical to SEQ ID NO: 212
  REGION 1..503: note = First Polypeptide Chain of "gpA33 mAb 1 x CD3 mAb 2 x EphA2 mAb 2" Tri-Specific Binding Molecule

SEQ ID NO: 261 - DNA, 1509 bp, synthetic construct; identical to SEQ ID NO: 213
  misc_feature 1..1509: note = Polynucleotide Encoding First Polypeptide Chain of "gpA33 mAb 1 x CD3 mAb 2 x EphA2 mAb 2" Tri-Specific Binding Molecule
  source 1..1509: mol_type = other DNA

SEQ ID NO: 262 - protein, 271 aa, synthetic construct; identical to SEQ ID NO: 214
  REGION 1..271: note = Second Polypeptide Chain of "gpA33 mAb 1 x CD3 mAb 2 x EphA2 mAb 2" Tri-Specific Binding Molecule

SEQ ID NO: 263 - DNA, 813 bp, synthetic construct; identical to SEQ ID NO: 215
  misc_feature 1..813: note = Polynucleotide Encoding Second Polypeptide Chain of "gpA33 mAb 1 x CD3 mAb 2 x EphA2 mAb 2" Tri-Specific Binding Molecule
  source 1..813: mol_type = other DNA

SEQ ID NO: 264 - protein, 448 aa, synthetic construct
  REGION 1..448: note = Third Polypeptide Chain of "gpA33 mAb 1 x CD3 mAb 2 x EphA2 mAb 2" Tri-Specific Binding Molecule
QIQLVQSGPELKKPGETVKISCKASGFTFTNYGMNWVKQAPGKGLKWMGWINTYIGEPTYADDFKGRFVFSLETSASTAYLQINNLKNEDMATYFCARELGPYYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 265 - DNA, 1344 bp, synthetic construct
  misc_feature 1..1344: note = Polynucleotide Encoding Third Polypeptide Chain of "gpA33 mAb 1 x CD3 mAb 2 x EphA2 mAb 2" Tri-Specific Binding Molecule
  source 1..1344: mol_type = other DNA
cagatccagttggtgcagtctggacctgagctgaagaagcctggagagacagtcaagatctcctgcaaggcttctgggtttaccttcacaaactatggaatgaactgggtgaagcaggctccaggaaagggtttaaagtggatgggctggataaacacctatattggagagccgacatatgctgatgacttcaagggacggtttgtcttctctttggaaacctctgccagcactgcctatttgcagatcaacaacctcaaaaatgaggacatggccacatatttctgtgcaagagaactgggaccatactactttgactactggggccaaggcaccactctcacagtctcctccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgagttgcgcagtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccgctacacgcagaagagcctctccctgtctccgggtaaa

SEQ ID NO: 266 - protein, 218 aa, synthetic construct
  REGION 1..218: note = Polynucleotide Encoding Fourth Polypeptide Chain of "gpA33 mAb 1 x CD3 mAb 2 x EphA2 mAb 2" Tri-Specific Binding Molecule
DVVMTQTPLSLPVSLGDQASISCRSSQSLVHSSGNTYLHWYLQKPGQSPKLLIYKVSNRFSGVPDRFSGSGSGTDFTLKISRVEAEDLGVYFCSQSTHVPTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 267 - DNA, 654 bp, synthetic construct
  misc_feature 1..654: note = Polynucleotide Encoding Fourth Polypeptide Chain of "gpA33 mAb 1 x CD3 mAb 2 x EphA2 mAb 2" Tri-Specific Binding Molecule
  source 1..654: mol_type = other DNA
gatgttgtgatgacccaaactccactctccctgcctgtcagtcttggagatcaagcctccatctcttgcagatctagtcagagccttgtacacagtagtggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttcccacgttcggctcggggacaaagttggaaataaaacgtacggtggctgcaccatcggtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 268 - protein, 503 aa, synthetic construct; identical to SEQ ID NO: 212
  REGION 1..503: note = First Polypeptide Chain of "gpA33 mAb 1 x CD3 mAb 2 x EphA2 mAb 3" Tri-Specific Binding Molecule

SEQ ID NO: 269 - DNA, 1509 bp, synthetic construct; identical to SEQ ID NO: 213
  misc_feature 1..1509: note = Polynucleotide Encoding First Polypeptide Chain of "gpA33 mAb 1 x CD3 mAb 2 x EphA2 mAb 3" Tri-Specific Binding Molecule
  source 1..1509: mol_type = other DNA

SEQ ID NO: 270 - protein, 271 aa, synthetic construct; identical to SEQ ID NO: 214
  REGION 1..271: note = Second Polypeptide Chain of "gpA33 mAb 1 x CD3 mAb 2 x EphA2 mAb 3" Tri-Specific Binding Molecule

SEQ ID NO: 271 - DNA, 813 bp, synthetic construct; identical to SEQ ID NO: 215
  misc_feature 1..813: note = Polynucleotide Encoding Second Polypeptide Chain of "gpA33 mAb 1 x CD3 mAb 2 x EphA2 mAb 3" Tri-Specific Binding Molecule
  source 1..813: mol_type = other DNA

SEQ ID NO: 272 - protein, 448 aa, synthetic construct
  REGION 1..448: note = Third Polypeptide Chain of "gpA33 mAb 1 x CD3 mAb 2 x EphA2 mAb 3" Tri-Specific Binding Molecule
EVQLVESGGGSVKPGGSLKLSCAASGFTFTDHYMYWVRQTPEKRLEWVATISDGGSFTSYPDSVKGRFTISRDIAKNNLYLQMSSLKSEDTAMYYCTRDESDRPFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 273 - DNA, 1344 bp, synthetic construct
  misc_feature 1..1344: note = Polynucleotide Encoding Third Polypeptide Chain of "gpA33 mAb 1 x CD3 mAb 2 x EphA2 mAb 3" Tri-Specific Binding Molecule
  source 1..1344: mol_type = other DNA
gaagtgcagctggtggagtctgggggaggctcagtgaagcctggagggtccctgaaactctcctgtgcagcctctggattcactttcactgaccattacatgtattgggttcgccagactccggaaaagaggctggagtgggtcgcaaccattagtgatggcggtagtttcacctcctatccagacagtgtgaaggggcgattcaccatctccagagacattgccaagaacaacctgtacctccaaatgagcagtctgaagtctgaggacacagccatgtattactgtacaagagatgagagcgataggccgtttccttactggggccaagggactctggtcactgtctcctccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgagttgcgcagtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccgctacacgcagaagagcctctccctgtctccgggtaaa

SEQ ID NO: 274 - protein, 214 aa, synthetic construct
  REGION 1..214: note = Fourth Polypeptide Chain of "gpA33 mAb 1 x CD3 mAb 2 x EphA2 mAb 3" Tri-Specific Binding Molecule
DIVLTQSHRSMSTSVGDRVNITCKASQDVTTAVAWYQQKPGQSPKLLIFWASTRHAGVPDRFTGSGSGTDFTLTISSVQAGDLALYYCQQHYSTPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 275 - DNA, 642 bp, synthetic construct
  misc_feature 1..642: note = Polynucleotide Encoding Fourth Polypeptide Chain of "gpA33 mAb 1 x CD3 mAb 2 x EphA2 mAb 3" Tri-Specific Binding Molecule
  source 1..642: mol_type = other DNA
gacattgtgctgacccagtctcacagatccatgtccacatcagtaggagacagggtcaacatcacctgcaaggccagtcaggatgtgactactgctgtagcctggtatcaacaaaaaccagggcaatctcctaaattactgattttctgggcatccacccggcacgctggagtccctgatcgcttcacaggcagtggatctgggacagattttactctcaccatcagcagtgtgcaggctggagacctggcactttattactgtcaacaacattatagcacaccgtacacattcggaggggggaccaagctggaaataaaacgtacggtggctgcaccatcggtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 276 - protein, 503 aa, synthetic construct; identical to SEQ ID NO: 228
  REGION 1..503: note = First Polypeptide Chain of "EphA2 mAb 1 x CD3 mAb 2 x gpA33 mAb 1" Tri-Specific Binding Molecule

SEQ ID NO: 277 - DNA, 1509 bp, synthetic construct
  misc_feature 1..1509: note = Polynucleotide Encoding First Polypeptide Chain of "EphA2 mAb 1 x CD3 mAb 2 x gpA33 mAb 1" Tri-Specific Binding Molecule
  source 1..1509: mol_type = other DNA
gatatccagatgacacagactacatcctccctgtctgcctctctgggagacagaatcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatattgccacttacttttgccaacagggttatacgctgtacacgttcggaggggggaccaagctggaaataaaaggtggaggatccggcggcggaggcgaggtgcagctggtggagtctgggggaggcttggtccagcctggagggtccctgagactctcctgtgcagcctctggattcaccttcagcacatacgctatgaattgggtccgccaggctccagggaaggggctggagtgggttggaaggatcaggtccaagtacaacaattatgcaacctactatgccgactctgtgaagggtagattcaccatctcaagagatgattcaaagaactcactgtatctgcaaatgaacagcctgaaaaccgaggacacggccgtgtattactgtgtgagacacggtaacttcggcaattcttacgtgtcttggtttgcttattggggacaggggacactggtgactgtgtcttccggaggatgtggcggtggagaagtggccgcactggagaaagaggttgctgctttggagaaggaggtcgctgcacttgaaaaggaggtcgcagccctggagaaaggcggcggggacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 278 - protein, 270 aa, synthetic construct; identical to SEQ ID NO: 230
  REGION 1..270: note = Second Polypeptide Chain of "EphA2 mAb 1 x CD3 mAb 2 x gpA33 mAb 1" Tri-Specific Binding Molecule

SEQ ID NO: 279 - DNA, 810 bp, synthetic construct; identical to SEQ ID NO: 231
  misc_feature 1..810: note = Polynucleotide Encoding Second Polypeptide Chain of "EphA2 mAb 1 x CD3 mAb 2 x gpA33 mAb 1" Tri-Specific Binding Molecule
  source 1..810: mol_type = other DNA

SEQ ID NO: 280 - protein, 449 aa, synthetic construct
  REGION 1..449: note = Third Polypeptide Chain of "EphA2 mAb 1 x CD3 mAb 2 x gpA33 mAb 1" Tri-Specific Binding Molecule
QVQLVQSGAEVKKPGASVKVSCKASGYTFTGSWMNWVRQAPGQGLEWIGRIYPGDGETNYNGKFKDRVTITADKSTSTAYMELSSLRSEDTAVYYCARIYGNNVYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 281 - DNA, 1347 bp, synthetic construct
  misc_feature 1..1347: note = Polynucleotide Encoding Third Polypeptide Chain of "EphA2 mAb 1 x CD3 mAb 2 x gpA33 mAb 1" Tri-Specific Binding Molecule
  source 1..1347: mol_type = other DNA
caggtccagctggtccagagcggggccgaagtcaaaaaacccggagcaagcgtgaaggtctcctgcaaagcatcaggctatacatttacaggcagctggatgaactgggtgaggcaggctccaggacagggactggagtggatcgggcgcatctaccctggagacggcgaaactaactataatggaaagttcaaagaccgagtgaccatcacagccgataagtctactagtaccgcctacatggagctgagctccctgcggtctgaagataccgccgtctactattgcgctagaatttacggaaacaatgtctattttgacgtgtgggggcagggaacaactgtgactgtctcctccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgagttgcgcagtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccgctacacgcagaagagcctctccctgtctccgggtaaa

SEQ ID NO: 282 - protein, 213 aa, synthetic construct
  REGION 1..213: note = Fourth Polypeptide Chain of "EphA2 mAb 1 x CD3 mAb 2 x gpA33 mAb 1" Tri-Specific Binding Molecule
DIQLTQSPSFLSASVGDRVTITCSARSSISFMYWYQQKPGKAPKLLIYDTSNLASGVPSRFSGSGSGTEFTLTISSLEAEDAATYYCQQWSSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 283 - DNA, 639 bp, synthetic construct
  misc_feature 1..639: note = Polynucleotide Encoding Fourth Polypeptide Chain of "EphA2 mAb 1 x CD3 mAb 2 x gpA33 mAb 1" Tri-Specific Binding Molecule
  source 1..639: mol_type = other DNA
gacattcagctgactcagtccccctcttttctgtccgcatccgtcggagatcgagtgactattacttgctctgctaggtcctcaatcagcttcatgtactggtatcagcagaagcccggcaaagcacctaagctgctgatctacgacacaagcaacctggcctccggggtgccatctcggttctctggcagtgggtcaggaactgagtttaccctgacaattagctccctggaggctgaagatgccgctacctactattgccagcagtggagcagctatcctctgaccttcggacaggggactaaactggaaatcaagcgtacggtggctgcaccatcggtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 284 - protein, 508 aa, synthetic construct; identical to SEQ ID NO: 236
  REGION 1..508: note = First Polypeptide Chain of "EphA2 mAb 2 x CD3 mAb 2 x gpA33 mAb 1" Tri-Specific Binding Molecule

SEQ ID NO: 285 - DNA, 1524 bp, synthetic construct; identical to SEQ ID NO: 237
  misc_feature 1..1524: note = Polynucleotide Encoding First Polypeptide Chain of "EphA2 mAb 2 x CD3 mAb 2 x gpA33 mAb 1" Tri-Specific Binding Molecule
  source 1..1524: mol_type = other DNA

SEQ ID NO: 286 - protein, 270 aa, synthetic construct; identical to SEQ ID NO: 238
  REGION 1..270: note = Second Polypeptide Chain of "EphA2 mAb 2 x CD3 mAb 2 x gpA33 mAb 1" Tri-Specific Binding Molecule

SEQ ID NO: 287 - DNA, 810 bp, synthetic construct; identical to SEQ ID NO: 239
  misc_feature 1..810: note = Polynucleotide Encoding Second Polypeptide Chain of "EphA2 mAb 2 x CD3 mAb 2 x gpA33 mAb 1" Tri-Specific Binding Molecule
  source 1..810: mol_type = other DNA

SEQ ID NO: 288 - protein, 449 aa, synthetic construct; identical to SEQ ID NO: 280
  REGION 1..449: note = Third Polypeptide Chain of "EphA2 mAb 2 x CD3 mAb 2 x gpA33 mAb 1" Tri-Specific Binding Molecule

SEQ ID NO: 289 - DNA, 1347 bp, synthetic construct; identical to SEQ ID NO: 281
  misc_feature 1..1347: note = Polynucleotide Encoding Third Polypeptide Chain of "EphA2 mAb 2 x CD3 mAb 2 x gpA33 mAb 1" Tri-Specific Binding Molecule
  source 1..1347: mol_type = other DNA

SEQ ID NO: 290 - protein, 213 aa, synthetic construct; identical to SEQ ID NO: 282
  REGION 1..213: note = Fourth Polypeptide Chain of "EphA2 mAb 2 x CD3 mAb 2 x gpA33 mAb 1" Tri-Specific Binding Molecule

SEQ ID NO: 291 - DNA, 639 bp, synthetic construct; identical to SEQ ID NO: 283
  misc_feature 1..639: note = Polynucleotide Encoding Fourth Polypeptide Chain of "EphA2 mAb 2 x CD3 mAb 2 x gpA33 mAb 1" Tri-Specific Binding Molecule
  source 1..639: mol_type = other DNA

SEQ ID NO: 292 - protein, 504 aa, synthetic construct; identical to SEQ ID NO: 244
  REGION 1..504: note = First Polypeptide Chain of "EphA2 mAb 3 x CD3 mAb 2 x gpA33 mAb 1" Tri-Specific Binding Molecule

SEQ ID NO: 293 - DNA, 1512 bp, synthetic construct; identical to SEQ ID NO: 245
  misc_feature 1..1512: note = Polynucleotide Encoding First Polypeptide Chain of "EphA2 mAb 3 x CD3 mAb 2 x gpA33 mAb 1" Tri-Specific Binding Molecule
  source 1..1512: mol_type = other DNA

SEQ ID NO: 294 - protein, 270 aa, synthetic construct; identical to SEQ ID NO: 246
  REGION 1..270: note = Second Polypeptide Chain of "EphA2 mAb 3 x CD3 mAb 2 x gpA33 mAb 1" Tri-Specific Binding Molecule

SEQ ID NO: 295 - DNA, 810 bp, synthetic construct; identical to SEQ ID NO: 247
  misc_feature 1..810: note = Polynucleotide Encoding Second Polypeptide Chain of "EphA2 mAb 3 x CD3 mAb 2 x gpA33 mAb 1" Tri-Specific Binding Molecule
  source 1..810: mol_type = other DNA

SEQ ID NO: 296 - protein, 449 aa, synthetic construct; identical to SEQ ID NO: 280
  REGION 1..449: note = Third Polypeptide Chain of "EphA2 mAb 3 x CD3 mAb 2 x gpA33 mAb 1" Tri-Specific Binding Molecule

SEQ ID NO: 297 - DNA, 1347 bp, synthetic construct; identical to SEQ ID NO: 281
  misc_feature 1..1347: note = Polynucleotide Encoding Third Polypeptide Chain of "EphA2 mAb 3 x CD3 mAb 2 x gpA33 mAb 1" Tri-Specific Binding Molecule
  source 1..1347: mol_type = other DNA

SEQ ID NO: 298 - protein, 213 aa, synthetic construct; identical to SEQ ID NO: 282
  REGION 1..213: note = Fourth Polypeptide Chain of "EphA2 mAb 3 x CD3 mAb 2 x gpA33 mAb 1" Tri-Specific Binding Molecule

SEQ ID NO: 299 - DNA, 639 bp, synthetic construct; identical to SEQ ID NO: 283
  misc_feature 1..639: note = Polynucleotide Encoding Fourth Polypeptide Chain of "EphA2 mAb 3 x CD3 mAb 2 x gpA33 mAb 1" Tri-Specific Binding Molecule
  source 1..639: mol_type = other DNA

SEQ ID NO: 300 - protein, 279 aa, synthetic construct
  REGION 1..279: note = First Polypeptide Chain of "4-4-20 x CD3 mAb 2" Diabody
DVVMTQTPFSLPVSLGDQASISCRSSQSLVHSNGNTYLRWYLQKPGQSPKVLIYKVSNRFSGVPDRFSGSGSGTDFTLKISRVEAEDLGVYFCSQSTHVPWTFGGGTKLEIKGGGSGGGGEVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSSGGCGGGEVAALEKEVAALEKEVAALEKEVAALEK

SEQ ID NO: 301 - protein, 270 aa, synthetic construct
  REGION 1..270: note = Second Polypeptide Chain of "4-4-20 x CD3 mAb 2" Diabody
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGEVKLDETGGGLVQPGRPMKLSCVASGFTFSDYWMNWVRQSPEKGLEWVAQIRNKPYNYETYYSDSVKGRFTISRDDSKSSVYLQMNNLRVEDMGIYYCTGSYYGMDYWGQGTSVTVSSGGCGGGKVAALKEKVAALKEKVAALKEKVAALKE

SEQ ID NO: 302 - protein, 108 aa, Mus musculus
  REGION 1..108: note = MISC_FEATURE - Murine Anti-Human CD317 Antibody ("HM1.24 mAb 1") Light Chain Variable Domain
DIVMTQSHKFMSTSVGDRVSITCKKASQDVNTAVAWYQQKPGQSPKLLIYSASNRYTGVPDRITGSGSGTDFTFTISSVQAEDLALTTCQQHYSTPFTFGSGTKLEIK

SEQ ID NO: 303 - protein, 120 aa, Mus musculus
  REGION 1..120: note = MISC_FEATURE - Murine Anti-Human CD317 Antibody ("HM1.24 mAb 1") Heavy Chain Variable Domain
QVQLQQSGAELARPGASVKLSCKASGYTFTPYWMQWVKQRPGQGLEWIGSIFPGDGDTRYSQKFKGKATLTADKSSSTAYMQLSILAFEDSAVYYCARGLRRGGYYFDYWGQGTTLTVSS

SEQ ID NO: 304 - protein, 107 aa, synthetic construct
  REGION 1..107: note = Murine Anti-Human CEACAM5 / CEACAM6 Antibody (16C3; EP 2585476) Light Chain Variable Domain
DIQMTQSPSSLSASVGDRVTITCGASENIYGALNWYQRKPGKSPKLLIWGASNLADGMPSRFSGSGSGRQYTLTISSLQPEDVATYYCQNVLSSPYTFGGGTKLEIK

SEQ ID NO: 305 - protein, 121 aa, synthetic construct
  REGION 1..121: note = Humanized Anti-Human CEACAM5 / CEACAM6 Antibody (16C3; EP 2585476) Heavy Chain Variable Domain
QVQLQQSGPEVVRPGVSVKISCKGSGYTFTDYAMHWVKQSHAKSLEWIGLISTYSGDTKYNQNFKGKATMTVDKSASTAYMELSSLRSEDTAVYYCARGDYSGSRYWFAYWGQGTLVTVSS

SEQ ID NO: 306 - protein, 107 aa, synthetic construct
  REGION 1..107: note = Humanized Anti-Human CEACAM5 / CEACAM6 Antibody (hMN15; WO 2011/034660) Light Chain Variable Domain
DIQLTQSPSSLSASVGDRVTMTCSASSRVSYIHWYQQKPGKAPKRWIYGTSTLASGVPARFSGSGSGTDFTFTISSLQPEDIATYYCQQWSYNPPTFGQGTKVEIKR

SEQ ID NO: 307 - protein, 121 aa, synthetic construct
  REGION 1..121: note = Humanized Anti-Human CEACAM5 / CEACAM6 Antibody (hMN15; WO 2011/034660) Heavy Chain Variable Domain
QVQLVESGGGVVQPGRSLRLSCSSSGFALTDYYMSWVRQAPGKGLEWLGFIANKANGHTTDYSPSVKGRFTISRDNSKNTLFLQMDSLRPEDTGVYFCARDMGIRWNFDVWGQGTPVTVSS

SEQ ID NO: 308 - protein, 107 aa, Mus musculus
  REGION 1..107: note = MISC_FEATURE - Murine Anti-Human 5T4 Antibody ("5T4 mAb 1") Light Chain Variable Domain
DIQMTQSPSSLSASVGDRVTITCRASQGISNYLAWFQQKPGKAPKSLIYRANRLQSGVPSRFSGSGSGTDFTLTISSLQPEDVATYYCLQYDDFPWTFGQGTKLEIK

SEQ ID NO: 309 - protein, 118 aa, Mus musculus
  REGION 1..118: note = MISC_FEATURE - Murine Anti-Human 5T4 Antibody ("5T4 mAb 1") Heavy Chain Variable Domain
QVQLVQSGAEVKKPGASVKVSCKASGYTFTSFWMHWVRQAPGQGLEWMGRIDPNRGGTEYNEKAKSRVTMTADKSTSTAYMELSSLRSEDTAVYYCAGGNPYYPMDYWGQGTTVTVSS

SEQ ID NO: 310 - protein, 112 aa, Mus musculus
  REGION 1..112: note = MISC_FEATURE - Murine Anti-Human 5T4 Antibody ("5T4 mAb 2") Light Chain Variable Domain
DVLMTQTPLSLPVSLGDQASISCRSSQSIVYSNGNTYLEWYLQKPGQSPKLLIYKVSNRFSGVPDRFSGSGSGTDFTLKISRVEAEDLGVYYCFQGSHVPFTFGSGTKLEIK

SEQ ID NO: 311 - protein, 127 aa, Mus musculus
  REGION 1..127: note = MISC_FEATURE - Murine Anti-Human 5T4 Antibody ("5T4 mAb 2") Heavy Chain Variable Domain
QVQLQQPGAELVKPGASVKMSCKASGYTFTSYWITWVKQRPGQGLEWIGDIYPGSGRANYNEKFKSKATLTVDTSSSTAYMQLSSLTSEDSAVYNCARYGPLFTTVVDPNSYAMDYWGQGTSVTVSS

SEQ ID NO: 312 - protein, 108 aa, synthetic construct
  REGION 1..108: note = Humanized Anti-Human ITGB6 Antibody (Derived from Antibody 3G9, PCT Publication No. WO 2007/008712) Light Chain Variable Domain
EIVLTQSPATLSLSPGERATLSCSASSSVSSSYLYWYQQKPGQAPRLLIYSTSNLASGIPARFSGSGSGTGFTLTISSLEPEDFAVYYCHQWSTYPPTFGGGTKVEIK

SEQ ID NO: 313 - protein, 120 aa, synthetic construct
  REGION 1..120: note = Humanized Anti-Human ITGB6 Antibody (Derived from Antibody 3G9, PCT Publication No. WO 2007/008712) Heavy Chain Variable Domain
EVQLVESGGGLVQPGGSLRLSCAASGFTFSRYWMSWVRQAPGKGLEWVASISSGGRMYYPFTVKGRFTISRDNAKNSLYLQMNSLRAEDTAVYYCARGSIYDGYYVFPYWGQGTLVTVSS

SEQ ID NO: 314 - protein, 111 aa, synthetic construct
  REGION 1..111: note = Humanized Anti-Human ITGB6 Antibody (Derived from Antibody 8G6; PCT Publication No. WO 2007/008712) Light Chain Variable Domain
EIVLTQSPATLSLSPGERATLSCRASQSVSTSSYSYMYWYQQKPGQAPRLLIYYASNLESGIPARFSGSGSGTDFTLTISSLEPEDFAVYYCQHNWEIPFTFGGGTKVEIK

SEQ ID NO: 315 - protein, 126 aa, synthetic construct
  REGION 1..126: note = Humanized Anti-Human ITGB6 Antibody (Derived from Antibody 8G6; PCT Publication No. WO 2007/008712) Heavy Chain Variable Domain
QVQLVQSGAEVKKPGASVKVSCKASGYTFTDYAMHWVRQAPGQGLEWMGVISTYYGNTNYNQKFKGRVTMTRDTSISTAYMELSRLRSDDTAVYYCARGGLRRGDRPSLQYAMDYWGQGTLVTVSS

SEQ ID NO: 316 - protein, 272 aa, synthetic construct
  REGION 1..272: note = First Polypeptide Chain of "gpA33 x CD3 mAb 2" Diabody
DIQLTQSPSFLSASVGDRVTITCSARSSISFMYWYQQKPGKAPKLLIYDTSNLASGVPSRFSGSGSGTEFTLTISSLEAEDAATYYCQQWSSYPLTFGQGTKLEIKGGGSGGGGEVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSSASTKGEVAACEKEVAALEKEVAALEKEVAALEK

SEQ ID NO: 317 - protein, 270 aa, synthetic construct
  REGION 1..270: note = Second Polypeptide Chain of "gpA33 x CD3 mAb 2" Diabody
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGQVQLVQSGAEVKKPGASVKVSCKASGYTFTGSWMNWVRQAPGQGLEWIGRIYPGDGETNYNGKFKDRVTITADKSTSTAYMELSSLRSEDTAVYYCARIYGNNVYFDVWGQGTTVTVSSASTKGKVAACKEKVAALKEKVAALKEKVAALKE

SEQ ID NO: 318 - DNA, 333 bp, Mus musculus
  misc_feature 1..333: note = Polynucleotide encoding Murine Anti-Human EphA2 Antibody ("EphA2 mAb 2") Light Chain Variable Domain (SEQ ID NO:163)
  source 1..333: mol_type = genomic DNA
gatgttgtgatgacccaaactccactctccctgcctgtcagtcttggagatcaagcctccatctcttgcagatctagtcagagccttgtacacagtagtggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttcccacgttcggctcggggacaaagttggaaataaaa

SEQ ID NO: 319 - DNA, 354 bp, Mus musculus
  misc_feature 1..354: note = Polynucelotide Encoding Murine Anti-Human EphA2 Antibody ("EphA2 mAb 3") Heavy Chain Variable Domain (SEQ ID NO:177)
  source 1..354: mol_type = genomic DNA
gaagtgcagctggtggagtctgggggaggctcagtgaagcctggagggtccctgaaactctcctgtgcagcctctggattcactttcactgaccattacatgtattgggttcgccagactccggaaaagaggctggagtgggtcgcaaccattagtgatggcggtagtttcacctcctatccagacagtgtgaaggggcgattcaccatctccagagacattgccaagaacaacctgtacctccaaatgagcagtctgaagtctgaggacacagccatgtattactgtacaagagatgagagcgataggccgtttccttactggggccaagggactctggtcactgtctcctcc

SEQ ID NO: 320 - protein, 11 aa, synthetic construct
  REGION 1..11: note = humanized Anti-Human DR5 Antibody ("DR5 mAb 2") Light Chain Variable Domain CDRL1
RASQDVNTAVA

SEQ ID NO: 321 - protein, 107 aa, synthetic construct
  REGION 1..107: note = humanized anti-IL13RA2 Antibody ("HU08") Light Chain Variable
DIQMTQSPSSLSASVGDRVTITCKASQDVGTAVAWYQQKPGKAPKLLIYSASYRSTGVPSRFSGSGSGTDFTLTISSLQPEDFATYYCQHHYSAPWTFGGGTKVEIK

SEQ ID NO: 322 - protein, 122 aa, synthetic construct
  REGION 1..122: note = humanized anti-IL13RA2 Antibody ("HU08") Heavy Chain Variable
EVQLVESGGGLVQPGGSLRLSCAASGFTFSRNGMSWVRQAPGKGLEWVATVSSGGSYIYYADSVKGRFTISRDNAKNSLYLQMNSLRAEDTAVYYCARQGTTALATRFFDVWGQGTLVTVSS

(19) United States
(12) Patent Application Publication
(10) Pub. No.: US 2024/0117036 A1
(43) Pub. Date: Apr. 11, 2024

(54) TRI-SPECIFIC BINDING MOLECULES THAT SPECIFICALLY BIND TO MULTIPLE CANCER ANTIGENS

(71) Applicant: MacroGenics, Inc., Rockville, MD (US)

(72) Inventors: Ezio BONVINI, Rockville, MD (US); Paul A. MOORE, Rockville, MD (US); Jonathan C. LI, Rockville, MD (US); Leslie S. JOHNSON, Rockville, MD (US); Kalpana SHAH, Rockville, MD (US)

(21) Appl. No.: 18/320,599

(22) Filed: May 19, 2023

Related U.S. Application Data
(63) Continuation of application No. 16/805,105, filed on Feb. 28, 2020, now Pat. No. 11,697,684.
Application No. 16/805,105 is a division of application No. 15/313,765, filed on Nov. 23, 2016, now Pat. No. 10,633,440.
(60) Provisional application No. 62/107,824, filed on Jan. 26, 2015.
(60) Provisional application No. 62/008,229, filed on Jun. 5, 2014.
(60) Provisional application No. 62/004,571, filed on May 29, 2014.

(51) Int. Cl. C07K 16/28; C07K 16/30

(52) U.S. Cl. CPC C07K 16/28; C07K 16/2803; C07K 16/2809; C07K 16/2815; C07K 16/2827; C07K 16/283; C07K 16/2878; C07K 16/30; C07K 2317/31; C07K 2317/524; C07K 2317/526; C07K 2317/55; C07K 2317/56; C07K 2317/565; C07K 2317/622; C07K 2317/626; C07K 2317/73; C07K 2317/92; C07K 2318/20; C07K 2319/00

(57) ABSTRACT

The present invention relates to Tri-Specific Binding Molecules, which are multi-chain polypeptide molecules that possess three Binding Domains and are thus capable of mediating coordinated binding to three epitopes. The Tri-Specific Binding Molecule is preferably characterized in possessing binding domains that permit it to immunospecifically bind to: (1) an epitope of a first Cancer Antigen, (2) an epitope of a second Cancer Antigen, and (3) an epitope of a molecule that is expressed on the surface of an immune system effector cell, and are thus capable of localizing an immune system effector cell to a cell that expresses a Cancer Antigen, so as to thereby facilitate the killing of such cancer cell.

### CROSS-REFERENCE TO RELATED APPLICATIONS

This patent application is a continuation application of U.S. application Ser. No. 16/805,105, filed Feb. 28, 2020, which is a divisional application of U.S. application Ser. No. 15/313,765, filed Nov. 23, 2016, now U.S. Pat. No. 10,633,440, which is a 35 U.S.C. 371 national phase patent application of International Application No. PCT/US2015/033081, filed May 29, 2015, which claims the benefit of U.S. Provisional Patent Application Nos. 62/107,824, filed Jan. 26, 2015, 62/008,229, filed Jun. 5, 2014, and 62/004,571, filed May 29, 2014. The contents of each of these applications are incorporated herein by reference in their entireties.

### REFERENCE TO SEQUENCE LISTING

The instant application contains a Sequence Listing (0260-0002US3_SL.xml; size 502 KB; and Date of Creation May 15, 2023) which has been submitted electronically and is hereby incorporated by reference in its entirety.

### BACKGROUND OF THE INVENTION

### Field of the Invention

The present invention relates to Tri-Specific Binding Molecules, which are multi-chain polypeptide molecules that possess three Binding Domains and are thus capable of mediating coordinated binding to three epitopes. The Tri-Specific Binding Molecule is preferably characterized in possessing binding domains that permit it to immunospecifically bind to: (1) an epitope of a first Cancer Antigen, (2) an epitope of a second Cancer Antigen, and (3) an epitope of a molecule that is expressed on the surface of an immune system effector cell, and are thus capable of localizing an immune system effector cell to a cell that expresses a Cancer Antigen, so as to thereby facilitate the killing of such cancer cell.

### Description of Related Art

### I. The Mammalian Immune System

The mammalian immune system serves as a defense against a variety of conditions, including, e.g., injury, infection and neoplasia. The efficiency with which humans and other mammals develop an immunological response to pathogens, foreign substances and cancer antigens rests on two characteristics: the exquisite specificity of the immune response for antigen recognition, and the immunological memory that allows for faster and more vigorous responses upon re-activation with the same antigen (Portolés, P. et al. (2009) “The TCR CD3 Complex: Opening the Gate to Successful Vaccination,” Current Pharmaceutical Design 15:3290-3300; Guy, C. S. et al. (2009) “Organization of Proximal Signal Initiation at the TCR:CD3 Complex,” Immunol Rev. 232(1):7-21).

The mammalian immune system is mediated by two separate but interrelated systems: the cellular and humoral immune systems. Generally speaking, the humoral system is mediated by soluble products (antibodies or immunoglobulins) that have the ability to combine with and neutralize products recognized by the system as being foreign to the body. In contrast, the cellular immune system involves the mobilization of certain cells, termed “T cells,” that serve a variety of therapeutic roles. T cells are lymphocytes that are derived from the thymus and circulate between the tissues, lymphatic system and the circulatory system. In response to the presence and recognition of foreign structures (antigens), T cells become “activated” to initiate an immune response. In many instances these foreign antigens are expressed on host cells as a result of neoplasia or infection. Although T cells do not themselves secrete antibodies, they are usually required for antibody secretion by the second class of lymphocytes, B cells (which derive from bone marrow). Critically, T cells exhibit extraordinary immunological specificity so as to be capable of discerning one antigen from another). Two types of T cells, “T helper cells” and “cytotoxic T cells,” are of particular relevance.

T helper cells are characterized by their expression of the glycoprotein, CD4 (i.e., they are “CD4⁺”). CD4⁺ T cells are the essential organizers of most mammalian immune and autoimmune responses (Dong, C. et al. (2003) “Immune Regulation by Novel Costimulatory Molecules,” Immunolog. Res. 28(1):39-48). The activation of CD4⁺ T cells has been found to be mediated through co-stimulatory interactions between an antigen:major histocompability class II (MHC II) molecule complex that is arrayed on the surface of an Antigen Presenting Cell (such as a B cell, a macrophage or a dendritic cell) and a complex of two molecules, the T Cell Receptor (“TCR”) and a CD3 cell surface receptor ligand, that are arrayed on surface of a naive CD4⁺ T cell. Activated T helper cells are capable of proliferating into Th1 cells that are capable of mediating an inflammatory response to the target cell.

Cytotoxic T cells are characterized by their expression of CD8 (i.e., they are “CD8+” as well as CD3⁺). The activation of CD8⁺ T cells has been found to be mediated through co-stimulatory interactions between an antigen:major histocompability class I (MHC I) molecule complex that is arrayed on the surface of a target cell and a complex of CD8 and the T Cell Receptor, that are arrayed on surface of the CD8⁺ T cell. Unlike MHC II molecules, which are expressed by only certain immune system cells, MHC I molecules are very widely expressed. Thus, cytotoxic T cells are capable of binding to a wide variety of cell types. Activated cytotoxic T cells mediate cell killing through their release of the cytotoxins perforin, granzymes, and granulysin. Through the action of perforin, granzymes enter the cytoplasm of the target cell and their serine protease function triggers the caspase cascade, which is a series of cysteine proteases that eventually lead to apoptosis (programmed cell death) of targeted cells.

The T cell receptor (“TCR”) is a covalently linked heterodimer of α and β chains (“TCRαβ”). These chains are class I membrane polypeptides of 259 (α) and 296 (β) amino acids in length. The CD3 molecule is a T cell co-receptor composed of five distinct polypeptide chains (a CD3 γ chain, a CD3 δ chain, two CD3 ε chains and two zeta chains). The individual polypeptide chains associate to form a complex of three dimers (εγ, εδ, ζζ) (Wucherpfennig, K. W. et al. (2010) “Structural Biology Of The T Cell Receptor: Insights into Receptor Assembly, Ligand Recognition, And Initiation of Signaling,” Cold Spring Harb. Perspect. Biol. 2(4):a005140; pages 1-14; Chetty, R. et al. (1994) “CD3: Structure, Function And The Role Of Immunostaining In Clinical Practice,” J. Pathol. 173:303-307; Guy, C. S. et al. (2009) “Organization of Proximal Signal Initiation at the TCR:CD3 Complex,” Immunol Rev. 232(1):7-21; Call, M. E. et al. (2007) “Common Themes In The Assembly And Architecture Of Activating Immune Receptors,” Nat. Rev. Immunol. 7:841-850; Weiss, A. (1993) “T Cell Antigen Receptor Signal Transduction: A Tale Of Tails And Cytoplasmic Protein-Tyrosine Kinases,” Cell 73:209-212). The CD3 complex associates with TCR in order to generate an activation signal in T lymphocytes. In the absence of CD3, TCRs do not assemble properly and are degraded (Thomas, S. et al. (2010) “Molecular Immunology Lessons From Therapeutic T Cell Receptor Gene Transfer,” Immunology 129(2):170-177). CD3 is found bound to the membranes of all mature T cells, and in virtually no other cell type (see, Janeway, C. A. et al. (2005) In: IMMUNOBIOLOGY: THE IMMUNE SYSTEM IN HEALTH AND DISEASE,” 6th ed. Garland Science Publishing, NY, pp. 214-216; Sun, Z. J. et al. (2001) “Mechanisms Contributing To T Cell Receptor Signaling And Assembly Revealed By The Solution Structure Of An Ectodomain Fragment Of The CD3ε:γ Heterodimer,” Cell 105(7):913-923; Kuhns, M. S. et al. (2006) “Deconstructing The Form And Function Of The TCR CD3 Complex,” Immunity. 2006 February; 24(2):133-139).

The TCR and CD3 complex, along with the CD3 (chain zeta chain (also known as T cell receptor T3 zeta chain or CD247) comprise the TCR complex (van der Merwe, P. A. etc. (epub Dec. 3, 2010) “Mechanisms For T Cell Receptor Triggering,” Nat. Rev. Immunol. 11:47-55; Wucherpfennig, K. W. et al. (2010) “Structural Biology of the T cell Receptor: Insights into Receptor Assembly, Ligand Recognition, and Initiation of Signaling,” Cold Spring Harb. Perspect. Biol. 2:a005140). The complex is particularly significant since it contains a large number (ten) of immunoreceptor tyrosine-based activation motifs (ITAMs).

Two interactions are required for T cell activation (Viglietta, V. et al. (2007) “Modulating Co-Stimulation,” Neurotherapeutics 4:666-675; Korman, A. J. et al. (2007) “Checkpoint Blockade in Cancer Immunotherapy,” Adv. Immunol. 90:297-339). In the first interaction, a Cell must display the relevant target antigen bound to the cell's major histocompatibility complex so that it can bind to the T cell Receptor (“TCR”) of a naive T lymphocyte. In the second interaction, a ligand of the Cell must bind to a co-receptor of the T lymphocyte (Dong, C. et al. (2003) “Immune Regulation by Novel Costimulatory Molecules,” Immunolog. Res. 28(1):39-48; Lindley, P. S. et al. (2009) “The Clinical Utility Of Inhibiting CD28-Mediated Costimulation,” Immunol. Rev. 229:307-321). T cells experiencing both stimulatory signals are then capable of responding to cytokines (such as Interleukin-2 and Interleukin-12). In the absence of both co-stimulatory signals during TCR engagement, T cells enter a functionally unresponsive state, referred to as clonal anergy (Khawli, L. A. et al. (2008) “Cytokine, Chemokine, and Co-Stimulatory Fusion Proteins for the Immunotherapy of Solid Tumors,” Exper. Pharmacol. 181:291-328). In pathologic states, T cells are the key players of various organ-specific autoimmune diseases, such as type I diabetes, rheumatoid arthritis, and multiple sclerosis (Dong, C. et al. (2003) “Immune Regulation by Novel Costimulatory Molecules,” Immunolog. Res. 28(1):39-48).

The need for two signals to activate T cells such that they achieve an adaptive immune response is believed to provide a mechanism for avoiding responses to self-antigens that may be present on an Antigen Presenting Cell at locations in the system where it can be recognized by a T cell. Where contact of a T cell with a Cell results in the generation of only one of two required signals, the T cell does not become activated and an adaptive immune response does not occur.

### II. Antibodies and Other Epitope-Binding Molecules

A. Antibodies

“Antibodies” are immunoglobulin molecules capable of specific binding to a target, such as a carbohydrate, polynucleotide, lipid, polypeptide, etc., through at least one antigen recognition site, located in the Variable Domain of the immunoglobulin molecule. As used herein, the term encompasses not only intact polyclonal or monoclonal antibodies, camelized antibodies, single-chain antibodies, and anti-idiotypic (anti-Id) antibodies (including, e.g., anti-Id and anti-anti-Id antibodies to antibodies of the invention), but also mutants thereof, naturally occurring variants, fusion proteins comprising an antibody portion with an antigen recognition site of the required specificity, humanized antibodies, and chimeric antibodies, and any other modified configuration of the immunoglobulin molecule that comprises an antigen recognition site of the required specificity. Throughout this application, the numbering of amino acid residues of the light and heavy chains of antibodies is according to the EU index as in Kabat et al. (1992) SEQUENCES OF PROTEINS OF IMMUNOLOGICAL INTEREST, National Institutes of Health Publication No. 91-3242. As used herein, an “antigen-binding fragment of an antibody” is a portion of an antibody that possesses an at least one antigen recognition site. As used herein, the term encompasses fragments (e.g., Fab, Fab′, F(ab′)₂ Fv), disulfide-linked bispecific Fvs (sdFv), intrabodies, and single-chain molecules (e.g., scFv). In particular, antibodies include immunoglobulin molecules and immunologically active fragments of immunoglobulin molecules, i.e., molecules that contain an antigen-binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass.

Natural antibodies (such as IgG antibodies) are composed of two Light Chains complexed with two Heavy Chains. Each Light Chain contains a Variable Domain (VL) and a Constant Domain (CL). Each heavy chain contains a Variable Domain (VH), three Constant Domains (CH1, CH2 and CH3), and a Hinge Domain located between the CH1 and CH2 Domains. The basic structural unit of naturally occurring immunoglobulins (e.g., IgG) is thus a tetramer having two light chains and two heavy chains, usually expressed as a glycoprotein of about 150,000 Da. The amino-terminal (“N”) portion of each chain includes a variable region of about 100 to 110 or more amino acids primarily responsible for antigen recognition. The carboxy-terminal (“C”) portion of each chain defines a constant region, with light chains having a single Constant Domain and heavy chains usually having three Constant Domains and a hinge region. Thus, the structure of the light chains of an IgG molecule is n-VL-CL-c and the structure of the IgG heavy chains is n-VH-CH1-H-CH2-CH3-c (where H is the hinge region, and n and c represent, respectively, the N-terminus and the C-terminus of the polypeptide).

The ability of an intact, unmodified antibody (e.g., an IgG antibody) to bind an epitope of an antigen depends upon the presence of Variable Domains on the immunoglobulin light and heavy chains (i.e., the VL Domain and VH Domain, respectively). Interaction of an antibody Light Chain and an antibody heavy chain and, in particular, interaction of its VL and VH Domains forms one of the epitope-binding sites of the antibody. The variable regions of an IgG molecule consist of the complementarity determining regions (CDR), which contain the residues in contact with epitope, and non-CDR segments, referred to as framework segments (FR), which in general maintain the structure and determine the positioning of the CDR loops so as to permit such contacting (although certain framework residues may also contact antigen). Thus, the VL and VH Domains have the structure n-FR1-CDR1-FR2-CDR2-FR3-CDR3-FR4-c. Polypeptides that are (or may serve as) the first, second and third CDR of an antibody Light Chain are herein respectively designated CDR_{L}1 Domain, CDR_{L}2 Domain, and CDR_{L}3 Domain. Similarly, polypeptides that are (or may serve as) the first, second and third CDR of an antibody heavy chain are herein respectively designated CDR_{H}1 Domain, CDR_{H}2 Domain, and CDR_{H}3 Domain. Thus, the terms CDR_{L}1 Domain, CDR_{L}2 Domain, CDR_{L}3 Domain, CDR_{H}1 Domain, CDR_{H}2 Domain, and CDR_{H}3 Domain are directed to polypeptides that when incorporated into a protein cause that protein to be able to bind to an specific epitope regardless of whether such protein is an antibody having light and heavy chains or a diabody or a single-chain binding molecule (e.g., an scFv, a BiTe, etc.), or is another type of protein. In contrast to such antibodies, the scFv construct comprises a VL and VH Domain of an antibody contained in a single polypeptide chain wherein the Domains are separated by a flexible linker of sufficient length to allow self-assembly of the two Domains into a functional epitope-binding site. Where self-assembly of the VL and VH Domains is rendered impossible due to a linker of insufficient length (less than about 12 amino acid residues), two of the scFv constructs may interact with one another other to form a bivalent molecule in which the VL of one chain associates with the VH of the other (reviewed in Marvin et al. (2005) “Recombinant Approaches To IgG-Like Bispecific Antibodies,” Acta Pharmacol. Sin. 26:649-658).

In addition to their known uses in diagnostics, antibodies have been shown to be useful as therapeutic agents. The last few decades have seen a revival of interest in the therapeutic potential of antibodies, and antibodies have become one of the leading classes of biotechnology-derived drugs (Chan, C. E. et al. (2009) “The Use Of Antibodies In The Treatment Of Infectious Diseases,” Singapore Med. J. 50(7):663-666). Nearly 200 antibody-based drugs have been approved for use or are under development.

The term “monoclonal antibody” refers to a homogeneous antibody population wherein the monoclonal antibody is comprised of amino acids (naturally occurring and non-naturally occurring) that are involved in the selective binding of an antigen. Monoclonal antibodies are highly specific, being directed against a single epitope (or antigenic site). The term “monoclonal antibody” encompasses not only intact monoclonal antibodies and full-length monoclonal antibodies, but also fragments thereof (such as Fab, Fab′, F(ab′)₂ Fv), single-chain (scFv), mutants thereof, fusion proteins comprising an antibody portion, humanized monoclonal antibodies, chimeric monoclonal antibodies, and any other modified configuration of the immunoglobulin molecule that comprises an antigen recognition site of the required specificity and the ability to bind to an antigen. It is not intended to be limited as regards to the source of the antibody or the manner in which it is made (e.g., by hybridoma, phage selection, recombinant expression, transgenic animals, etc.). The term includes whole immunoglobulins as well as the fragments etc. described above under the definition of “antibody.” Methods of making monoclonal antibodies are known in the art. One method which may be employed is the method of Kohler, G. et al. (1975) “Continuous Cultures Of Fused Cells Secreting Antibody Of Predefined Specificity,” Nature 256:495-497 or a modification thereof. Typically, monoclonal antibodies are developed in mice, rats or rabbits. The antibodies are produced by immunizing an animal with an immunogenic amount of cells, cell extracts, or protein preparations that contain the desired epitope. The immunogen can be, but is not limited to, primary cells, cultured cell lines, cancerous cells, proteins, peptides, nucleic acids, or tissue. Cells used for immunization may be cultured for a period of time (e.g., at least 24 hours) prior to their use as an immunogen. Cells may be used as immunogens by themselves or in combination with a non-denaturing adjuvant, such as Ribi (see, e.g., Jennings, V. M. (1995) “Review of Selected Adjuvants Used in Antibody Production,” ILAR J. 37(3):119-125).

In general, cells should be kept intact and preferably viable when used as immunogens. Intact cells may allow antigens to be better detected than ruptured cells by the immunized animal. Use of denaturing or harsh adjuvants, e.g., Freud's adjuvant, may rupture cells and therefore is discouraged. The immunogen may be administered multiple times at periodic intervals such as, bi weekly, or weekly, or may be administered in such a way as to maintain viability in the animal (e.g., in a tissue recombinant). Alternatively, existing monoclonal antibodies and any other equivalent antibodies that are immunospecific for a desired pathogenic epitope can be sequenced and produced recombinantly by any means known in the art. In one embodiment, such an antibody is sequenced and the polynucleotide sequence is then cloned into a vector for expression or propagation. The sequence encoding the antibody of interest may be maintained in a vector in a host cell and the host cell can then be expanded and frozen for future use. The polynucleotide sequence of such antibodies may be used for genetic manipulation to generate a chimeric antibody, a humanized antibody, or a caninized antibody, or to improve the affinity, or other characteristics of the antibody. The term “humanized” antibody refer to a chimeric molecule, generally prepared using recombinant techniques, having an antigen-binding site derived from an immunoglobulin from a non-human species and the remaining immunoglobulin structure of the molecule based upon the structure and/or sequence of a human immunoglobulin. The polynucleotide sequence of the variable domains of such antibodies may be used for genetic manipulation to generate such derivatives and to improve the affinity, or other characteristics of such antibodies. The general principle in humanizing an antibody involves retaining the basic sequence of the antigen-binding portion of the antibody, while swapping the non-human remainder of the antibody with human antibody sequences. There are four general steps to humanize a monoclonal antibody. These are: (1) determining the nucleotide and predicted amino acid sequence of the starting antibody light and heavy variable Domains (2) designing the humanized antibody or caninized antibody, i.e., deciding which antibody framework region to use during the humanizing or canonizing process (3) the actual humanizing or caninizing methodologies/techniques and (4) the transfection and expression of the humanized antibody. See, for example, U.S. Pat. Nos. 4,816,567; 5,807,715; 5,866,692; and 6,331,415.

The epitope-binding domain of such antibodies may comprise either complete Variable Domains fused onto Constant Domains or only the complementarity determining regions (CDRs) grafted onto appropriate framework regions in the Variable Domains. Antigen-binding sites may be wild-type or modified by one or more amino acid substitutions. This eliminates the constant region as an immunogen in human individuals, but the possibility of an immune response to the foreign variable region remains (LoBuglio, A. F. et al. (1989) “Mouse Human Chimeric Monoclonal Antibody In Man: Kinetics And Immune Response,” Proc. Natl. Acad. Sci. (U.S.A.) 86:4220-4224). Another approach focuses not only on providing human-derived constant regions, but modifying the variable regions as well so as to reshape them as closely as possible to human form. It is known that the variable regions of both heavy and light chains contain three complementarity determining regions (CDRs) which vary in response to the antigens in question and determine binding capability, flanked by four framework regions (FRs) which are relatively conserved in a given species and which putatively provide a scaffolding for the CDRs. When non-human antibodies are prepared with respect to a particular antigen, the variable regions can be “reshaped” or “humanized” by grafting CDRs derived from non-human antibody on the FRs present in the human antibody to be modified. Application of this approach to various antibodies has been reported by Sato, K. et al. (1993) Cancer Res 53:851-856. Riechmann, L. et al. (1988) “Reshaping Human Antibodies for Therapy,” Nature 332:323-327; Verhoeyen, M. et al. (1988) “Reshaping Human Antibodies: Grafting An Antilysozyme Activity,” Science 239:1534-1536; Kettleborough, C. A. et al. (1991) “Humanization Of A Mouse Monoclonal Antibody By CDR-Grafting: The Importance Of Framework Residues On Loop Conformation,” Protein Engineering 4:773-3783; Maeda, H. et al. (1991) “Construction Of Reshaped Human Antibodies With HIV-Neutralizing Activity,” Human Antibodies Hybridoma 2:124-134; Gorman, S. D. et al. (1991) “Reshaping A Therapeutic CD4 Antibody,” Proc. Natl. Acad. Sci. (U.S.A.) 88:4181-4185; Tempest, P. R. et al. (1991) “Reshaping A Human Monoclonal Antibody To Inhibit Human Respiratory Syncytial Virus Infection in vivo,” Bio/Technology 9:266-271; Co, M. S. et al. (1991) “Humanized Antibodies For Antiviral Therapy,” Proc. Natl. Acad. Sci. (U.S.A.) 88:2869-2873; Carter, P. et al. (1992) “Humanization Of An Anti-p185her2 Antibody For Human Cancer Therapy,” Proc. Natl. Acad. Sci. (U.S.A.) 89:4285-4289; and Co, M. S. et al. (1992) “Chimeric And Humanized Antibodies With Specificity For The CD33 Antigen,” J. Immunol. 148:1149-1154. In some embodiments, humanized antibodies preserve all CDR sequences (for example, a humanized mouse antibody which contains all six CDRs from the mouse antibodies). In other embodiments, humanized antibodies have one or more CDRs (one, two, three, four, five, or six) which differ in sequence relative to the original antibody.

A number of “humanized” antibody molecules comprising an antigen-binding site derived from a non-human immunoglobulin have been described, including chimeric antibodies having rodent or modified rodent V regions and their associated complementarity determining regions (CDRs) fused to human constant domains (see, for example, Winter et al. (1991) “Man-made Antibodies,” Nature 349:293-299; Lobuglio et al. (1989) “Mouse Human Chimeric Monoclonal Antibody In Man: Kinetics And Immune Response,” Proc. Natl. Acad. Sci. (U.S.A.) 86:4220-4224 (1989), Shaw et al. (1987) “Characterization Of A Mouse Human Chimeric Monoclonal Antibody (17-4A) To A Colon Cancer Tumor-Associated Antigen,” J. Immunol. 138:4534-4538, and Brown et al. (1987) “Tumor-Specific Genetically Engineered Murine Human Chimeric Monoclonal Antibody,” Cancer Res. 47:3577-3583). Other references describe rodent CDRs grafted into a human supporting framework region (FR) prior to fusion with an appropriate human antibody constant domain (see, for example, Riechmann, L. et al. (1988) “Reshaping Human Antibodies for Therapy,” Nature 332:323-327; Verhoeyen, M. et al. (1988) “Reshaping Human Antibodies: Grafting An Antilysozyme Activity,” Science 239:1534-1536; and Jones et al. (1986) “Replacing The Complementarity-Determining Regions In A Human Antibody With Those From A Mouse,” Nature 321:522-525). Another reference describes rodent CDRs supported by recombinantly veneered rodent framework regions. See, for example, European Patent Publication No. 519,596. These “humanized” molecules are designed to minimize unwanted immunological response toward rodent anti-human antibody molecules, which limits the duration and effectiveness of therapeutic applications of those moieties in human recipients. Other methods of humanizing antibodies that may also be utilized are disclosed by Daugherty et al. (1991) “Polymerase Chain Reaction Facilitates The Cloning, CDR-Grafting, And Rapid Expression Of A Murine Monoclonal Antibody Directed Against The CD18 Component Of Leukocyte Integrins,” Nucl. Acids Res. 19:2471-2476 and in U.S. Pat. Nos. 6,180,377; 6,054,297; 5,997,867; and 5,866,692.

B. Bi-Specific Antibodies, Multi-Specific Diabodies and DART™ Diabodies

Natural antibodies are capable of binding to only one epitope species (i.e., they are “mono-specific”), although they may be able to bind multiple copies of that species (i.e., they may exhibit bi-valency or multi-valency). A wide variety of recombinant bi-specific antibody formats have been developed (see, e.g., PCT Publication Nos. WO 2008/003116, WO 2009/132876, WO 2008/003103, WO 2007/146968, WO 2007/146968, WO 2009/018386, WO 2012/009544, WO 2013/070565), most of which use linker peptides either to fuse the antibody core (IgA, IgD, IgE, IgG or IgM) to a further binding protein (e.g., scFv, VL VH, etc.) to, or within, the antibody core, or to fuse multiple antibody portions or to fuse (e.g. two Fab fragments or scFv) to a Heterodimerization-Promoting Domain such as the CH2-CH3 Domain or alternative polypeptides (WO 2005/070966, WO 2006/107786A WO 2006/107617A, WO 2007/046893). Typically, such approaches involve compromises and trade-offs. For example, PCT Publications Nos. WO 2013/174873, WO 2011/133886 and WO 2010/136172 disclose that the use of linkers may cause problems in therapeutic settings, and teaches a tri-specific antibody in which the CL and CH1 Domains are switched from their respective natural positions and the VL and VH Domains have been diversified (WO 2008/027236; WO 2010/108127) to allow them to bind to more than one antigen. Thus, the molecules disclosed in these documents trade binding specificity for the ability to bind additional antigen species. PCT Publications Nos. WO 2013/163427 and WO 2013/119903 disclose modifying the CH2 Domain to contain a fusion protein adduct comprising a binding domain. The document notes that the CH2 Domain likely plays only a minimal role in mediating effector function. PCT Publications Nos. WO 2010/028797, WO2010028796 and WO 2010/028795 disclose recombinant antibodies whose Fc Domains have been replaced with additional VL and VH Domains, so as to form tri-valent binding molecules. PCT Publications Nos. WO 2003/025018 and WO2003012069 disclose recombinant diabodies whose individual chains contain scFv domains. PCT Publications No. WO 2013/006544 discloses multi-valent Fab molecules that are synthesized as a single polypeptide chain and then subjected to proteolysis to yield heterodimeric structures. Thus, the molecules disclosed in these documents trade all or some of the capability of mediating effector function for the ability to bind additional antigen species. PCT Publications Nos. WO 2014/022540, WO 2013/003652, WO 2012/162583, WO 2012/156430, WO 2011/086091, WO 2007/075270, WO 1998/002463, WO 1992/022583 and WO 1991/003493 disclose adding additional Binding Domains or functional groups to an antibody or an antibody portion (e.g., adding a diabody to the antibody's Light Chain, or adding additional VL and VH Domains to the antibody's light and heavy chains, or adding a heterologous fusion protein or chaining multiple Fab Domains to one another). Thus, the molecules disclosed in these documents trade native antibody structure for the ability to bind additional antigen species.

The art has additionally noted the capability to produce diabodies that differ from such natural antibodies in being capable of binding two or more different epitope species (i.e., exhibiting bi-specificity or multispecificity in addition to bi-valency or multi-valency) (see, e.g., Holliger et al. (1993) “‘Diabodies’: Small Bivalent And Bispecific Antibody Fragments,” Proc. Natl. Acad. Sci. (U.S.A.) 90:6444-6448; US 2004/0058400 (Hollinger et al.); US 2004/0220388 (Mertens et al.); Alt et al. (1999) FEBS Lett. 454(1-2):90-94; Lu, D. et al. (2005) “A Fully Human Recombinant IgG-Like Bispecific Antibody To Both The Epidermal Growth Factor Receptor And The Insulin-Like Growth Factor Receptor For Enhanced Antitumor Activity,” J. Biol. Chem. 280(20):19665-19672; WO 02/02781 (Mertens et al.); Olafsen, T. et al. (2004) “Covalent Disulfide-Linked Anti-CEA Diabody Allows Site-Specific Conjugation And Radiolabeling For Tumor Targeting Applications,” Protein Eng Des Sel. 17(1):21-27; Wu, A. et al. (2001) “Multimerization Of A Chimeric Anti-CD20 Single-chain Fv-Fv Fusion Protein Is Mediated Through Variable Domain Exchange,” Protein Engineering 14(2):1025-1033; Asano et al. (2004) “A Diabody For Cancer Immunotherapy And Its Functional Enhancement By Fusion Of Human Fc Domain,” Abstract 3P-683, J. Biochem. 76(8):992; Takemura, S. et al. (2000) “Construction Of A Diabody (Small Recombinant Bispecific Antibody) Using A Refolding System,” Protein Eng. 13(8):583-588; Baeuerle, P. A. et al. (2009) “Bispecific T-Cell Engaging Antibodies For Cancer Therapy,” Cancer Res. 69(12):4941-4944).

The design of a diabody is based on the structure of single-chain Variable Domain fragments (scFv). Such molecules are made by linking light and/or Heavy Chain Variable Domains to one another via a short linking peptide. Bird et al. (1988) (“Single-Chain Antigen-Binding Proteins,” Science 242:423-426) describes an example of linking peptides which bridge approximately 3.5 nm between the carboxy terminus of one Variable Domain and the amino terminus of the other Variable Domain. Linkers of other sequences have been designed and used (Bird et al. (1988) “Single-Chain Antigen-Binding Proteins,” Science 242:423-426). Linkers can in turn be modified for additional functions, such as attachment of drugs or attachment to solid supports. The single-chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

U.S. Pat. No. 7,585,952 and United States Patent Publication No. 2010-0173978 concern scFv molecules that are immunospecific for ErbB2. Bi-specific T cell engagers (“BiTEs”), a type of scFv molecule has been described (WO 05/061547; Baeuerle, P et al. (2008) “BiTE: A New Class Of Antibodies That Recruit T Cells,” Drugs of the Future 33: 137-147; Bargou, et al. 2008) “Tumor Regression in Cancer Patients by Very Low Doses of a T Cell-Engaging Antibody,” Science 321: 974-977). Such molecules are composed of a single polypeptide chain molecule having two Antigen-Binding Domains, one of which immunospecifically binds to a CD3 epitope and the second of which immunospecifically binds to an antigen present on the surface of a target cell.

The provision of non-mono-specific diabodies provides a significant advantage: the capacity to co-ligate and co-localize cells that express different epitopes. Bivalent diabodies thus have wide-ranging applications including therapy and immunodiagnosis. Bi-valency allows for great flexibility in the design and engineering of the diabody in various applications, providing enhanced avidity to multimeric antigens, the cross-linking of differing antigens, and directed targeting to specific cell types relying on the presence of both target antigens. Due to their increased valency, low dissociation rates and rapid clearance from the circulation (for diabodies of small size, at or below ˜50 kDa), diabody molecules known in the art have also shown particular use in the field of tumor imaging (Fitzgerald et al. (1997) “Improved Tumour Targeting By Disulphide Stabilized Diabodies Expressed In Pichia pastoris,” Protein Eng. 10:1221). Of particular importance is the co-ligating of differing cells, for example, the cross-linking of cytotoxic T cells to tumor cells (Staerz et al. (1985) “Hybrid Antibodies Can Target Sites For Attack By T Cells,” Nature 314:628-631, and Holliger et al. (1996) “Specific Killing Of Lymphoma Cells By Cytotoxic T-Cells Mediated By A Bispecific Diabody,” Protein Eng. 9:299-305).

Diabody epitope-binding domains may be directed to a surface determinant of any immune effector cell such as CD3, CD16, CD32, CD64, etc., which are expressed on T lymphocytes, Natural Killer (NK) cells or other mononuclear cells. In many studies, diabody binding to effector cell determinants, e.g., Fcγ receptors (FcγR), was also found to activate the effector cell (Holliger et al. (1996) “Specific Killing Of Lymphoma Cells By Cytotoxic T-Cells Mediated By A Bispecific Diabody,” Protein Eng. 9:299-305; Holliger et al. (1999) “Carcinoembryonic Antigen (CEA)-Specific T-cell Activation In Colon Carcinoma Induced By Anti-CD3×Anti-CEA Bispecific Diabodies And B7×Anti-CEA Bispecific Fusion Proteins,” Cancer Res. 59:2909-2916; WO 2006/113665; WO 2008/157379; WO 2010/080538; WO 2012/018687; WO 2012/162068). Normally, effector cell activation is triggered by the binding of an antigen bound antibody to an effector cell via Fc-FcγR interaction; thus, in this regard, diabody molecules may exhibit Ig-like functionality independent of whether they comprise an Fc Domain (e.g., as assayed in any effector function assay known in the art or exemplified herein (e.g., ADCC assay)). By cross-linking tumor and effector cells, the diabody not only brings the effector cell within the proximity of the tumor cells but leads to effective tumor killing (see e.g., Cao et al. (2003) “Bispecific Antibody Conjugates In Therapeutics,” Adv. Drug. Deliv. Rev. 55:171-197).

For example, U.S. Pat. No. 6,171,586, concerns the production of bi-specific antibodies by proteolytically cleaving two antibodies to obtain their F(ab′)₂ fragments, reducing such fragments under conditions for preventing intermolecular disulfide bond formation, and then mixing the fragments to generate the bi-specific antibody). U.S. Pat. Nos. 6,551,592; 6,994,853 and 8,277,806 and PCT Publications Nos. WO 2012/156430, WO 2002/020039, WO 2000/018806 and WO 1998/003670 concern the production of tri-specific antibodies capable of simultaneously binding to T cells and other antigens on a tumor cell, and, via the Fc portion of the bi-specific antibody, to the Fc receptor of cells possessing such a receptor. PCT Publications Nos. WO 2000/018806, WO 1998/003670 and WO 2006/072152 concern the production of tri-specific antibodies capable of simultaneously binding to T cells and other antigens. United States Patent Publication No. 2008-0057054 discloses bi-specific conjugates specific for a binding element against amyloid beta oligomers and a binding element against transmembrane protein telencephalin. United States Patent Publication No. 2010-0291112 concerns bi-specific and tri-specific single-chain Fv molecules that specifically bind to a one (or two) tumor antigen(s) and an effector cell antigen (such as CD3, CD16 CD32, CD64, etc.).

PCT Publication Nos. WO 1999/042597 and WO 1998/006749 disclose antibody derivatives that comprise human Major Histocompatibility Complex binding domains, with or without bound MHC binding peptides. PCT Publication No. WO 02/072141 concerns multi-specific binding molecules whose on-rates (rates at which they bind to target molecules) and off-rates (rates at which they release target molecules) differ so as to preferentially bind to one target compared to their binding to the other such target molecule. Tri-specific molecules, for example molecules having a monovalent first portion which is an Anti-CD3 or anti-CD28 antibody, and a second portion comprising a divalent immune function exerting moiety which immunospecifically binds to one or more target ligands on a target diseased cell or immune cell.

U.S. Pat. No. 7,695,936 and Patent Publication 2007/0196363 concern bi-specific antibodies that are formed from the heavy chains of two antibodies, one of which possess a protuberance engineered into its heavy chain and the second of which possess a complementary cavity engineered into its heavy chain. The presence of such complementary “knobs” and “holes” is taught to preferentially form bi-specific hetero-antibodies (having one heavy chain of each such antibody) relative to mono-specific homo-antibodies that contain two heavy chains of the same antibody. Various bi-specific hetero-antibodies are proposed, including those that are immunospecific for CD3 and a tumor cell antigen. Various tri-specific hetero-antibodies are also proposed, including some that are immunospecific for CD3, CD8 and CD37 (a transmembrane protein expressed predominantly on B cells that is involved the regulation of T cell proliferation (Robak, T. et al. (2014) “Anti-CD37 Antibodies For Chronic Lymphocytic Leukemia,” Expert Opin. Biol. Ther. 14(5):651-661), however, no mechanism for their production and no disclosure of their structure is provided.

PCT Publication WO2012-162561 concerns bi-specific, tetravalent binding molecules that comprise two polypeptides, each of which comprises two diabody structures, separated by an intervening CH2-CH3 Domain. The document also concerns tetravalent binding molecules composed of four polypeptide chains in which two of the polypeptide chains contain variable light and variable heavy Domains for two antigens, and in which the other two polypeptide chains contain the complementary variable heavy and variable light Domains for the antigens and a terminal CH2-CH3 Domain. The bi-specific, tetravalent binding molecules form through the association of their respective CH2-CH3 Domains. In the four polypeptide chain construct, the “light” chains are not covalently bound to the heavy chains, thus leading to instability (see, Lu, D. et al. (2005) “A Fully Human Recombinant IgG-like Bispecific Antibody To Both The Epidermal Growth Factor Receptor And The Insulin-Like Growth Factor Receptor For Enhanced Antitumor Activity,” J. Biol. Chem. 280(20):19665-19672). The document discloses a third construct in which the chains are altered to provide such covalent bonding, but at the cost of eliminating their bi-specificity (i.e., the molecules are mono-specific). Molecules having specificity for CD2, CD3, CD4, CD8, CD161, a chemokine receptor, CD95, CCR5, etc. are disclosed. A bi-specific molecule capable of binding to both CD3 and CD8 is not disclosed.

However, the above advantages come at salient cost. The formation of such non-mono-specific diabodies requires the successful assembly of two or more distinct and different polypeptides (i.e., such formation requires that the diabodies be formed through the heterodimerization of different polypeptide chain species). This fact is in contrast to mono-specific diabodies, which are formed through the homodimerization of identical polypeptide chains. Because at least two dissimilar polypeptides (i.e., two polypeptide species) must be provided in order to form a non-mono-specific diabody, and because homodimerization of such polypeptides leads to inactive molecules (Takemura, S. et al. (2000) “Construction Of A Diabody (Small Recombinant Bispecific Antibody) Using A Refolding System,” Protein Eng. 13(8):583-588), the production of such polypeptides must be accomplished in such a way as to prevent covalent bonding between polypeptides of the same species (Takemura, S. et al. (2000) “Construction Of A Diabody (Small Recombinant Bispecific Antibody) Using A Refolding System,” Protein Eng. 13(8):583-588). The art has therefore taught the non-covalent association of such polypeptides (see, e.g., Olafsen et al. (2004) “Covalent Disulfide-Linked Anti-CEA Diabody Allows Site-Specific Conjugation And Radiolabeling For Tumor Targeting Applications,” Prot. Engr. Des. Sel. 17:21-27; Asano et al. (2004) “A Diabody For Cancer Immunotherapy And Its Functional Enhancement By Fusion Of Human Fc Domain,” Abstract 3P-683, J. Biochem. 76(8):992; Takemura, S. et al. (2000) “Construction Of A Diabody (Small Recombinant Bispecific Antibody) Using A Refolding System,” Protein Eng. 13(8):583-588; Lu, D. et al. (2005) “A Fully Human Recombinant IgG-Like Bispecific Antibody To Both The Epidermal Growth Factor Receptor And The Insulin-Like Growth Factor Receptor For Enhanced Antitumor Activity,” J. Biol. Chem. 280(20):19665-19672).

However, the art has recognized that bi-specific diabodies composed of non-covalently associated polypeptides are unstable and readily dissociate into non-functional monomers (see, e.g., Lu, D. et al. (2005) “A Fully Human Recombinant IgG-Like Bispecific Antibody To Both The Epidermal Growth Factor Receptor And The Insulin-Like Growth Factor Receptor For Enhanced Antitumor Activity,” J. Biol. Chem. 280(20):19665-19672).

In the face of this challenge, the art has succeeded in developing stable, covalently bonded heterodimeric non-mono-specific diabodies, termed DARTs™ (see, e.g., United States Patent Publications No. 2013-0295121; 2010-0174053 and 2009-0060910; European Patent Publication No. EP 2714079; EP 2601216; EP 2376109; EP 2158221 and PCT Publications No. WO 2012/162068; WO 2012/018687; WO 2010/080538; and Moore, P. A. et al. (2011) “Application Of Dual Affinity Retargeting Molecules To Achieve Optimal Redirected T-Cell Killing Of B-Cell Lymphoma,” Blood 117(17):4542-4551; Veri, M. C. et al. (2010) “Therapeutic Control Of B Cell Activation Via Recruitment Of Fcgamma Receptor IIb (CD32B) Inhibitory Function With A Novel Bispecific Antibody Scaffold,” Arthritis Rheum. 62(7):1933-1943; Johnson, S. et al. (2010) “Effector Cell Recruitment With Novel Fv-Based Dual-Affinity Re-Targeting Protein Leads To Potent Tumor Cytolysis And in vivo B-Cell Depletion,” J. Mol. Biol. 399(3):436-449). Such diabodies comprise two or more covalently complexed polypeptides and involve engineering one or more cysteine residues into each of the employed polypeptide species that permit disulfide bonds to form and thereby covalently bond two polypeptide chains. For example, the addition of a cysteine residue to the C-terminus of such constructs has been shown to allow disulfide bonding between the polypeptide chains, stabilizing the resulting heterodimer without interfering with the binding characteristics of the bivalent molecule.

There are many DART™ embodiments. Each of the two polypeptides of the simplest DART™ embodiment comprises three Domains (FIG. 1). The first polypeptide comprises: (i) a first domain that comprises a binding region of a Light Chain Variable Domain of the a first immunoglobulin (VL1), (ii) a second domain that comprises a binding region of a Heavy Chain Variable Domain of a second immunoglobulin (VH2), and (iii) a third domain that contains a cysteine residue (or a Cysteine-Containing Domain) and a Heterodimerization-Promoting Domain that serves to promote heterodimerization with the second polypeptide chain. The cysteine residue (or a Cysteine-Containing Domain) of the third domain serves to promote the covalent bonding of the first polypeptide chain to the second polypeptide chain of the diabody. The second polypeptide contains: (i) a complementary first domain (a VL2-containing Domain), (ii) a complementary second domain (a VH1-containing Domain) and (iii) a third domain that contains a cysteine residue (or a Cysteine-Containing Domain) and, optionally, a complementary Heterodimerization-Promoting Domain that complexes with the Heterodimerization-Promoting Domain of the first polypeptide chain in order to promote heterodimerization with the first polypeptide chain. The cysteine residue (or a Cysteine-Containing Domain) of the third domain of the second polypeptide chain serves to promote the covalent bonding of the second polypeptide chain to the first polypeptide chain of the diabody. Such molecules are stable, potent and have the ability to simultaneously bind two or more antigens. They are able to promote re-directed T cell mediated killing of cells expressing target antigens.

In one embodiment, the third domains of the first and second polypeptides each contain a cysteine residue, which serves to bind the polypeptides together via a disulfide bond. The third domain of one or both of the polypeptides may additionally possesses the sequence of a CH2-CH3 Domain, such that complexing of the diabody polypeptides forms an Fc Domain that is capable of binding to the Fc receptor of cells (such as B lymphocytes, dendritic cells, Natural Killer cells, macrophages, neutrophils, eosinophils, basophils and mast cells) (FIGS. 2A-2B).

Many variations of such molecules have been described (see, e.g., United States Patent Publications No. 2013-0295121; 2010-0174053 and 2009-0060910; European Patent Publication No. EP 2714079; EP 2601216; EP 2376109; EP 2158221 and PCT Publications No. WO 2012/162068; WO 2012/018687; WO 2010/080538). These Fc-bearing DARTs may comprise three polypeptide chains (e.g., FIG. 2B). The first polypeptide chain of such a diabody contains three domains: (i) a VL1-containing Domain, (ii) a VH2-containing Domain and (iii) a domain containing a cysteine residue (or a Cysteine-Containing Domain) and a Heterodimerization-Promoting Domain, and (iv) a cysteine residue (or a Cysteine-Containing Domain and a CH2-CH3 Domain. The second polypeptide chain of such DART™ contains: (i) a VL2-containing Domain, (ii) a VH1-containing Domain and (iii) a Domain that contains a cysteine residue (or a Cysteine-Containing Domain) and a Heterodimerization-Promoting Domain that promotes heterodimerization with the first polypeptide chain. The cysteine residue (or a Cysteine-Containing Domain) of the third domain of the second polypeptide chain serves to promote the covalent bonding of the second polypeptide chain to the first polypeptide chain of the diabody. The third polypeptide of such DART™ comprises a cysteine residue (or a Cysteine-Containing Domain) and a CH2-CH3 Domain. Thus, the first and second polypeptide chains of such DART™ associate together to form a VL1/VH1 binding site that is capable of binding to the epitope, as well as a VL2/VH2 binding site that is capable of binding to the second epitope. The first and second polypeptides are bonded to one another through a disulfide bond involving cysteine residues in their respective third domains. Notably, the first and third polypeptide chains complex with one another to form an Fc Domain that is stabilized via a disulfide bond. Such diabodies have enhanced potency. Such Fc-bearing DARTs™ may have either of two orientations (Table 1):

TABLE 1

| | | |
|---|---|---|
| First | 3^{rd} Chain | NH₂—CH2—CH3—COOH |
| Orientation | 1^{st} Chain | NH₂-VL1-VH2-Cys-Heterodimer-Promoting |
| | | Domain-CH2—CH3—COOH |
| | 2^{nd} Chain | NH₂-VL2-VH1-Cys-Heterodimer-Promoting |
| | | Domain-COOH |
| Second | 3^{rd} Chain | NH₂—CH2—CH3—COOH |
| Orientation | 1^{st} Chain | NH₂—CH2—CH3-VL1-VH2-Cys-Heterodimer- |
| | | Promoting Domain-COOH |
| | 2^{nd} Chain | NH₂-VL2-VH1-Cys-Heterodimer-Promoting |
| | | Domain-COOH |

Even more complex DART™ diabodies, termed Ig-DART™ (FIGS. 3A-3B) and Fc-DART™ diabodies (FIG. 3C) have been described (WO 2012/018687). Fc-DARTs™ have four polypeptide chains. The first and third polypeptide chains of such a diabody contain three Domains: (i) a VL1-containing Domain, (ii) a VH2-containing Domain and (iii) a Domain containing a CH2-CH3 sequence. The second and fourth polypeptide of the Fc-DART™ contain: (i) a VL2-containing Domain, (ii) a VH1-containing Domain and (iii) a Domain that promotes heterodimerization and covalent bonding with the Fc-DART's™ first polypeptide chain. The third and fourth, and the first and second polypeptide chains may be the same or different so as to permit tetravalent binding that is either mono-specific, bi-specific or tetra-specific. Such more complex DART™ molecules also possess Cysteine-Containing Domains which function to form a covalently bonded complex. Fc-DART™ diabodies contain CH1 and CL Domains.

Alternative constructs are known in the art for applications where a tetravalent molecule is desirable but an Fc is not required including, but not limited to, tetravalent tandem antibodies, also referred to as “TandAbs” (see, e.g. United States Patent Publications Nos. 2005-0079170, 2007-0031436, 2010-0099853, 2011-020667 2013-0189263; European Patent Publication Nos. EP 1078004, EP 2371866, EP 2361936 and EP 1293514; PCT Publications Nos. WO 1999/057150, WO 2003/025018, and WO 2013/013700) which are formed by the homo-dimerization of two identical chains each possessing a VH1, VL2, VH2, and VL2 Domain.

However, despite all prior advances, a need remains for compositions that could provide improved therapeutic value to patients suffering from cancer or other diseases and conditions. The present invention is directed to this and other goals.

### SUMMARY OF THE INVENTION

The present invention relates to Tri-Specific Binding Molecules, which are multi-chain polypeptide molecules that possess three Binding Domains and are thus capable of mediating coordinated binding to three epitopes. The Tri-Specific Binding Molecule is preferably characterized in possessing binding domains that permit it to immunospecifically bind to: (1) an epitope of a first Cancer Antigen, (2) an epitope of a second Cancer Antigen, and (3) an epitope of a molecule that is expressed on the surface of an immune system effector cell, and are thus capable of localizing an immune system effector cell to a cell that expresses a Cancer Antigen, so as to thereby facilitate the killing of such cancer cell.

In detail, the invention provides a Tri-Specific Binding Molecule capable of immunospecifically binding to three different epitopes, said Epitopes being Epitope I, Epitope II, and Epitope III, wherein two of three epitopes are epitopes of Cancer Antigen(s), and the third of said epitopes is an epitope of an Effector Cell Antigen.

The invention particularly concerns the embodiment of such Tri-Specific Binding Molecule wherein the molecule comprises four different polypeptide chains covalently complexed together and comprises:
(I) an Antigen-Binding Domain I that is capable of immunospecifically binding to an Epitope I present on a first antigen, and an Antigen-Binding Domain II that is capable of immunospecifically binding to an Epitope II present on a second antigen, wherein the Antigen-Binding Domain I and the Antigen-Binding Domain II are both Diabody-Type Binding Domains;
(II) an Antigen-Binding Domain III that is capable of immunospecifically binding to an Epitope III present on a third antigen; and
(III) an Fc Domain that is formed by the complexing of two CH2-CH3 Domains to one another;
   wherein one of Epitope I, Epitope II or Epitope III is an epitope of an Effector Cell Antigen, a second of Epitope I, Epitope II or Epitope III is an epitope of a first Cancer Antigen, and the third of Epitope I, Epitope II or Epitope III is an epitope of a second Cancer Antigen, and wherein the Antigen-Binding Domains I, II and III of the Binding Molecules mediate coordinated binding of an immune system effector cell expressing the Effector Cell Antigen and a cancer cell expressing the first and second Cancer Antigens.

The invention particularly concerns the embodiment of such Tri-Specific Binding Molecules wherein the Fc Domain is capable of binding to an Fc Receptor arrayed on the surface of a cell.

The invention additionally concerns the embodiment of such Tri-Specific Binding Molecules wherein the Effector Cell Antigen is arrayed on the surface of an effector cell and wherein the Cancer Antigens are arrayed on the surface of a cancer cell, and wherein the immunospecific binding is sufficient to co-localize the Effector Cell Antigen, and the Cancer Antigens, thereby facilitating the activation of the effector cell against the cancer cell.

The invention additionally concerns the embodiment of such Tri-Specific Binding Molecules wherein the Effector Cell Antigen is selected from the group consisting of: CD2, CD3, CD16, CD19, CD20, CD22, CD32B, CD64, the B cell Receptor (BCR), the T cell Receptor (TCR), and the NKG2D Receptor.

The invention additionally concerns the embodiment of such Tri-Specific Binding Molecules wherein the first and second Cancer Antigens are independently selected from the group consisting of: colon cancer antigen 19.9; a gastric cancer mucin; antigen 4.2; glycoprotein A33 (gpA33); ADAM-9; gastric cancer antigen AH6; ALCAM; malignant human lymphocyte antigen APO-1; cancer antigen B1; B7-H3; beta-catenin; blood group ALe^{b}/Le^{y}; Burkitt's lymphoma antigen-38.13, colonic adenocarcinoma antigen C14; ovarian carcinoma antigen CA125; Carboxypeptidase M; CD5; CD19; CD20; CD22; CD23; CD25; CD27; CD30; CD33; CD36; CD45; CD46; CD52; CD79a/CD79b; CD103; CD317; CDK4; carcinoembryonic antigen (CEA); CEACAM5; CEACAM6; C017-1A; CO-43 (blood group Le^{b}); CO-514 (blood group Le^{a}); CTA-1; CTLA4; Cytokeratin 8; antigen D1.1; antigen D₁56-22; DR5; E₁ series (blood group B); EGFR (Epidermal Growth Factor Receptor); Ephrin receptor A2 (EphA2); ErbB1; ErbB3; ErbB4; GAGE-1; GAGE-2; GD2/GD3/GM2; lung adenocarcinoma antigen F3; antigen FC10.2; G49, ganglioside GD2; ganglioside GD3; ganglioside GM2; ganglioside GM3; G_{D2}; G_{D3}; GICA 19-9; G_{M2}; gp100; human leukemia T cell antigen Gp37; melanoma antigen gp75; gpA33; HER2 antigen (p185^{HER2}); human milk fat globule antigen (HMFG); human papillomavirus-E6/human papillomavirus-E7; high molecular weight melanoma antigen (HMW-MAA); I antigen (differentiation antigen) I(Ma); Integrin Alpha-V-Beta-6 Integrinβ6 (ITGB6); Interleukin-13; Receptor α2 (IL13Rα2); JAM-3; KID3; KID31; KS 1/4 pan-carcinoma antigen; human lung carcinoma antigens L6 and L20; LEA; LUCA-2; M1:22:25:8; M18; M39; MAGE-1; MAGE-3; MART; MUC-1; MUM-1; Myl; N-acetylglucosaminyltransferase; neoglycoprotein; NS-10; OFA-1; OFA-2; Oncostatin M; p15; melanoma-associated antigen p97; polymorphic epithelial mucin (PEM); polymorphic epithelial mucin antigen (PEMA); PIPA; prostate-specific antigen (PSA); prostate-specific membrane antigen (PSMA); prostatic acid phosphate; R₂₄; ROR1; sphingolipids; SSEA-1; SSEA-3; SSEA-4; sTn; T cell receptor derived peptide; T₅A₇; TAG-72; TL5 (blood group A); TNF-α receptor; TNF-β receptor; TNF-γ receptor; TRA-1-85 (blood group H); Transferrin Receptor; tumor-specific transplantation antigen (TSTA), oncofetal antigen-alpha-fetoprotein (AFP); VEGF; VEGFR, VEP8; VEP9; VIM-D5; and Y hapten, Le^{y}.

The invention additionally concerns the embodiment of such Tri-Specific Binding Molecules wherein the first and second Cancer Antigens are selected from the group consisting of: CD2, CD317, CEACAM5, CEACAM6, DR5, EphA2, gpA33, Her2, B7-H3; EGF, EGFR, VEGF and VEGFR.

The invention additionally concerns the embodiment of such Tri-Specific Binding Molecules wherein the Non-Diabody-Type Binding Domain III comprises the Fab-Type Binding Domain (VL_{III}/VH_{III}) that is capable of immunospecifically binding to an Epitope III, wherein the molecule comprises:
(A) a first polypeptide chain that comprises in the N-terminus to C-terminus direction:
   (1) a light chain variable Domain of an immunoglobulin capable of binding to a first of the three epitopes (VL_{I});
   (2) a heavy chain variable Domain of an immunoglobulin capable of binding to a second of the three epitopes (VH_{II});
   (3) a Heterodimer-Promoting Domain; and
   (4) CH2 and CH3 Domains of an IgG;
(B) a second polypeptide chain that comprises, in the N-terminus to C-terminus direction:
   (1) a light chain variable Domain of an immunoglobulin capable of binding to the second of the three epitopes (VL_{II});
   (2) a heavy chain variable Domain of an immunoglobulin capable of binding to the first of the three epitopes (VH_{I}); and
   (3) a complementary Heterodimer-Promoting Domain;
(C) a third polypeptide chain that comprises, in the N-terminus to C-terminus direction:
   (1) a heavy chain variable Domain of an immunoglobulin capable of binding to a third of the three epitopes (VH_{III}); and
   (2) a CH1 Domain, a Hinge Domain, and a CH2-CH3 Domain of an IgG;
and
(D) a fourth polypeptide chain that comprises, in the N-terminus to C-terminus direction:
   (1) a light chain variable Domain of an immunoglobulin capable of binding to the third of the three epitopes (VL_{III}); and
   (2) a light chain constant Domain (CL);
wherein:
   (i) the VL_{I} and VH₁ Domains associate to form a Domain capable of binding the first epitope;
   (ii) the VL_{II} and VH_{II} Domains associate to form a Domain capable of binding the second epitope;
   (iii) the VL_{III} and VH_{III} Domains associate to form a Domain capable of binding the third epitope;
   (iv) the CH2-CH3 Domain of the first polypeptide chain and the CH2-CH3 Domain of the third polypeptide chain associate to form an Fc Domain;
   (v) the first and second polypeptide chains are covalently bonded to one another;
   (vi) the first and third polypeptide chains are covalently bonded to one another; and
   (vii) the third and fourth polypeptide chains are covalently bonded to one another.

The invention additionally concerns the embodiment of such Tri-Specific Binding Molecules wherein:
(A) the Heterodimer-Promoting Domain is an E-coil and the complementary Heterodimer-Promoting Domain is a K-coil; or
(B) the Heterodimer-Promoting Domain is a K-coil and the complementary Heterodimer-Promoting Domain is an E-coil.

The invention additionally concerns the embodiment of such Tri-Specific Binding Molecules wherein:
(A) the CH2-CH3 Domains of the first and third polypeptide chains each have the sequence of SEQ ID NO:1, such that the Fc Domain formed from their association exhibits normal FcγR-mediated effector function; or
(B) the CH2-CH3 Domain of the first and third polypeptide chains comprise at least one amino acid substitution, relative to the sequence of SEQ ID NO:1, such that the Fc Domain formed from their association exhibits altered FcγR-mediated effector function.

The invention additionally concerns the embodiment of such Tri-Specific Binding Molecules wherein the at least one amino acid substitution comprises at least one amino acid substitution selected from the group consisting of: L235V, F243L, R292P, Y300L, V305I, and P396L, wherein the numbering is that of the EU index as in Kabat.

The invention additionally concerns the embodiment of such Tri-Specific Binding Molecules wherein the at least one amino acid substitution comprises:
(A) at least one substitution selected from the group consisting of F243L, R292P, Y300L, V305I, and P396L;
(B) at least two substitutions selected from the group consisting of:
   (1) F243L and P396L;
   (2) F243L and R292P; and
   (3) R292P and V305I;
(C) at least three substitutions selected from the group consisting of:
   (1) F243L, R292P and Y300L;
   (2) F243L, R292P and V305I;
   (3) F243L, R292P and P396L; and
   (4) R292P, V305I and P396L;
(D) at least four substitutions selected from the group consisting of:
   (1) F243L, R292P, Y300L and P396L; and
   (2) F243L, R292P, V305I and P396L;
or
(E) at least the five substitutions selected from the group consisting of:
   (1) F243L, R292P, Y300L, V305I and P396L; and
   (2) L235V, F243L, R292P, Y300L and P396L.

The invention additionally concerns the embodiment of such Tri-Specific Binding Molecules wherein the CH2-CH3 Domain of the first and third polypeptide chains differ from one another and have an amino acid sequence selected from the group consisting of SEQ ID NO:52 and SEQ ID NO:53.

The invention additionally concerns the embodiment of such Tri-Specific Binding Molecules wherein:
(A) the Epitope I, Epitope II and Epitope III are, respectively, an epitope of the first Cancer Antigen, an epitope of the second Cancer Antigen and an epitope of the Effector Cell Antigen;
(B) the Epitope I, Epitope II and Epitope III are, respectively, an epitope of the first Cancer Antigen, an epitope of the Effector Cell Antigen and an epitope of the second Cancer Antigen;
(C) the Epitope I, Epitope II and Epitope III are, respectively, an epitope of second Cancer Antigen, an epitope of the first Cancer Antigen, and an epitope of the Effector Cell Antigen;
(D) the Epitope I, Epitope II and Epitope III are, respectively, an epitope of the second Cancer Antigen, an epitope of the Effector Cell Antigen and an epitope of the first Cancer Antigen;
(E) the Epitope I, Epitope II and Epitope III are, respectively, an epitope of the Effector Cell Antigen, an epitope of the first Cancer Antigen, and an epitope of the second Cancer Antigen;
and
(F) the Epitope I, Epitope II and Epitope III are, respectively, an epitope of the Effector Cell Antigen, an epitope of second Cancer Antigen, and an epitope of the first Cancer Antigen.

The invention additionally concerns the embodiment of such Tri-Specific Binding Molecules wherein:
(A) the epitope of an Effector Cell Antigen is a CD2 epitope recognized by antibody Lo-CD2a;
(B) the epitope of an Effector Cell Antigen is a CD3 epitope recognized by antibody OKT3, M291, YTH12.5, Anti-CD3 mAb 1 or Anti-CD3 mAb 2;
(C) the epitope of an Effector Cell Antigen is a CD16 epitope recognized by antibody 3G8 or A9;
(D) the epitope of an Effector Cell Antigen is a CD19 epitope recognized by antibody MD1342, MEDI-551, blinatumomab or HD37;
(E) the epitope of an Effector Cell Antigen is a CD20 epitope recognized by antibody rituximab, ibritumomab, ofatumumab, and tositumomab;
(F) the epitope of an Effector Cell Antigen is a CD22 epitope recognized by antibody epratuzumab;
(G) the epitope of an Effector Cell Antigen is a CD32B epitope recognized by antibody CD32B mAb 1;
(H) the epitope of an Effector Cell Antigen is a CD64 epitope recognized by antibody CD64 mAb 1;
(I) the epitope of an Effector Cell Antigen is a BCR/CD79 epitope recognized by antibody CD79 mAb 1;
(J) the epitope of an Effector Cell Antigen is a TCR epitope recognized by antibody BMA 031;
or
(K) the epitope of an Effector Cell Antigen is a NKG2D Receptor epitope recognized by antibody KYK-2.0.

The invention additionally concerns a pharmaceutical composition that comprises any of the above-described Tri-Specific Binding Molecules, and a pharmaceutically acceptable carrier, excipient or diluent.

The invention additionally concerns the embodiment of such pharmaceutical composition or of any such Tri-Specific Binding Molecules wherein the Tri-Specific Binding Molecule is used in the treatment of cancer.

The invention additionally concerns the embodiment of such pharmaceutical compositions or such Tri-Specific Binding Molecules wherein the cancer is characterized by the presence of a cancer cell selected from the group consisting of a cell of: an adrenal gland tumor, an AIDS-associated cancer, an alveolar soft part sarcoma, an astrocytic tumor, bladder cancer, bone cancer, a brain and spinal cord cancer, a metastatic brain tumor, a breast cancer, a carotid body tumors, a cervical cancer, a chondrosarcoma, a chordoma, a chromophobe renal cell carcinoma, a clear cell carcinoma, a colon cancer, a colorectal cancer, a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, gastric cancer, a gestational trophoblastic disease, a germ cell tumor, a head and neck cancer, hepatocellular carcinoma, an islet cell tumor, a Kaposi's Sarcoma, a kidney cancer, a leukemia, a lipoma/benign lipomatous tumor, a liposarcoma/malignant lipomatous tumor, a liver cancer, a lymphoma, a lung cancer, a medulloblastoma, a melanoma, a meningioma, a multiple endocrine neoplasia, a multiple myeloma, a myelodysplastic syndrome, a neuroblastoma, a neuroendocrine tumors, an ovarian cancer, a pancreatic cancer, a papillary thyroid carcinoma, a parathyroid tumor, a pediatric cancer, a peripheral nerve sheath tumor, a phaeochromocytoma, a pituitary tumor, a prostate cancer, a posterious uveal melanoma, a rare hematologic disorder, a renal metastatic cancer, a rhabdoid tumor, a rhabdomysarcoma, a sarcoma, a skin cancer, a soft-tissue sarcoma, a squamous cell cancer, a stomach cancer, a synovial sarcoma, a testicular cancer, a thymic carcinoma, a thymoma, a thyroid metastatic cancer, and a uterine cancer.

The invention additionally concerns the embodiment of such pharmaceutical compositions or such Tri-Specific Binding Molecules wherein the cancer is acolorectal cancer, hepatocellular carcinoma, glioma, kidney cancer, breast cancer, multiple myeloma, bladder cancer, neuroblastoma; sarcoma, non-Hodgkin's lymphoma, non-small cell lung cancer, ovarian cancer, pancreatic cancer or a rectal cancer.

The invention additionally concerns the embodiment of such pharmaceutical compositions or such Tri-Specific Binding Molecules the cancer is acute myeloid leukemia (AML), chronic myelogenous leukemia (CML), acute B lymphoblastic leukemia (B-ALL), chronic lymphocytic leukemia (CLL), hairy cell leukemia (HCL), blastic plasmacytoid dendritic cell neoplasm (BPDCN), non-Hodgkin's lymphomas (NHL), including mantel cell leukemia (MCL), and small lymphocytic lymphoma (SLL), Hodgkin's lymphoma, systemic mastocytosis, or Burkitt's lymphoma.

### BRIEF DESCRIPTION OF THE DRAWINGS

FIGS. 1A-1B show diagrammatic representation of the Domains of DART™ diabodies. FIG. 1A shows a diagrammatic representation of the Domains of a basic DART™ diabody. FIG. 1B provides a schematic of a covalently bonded diabody composed of two polypeptide chains, each having a Heterodimer-Promoting Domain VL and VH domains that recognize the same epitope are shown using the same shading.

FIGS. 2A-2B provide a schematic of covalently bonded diabodies composed of two polypeptide chains, each having a CH2 and CH3 Domain (FIG. 2A) or in which only one has a CH2 and CH3 Domain (FIG. 2B), such that the associated chains form an Fc Domain that comprises all or part of a naturally occurring Fc Domain. VL and VH domains that recognize the same epitope are shown using the same shading.

FIGS. 3A-3C provide schematics showing tetravalent diabodies composed of two pairs of polypeptide chains. The pairs are different, thus resulting in a bi-specific molecule that is bivalent with respect to each of two epitopes, in which one is an epitope of DR5 and the other is an epitope of a molecule present on the surface of an effector cell. One polypeptide of each pair possesses a CH2 and CH3 Domain, such that the associated chains form an Fc Domain that comprises all or part of a naturally occurring Fc Domain. VL and VH domains that recognize the same epitope are shown using the same shading. Only one pair of epitopes (shown with the same shading) is capable of binding to DR5. FIG. 3A shows an Ig diabody. FIG. 3B shows an Ig diabody, which contains E-coil and K-coil heterodimer-promoting domains. FIG. 3C, shows an Fc-DART™ diabody that contains antibody CH1 and CL domains. The notation “VL1” and “VH1” denote respectively, the Variable Light Chain Domain and Variable Heavy Chain Domain that bind the “first” epitope. Similarly, the notation “VL2” and “VH2” denote respectively, the Variable Light Chain Domain and Variable Heavy Chain Domain that bind the “second” epitope.

FIGS. 4A-4G provide a diagrammatic representation of the Domains of preferred Tri-Specific Binding Molecules of the present invention. The Figures illustrate schematically the order and orientation of the Domains of embodiments of the preferred Tri-Specific Binding Molecules of the present invention. FIGS. 4A, 4B and 4G illustrate embodiments in which the Tri-Specific Binding Molecule is composed of four polypeptide chains. FIGS. 4C, 4D, 4E and 4F illustrate embodiments in which the binding molecule is composed of three polypeptide chains. The molecule may possess Hinge and/or CL domains (FIGS. 4A, 4B, 4C, 4E) or may contain an alternative linker peptide (FIG. 4D, 4F, 4G).

FIGS. 5A-5E provide a diagrammatic representation of the Domains of an alternative embodiment of the Tri-Specific Binding Molecules of the present invention, in which the Effector Cell-Binding Domain is composed of an Effector Cell Receptor-Type Binding Domain rather than a Diabody-Type Binding Domain or a Fab-Type Binding Domain. FIGS. 5A and 5B illustrate embodiments in which the Tri-Specific Binding Molecule is composed of four polypeptide chains. FIG. 5C and FIG. 5E illustrate an embodiment in which the binding molecule is composed of three polypeptide chains. FIG. 5D illustrates an embodiment in which the binding molecule is composed of five polypeptide chains. The molecule may possess Hinge and/or CL domains or may contain alternative linker peptides.

FIG. 6 shows the ability of anti-human DR5 monoclonal antibodies DR5 mAb 1 and DR5 mAb 2 to bind to human DR5 and to the DR5 of cynomolgus monkey.

FIG. 7, Panels A-H, show the kinetics of binding of DR5 mAb 1 (Panels A and E), DR5 mAb 2 (Panels B and F), DR5 mAb 3 (Panels C and G) and DR5 mAb 4 (Panels D and H) for human DR 5 (Panels A-D) and for cynomolgus monkey DR5 (Panels E-H).

FIG. 8 shows the unexpected superiority of DR5 mAb 1 and DR5 mAb 2. Superiority was assessed by comparing the ability of DR5×CD3 diabodies having the VL and VH Domains of DR5 mAb 1, DR5 mAb 2, DR5 mAb 3, or DR5 mAb 4, to mediate the cytotoxicity of A549 adenocarcinomic human alveolar basal epithelial tumor cells.

FIGS. 9A-9C demonstrate the synergistic enhancement in target cell binding that is attained when both of the two Cancer Antigen-Binding Domains of a Tri-Specific Binding Molecule of the present invention are able to bind to a target cell. FIG. 9A shows the binding obtained when trispecific molecules: EphA2 mAb 1×CD3 mAb 2×DR5 mAb 1; EphA2 mAb 1×CD3 mAb 2×gpA33 mAb 1; and gpA33 mAb 1×CD3 mAb 2×DR5 mAb 1 are incubated in the presence of EphA2-expressing CHO cells. FIG. 9B shows the binding obtained when such trispecific molecules are incubated in the presence of DR5-expressing CHO cells. FIG. 9C shows the binding obtained when such trispecific molecules are incubated in the presence of DU145 human prostate cells that express EphA2 and DR5, but not gpA33.

FIGS. 10A-10C demonstrate the synergistic enhancement in target cell cytotoxicity that is attained when both of the two Cancer Antigen-Binding Domains of a Tri-Specific Binding Molecule of the present invention are able to bind to a target cell. FIG. 10A shows the percent cytotoxicity obtained by incubating trispecific molecules: EphA2 mAb 1×CD3 mAb 2×DR5 mAb 1; EphA2 mAb 1×CD3 mAb 2×gpA33 mAb 1; and gpA33 mAb 1×CD3 mAb 2×DR5 mAb 1 are incubated in the presence of EphA2-expressing CHO cells and cytotoxic lymphocytes. FIG. 10B shows the percent cytotoxicity obtained when such trispecific molecules are incubated in the presence of DR5-expressing CHO cells and cytotoxic lymphocytes. FIG. 10C shows the cytotoxicity obtained when such trispecific molecules are incubated in the presence of DU145 human prostate cells and cytotoxic lymphocytes. DU145 cells express EphA2 and DR5, but not gpA33. Cytotoxicity was measured by the increase in luminescence caused by the release of luciferase upon cell lysis.

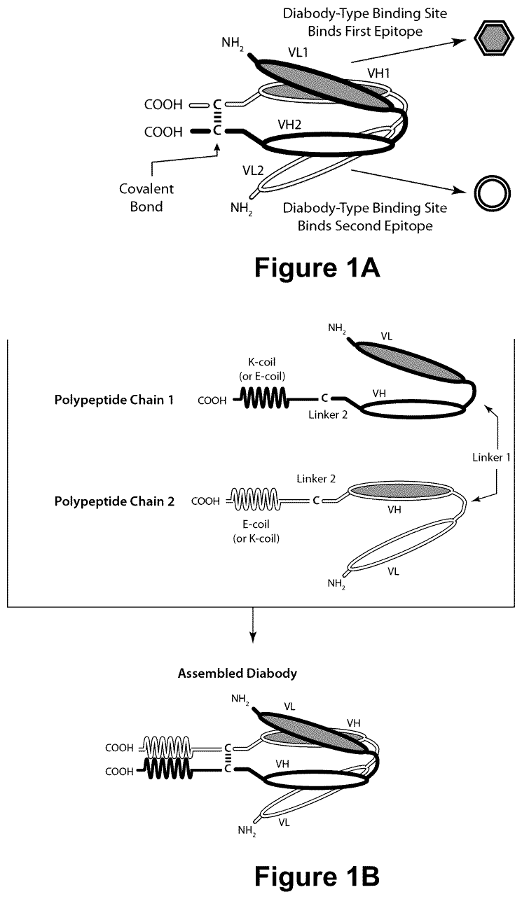

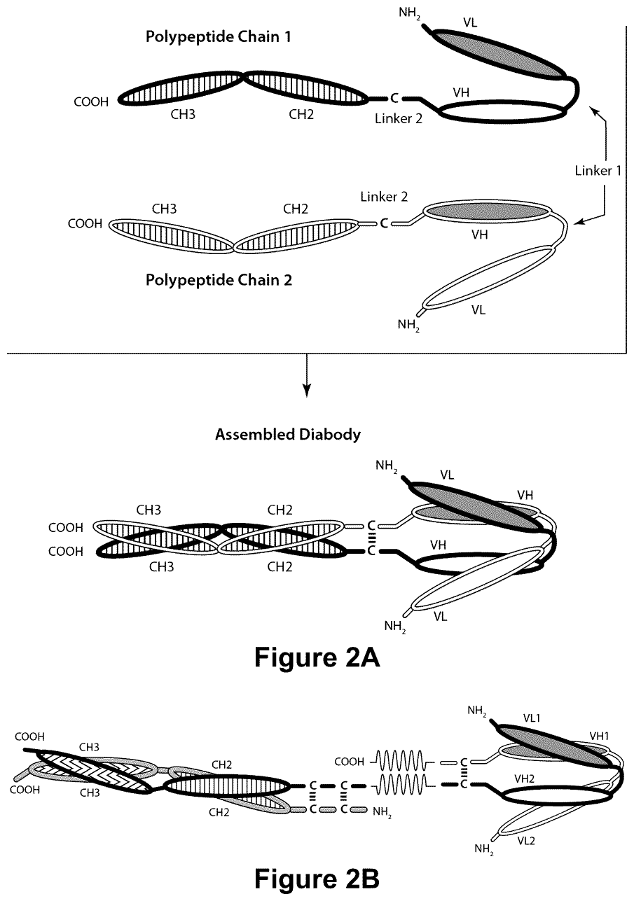

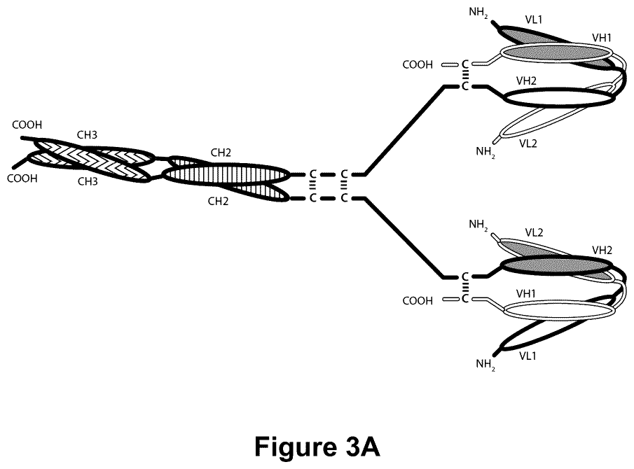

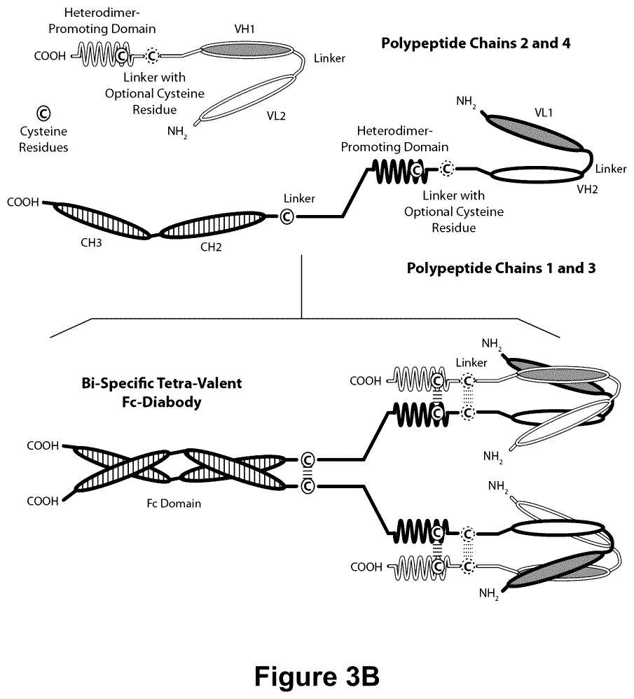

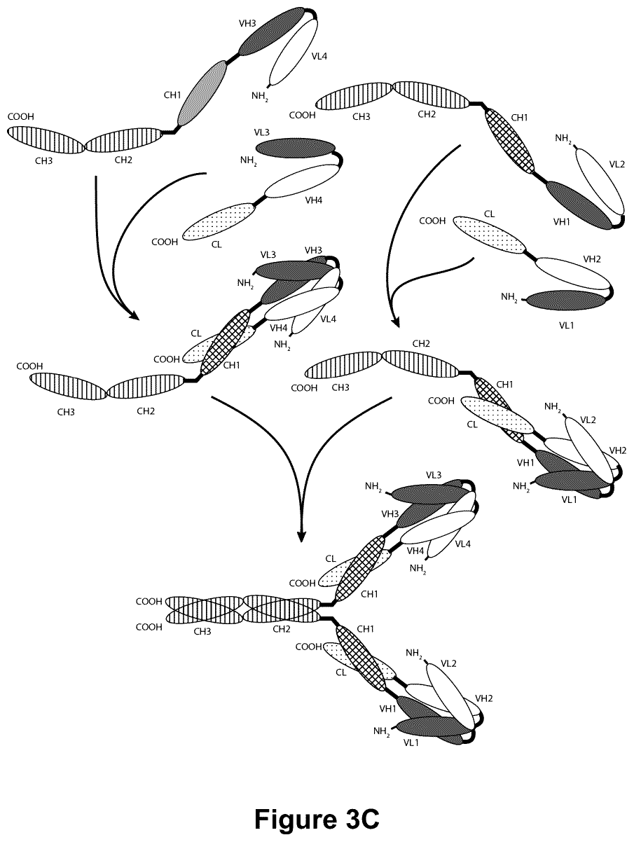

### DETAILED DESCRIPTION OF THE INVENTION

The present invention relates to Tri-Specific Binding Molecules, which are multi-chain polypeptide molecules that possess three Binding Domains and are thus capable of mediating coordinated binding to three epitopes. The Tri-Specific Binding Molecule is preferably characterized in possessing binding domains that permit it to immunospecifically bind to: (1) an epitope of a first Cancer Antigen, (2) an epitope of a second Cancer Antigen, and (3) an epitope of a molecule that is expressed on the surface of an immune system effector cell, and are thus capable of localizing an immune system effector cell to a cell that expresses a Cancer Antigen, so as to thereby facilitate the killing of such cancer cell.

The Tri-Specific Binding Molecules of the present invention may include Epitope-Binding Domains of humanized, chimeric or caninized derivatives of the above-discussed antibodies, for example, DR5 mAb 1 or DR5 mAb 2.

### I. General Techniques and General Definitions

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, MOLECULAR CLONING: A LABORATORY MANUAL, Third Edition (Sambrook et al. Eds., 2001) Cold Spring Harbor Press, Cold Spring Harbor, NY; OLIGONUCLEOTIDE SYNTHESIS: METHODS AND APPLICATIONS (Methods in Molecular Biology), Herdewijn, P., Ed., Humana Press, Totowa, NJ; OLIGONUCLEOTIDE SYNTHESIS (Gait, M. J., Ed., 1984); METHODS IN MOLECULAR BIOLOGY, Humana Press, Totowa, NJ; CELL BIOLOGY: A LABORATORY NOTEBOOK (Cellis, J. E., Ed., 1998) Academic Press, New York, NY; ANIMAL CELL CULTURE (Freshney, R. I., Ed., 1987); INTRODUCTION TO CELL AND TISSUE CULTURE (Mather, J. P. and Roberts, P. E., Eds., 1998) Plenum Press, New York, NY; CELL AND TISSUE CULTURE: LABORATORY PROCEDURES (Doyle, A. et al., Eds., 1993-8) John Wiley and Sons, Hoboken, NJ; METHODS IN ENZYMOLOGY (Academic Press, Inc.) New York, NY; WEIR'S HANDBOOK OF EXPERIMENTAL IMMUNOLOGY (Herzenberg, L. A. et al. Eds. 1997) Wiley-Blackwell Publishers, New York, NY; GENE TRANSFER VECTORS FOR MAMMALIAN CELLS (Miller, J. M. et al. Eds., 1987) Cold Spring Harbor Press, Cold Spring Harbor, NY; CURRENT PROTOCOLS IN MOLECULAR BIOLOGY (Ausubel, F. M. et al., Eds., 1987) Greene Pub. Associates, New York, NY; PCR: THE POLYMERASE CHAIN REACTION, (Mullis, K. et al., Eds., 1994) Birkhauser, Boston MA; CURRENT PROTOCOLS IN IMMUNOLOGY (Coligan, J. E. et al., eds., 1991) John Wiley and Sons, Hoboken, NJ; SHORT PROTOCOLS IN MOLECULAR BIOLOGY (John Wiley and Sons, 1999) Hoboken, NJ; IMMUNOBIOLOGY 7 (Janeway, C. A. et al. 2007) Garland Science, London, UK; Antibodies (P. Finch, 1997) Stride Publications, Devoran, UK; ANTIBODIES: A PRACTICAL APPROACH (D. Catty., ed., 1989) Oxford University Press, USA, New York NY); MONOCLONAL ANTIBODIES: A PRACTICAL APPROACH (Shepherd, P. et al. Eds., 2000) Oxford University Press, USA, New York NY; USING ANTIBODIES: A LABORATORY MANUAL (Harlow, E. et al. Eds., 1998) Cold Spring Harbor Laboratory Press, Cold Spring Harbor, NY; THE ANTIBODIES (Zanetti, M. et al. Eds. 1995) Harwood Academic Publishers, London, UK); and DEVITA, HELLMAN, AND ROSENBERG'S CANCER: PRINCIPLES & PRACTICE OF ONCOLOGY, EIGHTH EDITION, DeVita, V. et al. Eds. 2008, Lippincott Williams & Wilkins, Philadelphia, PA.

### II. Preferred Tri-Specific Binding Molecules of the Present Invention

### A. Binding Capabilities

The preferred Tri-Specific Binding Molecules of the present invention are able to coordinately and simultaneously bind to three different epitopes. The preferred Tri-Specific Binding Molecules of the present invention comprise:
(I) a “Binding Domain I” that is capable of immunospecifically binding to an “Epitope I” present on a first antigen, and a “Binding Domain II” that is capable of immunospecifically binding to an “Epitope II” present on a second antigen, wherein said Binding Domain I and said Binding Domain II are both “Diabody-Type Binding Domains;”
(II) a “Binding Domain III” that is capable of immunospecifically binding to an “Epitope III” present on a third antigen; and
(III) an Fc Domain that is formed by the complexing of two CH2-CH3 Domains to one another;
   wherein:
(A) one of Epitope I, Epitope II or Epitope III is an epitope of a first “Cancer Antigen” Cancer Antigen;
(B) a second of Epitope I, Epitope II or Epitope III is an epitope of a second Cancer Antigen; and
(C) the third of Epitope I, Epitope II or Epitope III is an epitope of a molecule expressed on the surface of an immune system effector cell (“Effector Cell Antigen”);
   and wherein the Binding Domains I, II and III of the binding molecules mediate coordinated binding of the immune system effector cell and a cell expressing both the first and second Cancer Antigens to thereby co-localize such cells.

Diabody Epitope-Binding Domains may also be directed to a surface determinant of a B cell, such as CD19, CD20, CD22, CD30, CD37, CD40, and CD74 (Moore, P. A. et al. (2011) “Application Of Dual Affinity Retargeting Molecules To Achieve Optimal Redirected T-Cell Killing Of B-Cell Lymphoma,” Blood 117(17):4542-4551; Cheson, B. D. et al. (2008) “Monoclonal Antibody Therapy For B-Cell Non-Hodgkin's Lymphoma,” N. Engl. J. Med. 359(6):613-626; Castillo, J. et al. (2008) “Newer monoclonal antibodies for hematological malignancies,” Exp. Hematol. 36(7):755-768. In many studies, diabody binding to effector cell determinants, e.g., Fcγ receptors (FcγR), was also found to activate the effector cell (Holliger et al. (1996) “Specific Killing Of Lymphoma Cells By Cytotoxic T-Cells Mediated By A Bi-specific Diabody,” Protein Eng. 9:299-305; Holliger et al. (1999) “Carcinoembryonic Antigen (CEA)-Specific T-Cell Activation In Colon Carcinoma Induced By Anti-CD3×Anti-CEA Bi-specific Diabodies And B7×Anti-CEA Bi-specific Fusion Proteins,” Cancer Res. 59:2909-2916; WO 2006/113665; WO 2008/157379; WO 2010/080538; WO 2012/018687; WO 2012/162068). Normally, effector cell activation is triggered by the binding of an antigen bound antibody to an effector cell via Fc-FcγR interaction; thus, in this regard, diabody molecules may exhibit Ig-like functionality independent of whether they comprise an Fc Domain (e.g., as assayed in any effector function assay known in the art or exemplified herein (e.g., ADCC assay)). By cross-linking tumor and effector cells, the diabody not only brings the effector cell within the proximity of the tumor cells but leads to effective tumor killing (see e.g., Cao et al. (2003) “Bi-specific Antibody Conjugates In Therapeutics,” Adv. Drug. Deliv. Rev. 55:171-197).

Although such Tri-Specific Binding Molecules are particularly preferred, the invention additionally specifically contemplates Tri-Specific Binding Molecules that comprise any combination of Binding Domains sufficient to produce a molecule having three binding specificities, of which two are binding specificities directed against Cancer Antigens, and one is a binding specificity directed against an Effector Cell Antigen. Thus, for example, the invention contemplates: a Tri-Specific Binding Molecule that comprises three Fab-Type Binding Domains, a Tri-Specific Binding Molecule that comprises one bivalent, bi-specific antibody domain (formed for example, by complexing two different light chains and two different heavy chains) and one Fab-Type Binding Domain, a Tri-Specific Binding Molecule that comprises two bivalent, bi-specific antibody domains (formed for example, by complexing four different light chains and two different heavy chains), but in which one of antibody domains has been rendered inactive, etc.

The terms “polypeptide,” “polypeptide chain,” and “peptide” are used interchangeably herein to refer to polymers of amino acids of any length, but especially lengths greater than 3, 5, 10, 15, 20 or 25 amino acid residues, in which two, and more preferably all, amino acid residues are joined via an amide (peptide) bond (—NH—C(O)—). The polymer may however be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. The polypeptides of this invention can occur as single-chains or as complexed chains.

A “Diabody-Type Binding Domain” is the Epitope-Binding Domain of a diabody, and especially, a DART® diabody. The terms “diabody” and “DART® diabody” has been discussed above, and refers to a molecule that comprises at least two polypeptide chains that preferably complex with one another through a covalent interaction to form at least two epitope binding sites, which may recognize the same or different epitopes. Two of the polypeptide chains of a diabody or DART® diabody each comprise immunoglobulin Light Chain Variable Region and an immunoglobulin Heavy Chain Variable Region, but these regions do not interact to form an epitope binding site (i.e., they are not mutually “complementary”). Rather, the immunoglobulin Heavy Chain Variable Region of one (e.g., the first) of the diabody, or DART® diabody, chains interacts with the immunoglobulin Light Chain Variable Region of a different (e.g., the second) diabody or, DART® diabody, polypeptide chain to form an epitope binding site. Similarly, the immunoglobulin Light Chain Variable Region of one (e.g., the first) of the diabody, or DART® diabody, polypeptide chains interacts with the immunoglobulin Heavy Chain Variable Region of a different (e.g., the second) diabody, or DART® diabody, polypeptide chain to form an epitope binding site. DART® diabody molecules are disclosed in United States Patent Publications No. 2013-0295121; 2010-0174053 and 2009-0060910; European Patent Publication No. EP 2714079; EP 2601216; EP 2376109; EP 2158221 and PCT Publications No. WO 2012/162068; WO 2012/018687; WO 2010/080538; WO 2006/113665, WO 2008/157379 and Moore, P. A. et al. (2011) “Application Of Dual Affinity Retargeting Molecules To Achieve Optimal Redirected T-Cell Killing Of B-Cell Lymphoma,” Blood 117(17):4542-4551; Veri, M. C. et al. (2010) “Therapeutic Control Of B Cell Activation Via Recruitment Of Fcgamma Receptor IIb (CD32B) Inhibitory Function With A Novel Bi-specific Antibody Scaffold,” Arthritis Rheum. 62(7):1933-1943; and Johnson, S. et al. (2010) “Effector Cell Recruitment With Novel Fv-Based Dual-Affinity Re-Targeting Protein Leads To Potent Tumor Cytolysis And in vivo B-Cell Depletion,” J. Mol. Biol. 399(3):436-449.

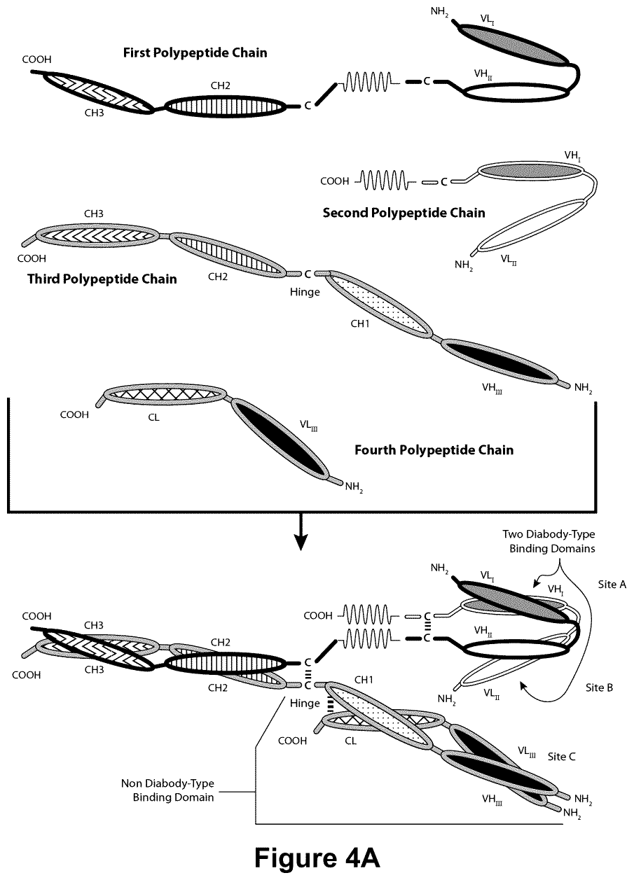

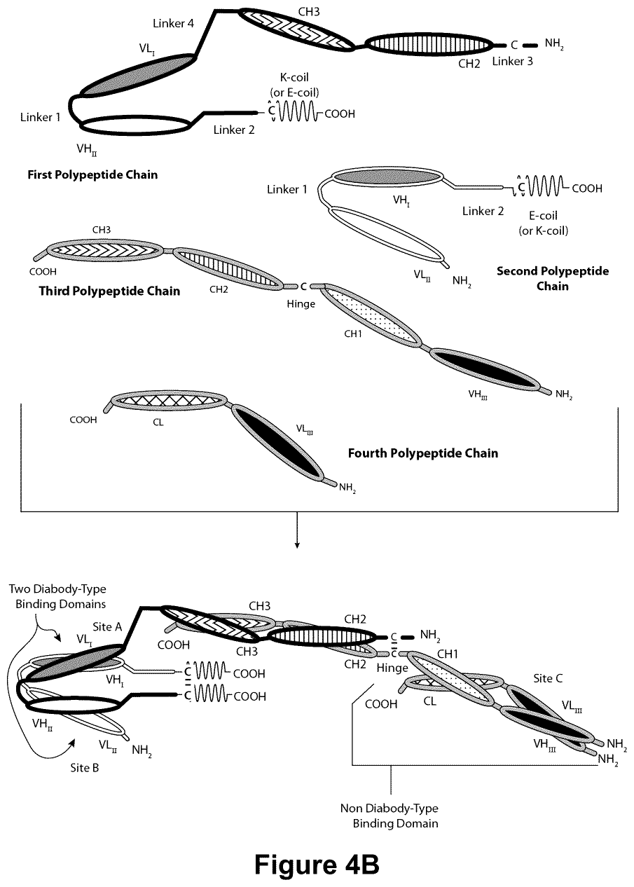

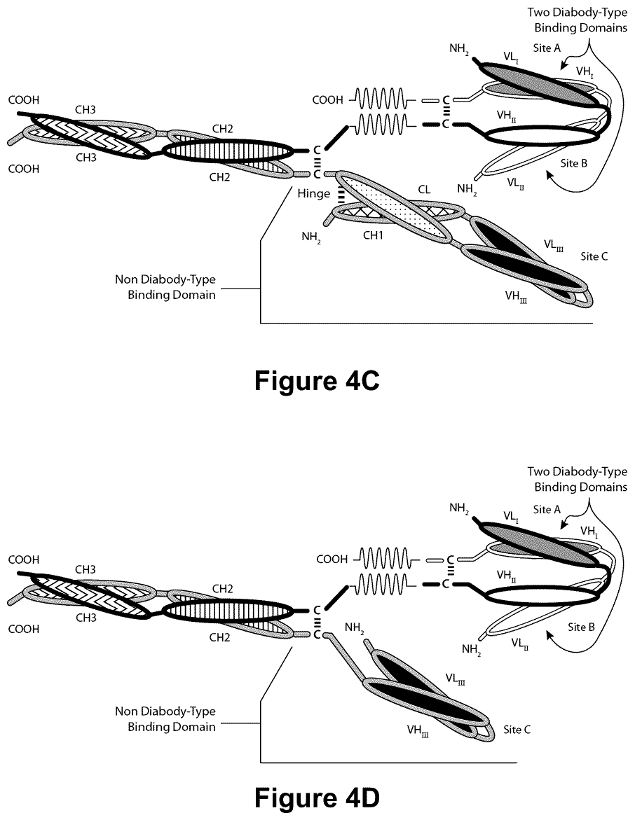

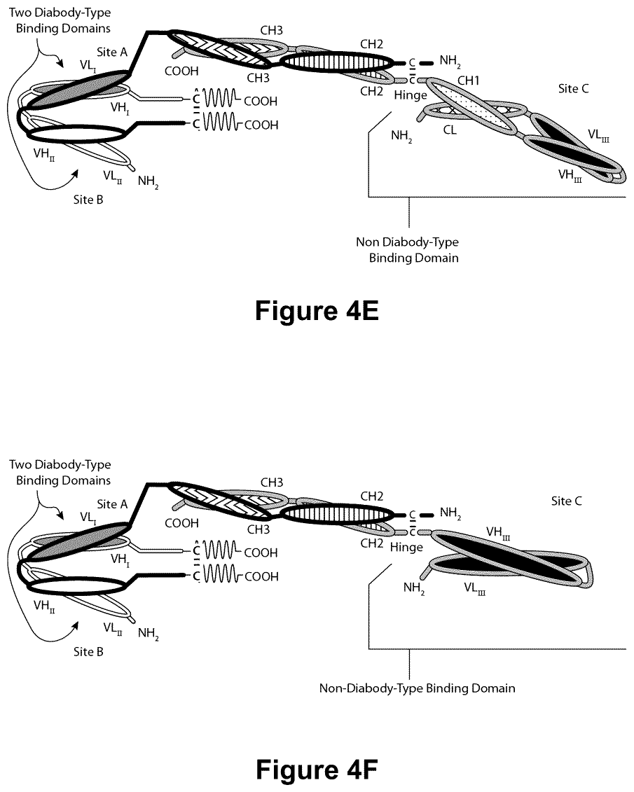

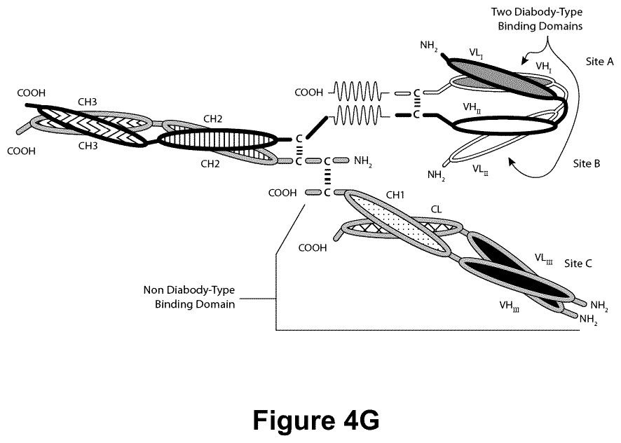

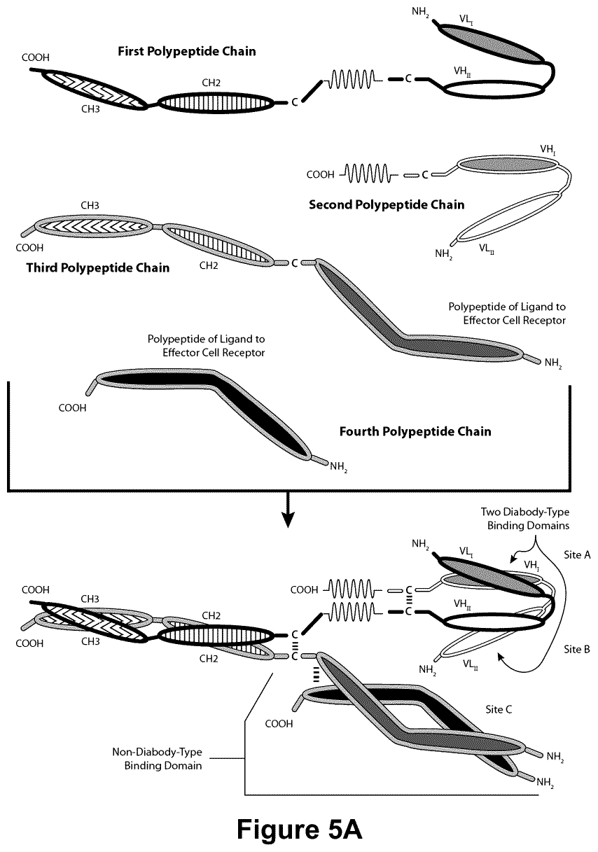

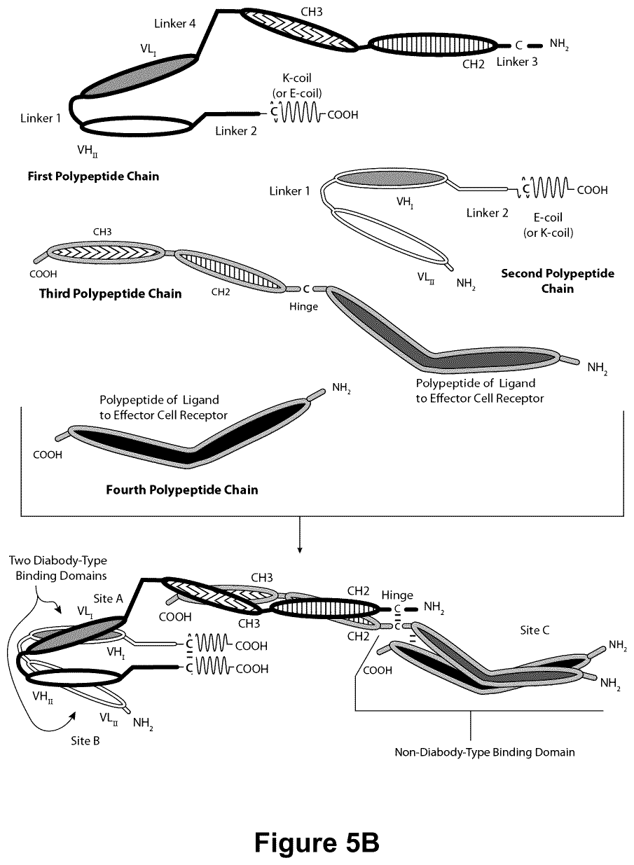

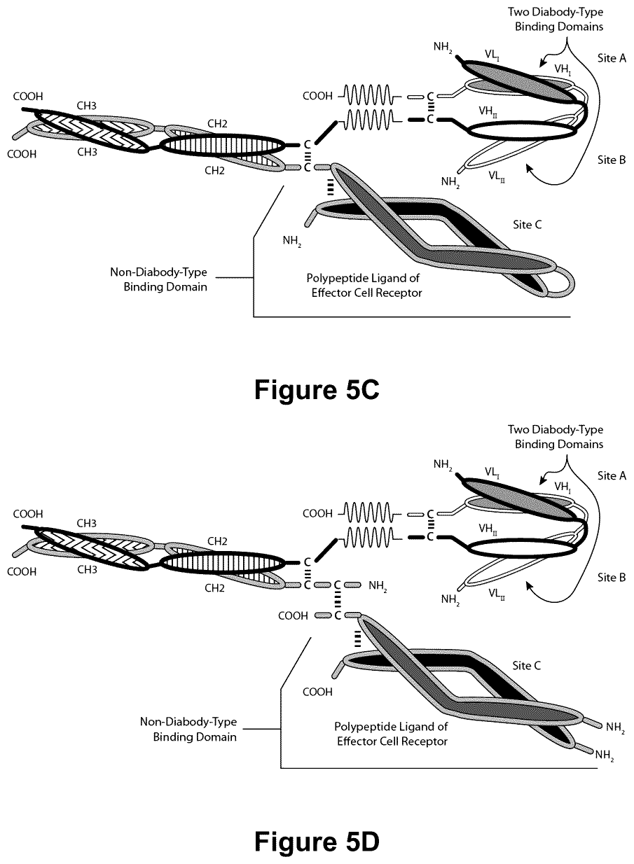

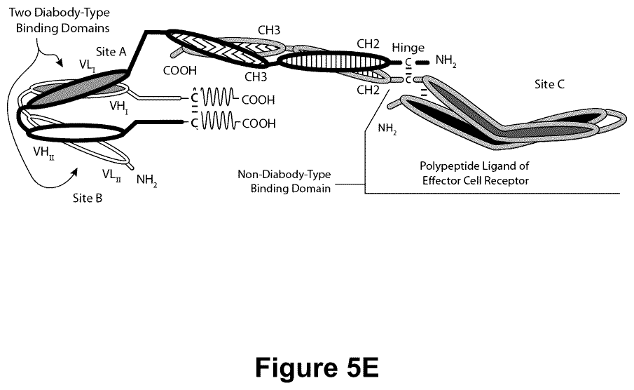

Binding Domain III is preferably a “Non-Diabody-Type” Binding Domain, which is intended to denote that Binding Domain III does not have the structure of a Diabody-Type Binding Domain. Preferably, Binding Domain III is a Non-Diabody-Type Binding Domain that is a Fab-Type Binding Domain or an Effector Cell Receptor-Type Binding Domain. Thus, in one embodiment, exemplified in FIGS. 4A-4G, the Binding Domain III is a Fab-Type Binding Domain. FIGS. 5A-5E exemplify the embodiment in which Binding Domain III is an Effector Cell Receptor-Type Binding Domain. As used herein, the term an “Fab-Type Binding Domain” refers to an epitope Binding Domain that is formed by the interaction of the VL Domain of an immunoglobulin light chain and a complementing VH Domain of an immunoglobulin heavy chain. Fab-Type Binding Domains differ from Diabody-Type Binding Domain in that the two polypeptide chains that form a Fab-Type Binding Domain comprise only a single epitope Binding Domain, whereas the two polypeptide chains that form a Diabody-Type Binding Domain comprise at least two epitope Binding Domains. Thus, as used herein Fab-Type Binding Domains are distinct from Diabody-Type Binding Domain. When a binding domain is a Fab-Type Binding Domain or a Diabody-Type Binding Domain, it will be composed of a VL Domain and a VH Domain, which may be located on the same or on different polypeptide chains. The selection of such VL and VH domains is coordinated, such that the domains form an epitope binding domain. As used herein, the term “Effector Cell Receptor-Type Binding Domain” refers to an epitope binding domain that is formed by the interaction of a variable domain of a T Cell Receptor alpha chain and a variable domain of a T Cell Receptor beta chain. Such receptors recognize peptides displayed in the context of MHC and are thus capable of recognizing intracellular epitopes.

The Tri-Specific Binding Molecules of the present invention are thus distinguished from tetravalent binding molecules, such as those produced from the dimerization of a bivalent antibody, and preferably possess three and not four Binding Domains. As discussed below, the trispecific molecules of the present invention may possess additional binding domains (such as an Albumin-Binding Domain, an FcγR-Binding Domain, etc.). Such additional Binding Domains are not intended to be considered or counted as being one of the three Binding Domains of the Tri-Specific Binding Molecules of the present invention.

As used herein, the terms “association” or “associating,” with regard to polypeptides (e.g., one diabody polypeptide to another, an immunoglobulin light chain to an immunoglobulin heavy chain, one CH2-CH3 Domain to another CH2-CH3 Domain, etc.) is intended to denote a non-covalent combining of the polypeptides. The terms “complexes” or “complexing” are intended to denote a covalent combining of the polypeptides.

As used herein, Binding Domains of a Binding Molecule of the invention is said to mediate “coordinated binding” if at least two of its Binding Domains and preferably all of its Binding Domains, are capable of concurrently being bound to their respective recognized epitopes or binding ligand. Such binding may be simultaneous. However, one aspect of the present invention relates to modifying the “on” and/or “off” rates with which such Binding Domains bind to their recognized epitopes. As used here, the “on rate” of binding is a measure of the affinity with which such Binding Domains recognize and initiate binding to their recognized epitopes. In contrast, the “off rate” of binding is a measure of the degree of stability of the Binding Domain:epitope complex. The “on” and/or “off” rates of binding can be modified by altering the amino acid sequence of the CDRs of a Binding Domain. As discussed below, independent of any CDR modifications, the extent of coordinated binding of the molecules of the present invention may be modulated by changing the configuration of the their Binding Domains so that a particular Binding Domain (i.e., a VLx/VHx Domain) is present as Binding Domain III or as an internal or external Diabody-Type Binding Domain relative to Binding Domain III (discussed in detail below).

The on- and off-rates of the Binding Domains of the Binding Molecules of the present invention can be readily measured by methods well-known in the art, for example by BIACORE® analysis (Jason-Moller, L. et al. (2006) “Overview Of Biacore Systems And Their Applications,” Curr. Protoc. Protein Sci. Chapter 19:Unit 19.13; Swanson, S. J. (2005) “Characterization Of An Immune Response,” Dev. Biol. (Basel). 122:95-101; Buijs, J. et al. (2005) “SPR-MS In Functional Proteomics,” Brief Funct. Genomic Proteomic. 4(1):39-47; Karlsson, R. et al. (2004) “SPR For Molecular Interaction Analysis: A Review Of Emerging Application Areas,” J. Mol. Recognit. 17(3):151-161; Van Regenmortel, M. H. (2003) “Improving The Quality Of BIACORE-Based Affinity Measurements,” Dev. Biol. (Basel) 112:141-151; Malmqvist, M. (1999) “BIACORE: An Affinity Biosensor System For Characterization Of Biomolecular Interactions,” Biochem. Soc. Trans. 27(2):335-340; Malmqvist, M. et al. (1997) “Biomolecular Interaction Analysis: Affinity Biosensor Technologies For Functional Analysis Of Proteins,” Curr. Opin. Chem. Biol. 1(3):378-383; Fivash, M. et al. (1998) “Biacore For Macromolecular Interaction,” Curr. Opin. Biotechnol. 9(1):97-101; Malmborg, A. C. et al. (1995) “Biacore As A Tool In Antibody Engineering,” J. Immunol. Methods. 183(1):7-13). The on- and off-rates of the Binding Domains of the Binding Molecules of the present invention can be readily altered by random or directed mutagenesis of nucleic acid molecules that encode such Binding Domains, followed by the routine screening of recovered nucleic acid molecules for their ability to encode mutated proteins that exhibit such altered binding kinetics.

The Binding Domains of the Tri-Specific Binding Molecules of the present invention bind to epitopes in an “immunospecific” manner. As used herein, an antibody, diabody or other epitope binding molecule is said to “immunospecifically” bind a region of another molecule (i.e., an epitope) if it reacts or associates more frequently, more rapidly, with greater duration and/or with greater affinity with that epitope relative to alternative epitopes. For example, an antibody that immunospecifically binds to a viral epitope is an antibody that binds this viral epitope with greater affinity, avidity, more readily, and/or with greater duration than it immunospecifically binds to other viral epitopes or non-viral epitopes. It is also understood by reading this definition that, for example, an antibody (or moiety or epitope) that immunospecifically binds to a first target may or may not specifically or preferentially bind to a second target. As such, “specific binding” does not necessarily require (although it can include) exclusive binding. Generally, but not necessarily, reference to binding means “specific” binding. Two molecules are said to be capable of binding to one another in a “physiospecific” manner, if such binding exhibits the specificity with which receptors bind to their respective ligands.

The functionality of antibodies can be enhanced by generating multispecific antibody-based molecules that can simultaneously bind two separate and distinct antigens (or different epitopes of the same antigen) and/or by generating antibody-based molecule having higher valency (i.e., more than two binding sites) for the same epitope and/or antigen.

Thus, in their simplest embodiment, the preferred binding molecules of the present invention are at least trispecific. Significantly, such molecules have at least three Sites that are capable of binding antigen: an “external” Diabody-Type Binding Domain that is located furthest from Binding Domain III, an “internal” Diabody-Type Binding Domain that is located nearest to Binding Domain III, and Binding Domain III itself. The positions of such Domains are respectively designated as “Site A,” Site B” and “Site C” (FIGS. 4A-4G; FIGS. 5A-5E).

The Tri-Specific Binding Molecules of the present invention are able to coordinately bind to three different epitopes by comprising three binding domains. Two of the binding domains of such molecules are capable of binding to epitopes of “Cancer Antigens,” such that the molecule is capable of binding to two different Cancer Antigens. The third binding domain of such molecules is capable of binding to an epitope of a molecule expressed on the surface of an immune system effector cell (i.e., an “Effector Cell Antigen”). Thus, the Tri-Specific Binding Molecules of the present invention are able to mediate coordinated and simultaneous binding to a cancer cell expressing two Cancer Antigens and to an immune system effector cell expressing the Effector Cell Antigen. The epitopes recognized by the Tri-Specific Binding Molecules of the present invention may be continuous or discontinuous (e.g., conformational).

The first and second Cancer Antigens that are bound by the Cancer Antigen-Binding Domains of the trispecific binding molecules of the present invention may be selected from any molecule that is characteristically present on the surface of a cancer cell. One aspect of the present invention relates to the ability to target “Low Expression Cancer Antigens” (i.e., a Cancer Antigen that may be expressed on a cancer cell at a level too low to permit a monospecific binding molecule to provide an effective cancer therapy). In contrast to such monospecific binding molecules, the Tri-Specific Binding Molecules of the present invention, by targeting two Cancer Antigens instead of one, exhibit synergistic and cooperative enhanced binding avidity that may compensate for low affinity of binding and thus may be advantageously used to target cancers characterized even by a Low Expression Cancer Antigen. A second aspect of the present invention relates to the ability to target “Low Specificity Cancer Antigens” (i.e., a Cancer Antigen that may be expressed on a normal cell in addition to being expressed on a cancer cell). The Tri-Specific Binding Molecules of the present invention, by providing synergistic and cooperative enhanced binding avidity to two Cancer Antigens, exhibits higher avidity of binding even for Low Specificity Cancer Antigens and thus provides a means for treating cancers that are characterized by such Cancer Antigens. Thus, the Tri-Specific Binding Molecules of the present invention may be used to impart an anti-cancer therapy even in circumstances where one or both of the target Cancer Antigens is ineffective on its own to provide such therapy.

For example, CD32B (the FcγRIIB receptor) is widely expressed on hematopoietic cells, including monocytes, macrophages, B cells, NK cells, neutrophils, mast cells, and platelets. Upon binding to IgG Fc Domain, CD32B inhibits the host immune system to thereby depress an ongoing immune response. Although such inhibition is desirable in helping the host recover from inflammatory reactions, it serves to exacerbate the immune deficiencies of subjects suffering from cancer or infectious disease. Antibodies that bind to CD32B, so as to block the binding of IgG Fc molecules, serve to prevent such inhibition and thus have utility as adjunct molecules in the treatment of cancer and infectious disease (Veri, M. C. et al. (2007) “Monoclonal Antibodies Capable Of Discriminating The Human Inhibitory Fcgamma-Receptor IIB (CD32B) From The Activating Fcgamma-Receptor IIA (CD32A): Biochemical, Biological And Functional Characterization,” Immunology 121(3):392-404). Unfortunately, CD32B is also expressed on liver sinusoidal endothelial cells (“LSE cells”) (Shahani, T. et al. (2014) “Human Liver Sinusoidal Endothelial Cells But Not Hepatocytes Contain Factor VIII,” J. Thromb. Haemost. 12(1):36-42; Geraud, C. et al. (2013) “Endothelial Transdifferentiation In Hepatocellular Carcinoma: Loss Of Stabilin-2 Expression In Peri-Tumourous Liver Correlates With Increased Survival,” Liver Int. 33(9):1428-1440; Takabe, Y. et al. (2012) “Immunomagnetic Exclusion Of E-Cadherin-Positive Hepatoblasts In Fetal Mouse Liver Cell Cultures Impairs Morphogenesis And Gene Expression Of Sinusoidal Endothelial Cells,” J. Anat. 221(3):229-239). Thus, antibodies that bind CD32B attack LSE cells. However, by forming a Tri-Specific Binding Molecule of the present invention that binds to CD32B and to antigens (i.e., the first and second Cancer Antigens) that are not expressed on LSE cells, or are expressed at low levels by such cells (i.e., Low Expression Cancer Antigen(s)), or are expressed with low specificity on cancer cells and such LSE cells (i.e., Low Specificity Cancer Antigen(s)), the present invention provides compositions and methods that would be used to depress CD32B-mediated immune system inhibition.

### B. Exemplary Cancer Antigen-Binding Domains

Examples of suitable Cancer Antigens include: 19.9 as found in colon cancer, gastric cancer mucins; 4.2; A33 (a colorectal carcinoma antigen; Almqvist, Y. 2006, Nucl Med Biol. November; 33(8):991-998); ADAM-9 (United States Patent Publication No. 2006/0172350; PCT Publication No. WO 06/084075); AH6 as found in gastric cancer; ALCAM (PCT Publication No. WO 03/093443); APO-1 (malignant human lymphocyte antigen) (Trauth et al. (1989) “Monoclonal Antibody-Mediated Tumor Regression By Induction Of Apoptosis,” Science 245:301-304); B1 (Egloff, A. M. et al. 2006, Cancer Res. 66(1):6-9); BAGE (Bodey, B. 2002 Expert Opin Biol Ther. 2(6):577-84); B7-H3; beta-catenin (Prange W. et al. 2003 J Pathol. 201(2):250-9); blood group ALe^{b}/Le^{y} as found in colonic adenocarcinoma; Burkitt's lymphoma antigen-38.13, C14 as found in colonic adenocarcinoma; CA125 (ovarian carcinoma antigen) (Bast, R. C. Jr. et al. 2005 Int J Gynecol Cancer 15 Suppl 3:274-81; Yu et al. (1991) “Coexpression Of Different Antigenic Markers On Moieties That Bear CA 125 Determinants,” Cancer Res. 51(2):468-475); Carboxypeptidase M (United States Patent Publication No. 2006/0166291); CD5 (Calin, G. A. et al. 2006 Semin Oncol. 33(2):167-73; CD19 (Ghetie et al. (1994) “Anti-CD19Inhibits The Growth Of Human B-Cell Tumor Lines In Vitro And Of Daudi Cells In SCID Mice By Inducing Cell Cycle Arrest,” Blood 83:1329-1336; Troussard, X. et al. 1998 Hematol Cell Ther. 40(4):139-48); CD20 (Thomas, D. A. et al. 2006 Hematol Oncol Clin North Am. 20(5):1125-36); CD22 (Kreitman, R. J. 2006 AAPS J. 18; 8(3):E532-51); CD23 (Rosati, S. et al. 2005 Curr Top Microbiol Immunol. 5; 294:91-107); CD25 (Troussard, X. et al. 1998 Hematol Cell Ther. 40(4):139-48); CD27 (Bataille, R. 2006 Haematologica 91(9):1234-40); CD28 (Bataille, R. 2006 Haematologica 91(9):1234-40); CD33 (Sgouros et al. (1993) “Modeling And Dosimetry Of Monoclonal Antibody M195 (Anti-CD33) In Acute Myelogenous Leukemia,” J. Nucl. Med. 34:422-430); CD36 (Ge, Y. 2005 Lab Hematol. 11(1):31-7); CD40/CD154 (Messmer, D. et al. 2005 Ann N Y Acad Sci. 1062:51-60); CD45 (Jurcic, J. G. 2005 Curr Oncol Rep. 7(5):339-46); CD56 (Bataille, R. 2006 Haematologica 91(9):1234-40); CD46 (U.S. Pat. No. 7,148,038; PCT Publication No. WO 03/032814); CD79a/CD79b (Troussard, X. et al. 1998 Hematol Cell Ther. 40(4):139-48; Chu, P. G. et al. 2001 Appl Immunohistochem Mol Morphol. 9(2):97-106); CD103 (Troussard, X. et al. 1998 Hematol Cell Ther. 40(4):139-48); CDK4 (Lee, Y. M. et al. 2006 Cell Cycle 5(18):2110-4); CEA (carcinoembryonic antigen) (Foon et al. (1995) “Immune Response To The Carcinoembryonic Antigen In Patients Treated With An Anti-Idiotype Antibody Vaccine,” J. Clin. Invest. 96(1):334-42); CEA (carcinoembryonic antigen; Mathelin, C. 2006 Gynecol Obstet Fertil. 34(7-8):638-46; Tellez-Avila, F. I. et al. 2005 Rev Invest Clin. 57(6):814-9); C017-1A (Ragnhammar et al. (1993) “Effect Of Monoclonal Antibody 17-1A And GM-CSF In Patients With Advanced Colorectal Carcinoma—Long-Lasting, Complete Remissions Can Be Induced,” Int. J. Cancer 53:751-758); CO-43 (blood group Le^{b}); CO-514 (blood group Le^{a}) as found in adenocarcinoma; CTA-1; CTLA4 (Peggs, K. S. et al. 2006 Curr Opin Immunol. 18(2):206-13); Cytokeratin 8 (PCT Publication No. WO 03/024191); D1.1; D156-22; DR5 (Abdulghani, J. et al. (2010) “TRAIL Receptor Signaling And Therapeutics,” Expert Opin. Ther. Targets 14(10):1091-1108; Andera, L. (2009) “Signaling Activated By The Death Receptors Of The TNFR Family,” Biomed. Pap. Med. Fac. Univ. Palacky Olomouc Czech. Repub. 153(3):173-180; Carlo-Stella, C. et al. (2007) “Targeting TRAIL Agonistic Receptors for Cancer Therapy,” Clin, Cancer 13(8):2313-2317; Chaudhari, B. R. et al. (2006) “Following the TRAIL to Apoptosis,” Immunologic Res. 35(3):249-262); E1 series (blood group B) as found in pancreatic cancer; EGFR (Epidermal Growth Factor Receptor) (Adenis, A. et al. 2003 Bull Cancer. 90 Spec No:S228-32); Ephrin receptors (and in particular EphA2 (U.S. Pat. No. 7,569,672; PCT Publication No. WO 06/084226); Erb (ErbB1; ErbB3; ErbB4; Zhou, H. et al. 2002 Oncogene 21(57):8732-8740; Rimon, E. et al. 2004 Int J Oncol. 24(5):1325-1338); GAGE (GAGE-1; GAGE-2; Akcakanat, A. et al. 2006 Int J Cancer. 118(1):123-128); GD2/GD3/GM2 (Livingston, P. O. et al. 2005 Cancer Immunol Immunother. 54(10):1018-1025); F3 as found in lung adenocarcinoma; FC10.2 as found in embryonal carcinoma cells and gastric adenocarcinoma; G49, ganglioside GD2 (Saleh et al. (1993) “Generation Of A Human Anti-Idiotypic Antibody That Mimics The GD2 Antigen,” J. Immunol., 151, 3390-3398); ganglioside GD3 (Shitara et al. (1993) “A Mouse Human Chimeric Anti-(Ganglioside GD3) Antibody With Enhanced Antitumor Activities,” Cancer Immunol. Immunother. 36:373-380); ganglioside GM2 (Livingston et al. (1994) “Improved Survival In Stage III Melanoma Patients With GM2 Antibodies: A Randomized Trial Of Adjuvant Vaccination With GM2 Ganglioside,” J. Clin. Oncol. 12:1036-1044); ganglioside GM3 (Hoon et al. (1993) “Molecular Cloning Of A Human Monoclonal Antibody Reactive To Ganglioside GM3 Antigen On Human Cancers,” Cancer Res. 53:5244-5250); G_{D2}; G_{D3}; GICA 19-9 (Herlyn et al. (1982) “Monoclonal Antibody Detection Of A Circulating Tumor-Associated Antigen. I. Presence Of Antigen In Sera Of Patients With Colorectal, Gastric, And Pancreatic Carcinoma,” J. Clin. Immunol. 2:135-140); GM2; gp100 (Lotem, M. et al. 2006 J Immunother. 29(6):616-27); Gp37 (human leukemia T cell antigen) (Bhattacharya-Chatterjee et al. (1988) “Idiotype Vaccines Against Human T Cell Leukemia. IL Generation And Characterization Of A Monoclonal Idiotype Cascade (Ab1, Ab2, and Ab3),” J. Immunol. 141:1398-1403); gp75 (melanoma antigen) (Vijayasardahl et al. (1990) “The Melanoma Antigen Gp75 Is The Human Homologue Of The Mouse B (Brown) Locus Gene Product,” J. Exp. Med. 171(4):1375-1380); gpA33; HER2 antigen (p185^{HER2}) (Kumar, Pal S et al. 2006 Semin Oncol. 33(4):386-91); human B-lymphoma antigen-CD20 (Reff et al. (1994) “Depletion Of B Cells In Vivo By A Chimeric Mouse Human Monoclonal Antibody To CD20,” Blood 83:43 5-445); human milk fat globule antigen; human papillomavirus-E6/human papillomavirus-E7 (DiMaio, D. et al. 2006 Adv Virus Res. 66:125-59; HMW-MAA (high molecular weight melanoma antigen) (Natali et al. (1987) “Immunohistochemical Detection Of Antigen In Human Primary And Metastatic Melanomas By The Monoclonal Antibody 140.240 And Its Possible Prognostic Significance,” Cancer 59:55-63; Mittelman et al. (1990) “Active Specific Immunotherapy In Patients With Melanoma. A Clinical Trial With Mouse Antiidiotypic Monoclonal Antibodies Elicited With Syngeneic Anti-High-Molecular-Weight-Melanoma-Associated Antigen Monoclonal Antibodies,” J. Clin. Invest. 86:2136-2144); I antigen (differentiation antigen) (Feizi (1985) “Demonstration By Monoclonal Antibodies That Carbohydrate Structures Of Glycoproteins And Glycolipids Are Onco-Developmental Antigens,” Nature 314:53-57) such as I(Ma) as found in gastric adenocarcinomas; Integrin Alpha-V-Beta-6 (PCT Publication No. WO 03/087340); JAM-3 (PCT Publication No. WO 06/084078); KID3 (PCT Publication No. WO 05/028498); KID31 (PCT Publication No. WO 06/076584); KS 1/4 pan-carcinoma antigen (Perez et al. (1989) “Isolation And Characterization Of A cDNA Encoding The Ks1/4 Epithelial Carcinoma Marker,” J. Immunol. 142:3662-3667; Möller et al. (1991) “Bi-specific-Monoclonal-Antibody-Directed Lysis Of Ovarian Carcinoma Cells By Activated Human T Lymphocytes,” Cancer Immunol. Immunother. 33(4):210-216; Ragupathi, G. 2005 Cancer Treat Res. 123:157-80); L6 and L20 (human lung carcinoma antigens) (Hellström et al. (1986) “Monoclonal Mouse Antibodies Raised Against Human Lung Carcinoma,” Cancer Res. 46:3917-3923); LEA; LUCA-2 (United States Patent Publication No. 2006/0172349; PCT Publication No. WO 06/083852); M1:22:25:8; M18; M39; MAGE (MAGE-1; MAGE-3; (Bodey, B. 2002 Expert Opin Biol Ther. 2(6):577-84); MART (Kounalakis, N. et al. 2005 Curr Oncol Rep. 7(5):377-82; MUC-1 (Mathelin, C. 2006 Gynecol Obstet Fertil. 34(7-8):638-46); MUM-1 (Castelli, C. et al. 2000J Cell Physiol. 182(3):323-31); Myl; N-acetylglucosaminyltransferase (Dennis, J. W. 1999 Biochim Biophys Acta. 6; 1473(1):21-34); neoglycoprotein; NS-10 as found in adenocarcinomas; OFA-1; OFA-2; Oncostatin M (Oncostatin Receptor Beta) (U.S. Pat. No. 7,572,896; PCT Publication No. WO 06/084092); p15 (Gil, J. et al. 2006 Nat Rev Mol Cell Biol. 7(9):667-77); p97 (melanoma-associated antigen) (Estin et al. (1989) “Transfected Mouse Melanoma Lines That Express Various Levels Of Human Melanoma-Associated Antigen p97,” J. Natl. Cancer Instit. 81(6):445-454); PEM (polymorphic epithelial mucin) (Hilkens et al. (1992) “Cell Membrane-Associated Mucins And Their Adhesion-Modulating Property,” Trends in Biochem. Sci. 17:359-363); PEMA (polymorphic epithelial mucin antigen); PIPA (U.S. Pat. No. 7,405,061; PCT Publication No. WO 04/043239); PSA (prostate-specific antigen) (Henttu et al. (1989) “cDNA Coding For The Entire Human Prostate Specific Antigen Shows High Homologies To The Human Tissue Kallikrein Genes,” Biochem. Biophys. Res. Comm. 10(2):903-910; Israeli et al. (1993) “Molecular Cloning Of A Complementary DNA Encoding A Prostate-Specific Membrane Antigen,” Cancer Res. 53:227-230; Cracco, C. M. et al. 2005 Minerva Urol Nefrol. 57(4):301-11); PSMA (prostate-specific membrane antigen) (Ragupathi, G. 2005 Cancer Treat Res. 123:157-180); prostatic acid phosphate (Tailor et al. (1990) “Nucleotide Sequence Of Human Prostatic Acid Phosphatase Determined From A Full-Length cDNA Clone,” Nucl. Acids Res. 18(16):4928); R₂₄ as found in melanoma; ROR1 (U.S. Pat. No. 5,843,749); sphingolipids; SSEA-1; SSEA-3; SSEA-4; sTn (Holmberg, L. A. 2001 Expert Opin Biol Ther. 1(5):881-91); T cell receptor derived peptide from a cutaneous T cell lymphoma (see Edelson (1998) “Cutaneous T-Cell Lymphoma: A Model For Selective Immunotherapy,” Cancer J Sci Am. 4:62-71); T5A7 found in myeloid cells; TAG-72 (Yokota et al. (1992) “Rapid Tumor Penetration Of A Single-Chain Fv And Comparison With Other Immunoglobulin Forms,” Cancer Res. 52:3402-3408); TL5 (blood group A); TNF-receptor (TNF-α receptor, TNF-β receptor; TNF-γ receptor (van Horssen, R. et al. 2006 Oncologist. 11(4):397-408; Gardnerova, M. et al. 2000 Curr Drug Targets. 1(4):327-64); TRA-1-85 (blood group H); Transferrin Receptor (U.S. Pat. No. 7,572,895; PCT Publication No. WO 05/121179); TSTA (tumor-specific transplantation antigen) such as virally-induced tumor antigens including T-antigen DNA tumor viruses and envelope antigens of RNA tumor viruses, oncofetal antigen-alpha-fetoprotein such as CEA of colon, bladder tumor oncofetal antigen (Hellström et al. (1985) “Monoclonal Antibodies To Cell Surface Antigens Shared By Chemically Induced Mouse Bladder Carcinomas,” Cancer. Res. 45:2210-2188); VEGF receptor (O'Dwyer. P. J. 2006 Oncologist. 11(9):992-998); VEP8; VEP9; VIM-D5; and Y hapten, Le^{y} as found in embryonal carcinoma cells.

1. Campath-1 (CD52) Binding Domain (Alemtuzumab)

The amino acid sequence of the VL Domain of the humanized anti-CD52 antibody “Alemtuzumab” (SEQ ID NO:205) is shown below (CDR residues are shown underlined):

DIQMTQSPSS LSASVGDRVT ITCKASQNID KYLNWYQQKP
GKAPKLLIYN TNNLQTGVPS RFSGSGSGTD FTFTISSLQP
EDIATYYCLQ HISRPRTFGQ GTKVEIKR

The amino acid sequence of the VH Domain of the humanized anti-CD52 antibody “Alemtuzumab” (SEQ ID NO:206) is shown below (CDR residues are shown underlined):

QVQLQESGPG LVRPSQTLSL TCTVSGFTFT DFYMNWVRQP
PGRGLEWIGF IRDKAKGYTT EYNPSVKGRV TMLVDTSKNQ
FSLRLSSVTA ADTAVYYCAR EGHTAAPFDY WGQGSLVTVS
S

2. CD317 (BMST2)-Binding Domains

CD317 (also known as Bone Marrow Stromal Cell Antigen 2; BMST) is overexpressed on various cancer cells isolated from breast, lung, kidney, endometrium, and skin (Kawai, S. et al. (2008) “Interferon-α enhances CD317 expression and the antitumor activity of anti-CD317 monoclonal antibody in renal cell carcinoma xenograft models,” Cancer Science 99(12):2461-2466; Cai, D. et al. (2009) “Up-Regulation Of Bone Marrow Stromal Protein 2 (BST2) In Breast Cancer With Bone Metastasis,” BMC Cancer 9:102, pp. 1-10; Wang, W. et al. (2009) HM1.24 (CD317) Is A Novel Target Against Lung Cancer For Immunotherapy Using Anti-HM1.24 Antibody,” Cancer Immunology, Immunotherapy 58(6):967-976; Wang, W. et al. (2009) “Chimeric And Humanized Anti-HM1.24 Antibodies Mediate Antibody-Dependent Cellular Cytotoxicity Against Lung Cancer Cells. Lung Cancer,” 63(1):23-31; Sayeed, A. et al. (2013) “Aberrant Regulation Of The BST2 (Tetherin) Promoter Enhances Cell Proliferation And Apoptosis Evasion In High Grade Breast Cancer Cells,” PLoS ONE 8(6)e67191, pp. 1-10; Yi, E. H. et al. (2013) “BST-2 Is A Potential Activator Of Invasion And Migration In Tamoxifen-Resistant Breast Cancer Cells,” Biochem. Biophys. Res. Commun. 435(4):685-690; Staudinger, M. (2014) “The Novel Immunotoxin HM1.24-ETA′ Induces Apoptosis In Multiple Myeloma Cells,” Blood Cancer J. 13; 4:e219, pp. 1-11). Antibodies that immunospecifically bind to CD317 are commercially available (Novus Biologicals LLC; BioLegend, Inc.; see also U.S. Pat. No. 8,834,876, which references the deposit of the heavy and light chains of antibody HM1.24 as FERM BP-5644 and FERM BP-5646; see also U.S. Pat. No. 8,394,374). The amino acid sequence of the VL Domain of the anti-CD317 antibody “HM1.24” (SEQ ID NO:302) is shown below (CDR residues are shown underlined):

DIVMTQSHKF MSTSVGDRVS ITCKKASQDV NTAVAWYQQK
PGQSPKLLIY SASNRYTGVP DRITGSGSGT DFTFTISSVQ
AEDLALTTCQ QHYSTPFTFG SGTKLEIK

The amino acid sequence of the VH Domain of the anti-CD317 antibody “HM1.24” (SEQ ID NO:303) is shown below (CDR residues are shown underlined):

QVQLQQSGAE LARPGASVKL SCKASGYTFT PYWMQWVKQR
PGQGLEWIGS IFPGDGDTRY SQKFKGKATL TADKSSSTAY
MQLSILAFED SAVYYCARGL RRGGYYFDYW GQGTTLTVSS

3. CEACAM5- and CEACAM6-Binding Domains

Carcinoembryonic Antigen-Related Cell Adhesion Molecules 5 (CEACAM5) and 6 (CEACAM6) have been found to be associated with various types of cancers including medullary thyroid cancer, colorectal cancer, pancreatic cancer, hepatocellular carcinoma, gastric cancer, lung cancer, head and neck cancers, urinary bladder cancer, prostate cancer, uterine cancer, endometrial cancer, breast cancer, hematopoietic cancer, leukemia and ovarian cancer (PCT Publication No. WO 2011/034660), and particularly colorectal, gastrointestinal, pancreatic, non-small cell lung cancer (NSCL), breast, thyroid, stomach, ovarian and uterine carcinomas (Zheng, C. et al. (2011) “A Novel Anti-CEACAM5 Monoclonal Antibody, CC4, Suppresses Colorectal Tumor Growth and Enhances NK Cells-Mediated Tumor Immunity,” PLoS One 6(6):e21146, pp. 1-11).

CEACAM5 has been found to be overexpressed in 90% of gastrointestinal, colorectal and pancreatic cancers, 70% of non-small cell lung cancer cells and 50% of breast cancers (Thompson, J. A. et al. (1991) “Carcinoembryonic Antigen Gene Family: Molecular Biology And Clinical Perspectives,” J. Clin. Lab. Anal. 5:344-366).

Overexpressed carcinoembryonic antigen-related cellular adhesion molecule 6 (CEACAM6) plays important roles in the invasion and metastasis of a variety of human cancers, including medullary thyroid cancer, colorectal cancer, pancreatic cancer, hepatocellular carcinoma, gastric cancer, lung cancer, head and neck cancers, urinary bladder cancer, prostate cancer, uterine cancer, endometrial cancer, breast cancer, hematopoietic cancer, leukemia and ovarian cancer (PCT Publication No. WO 2011/034660; Deng, X. et al. (2014) “Expression Profiling Of CEACAM6 Associated With The Tumorigenesis And Progression In Gastric Adenocarcinoma,” Genet. Mol. Res. 13(3):7686-7697; Cameron, S. et al. (2012) “Focal Overexpression Of CEACAM6 Contributes To Enhanced Tumourigenesis In Head And Neck Cancer Via Suppression Of Apoptosis,” Mol. Cancer 11:74, pp. 1-11; Chapin, C. et al. (2012) “Distribution And Surfactant Association Of Carcinoembryonic Cell Adhesion Molecule 6In Human Lung,” Amer. J. Physiol. Lung Cell. Mol. Physiol. 302(2):L216-L25; Riley, C. J. et al. (2009) “Design And Activity Of A Murine And Humanized Anti-CEACAM6 Single-Chain Variable Fragment In The Treatment Of Pancreatic Cancer,” Cancer Res. 69(5):1933-1940; Lewis-Wambi, J. S. et al. (2008) “Overexpression Of CEACAM6 Promotes Migration And Invasion Of Oestrogen-Deprived Breast Cancer Cells,” Eur. J. Cancer 44(12):1770-1779; Blumenthal, R. D. et al. (2007) “Expression Patterns Of CEACAM5 And CEACAM6 In Primary And Metastatic Cancers,” BMC Cancer. 7:2, pp. 1-15). Antibodies that immunospecifically bind to CEACAM5 and CEACAM6 are commercially available (Santa Cruz Biotechnology, Inc., Novus Biologicals LLC; Abnova Corporation). The amino acid sequence of the VL Domain of the humanized anti-CEACAM5/ANTI-CEACAM6 antibody 16C3 (EP 2585476) (SEQ ID NO:304) is shown below (CDR residues are shown underlined):

DIQMTQSPSS LSASVGDRVT ITCGASENIY GALNWYQRKP
GKSPKLLIWG ASNLADGMPS RFSGSGSGRQ YTLTISSLQP
EDVATYYCQN VLSSPYTFGG GTKLEIK

The amino acid sequence of the VH Domain of the humanized anti-CEACAM5/ANTI-CEACAM6 antibody 16C3 (EP 2585476) (SEQ ID NO:305) is shown below (CDR residues are shown underlined):

QVQLQQSGPE VVRPGVSVKI SCKGSGYTFT DYAMHWVKQS
HAKSLEWIGL ISTYSGDTKY NQNFKGKATM TVDKSASTAY
MELSSLRSED TAVYYCARGD YSGSRYWFAY WGQGTLVTVS
S

The amino acid sequence of the VL Domain of the humanized anti-CEACAM5/CEACAM6 antibody hMN15 (WO 2011/034660) (SEQ ID NO:306) is shown below (CDR residues are shown underlined):

DIQLTQSPSS LSASVGDRVT MTCSASSRVS YIHWYQQKPG
KAPKRWIYGT STLASGVPAR FSGSGSGTDF TFTISSLQPE
DIATYYCQQW SYNPPTFGQG TKVEIKR

The amino acid sequence of the VH Domain of the humanized anti-CEACAM5/CEACAM6 antibody hMN15 (WO 2011/034660) (SEQ ID NO:307) is shown below (CDR residues are shown underlined):

QVQLVESGGG VVQPGRSLRL SCSSSGFALT DYYMSWVRQA
PGKGLEWLGF IANKANGHTT DYSPSVKGRF TISRDNSKNT
LFLQMDSLRP EDTGVYFCAR DMGIRWNFDV WGQGTPVTVS
S

4. DR5-Binding Domains

DR5 is a preferred Cancer Antigen of the present invention. The preferred anti-human DR5-binding molecules of the present invention possess the VL and/or VH Domains of murine anti-human DR5 monoclonal antibodies “DR5 mAb 1” and/or “DR5 mAb 2,” and more preferably possess 1, 2 or all 3 of the CDRs of the VL Domain and/or 1, 2 or all 3 of the CDRs of the VH Domain of such anti-human DR5 monoclonal antibodies. Alternatively, any anti-human DR5 monoclonal antibody may be employed, particularly: drozitumab (designated herein as “DR5 mAb 3”), conatumumab (designated herein as “DR5 mAb 4”), tigatuzumab (designated herein as “DR5 mAb 5”), LBY135-1 (designated herein as “DR5 mAb 6”), LBY135-2 (designated herein as “DR5 mAb 7”) and KMTR2 (designated herein as “DR5 mAb 8”).

a. The Anti-Human DR5 Antibody DR5 mAb 1

DR5 has potential utility in the treatment of a wide range of cancers (e.g., colorectal cancer, hepatocellular carcinoma, glioma, kidney cancer, breast cancer, multiple myeloma, bladder cancer, neuroblastoma; sarcoma, non-Hodgkin's lymphoma, non-small cell lung cancer, ovarian cancer, pancreatic cancer and rectal cancer. The amino acid sequence of human DR5 precursor (NCBI Sequence NP_003833.4) (SEQ ID NO:2) is:

MEQRGQNAPA ASGARKRHGP GPREARGARP GLRVPKTLVL
VVAAVLLLVS AESALITQQD LAPQQRVAPQ QKRSSPSEGL
CPPGHHISED GRDCISCKYG QDYSTHWNDL LFCLRCTRCD
SGEVELSPCT TTRNTVCQCE EGTFREEDSP EMCRKCRTGC
PRGMVKVGDC TPWSDIECVH KESGTKHSGE APAVEETVTS
SPGTPASPCS LSGIIIGVTV AAVVLIVAVF VCKSLLWKKV
LPYLKGICSG GGGDPERVDR SSQRPGAEDN VLNEIVSILQ
PTQVPEQEME VQEPAEPTGV NMLSPGESEH LLEPAEAERS
QRRRLLVPAN EGDPTETLRQ CFDDFADLVP FDSWEPLMRK
LGLMDNEIKV AKAEAAGHRD TLYTMLIKWV NKTGRDASVH
TLLDALETLG ERLAKQKIED HLLSSGKFMY LEGNADSAMS

The amino acid sequence of the VL Domain of DR5 mAb 1 (SEQ ID NO:3) is shown below (CDR residues are shown underlined):

DIVLTQSPAS LAVSLGQRAT ISCRASKSVS SSGYSYMHWY
QQKPGQPPKV LIFLSSNLDS GVPARFSGSG SGTDFTLNIH
PVEDGDAATY YCQHSRDLPP TFGGGTKLEI K
CDR_{L}1 of DR5 mAb 1 (SEQ ID NO: 4):
RASKSVSSSGYSYMH
CDR_{L}2 of DR5 mAb 1 (SEQ ID NO: 5):
LSSNLDS
CDR_{L}3 of DR5 mAb 1 (SEQ ID NO: 6):
QHSRDLPPT

The VL Domain of DR5 mAb 1 is preferably encoded by a polynucleotide (SEQ ID NO:7) having the sequence shown below (polynucleotides encoding the CDRs are shown in underline):

gacattgtgc tgacacagtc tcctgcttcc ttagctgtat
ctctcgggca gagggccacc atctcatgca gggccagcaa
aagtgtcagt tcctctggct atagttatat gcactggtac
caacagaaac caggacagcc acccaaagtc ctcatctttc
tttcatccaa cctagattct ggggtccctg ccaggttcag
tggcagtggg tctgggacag acttcaccct caacatccat
cctgtggagg atggggatgc tgcaacctat tactgtcagc
acagtaggga tcttcctccg acgttcggtg gaggcaccaa
gctggaaatc aaa

The amino acid sequence of the VH Domain of DR5 mAb 1 (SEQ ID NO:8) is shown below (CDR residues are shown underlined). The C-terminal amino acid may be substituted with alanine to facilitate subcloning of this VH Domain.

EVKFLESGGG LVQPGGSLKL SCVASGFDFS RYWMSWVRQA
PGKGLEWIGE INPDSNTINY TPSLKDKFII SRDNAKNTLY
LQMTKVRSED TALYYCTRRA YYGNPAWFAY WGQGTLVTVSS
CDR_{H}1 of DR5 mAb 1 (SEQ ID NO: 9):
GFDFSRYWMS
CDR_{H}2 of DR5 mAb 1 (SEQ ID NO: 10):
EINPDSNTINYTPSLKD
CDR_{H}3 of DR5 mAb 1 (SEQ ID NO: 11):
RAYYGNPAWFAY

The VH Domain of DR5 mAb 1 is preferably encoded by a polynucleotide (SEQ ID NO:12) having the sequence shown below (polynucleotides encoding the CDRs are shown in underline):

gaggtgaagt ttctcgagtc tggaggtggc ctggtgcagc
ctggaggatc cctgaaactc tcctgtgtag cctcaggatt
cgattttagt agatactgga tgagttgggt ccggcaggct
ccagggaaag ggctagaatg gattggagaa attaatccag
atagcaatac gataaactat acgccatctc taaaggataa
attcatcatc tccagagaca acgccaaaaa tacgctgtat
ctgcaaatga ccaaagtgag atctgaggac acagcccttt
attattgtac aagaagggcc tactatggta acccggcctg
gtttgcttac tggggccaag ggactctggt cactgtctct
tcc

b. The Anti-Human DR5 Antibody DR5 mAb 2

(1) Murine Anti-Human Antibody DR5 mAb 2

The amino acid sequence of the VL Domain of DR5 mAb 2 (SEQ ID NO:13) is shown below (CDR residues are shown underlined):

DIVMTQSHKF MSTSVGDRVS ITCKASQDVN TAVAWYQQKP
GQSPKLLIYW ASTRHTGVPD RFTGSGSGTD YTLTIKSVQA
EDLTLYYCQQ HYITPWTFGG GTKLEIK
CDRL1 of DR5 mAb 2 (SEQ ID NO: 14):
KASQDVNTAVA
CDRL2 of DR5 mAb 2 (SEQ ID NO: 15):
WASTRHT
CDRL3 of DR5 mAb 2 (SEQ ID NO: 16):
QQHYITPWT

The VL Domain of DR5 mAb 2 is preferably encoded by a polynucleotide (SEQ ID NO:17) having the sequence shown below (polynucleotides encoding the CDRs are shown in underline):

gacattgtga tgacccagtc tcacaaattc atgtccactt
cagtaggaga cagggtcagc atcacctgca aggccagtca
ggatgtgaat actgctgtag cctggtatca acaaaaacca
gggcaatctc ctaaactact gatttactgg gcatccaccc
ggcacactgg agtccctgat cgcttcacag gcagtggatc
tgggacagat tatacactca ccatcaaaag tgtgcaggct
gaagacctga cactttatta ctgtcagcaa cactatatca
ctccgtggac gttcggtgga ggcaccaagc tggaaatcaaa

The amino acid sequence of the VH Domain of DR5 mAb 2 (SEQ ID NO:18) is shown below (CDR residues are shown underlined):

KVQLQQSGAE LVKPGASVKL SCKASGYTFT EYILHWVKQK
SGQGLEWIGW FYPGNNNIKY NEKFKDKATL TADKSSSTVY
MELSRLTSED SAVYFCARHE QGPGYFDYWG QGTTLTVSS
CDRH1 of DR5 mAb 2 (SEQ ID NO: 19):
GYTFTEYILH
CDRH2 of DR5 mAb 2 (SEQ ID NO: 20):
WFYPGNNNIKYNEKFKD
CDRH3 of DR5 mAb 2 (SEQ ID NO: 21):
HEQGPGYFDY

The VH Domain of DR5 mAb 2 is preferably encoded by a polynucleotide (SEQ ID NO:22) having the sequence shown below (polynucleotides encoding the CDRs are shown in underline):

aaggtccagc tgcagcagtc tggagctgaa ctggtgaaac
ccggggcatc agtgaagctg tcctgcaagg cttctgggta
caccttcact gagtatattt tacactgggt aaagcagaag
tctggacagg gtcttgagtg gattgggtgg ttttatcctg
gaaataataa tataaagtac aatgagaaat tcaaggacaa
ggccacactg actgcggaca aatcctccag cacagtctat
atggaactta gtagattgac atctgaagac tctgcggtct
atttctgtgc aagacacgaa caaggaccag gttactttga
ctactggggc caaggcacca ctctcacagt ctcctcc

(2) Humanized DR5 mAb 2 (“hDR5 mAb 2”)

The above-described murine anti-human DR5 antibody DR5 mAb 2 was humanized in order to demonstrate the capability of humanizing an anti-human DR5 antibody so as to decrease its antigenicity upon administration to a human recipient. The humanization yielded four humanized VL Domains designated herein as “hDR5 mAb 2 VL-2,” “hDR5 mAb 2 VL-3,” “hDR5 mAb 2 VL-4,” and “hDR5 mAb 2 VL-5,” and one humanized VH Domain, designated herein as “hDR5 mAb 2 VH-2.” Any of the humanized VL Domains may be paired with the humanized VH Domain. Accordingly, any antibody comprising one of the humanized VL Domains paired with the humanized VH Domain is referred to generically as “hDR5 mAb 2,” and particular combinations of humanized VL/VH Domains are referred to by reference to the VL domain.

The amino acid sequence of the VL Domain of hDR5 mAb 2 VL-2 (SEQ ID NO:23) is shown below (CDR residues are shown underlined):

DIQMTQSPSF LSASVGDRVT ITCKASQDVN TAVAWYQQKP
GKAPKLLIYW ASTRHTGVPS RFSGSGSGTD FTLTISSLQP
EDVATYYCQQ HYITPWTFGG GTKLEIK

hDR5 mAb 2 VL-2 is preferably encoded by a polynucleotide (SEQ ID NO:24) having the sequence shown below:

gatattcaga tgacccagag tccctcattt ctgtccgcct
ccgtcggtga ccgcgtgact attacttgta aagcttctca
ggatgtcaac accgccgtgg cttggtacca gcagaagccc
ggtaaagcac ctaagctgct gatctattgg gccagcactc
ggcacaccgg agtcccatct aggttctctg gcagtggatc
agggacagac tttaccctga caattagctc cctgcagccc
gaggatgtgg ctacttacta ttgtcagcag cactacatca
ctccttggac cttcggcggg ggcacaaaac tggaaatcaa
a

The amino acid sequence of the VL Domain of hDR5 mAb 2 VL-3 (SEQ ID NO:25) is shown below (CDR residues are shown underlined):

DIQMTQSPSF LSASVGDRVT ITCRASQDVN TAVAWYQQKP
GKAPKLLIYW ASTRHTGVPD RFSGSGSGTD FTLTISSLQP
EDVATYYCQQ HYITPWTFGG GTKLEIK

hDR5 mAb 2 VL-3 is preferably encoded by a polynucleotide (SEQ ID NO:26) having the sequence shown below:

gatattcaga tgacccagag tccctcattt ctgtccgcct
ccgtcggtga ccgcgtgact attacttgtc gggcttctca
ggatgtcaac accgccgtgg cttggtacca gcagaagccc
ggtaaagcac ctaagctgct gatctattgg gccagcactc
ggcacaccgg agtcccagat aggttctctg gcagtggatc
agggacagac tttaccctga caattagctc cctgcagccc
gaggatgtgg ctacttacta ttgtcagcag cactacatca
ctccttggac cttcggcggg ggcacaaaac tggaaatcaa
a

The amino acid sequence of the VL Domain of hDR5 mAb 2 VL-4 (SEQ ID NO:27) is shown below (CDR residues are shown underlined):

DIQMTQSPSF LSASVGDRVT ITCRASQDVN TAVAWYQQKP
GKAPKLLIYW ASTRHTGVPS RFSGSGSGTD FTLTISSLQP
EDIATYYCQQ HYITPWTFGG GTKLEIK

hDR5 mAb 2 VL-4 is preferably encoded by a polynucleotide (SEQ ID NO:28) having the sequence shown below:

gatattcaga tgacccagag tccctcattt ctgtccgcct
ccgtcggtga ccgcgtgact attacttgtc gggcttctca
ggatgtcaac accgccgtgg cttggtacca gcagaagccc
ggtaaagcac ctaagctgct gatctattgg gccagcactc
ggcacaccgg agtcccatct aggttctctg gcagtggatc
agggacagac tttaccctga caattagctc cctgcagcca
gaggatatcg ctacatacta ttgtcagcag cactacatca
ctccttggac cttcggcggg ggcacaaaac tggaaatcaa
a

The amino acid sequence of the VL Domain of hDR5 mAb 2 VL-5 (SEQ ID NO:29) is shown below (CDR residues are shown underlined):

DIQMTQSPSF LSASVGDRVT ITCRASQDVN TAVAWYQQKP
GKAPKLLIYW ASTRHTGVPD RFSGSGSGTD FTLTISSLQP
EDIATYYCQQ HYITPWTFGG GTKLEIK

hDR5 mAb 2 VL-5 is preferably encoded by a polynucleotide (SEQ ID NO:30) having the sequence shown below:

gatattcaga tgacccagag tccctcattt ctgtccgcct
ccgtcggtga ccgcgtgact attacttgtc gggcttctca
ggatgtcaac accgccgtgg cttggtacca gcagaagccc
ggtaaagcac ctaagctgct gatctattgg gccagcactc
ggcacaccgg agtcccagat aggttctctg gcagtggatc
agggacagac tttaccctga caattagctc cctgcagccc
gaggatatcg ctacttacta ttgtcagcag cactacatca
ctccttggac cttcggcggg ggcacaaaac tggaaatcaa
a

The amino acid sequence of the VH Domain of hDR5 mAb 2 VH-2 (SEQ ID NO:31) is shown below (CDR residues are shown underlined):

QVQLVQSGAE VKKPGASVKV SCKASGYTFT EYILHWVRQA
PGQGLEWMGW FYPGNNNIKY NEKFKDRVTI TADKSTSTVY
MELSSLRSED TAVYYCARHE QGPGYFDYWG QGTLVTVSS

hDR5 mAb 2 VH-2 is preferably encoded by a polynucleotide (SEQ ID NO:32) having the sequence shown below:

caggtccagc tggtgcagag tggggcagag gtgaaaaagc
caggggcatc agtgaaagtg tcttgtaaag catcaggtta
tacatttact gagtacatcc tgcactgggt gcgacaggca
ccaggacagg gactggaatg gatggggtgg ttctaccctg
gcaacaacaa cattaagtac aacgagaagt ttaaagaccg
ggtgaccatc acagcggata agtctaccag tacagtctat
atggagctga gctccctgag aagcgaagac accgccgtct
actattgcgc tcgccacgaa cagggtccag gttactttga
ttattggggg cagggaactc tggtcacagt cagctcc

The CDR1 of the VL Domain of hDR5 mAb 2 VL-3, hDR5 mAb 2 VL-4 and hDR5 mAb VL-5 has the amino acid sequence: RASQDVNTAVA (SEQ ID NO:320).

c. Drozitumab (“DR5 mAb 3”)

The amino acid sequence of the VL Domain of drozitumab (“DR5 mAb 3”) (SEQ ID NO:54) is shown below (CDR residues are shown underlined):

SELTQDPAVS VALGQTVRIT CSGDSLRSYY ASWYQQKPG
QAPVLVIYGA NNRPSGIPDR FSGSSSGNTA SLTITGAQAE
DEADYYCNSA DSSGNHVVFG GGTKLTVLG
CDRL1 of DR5 mAb 3 (SEQ ID NO: 55):
SGDSLRSYYAS
CDRL2 of DR5 mAb 3 (SEQ ID NO: 56):
GANNRPS
CDRL3 of DR5 mAb 3 (SEQ ID NO: 57):
NSADSSGNHVV

The amino acid sequence of the VH Domain of drozitumab (“DR5 mAb 3”) (SEQ ID NO:58) is shown below (CDR residues are shown underlined):

EVQLVQSGGG VERPGGSLRL SCAASGFTFD DYAMSWVRQA
PGKGLEWVSG INWQGGSTGY ADSVKGRVTI SRDNAKNSLY
LQMNSLRAED TAVYYCAKIL GAGRGWYFDY WGKGTTVTVS
S
CDR_{H}1 of DR5 mAb 3 (SEQ ID NO: 59):
GFTFDDYAMS
CDR_{H}2 of DR5 mAb 3 (SEQ ID NO: 60):
INWQGGSTGYADSVKG
CDR_{H}3 of DR5 mAb 3 (SEQ ID NO: 61):
ILGAGRGWYFDY

d. Conatumumab (“DR5 mAb 4”)

The amino acid sequence of the VL Domain of conatumumab (“DR5 mAb 4”) (SEQ ID NO:62) is shown below (CDR residues are shown underlined):

EIVLTQSPGT LSLSPGERAT LSCRASQGIS RSYLAWYQQK
PGQAPSLLIY GASSRATGIP DRFSGSGSGT DFTLTISRLE
PEDFAVYYCQ QFGSSPWTFG QGTKVEIK
CDR_{L}1 of DR5 mAb 4 (SEQ ID NO: 63):
RASQGISRSYLA
CDR_{L}2 of DR5 mAb 4 (SEQ ID NO: 64):
GASSRAT
CDR_{L}3 of DR5 mAb 4 (SEQ ID NO: 65):
QQFGSSPWT

The amino acid sequence of the VH Domain of conatumumab (“DR5 mAb 4”) (SEQ ID NO:66) is shown below (CDR residues are shown underlined):

QVQLQESGPG LVKPSQTLSL TCTVSGGSIS SGDYFWSWIR
QLPGKGLEWI GHIHNSGTTY YNPSLKSRVT ISVDTSKKQF
SLRLSSVTAA DTAVYYCARD RGGDYYYGMD VWGQGTTVTV
SS
CDR_{H}1 of DR5 mAb 4 (SEQ ID NO: 67):
GGSISSGDYFWS
CDR_{H}2 of DR5 mAb 4 (SEQ ID NO: 68):
HIHNSGTTYYNPSLKS
CDR_{H}3 of DR5 mAb 4 (SEQ ID NO: 69):
DRGGDYYYGMDV

e. Tigatumumab (“DR5 mAb 5”)

The amino acid sequence of the VL Domain of tigatumumab (“DR5 mAb 5”) (SEQ ID NO:70) is shown below (CDR residues are shown underlined):

DIQMTQSPSS LSASVGDRVT ITCKASQDVG TAVAWYQQKP
GKAPKLLIYW ASTRHTGVPS RFSGSGSGTD FTLTISSLQP
EDFATYYCQQ YSSYRTFGQG TKVEIK
CDR_{L}1 of DR5 mAb 5 (SEQ ID NO: 71):
KASQDVGTAVA
CDR_{L}2 of DR5 mAb 5 (SEQ ID NO: 72):
WASTRHT
CDR_{L}3 of DR5 mAb 5 (SEQ ID NO: 73):
QQYSSYRT

The amino acid sequence of the VH Domain of tigatumumab (“DR5 mAb5”) (SEQ ID NO:74) is shown below (CDR residues are shown underlined):

EVQLVESGGG LVQPGGSLRL SCAASGFTFS SYVMSWVRQA
PGKGLEWVAT ISSGGSYTYY PDSVKGRFTI SRDNAKNTLY
LQMNSLRAED TAVYYCARRG DSMITTDYWG QGTLVTVSS
CDR_{H}1 of DR5 mAb 5 (SEQ ID NO: 75):
GFTFSSYVMS
CDR_{H}2 of DR5 mAb 5 (SEQ ID NO: 76):
TISSGGSYTYYPDSVKG
CDR_{H}3 of DR5 mAb 5 (SEQ ID NO: 77):
RGDSMITTDY

f. LBY135-1 (“DR5 mAB 6”)

The amino acid sequence of the VL Domain of LBY135-1 (“DR5 mAb 6”)

DIAMTQSHKF MSTLVGDRVS ITCKASQDVN TAIAWYQQKP
GQSPKLLIYW ASTRHTGVPD RFYGSGSGTD YTLTISSMEA
EDAATYYCQQ WSSNPLTFGA GTKLELKRA
CDR_{L}1 of DR5 mAb 6 (SEQ ID NO: 79):
QDVNTAIA
CDR_{L}2 of DR5 mAb 6 (SEQ ID NO: 80):
WASTRHT
CDR_{L}3 of DR5 mAb 6 (SEQ ID NO: 81):
QQWSSNPLT

The amino acid sequence of the VH Domain of LBY135-1 (“DR5 mAb 6”) (SEQ ID NO:82) is shown below (CDR residues are shown underlined):

KVQLQQSGAE LVKPGASVKL SCKASGYTFT DYTIHWVKQR
SGQGLEWIGW FYPGGGYIKY NEKFKDRATL TADKSSNTVY
MELSRLTSEG SAVYFCARHE EGIYFDYWGQ GTTLVTVSS
CDR_{H}1 of DR5 mAb 6 (SEQ ID NO: 83):
GYTFTDYTIH
CDR_{H}2 of DR5 mAb 6 (SEQ ID NO: 84):
WFYPGGGYIKYNEKFKD
CDR_{H}3 of DR5 mAb 6 (SEQ ID NO: 85):
HEEGIYFDY

g. LBY135-2 (“DR5 mAB 7”)

The amino acid sequence of the VL Domain of LBY135-2 (“DR5 mAb 7”) (SEQ ID NO:86) is shown below (CDR residues are shown underlined):

DIVMTQSHKF MSTSVGDRVS ITCKASQDVN TAIAWYQQKP
GQSPKLLIYW ASTRHTGVPD RFTGSGSGTD YTLTISSVQA
EDLALYYCQQ HYTTPFTFGS GTKL
CDR_{L}1 of DR5 mAb 7 (SEQ ID NO: 87):
KASQDVNTAIA
CDR_{L}2 of DR5 mAb 7 (SEQ ID NO: 88):
WASTRHT
CDR_{L}3 of DR5 mAb 7 (SEQ ID NO: 89):
QQHYTTPFT

The amino acid sequence of the VH Domain of LBY135-2 (“DR5 mAb 7”) (SEQ ID NO:90) is shown below (CDR residues are shown underlined):

KVQLQQSGAE LVKPGASVKL SCKASGYTFT DYTIHWVKQR
SGQGLEWIGW FYPGGGYIKY NEKFKDRATL TADKSSNTVY
MELSRLTSED SAVYFCARHE EGIYFDYWGQ GTTLTVSS
CDR_{H}1 of DR5 mAb 7 (SEQ ID NO: 91):
GYTFTDYTIH
CDR_{H}2 of DR5 mAb 7 (SEQ ID NO: 92):
WFYPGGGYIKYNEKFKD
CDR_{H}3 of DR5 mAb 7 (SEQ ID NO: 93):
HEEGIYFDY

h. KMTR2 (“DR5 mAB 8”)

The amino acid sequence of the VL Domain of KMTR2 (“DR5 mAb 8”) (SEQ ID NO:94) is shown below (CDR residues are shown underlined):

EIVLTQSPAT LSLSPGERAT LSCRASQSVS SYLAWYQQKP
GQAPRLLIYD ASNRATGIPA RFSGSGSGTD FTLTISSLEP
EDFAVYYCQQ RSNWPLTFGG GTKVEIKR
CDR_{L}1 of DR5 mAb 8 (SEQ ID NO: 95):
RASQSVSSYLA
CDR_{L}2 of DR5 mAb 8 (SEQ ID NO: 96):
DASNRAT
CDR_{L}3 of DR5 mAb 8 (SEQ ID NO: 97):
QQRSNWPLT

The amino acid sequence of the VH Domain of KMTR2 (“DR5 mAb 8”) (SEQ ID NO:98) is shown below (CDR residues are shown underlined):

QVQLVQSGAE MKKPGASVKV SCKTSGYTFT NYKINWVRQA
PGQGLEWMGW MNPDTDSTGY PQKFQGRVTM TRNTSISTAY
MELSSLRSED TAVYYCARSY GSGSYYRDYY YGMDVWGQGT
TVTVSS
CDR_{H}1 of DR5 mAb 8 (SEQ ID NO: 99):
GYTFTNYKIN
CDR_{H}2 of DR5 mAb 8 (SEQ ID NO: 100):
WMNPDTDSTGYPQKFQG
CDR_{H}3 of DR5 mAb 8 (SEQ ID NO: 101):
SYGSGSYYRDYYYGMDV

5. EphA2-Binding Domains

The receptor tyrosine kinase, ephrin type-A receptor 2 (EphA2) is a preferred cancer antigen of the present invention. EphA2 is normally expressed at sites of cell-to-cell contact in adult epithelial tissues, however, recent studies have shown that it is also overexpressed in various types of epithelial carcinomas, with the greatest level of EphA2 expression observed in metastatic lesions. High expression levels of EphA2 have been found in a wide range of cancers and in numerous tumor cell lines, including prostate cancer, breast cancer, non-small cell lung cancer and melanoma (Xu, J. et al. (2014) “High Epha2 Protein Expression In Renal Cell Carcinoma Is Associated With A Poor Disease Outcome,” Oncol. Lett. August 2014; 8(2): 687-692; Miao, B. et al. (2014) “EphA2 is a Mediator of Vemurafenib Resistance and a Novel Therapeutic Target in Melanoma,” Cancer Discov. pii: CD-14-0295. EphA2 does not appear to be merely a marker for cancer, but rather appears to be persistently overexpressed and functionally changed in numerous human cancers (Chen, P. et al. (2014) “Epha2 Enhances The Proliferation And Invasion Ability Of Lncap Prostate Cancer Cells,” Oncol. Lett. 8(1):41-46).

The invention particularly contemplates the selection of EphA2 as a Cancer Antigen, and the use of anti-EphA2 antibodies to provide the Cancer Antigen-Binding Domain of the Tri-Specific Binding Molecules of the present invention. Exemplary anti-EphA2 antibodies include “EphA2 mAb 1,” “EphA2 mAb 2” and “EphA2 mAb 3.”

a. EphA2 mAb 1

The amino acid sequence of the VL Domain of a preferred anti-human EphA2 antibody (“EphA2 mAb 1”) (SEQ ID NO:153) is shown below (CDR residues are shown underlined):

DIQMTQTTSS LSASLGDRIT ISCRASQDIS NYLNWYQQKP
DGTVKLLIYY TSRLHSGVPS RFSGSGSGTD YSLTISNLEQ
EDIATYFCQQ GYTLYTFGGG TKLEIK
CDR_{L}1 of EphA2 mAb 1 (SEQ ID NO: 154):
RASQDISNYLN
CDR_{L}2 of EphA2 mAb 1 (SEQ ID NO: 155):
YTSRLHS
CDR_{L}3 of EphA2 mAb 1 (SEQ ID NO: 156):
QQGYTLYT

The VL Domain of EphA2 mAb 1 is preferably encoded by a polynucleotide (SEQ ID NO:157) having the sequence shown below (polynucleotides encoding the CDRs are shown in underline):

gatatccaga tgacacagac tacatcctcc ctgtctgcct ctctgggaga cagaatcacc
atcagttgca gggcaagtca ggacattagc aattatttaa actggtatca gcagaaacca
gatggaactg ttaaactcct gatctactac acatcaagat tacactcagg agtcccatca
aggttcagtg gcagtgggtc tggaacagat tattctctca ccattagcaa cctggagcaa
gaagatattg ccacttactt ttgccaacag ggttatacgc tgtacacgtt cggagggggg
accaagctgg aaataaaa

The amino acid sequence of the VH Domain of EphA2 mAb 1 (SEQ ID NO:158) is shown below (CDR residues are shown underlined):

QVQLKESGPG LVAPSQSLSI TCTVSGFSLS RYSVHWVRQP
PGKGLEWLGM IWGGGSTDYN SALKSRLSIS KDNSKSQVFL
KMNSLQTDDT AMYYCARKHG NYYTMDYWGQ GTSVTVSS
CDR_{H}1 of EphA2 mAb 1 (SEQ ID NO: 159):
GFSLSRYSVH
CDR_{H}2 of EphA2 mAb 1 (SEQ ID NO: 160):
MIWGGGSTDYNSALKS
CDR_{H}3 of EphA2 mAb 1 (SEQ ID NO: 161):
KHGNYYTMDY

The VH Domain of EphA2 mAb 1 is preferably encoded by a polynucleotide (SEQ ID NO:162) having the sequence shown below (polynucleotides encoding the CDRs are shown in underline):

caggtgcagc tgaaggagtc aggacctggc ctggtggcac cctcacagag cctgtccatc acatgcactg
tctctgggtt ctcattatcc agatatagtg tacactgggt tcgccagcct ccaggaaagg gtctggagtg
gctgggaatg atatggggtg gtggaagcac agactataat tcagctctca aatccagact gagtatcagc
aaggacaact ccaagagcca agttttctta aaaatgaaca gtctgcaaac tgatgacaca gccatgtact
actgtgccag aaaacatggt aactactata ctatggacta ctggggtcaa ggaacctcag tcaccgtctc
ctcc

b. EphA2 mAb 2

The amino acid sequence of the VL Domain of a second preferred anti-human EphA2 antibody (“EphA2 mAb 2”) (SEQ ID NO:163) is shown below (CDR residues are shown underlined):

DVVMTQTPLS LPVSLGDQAS ISCRSSQSLV HSSGNTYLHW
YLQKPGQSPK LLIYKVSNRF SGVPDRFSGS GSGTDFTLKI
SRVEAEDLGV YFCSQSTHVP TFGSGTKLEI K
CDR_{L}1 of EphA2 mAb 2 (SEQ ID NO: 164):
RSSQSLVHSSGNTYLH
CDR_{L}2 of EphA2 mAb 2 (SEQ ID NO: 165):
KVSNRFS
CDR_{L}3 of EphA2 mAb 2 (SEQ ID NO: 166):
SQSTHVPT

The VL Domain of EphA2 mAb 2 is preferably encoded by a polynucleotide (SEQ ID NO:318) having the sequence shown below (polynucleotides encoding the CDRs are shown in underline):

gatgttgtga tgacccaaac tccactctcc ctgcctgtca
gtcttggaga tcaagcctcc atctcttgca gatctagtca
gagccttgta cacagtagtg gaaacaccta tttacattgg
tacctgcaga agccaggcca gtctccaaag ctcctgatct
acaaagtttc caaccgattt tctggggtcc cagacaggtt
cagtggcagt ggatcaggga cagatttcac actcaagatc
agcagagtgg aggctgagga tctgggagtt tatttctgct
ctcaaagtac acatgttccc acgttcggct cggggacaaa
gttggaaata aaa

The amino acid sequence of the VH Domain of EphA2 mAb 2 (SEQ ID NO:167) is shown below (CDR residues are shown underlined):

QIQLVQSGPE LKKPGETVKI SCKASGFTFT NYGMNWVKQA
PGKGLKWMGW INTYIGEPTY ADDFKGRFVF SLETSASTAY
LQINNLKNED MATYFCAREL GPYYFDYWGQ GTTLTVSS
CDR_{H}1 of EphA2 mAb 2 (SEQ ID NO: 168):
GFTFTNYGMN
CDR_{H}2 of EphA2 mAb 2 (SEQ ID NO: 169):
WINTYIGEPTYADDFKG
CDR_{H}3 of EphA2 mAb 2 (SEQ ID NO: 170):
ELGPYYFDY

The VH Domain of EphA2 mAb 2 is preferably encoded by a polynucleotide (SEQ ID NO:171) having the sequence shown below (polynucleotides encoding the CDRs are shown in underline):

cagatccagt tggtgcagtc tggacctgag ctgaagaagc
ctggagagac agtcaagatc tcctgcaagg cttctgggtt
taccttcaca aactatggaa tgaactgggt gaagcaggct
ccaggaaagg gtttaaagtg gatgggctgg ataaacacct
atattggaga gccgacatat gctgatgact tcaagggacg
gtttgtcttc tctttggaaa cctctgccag cactgcctat
ttgcagatca acaacctcaa aaatgaggac atggccacat
atttctgtgc aagagaactg ggaccatact actttgacta
ctggggccaa ggcaccactc tcacagtctc ctcc

c. EphA2 mAb 3

The amino acid sequence of the VL Domain of a further preferred anti-human EphA2 antibody (“EphA2 mAb 3”) (SEQ ID NO:172) is shown below (CDR residues are shown underlined):

DIVLTQSHRS MSTSVGDRVN ITCKASQDVT TAVAWYQQKP
GQSPKLLIFW ASTRHAGVPD RFTGSGSGTD FTLTISSVQA
GDLALYYCQQ HYSTPYTFGG GTKLEIK
CDR_{L}1 of EphA2 mAb 3 (SEQ ID NO: 173):
KASQDVTTAVA
CDR_{L}2 of EphA2 mAb 3 (SEQ ID NO: 174):
WASTRHA
CDR_{L}3 of EphA2 mAb 3 (SEQ ID NO: 175):
QQHYSTPYT

The VL Domain of EphA2 mAb 3 is preferably encoded by a polynucleotide (SEQ ID NO:176) having the sequence shown below (polynucleotides encoding the CDRs are shown in underline):

gacattgtgc tgacccagtc tcacagatcc atgtccacat
cagtaggaga cagggtcaac atcacctgca aggccagtca
ggatgtgact actgctgtag cctggtatca acaaaaacca
gggcaatctc ctaaattact gattttctgg gcatccaccc
ggcacgctgg agtccctgat cgcttcacag gcagtggatc
tgggacagat tttactctca ccatcagcag tgtgcaggct
ggagacctgg cactttatta ctgtcaacaa cattatagca
caccgtacac attcggaggg gggaccaagc tggaaataaa
a

The amino acid sequence of the VH Domain of EphA2 mAb 3 (SEQ ID NO:177) is shown below (CDR residues are shown underlined):

EVQLVESGGG SVKPGGSLKL SCAASGFTFT DHYMYWVRQT
PEKRLEWVAT ISDGGSFTSY PDSVKGRFTI SRDIAKNNLY
LQMSSLKSED TAMYYCTRDE SDRPFPYWGQ GTLVTVSS
CDR_{H}1 of EphA2 mAb 3 (SEQ ID NO: 178):
GFTFTDHYMY
CDR_{H}2 of EphA2 mAb 3 (SEQ ID NO: 179):
TISDGGSFTSYPDSVKG
CDR_{H}3 of EphA2 mAb 3 (SEQ ID NO: 180):
DESDRPFPY

The VH Domain of EphA2 mAb 3 is preferably encoded by a polynucleotide (SEQ ID NO:319) having the sequence shown below (polynucleotides encoding the CDRs are shown in underline):

gaagtgcagc tggtggagtc tgggggaggc tcagtgaagc
ctggagggtc cctgaaactc tcctgtgcag cctctggatt
cactttcact gaccattaca tgtattgggt tcgccagact
ccggaaaaga ggctggagtg ggtcgcaacc attagtgatg
gcggtagttt cacctcctat ccagacagtg tgaaggggcg
attcaccatc tccagagaca ttgccaagaa caacctgtac
ctccaaatga gcagtctgaa gtctgaggac acagccatgt
attactgtac aagagatgag agcgataggc cgtttcctta
ctggggccaa gggactctgg tcactgtctc ctcc

6. gpA33-Binding Domains

gpA33 is also a preferred cancer antigen of the present invention. Colorectal cancer is among the most common malignancies of the Western world and is a leading cause of cancer deaths (Silverberg, E. et al. (1989) “Cancer Statistics, 1989,” CA Cancer J Clin. 39(1):3-20). One potentially useful target for colon cancer is the 43 kD transmembrane glycoprotein A33 (gpA33), which is expressed in >95% of all colorectal carcinomas (Heath, J. K. et al. (1997) “The Human A33 Antigen Is A Transmembrane Glycoprotein And A Novel Member Of The Immunoglobulin Superfamily,” Proc. Natl. Acad. Sci. (U.S.A.) 94(2):469-474; Ritter, G. et al. (1997) “Characterization Of Posttranslational Modifications Of Human A33 Antigen, A Novel Palmitoylated Surface Glycoprotein Of Human Gastrointestinal Epithelium,” Biochem. Biophys. Res. Commun. 236(3):682-686; Wong, N. A. et al. (2006) “EpCAM and gpA33 Are Markers Of Barrett's Metaplasia,” J. Clin. Pathol. 59(3):260-263). gpA33 was first discovered through raising monoclonal murine antibodies against the human pancreatic carcinoma derived cell line ASPC1.

The invention particularly contemplates the selection of gpA33 as a Cancer Antigen, and the use of anti-gpA33 antibodies to provide the Cancer Antigen-Binding Domain of the Tri-Specific Binding Molecules of the present invention. An exemplary anti-gpA33 antibody is “gpA33 mAb 1.”

The amino acid sequence of the VL Domain of a preferred anti-human gpA33 antibody (“gpA33 mAb 1”) (SEQ ID NO:181) is shown below (CDR residues are shown underlined):

DIQLTQSPSF LSASVGDRVT ITCSARSSIS FMYWYQQKPG
KAPKLLIYDT SNLASGVPSR FSGSGSGTEF TLTISSLEAE
DAATYYCQQW SSYPLTFGQG TKLEIK
CDR_{L}1 of gpA33 mAb 1 (SEQ ID NO: 182):
SARSSISFMY
CDR_{L}2 of gpA33 mAb 1 (SEQ ID NO: 183):
DTSNLAS
CDR_{L}3 of gpA33 mAb 1 (SEQ ID NO: 184):
QQWSSYPLT

The VL Domain of gpA33 mAb 1 is preferably encoded by a polynucleotide (SEQ ID NO:185) having the sequence shown below (polynucleotides encoding the CDRs are shown in underline):

gacattcagc tgactcagtc cccctctttt ctgtccgcat
ccgtcggaga tcgagtgact attacttgct ctgctaggtc
ctcaatcagc ttcatgtact ggtatcagca gaagcccggc
aaagcaccta agctgctgat ctacgacaca agcaacctgg
cctccggggt gccatctcgg ttctctggca gtgggtcagg
aactgagttt accctgacaa ttagctccct ggaggctgaa
gatgccgcta cctactattg ccagcagtgg agcagctatc
ctctgacctt cggacagggg actaaactgg aaatcaag

The amino acid sequence of the VH Domain of gpA33 mAb 1 (SEQ ID NO:186) is shown below (CDR residues are shown underlined):

QVQLVQSGAE VKKPGASVKV SCKASGYTFT GSWMNWVRQA
PGQGLEWIGR IYPGDGETNY NGKFKDRVTI TADKSTSTAY
MELSSLRSED TAVYYCARIY GNNVYFDVWG QGTTVTVSS
CDR_{H}1 of gpA33 mAb 1 (SEQ ID NO: 187):
GYTFTGSWMN
CDR_{H}2 of gpA33 mAb 1 (SEQ ID NO: 188):
RIYPGDGETNYNGKFKD
CDR_{H}3 of gpA33 mAb 1 (SEQ ID NO: 189):
IYGNNVYFDV

The VH Domain of gpA33 mAb 1 is preferably encoded by a polynucleotide (SEQ ID NO:190) having the sequence shown below (polynucleotides encoding the CDRs are shown in underline):

caggtccagc tggtccagag cggggccgaa gtcaaaaaac
ccggagcaag cgtgaaggtc tcctgcaaag catcaggcta
tacatttaca ggcagctgga tgaactgggt gaggcaggct
ccaggacagg gactggagtg gatcgggcgc atctaccctg
gagacggcga aactaactat aatggaaagt tcaaagaccg
agtgaccatc acagccgata agtctactag taccgcctac
atggagctga gctccctgcg gtctgaagat accgccgtct
actattgcgc tagaatttac ggaaacaatg tctattttga
cgtgtggggg cagggaacaa ctgtgactgt ctcctcc

7. Her2-Binding Domains

The invention also particularly contemplates the selection of Her2 as a Cancer Antigen, and the use of anti-Her2 antibodies to provide the Cancer Antigen-Binding Domain of the Tri-Specific Binding Molecules of the present invention. Exemplary anti-Her2 antibodies include “Her2 mAb 1” and Trastuzumab.

a. Her2 mAb 1

The amino acid sequence of the VL Domain of anti-Her2 antibody “Her2 mAb 1” (SEQ ID NO:191) is shown below (CDR residues are shown underlined):

DIQMTQSPSS LSASVGDRVT ITCRASQDVN TAVAWYQQKP
GKAPKLLIYS ASFLESGVPS RFSGSRSGTD FTLTISSLQP
EDFATYYCQQ HYTTPPTFGQ GTKVEIKRT

The amino acid sequence of the VH Domain of anti-Her2 antibody “Her2 mAb 1” (SEQ ID NO:192) is shown below (CDR residues are shown underlined):

QVQLQQSGPE LVKPGASLKL SCTASGFNIK DTYIHWVKQR
PEQGLEWIGR IYPTNGYTRY DPKFQDKATI TADTSSNTAY
LQVSRLTSED TAVYYCSRWG GDGFYAMDYW GQGASVTVSS

b. Trastuzumab

The amino acid sequence of the VL Domain of the humanized anti-Her2 antibody “Trastuzumab” (SEQ ID NO:193) is shown below (CDR residues are shown underlined):

DIQMTQSPSS LSASVGDRVT ITCRASQDVN TAVAWYQQKP
GKAPKLLIYS ASFLYSGVPS RFSGSRSGTD FTLTISSLQP
EDFATYYCQQ HYTTPPTFGQ GTKVEIKR

The amino acid sequence of the VH Domain of the humanized anti-Her2 antibody “Trastuzumab” (SEQ ID NO:194) is shown below (CDR residues are shown underlined):

EVQLVESGGG LVQPGGSLRL SCAASGFNIK DTYIHWVRQA
PGKGLEWVAR IYPTNGYTRY ADSVKGRFTI SADTSKNTAY
LQMNSLRAED TAVYYCSRWG GDGFYAMDYW GQGTLVTVSS

8. B7-H3-Binding Domains

In addition to its expression on neuroblastoma cells, human B7-H3 is also known to be expressed on a variety of other cancer cells (e.g., gastric, ovarian and non-small cell lung cancers). B7-H3 protein expression has been immunohistologically detected in tumor cell lines (Chapoval, A. et al. (2001) “B7-H3: A Costimulatory Molecule For T Cell Activation and IFN-γ Production,” Nature Immunol. 2:269-274; Saatian, B. et al. (2004) “Expression Of Genes For B7-H3 And Other T Cell Ligands By Nasal Epithelial Cells During Differentiation And Activation,” Amer. J. Physiol. Lung Cell. Mol. Physiol. 287:L217-L225; Castriconi et al. (2004) “Identification Of 4Ig-B7-H3 As A Neuroblastoma-Associated Molecule That Exerts A Protective Role From An NK Cell-Mediated Lysis,” Proc. Natl. Acad. Sci. (U.S.A.) 101(34):12640-12645); Sun, M. et al. (2002) “Characterization of Mouse and Human B7-H3 Genes,” J. Immunol. 168:6294-6297). mRNA expression has been found in heart, kidney, testes, lung, liver, pancreas, prostate, colon, and osteoblast cells (Collins, M. et al. (2005) “The B7 Family Of Immune-Regulatory Ligands,” Genome Biol. 6:223.1-223.7). At the protein level, B7-H3 is found in human liver, lung, bladder, testis, prostate, breast, placenta, and lymphoid organs (Hofmeyer, K. et al. (2008) “The Contrasting Role Of B7-H3,” Proc. Natl. Acad. Sci. (U.S.A.) 105(30):10277-10278).

The invention also particularly contemplates the selection of B7-H3 as a Cancer Antigen, and the use of anti-B7-H3 antibodies to provide the Cancer Antigen-Binding Domain of the Tri-Specific Binding Molecules of the present invention. Exemplary anti-B7-H3 antibodies include “B7-H3 mAb 1,” “B7-H3 mAb 2,” and “B7-H3 mAb 3.”

a. B7-H3 mAb 1

The amino acid sequence of the VL Domain of anti-B7-H3 antibody “B7-H3 mAb 1” (SEQ ID NO:195) is shown below (CDR residues are shown underlined):

DIAMTQSQKF MSTSVGDRVS VTCKASQNVD TNVAWYQQKP
GQSPKALIYS ASYRYSGVPD RFTGSGSGTD FTLTINNVQS
EDLAEYFCQQ YNNYPFTFGS GTKLEIK

The amino acid sequence of the VH Domain of anti-B7-H3 antibody “B7-H3 mAb 1” (SEQ ID NO:196) is shown below (CDR residues are shown underlined):

DVQLVESGGG LVQPGGSRKL SCAASGFTFS SFGMHWVRQA
PEKGLEWVAY ISSDSSAIYY ADTVKGRFTI SRDNPKNTLF
LQMTSLRSED TAMYYCGRGR ENIYYGSRLD YWGQGTTLTV
SS

b. B7-H3 mAb 2

The amino acid sequence of the VL Domain of anti-B7-H3 antibody “B7-H3 mAb 2” (SEQ ID NO:197) is shown below (CDR residues are shown underlined):

DIQMTQTTSS LSASLGDRVT ISCRASQDIS NYLNWYQQKP
DGTVKLLIYY TSRLHSGVPS RFSGSGSGTD YSLTIDNLEQ
EDIATYFCQQ GNTLPPTFGG GTKLEIK

The amino acid sequence of the VH Domain of anti-B7-H3 antibody “B7-H3 mAb 2” (SEQ ID NO:198) is shown below (CDR residues are shown underlined):

QVQLQQSGAE LARPGASVKL SCKASGYTFT SYWMQWVKQR
PGQGLEWIGT IYPGDGDTRY TQKFKGKATL TADKSSSTAY
MQLSSLASED SAVYYCARRG IPRLWYFDVW GAGTTVTVSS

c. B7-H3 mAb 3

The amino acid sequence of the VL Domain of anti-B7-H3 antibody “B7-H3 mAb 3” (SEQ ID NO:199) is shown below (CDR residues are shown underlined):

DIQMTQSPAS LSVSVGETVT ITCRASESIY SYLAWYQQKQ
GKSPQLLVYN TKTLPEGVPS RFSGSGSGTQ FSLKINSLQP
EDFGRYYCQH HYGTPPWTFG GGTNLEIK

The amino acid sequence of the VH Domain of anti-B7-H3 antibody “B7-H3 mAb 3” (SEQ ID NO:200) is shown below (CDR residues are shown underlined):

EVQQVESGGD LVKPGGSLKL SCAASGFTFS SYGMSWVRQT
PDKRLEWVAT INSGGSNTYY PDSLKGRFTI SRDNAKNTLY
LQMRSLKSED TAMYYCARHD GGAMDYWGQG TSVTVSS

9. EGF Receptor-Binding Domains (Cetuximab)

The amino acid sequence of the VL Domain of the chimeric anti-EGFR antibody “Cetuximab” (SEQ ID NO:201) is shown below (CDR residues are shown underlined):

DILLTQSPVI LSVSPGERVS FSCRASQSIG TNIHWYQQRT
NGSPRLLIKY ASESISGIRS RFSGSGSGTD FTLSINSVES
EDIADYYCQQ NNNWPTTFGA GTKLELKR

The amino acid sequence of the VH Domain of the chimeric anti-EGFR antibody “Cetuximab” (SEQ ID NO:202) is shown below (CDR residues are shown underlined):

QVQLKQSGPG LVQPSQSLSI TCTVSGFSLT NYGVHWVRQS
PGKGLEWLGV IWSGGNTDYN TPFTSRLSIN KDNSKSQVFF
KMNSLQSNDT AIYYCARALT YYDYEFAYWG QGTLVTVSA

Panitumumab (e.g., VECTIBIX®, Amgen) is an alternative EGF receptor-binding antibody that may be used in accordance with the present invention.

10. VEGF-Binding Domains (Bevacizumab)

The amino acid sequence of the VL Domain of the humanized anti-VEGF antibody “Bevacizumab” (SEQ ID NO:203) is shown below (CDR residues are shown underlined):

DIQMTQSPSS LSASVGDRVT ITCSASQDIS NYLNWYQQKP
GKAPKVLIYF TSSLHSGVPS RFSGSGSGTD FTLTISSLQP
EDFATYYCQQ YSTVPWTFGQ GTKVEIKR

The amino acid sequence of the VH Domain of the humanized anti-VEGF antibody “Bevacizumab” (SEQ ID NO:204) is shown below (CDR residues are shown underlined):

EVQLVESGGG LVQPGGSLRL SCAASGYTFT NYGMNWVRQA
PGKGLEWVGW INTYTGEPTY AADFKRRFTF SLDTSKSTAY
LQMNSLRAED TAVYYCAKYP HYYGSSHWYF DVWGQGTLVT
VSS

11. 5T4-Binding Domains

The oncofetal protein, 5T4, is a tumor-associated protein displayed on the cell membrane of many carcinomas, including kidney, colon, prostate, lung, carcinoma and in acute lymphoblastic leukemia (see, Boghaert, E. R. et al. (2008) “The Oncofetal Protein, 5T4, Is A Suitable Target For Antibody-Guided Anti-Cancer Chemotherapy With Calicheamicin,” Int. J. Oncol. 32(1):221-234; Eisen, T. et al. (2014) “Naptumomab Estafenatox: Targeted Immunotherapy with a Novel Immunotoxin,” Curr. Oncol. Rep. 16:370, pp. 1-6). The amino acid sequence of the Light Chain Variable Domain of an exemplary anti-5T4 antibody (“5T4 mAb 1”) is shown below (CDR residues are shown underlined): (SEQ ID NO:308):

DIQMTQSPSS LSASVGDRVT ITCRASQGIS NYLAWFQQKP
GKAPKSLIYR ANRLQSGVPS RFSGSGSGTD FTLTISSLQP
EDVATYYCLQ YDDFPWTFGQ GTKLEIK

The amino acid sequence of the Heavy Chain Variable Domain of such exemplary anti-5T4 antibody is shown below (CDR residues are shown underlined): (SEQ ID NO:309):

QVQLVQSGAE VKKPGASVKV SCKASGYTFT SFWMHWVRQA
PGQGLEWMGR IDPNRGGTEY NEKAKSRVTM TADKSTSTAY
MELSSLRSED TAVYYCAGGN PYYPMDYWGQ GTTVTVSS

The amino acid sequence of the Light Chain Variable Domain of a second exemplary anti-5T4 antibody (“5T4 mAb 2”) is shown below (CDR residues are shown underlined): (SEQ ID NO:310):

DVLMTQTPLS LPVSLGDQAS ISCRSSQSIV YSNGNTYLEW
YLQKPGQSPK LLIYKVSNRF SGVPDRFSGS GSGTDFTLKI
SRVEAEDLGV YYCFQGSHVP FTFGSGTKLE IK

The amino acid sequence of the Heavy Chain Variable Domain of such second exemplary anti-5T4 antibody is shown below (CDR residues are shown underlined) (SEQ ID NO:311):

QVQLQQPGAE LVKPGASVKM SCKASGYTFT SYWITWVKQR
PGQGLEWIGD IYPGSGRANY NEKFKSKATL TVDTSSSTAY
MQLSSLTSED SAVYNCARYG PLFTTVVDPN SYAMDYWGQG
TSVTVSS

12. IL13Rα2-Binding Domains

Interleukin-13 Receptor α2 (IL13Rα2) is overexpressed in a variety of cancers, including glioblastoma, colorectal cancer, cervical cancer, pancreatic cancer, multiple melanoma, osteosarcoma, leukemia, lymphoma, prostate cancer and lung cancer (PCT Publication No. WO 2008/146911;

Brown, C. E. et al. (2013) “Glioma IL13Rα2 Is Associated With Mesenchymal Signature Gene Expression And Poor Patient Prognosis,” PLoS One. 18; 8(10):e77769; Barderas, R. et al. (2012) “High Expression Of IL-13 Receptor A2 In Colorectal Cancer Is Associated With Invasion, Liver Metastasis, And Poor Prognosis,” Cancer Res. 72(11):2780-2790; Kasaian, M. T. et al. (2011) “IL-13 Antibodies Influence IL-13 Clearance In Humans By Modulating Scavenger Activity Of IL-13Rα2,” J. Immunol. 187(1):561-569; Bozinov, O. et al. (2010) “Decreasing Expression Of The Interleukin-13 Receptor IL-13Ralpha2 In Treated Recurrent Malignant Gliomas,” Neurol. Med. Chir. (Tokyo) 50(8):617-621; Fujisawa, T. et al. (2009) “A novel role of interleukin-13 receptor alpha2 in pancreatic cancer invasion and metastasis,” Cancer Res. 69(22):8678-8685). Antibodies that immunospecifically bind to IL13Rα2 are commercially available (Abnova Corporation, Biorbyt, LifeSpan BioSciences, United States Biologicals; see also PCT Publication No. WO 2008/146911). The amino acid sequence of the Light Chain Variable Domain of an exemplary anti-IL13Rα2 antibody (“hu08,” PCT Publication No. WO 2014/072888) is shown below (CDR residues are shown underlined): (SEQ ID NO:321):

DIQMTQSPSS LSASVGDRVT ITCKASQDVG TAVAWYQQKP
GKAPKLLIYS ASYRSTGVPS RFSGSGSGTD FTLTISSLQP
EDFATYYCQH HYSAPWTFGG GTKVEIK

The amino acid sequence of the Heavy Chain Variable Domain of such exemplary anti-IL13Rα2 antibody (“hu08,” PCT Publication No. WO 2014/072888) is shown below (CDR residues are shown underlined): (SEQ ID NO:322):

EVQLVESGGG LVQPGGSLRL SCAASGFTFS RNGMSWVRQA
PGKGLEWVAT VSSGGSYIYY ADSVKGRFTI SRDNAKNSLY
LQMNSLRAED TAVYYCARQG TTALATRFFD VWGQGTLVTV
SS

13. Integrin Beta6-Binding Domains

Integrinβ6 (ITGB6) is a subtype of integrin that is expressed exclusively on the surfaces of epithelial cells and is a receptor for extracellular matrix (ECM) proteins. ITGB6 expression is specifically expressed in tumor tissues (such as those of colon, prostate, kidney cancer), but is generally undetectable in healthy epithelial tissue (Liang, B. et al. (2014) “Integrinβ6-targeted Immunoliposomes Mediate Tumor Specific Drug Delivery and Enhance Therapeutic Efficacy in Colon Carcinoma,” Clin. Cancer Res. December 30. pii: clincanres.1194.2014). Monoclonal antibodies that immunospecifically bind to ITGB6 are available commercially (e.g., MAB2075Z clone R6G9, EMD Millipore; see also, Weinacker, A. et al. (1994) “Role Of The Integrin Alpha V Beta 6 In Cell Attachment To Fibronectin. Heterologous Expression Of Intact And Secreted Forms Of The Receptor,” J. Biol. Chem. 269:6940-6948). Anti-ITGB6 monoclonal antibodies 3G9 and 8G6, and variants thereof are disclosed in PCT Publication Nos. WO 03/100033 and WO 2007/008712.

The amino acid sequence of the Light Chain Variable Domain of an exemplary humanized anti-ITGB6 antibody (derived from antibody 3G9, PCT Publication No. WO 2007/008712) is shown below (CDR residues are shown underlined): (SEQ ID NO:312):

EIVLTQSPAT LSLSPGERAT LSCSASSSVS SSYLYWYQQK
PGQAPRLLIY STSNLASGIP ARFSGSGSGT GFTLTISSLE
PEDFAVYYCH QWSTYPPTFG GGTKVEIK

The amino acid sequence of the Heavy Chain Variable Domain of such exemplary humanized anti-ITGB6 antibody (derived from antibody 3G9, PCT Publication No. WO 2007/008712) is shown below (CDR residues are shown underlined): (SEQ ID NO:313):

EVQLVESGGG LVQPGGSLRL SCAASGFTFS RYWMSWVRQA
PGKGLEWVAS ISSGGRMYYP FTVKGRFTIS RDNAKNSLYL
QMNSLRAEDT AVYYCARGSI YDGYYVFPYW GQGTLVTVSS

The amino acid sequence of the Light Chain Variable Domain of an exemplary anti-ITGB6 antibody (derived from antibody 8G6, PCT Publication No. WO 2007/008712) is shown below (CDR residues are shown underlined): (SEQ ID NO:314):

EIVLTQSPAT LSLSPGERAT LSCRASQSVS TSSYSYMYWY
QQKPGQAPRL LIYYASNLES GIPARFSGSG SGTDFTLTIS
SLEPEDFAVY YCQHNWEIPF TFGGGTKVEI K

The amino acid sequence of the Heavy Chain Variable Domain of such exemplary anti-ITGB6 antibody (derived from antibody 8G6, PCT Publication No. WO 2007/008712) is shown below (CDR residues are shown underlined): (SEQ ID NO:315):

QVQLVQSGAEVKKPGASVKVSCKASGYTFTDYAMHWVRQAPGQGLEWMGV
ISTYYGNTNYNQKFKGRVTMTRDTSISTAYMELSRLRSDDTAVYYCARGG
LRRGDRPSLQYAMDYWGQGTLVTVSS

14. Additional Anti-Cancer Antigen-Binding Domains

Additional anti-cancer antigen antibodies that may be used in accordance with the present invention include the following commercially available antibodies: Brentuximab (e.g., ADCETRIS®), which binds to CD30; Gemtuzumab (e.g., MYLOTARG®, Wyeth), which binds to CD33; and Ipilimumab (e.g., YERVOY®), which binds to CTLA-4.

### C. Exemplary Effector Cell-Binding Domains

Antibodies that are capable of binding to immune system effector cells may be used to provide the Effector Cell-Binding Domains of the Tri-Specific Binding Molecules of the present invention. Particularly suitable are antibodies that bind to CD2, CD3, CD16, CD19, CD20, CD22, CD32B, CD64, the B cell Receptor (BCR), the T cell Receptor (TCR), and the NKG2D Receptor.

1. CD2-Binding Domains

CD2 is a cell adhesion molecule found on the surface of T cells and natural killer (NK) cells. CD2 enhances NK cell cytotoxicity, possibly as a promoter of NK cell nanotube formation (Mace, E. M. et al. (2014) “Cell Biological Steps And Checkpoints In Accessing NK Cell Cytotoxicity,” Immunol. Cell. Biol. 92(3):245-255; Comerci, C. J. et al. (2012) “CD2 Promotes Human Natural Killer Cell Membrane Nanotube Formation,” PLoS One 7(10):e47664:1-12). The amino acid sequence of the VL Domain of anti-CD2 antibody (Lo-CD2a; ATCC Accession No: 11423) is (SEQ ID NO:102) (CDR residues are shown underlined):

DVVLTQTPPT LLATIGQSVS ISCRSSQSLL HSSGNTYLNW
LLQRTGQSPQ PLIYLVSKLE SGVPNRFSGS GSGTDFTLKI
SGVEAEDLGV YYCMQFTHYP YTFGAGTKLE LK

The amino acid sequence of the VH Domain of anti-CD2 antibody (Lo-CD2a; ATCC Accession No: 11423) is (SEQ ID NO:103) (CDR residues are shown underlined):

EVQLQQSGPE LQRPGASVKL SCKASGYIFT EYYMYWVKQR
PKQGLELVGR IDPEDGSIDY VEKFKKKATL TADTSSNTAY
MQLSSLTSED TATYFCARGK FNYRFAYWGQ GTLVTVSS

2. CD3-Binding Domains

In a preferred embodiment, the second epitope that is bound by the Tri-Specific Binding Molecules of the present invention will be an epitope of CD3. CD3 is a T cell co-receptor composed of four distinct chains (Wucherpfennig, K. W. et al. (2010) “Structural Biology Of The T-Cell Receptor: Insights Into Receptor Assembly, Ligand Recognition, And Initiation Of Signaling,” Cold Spring Harb. Perspect. Biol. 2(4):a005140; pages 1-14). In mammals, the complex contains a CD3γ chain, a CD3δ chain, and two CD3ε chains. These chains associate with a molecule known as the T cell receptor (TCR) in order to generate an activation signal in T lymphocytes. In the absence of CD3, TCRs do not assemble properly and are degraded (Thomas, S. et al. (2010) “Molecular Immunology Lessons From Therapeutic T-Cell Receptor Gene Transfer,” Immunology 129(2):170-177). CD3 is found bound to the membranes of all mature T cells, and in virtually no other cell type (see, Janeway, C. A. et al. (2005) In: IMMUNOBIOLOGY: THE IMMUNE SYSTEM IN HEALTH AND DISEASE,” 6th ed. Garland Science Publishing, NY, pp. 214-216; Sun, Z. J. et al. (2001) “Mechanisms Contributing To T Cell Receptor Signaling And Assembly Revealed By The Solution Structure Of An Ectodomain Fragment Of The CD3ε:γ Heterodimer,” Cell 105(7):913-923; Kuhns, M. S. et al. (2006) “Deconstructing The Form And Function Of The TCR CD3 Complex,” Immunity. 2006 February; 24(2):133-139).

As discussed below, in order to illustrate the present invention, bi-specific anti-human CD3×anti-human DR5-binding molecules were produced. An anti-human CD3 antibody used for such constructs is designated herein as “CD3 mAb 2.” The amino acid sequence of the VL Domain of CD3 mAb 2 (SEQ ID NO:104) is shown below (CDR residues are shown underlined):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG
CDR_{L}1 of CD3 mAb 2 (SEQ ID NO: 105):
RSSTGAVTTSNYAN
CDR_{L}2 of CD3 mAb 2 (SEQ ID NO: 106):
GTNKRAP
CDR_{L}3 of CD3 mAb 2 (SEQ ID NO: 107):
ALWYSNLWV

The amino acid sequence of the VH Domain of CD3 mAb 2 (SEQ ID NO:108) is shown below (CDR residues are shown underlined):

EVQLVESGGG LVQPGGSLRL SCAASGFTFS TYAMNWVRQA
PGKGLEWVGR IRSKYNNYAT YYADSVKDRF TISRDDSKNS
LYLQMNSLKT EDTAVYYCVR HGNFGNSYVS WFAYWGQGTL
VTVSS
CDR_{H}1 of CD3 mAb 2 (SEQ ID NO: 109):
TYAMN
CDR_{H}2 of CD3 mAb 2 (SEQ ID NO: 110):
RIRSKYNNYATYYADSVKD
CDR_{H}3 of CD3 mAb 2 (SEQ ID NO: 111):
HGNFGNSYVSWFAY

In some of the CD3 constructs, a variant VH Domain was employed for CD3 mAb 2. The variant VH domain possesses a D65G substitution, thus having the amino acid sequence shown below (SEQ ID NO:112) (CDR residues are shown underlined):

EVQLVESGGG LVQPGGSLRL SCAASGFTFS TYAMNWVRQA
PGKGLEWVGR IRSKYNNYAT YYADSVKDRF TISRDDSKNS
LYLQMNSLKT EDTAVYYCVR HGNFGNSYVS WFAYWGQGTL
VTVSS

The substitution causes the CDR_{H}2 to have the amino acid sequence (SEQ ID NO:113) RIRSKYNNYATYYADSVKG. The substituted position (D65G) is shown in double underline.

A second anti-CD3 antibody used herein is antibody Muromonab-CD3 “OKT3” (Xu et al. (2000) “In Vitro Characterization Of Five Humanized OKT3 Effector Function Variant Antibodies,” Cell. Immunol. 200:16-26); Norman, D. J. (1995) “Mechanisms Of Action And Overview Of OKT3,” Ther. Drug Monit. 17(6):615-620; Canafax, D. M. et al. (1987) “Monoclonal Antilymphocyte Antibody (OKT3) Treatment Of Acute Renal Allograft Rejection,” Pharmacotherapy 7(4):121-124; Swinnen, L. J. et al. (1993) “OKT3 Monoclonal Antibodies Induce Interleukin-6 And Interleukin-10: A Possible Cause Of Lymphoproliferative Disorders Associated With Transplantation,” Curr. Opin. Nephrol. Hypertens. 2(4):670-678). The amino acid sequence of the VL Domain of OKT3 (SEQ ID NO:114) is shown below (CDR residues are shown underlined):

QIVLTQSPAI MSASPGEKVT MTCSASSSVS YMNWYQQKSG
TSPKRWIYDT SKLASGVPAH FRGSGSGTSY SLTISGMEAE
DAATYYCQQW SSNPFTFGSG TKLEINR

The amino acid sequence of the VH Domain of OKT3 (SEQ ID NO:115) is shown below (CDR residues are shown underlined):

QVQLQQSGAE LARPGASVKM SCKASGYTFT RYTMHWVKQR
PGQGLEWIGY INPSRGYTNY NQKFKDKATL TTDKSSSTAY
MQLSSLTSED SAVYYCARYY DDHYCLDYWG QGTTLTVSS

3. CD16-Binding Domains

CD16 is the FcγRIIIA receptor. CD16 is expressed by neutrophils, eosinophils, natural killer (NK) cells, and tissue macrophages that bind aggregated but not monomeric human IgG (Peltz, G. A. et al. (1989) “Human Fc Gamma RI: Cloning, Expression, And Identification Of The Chromosomal Locus Of Two Fc Receptors For IgG,” Proc. Natl. Acad. Sci. (U.S.A.) 86(3):1013-1017; Bachanova, V. et al. (2014) “NK Cells In Therapy Of Cancer,” Crit. Rev. Oncog. 19(1-2):133-141; Miller, J. S. (2013) “Therapeutic Applications: Natural Killer Cells In The Clinic,” Hematology Am. Soc. Hematol. Educ. Program. 2013:247-253; Youinou, P. et al. (2002) “Pathogenic Effects Of Anti-Fc Gamma Receptor IIIB (CD16) On Polymorphonuclear Neutrophils In Non-Organ-Specific Autoimmune Diseases,” Autoimmun Rev. 1(1-2):13-19; Peipp, M. et al. (2002) “Bi-specific Antibodies Targeting Cancer Cells,” Biochem. Soc. Trans. 30(4):507-511).

The amino acid sequence of a Variable Light Chain Domain of anti-CD16 antibody 3G8 is (SEQ ID NO:116) (CDR residues are shown underlined):

DTVLTQSPAS LAVSLGQRAT ISCKASQSVD FDGDSFMNWY
QQKPGQPPKL LIYTTSNLES GIPARFSASG SGTDFTLNIH
PVEEEDTATY YCQQSNEDPY TFGGGTKLEI K

The amino acid sequence of the Variable Heavy Chain Domain of anti-CD16 antibody 3G8 is (SEQ ID NO:117) (CDR residues are shown underlined):

QVTLKESGPG ILQPSQTLSL TCSFSGFSLR TSGMGVGWIR
QPSGKGLEWL AHIWWDDDKR YNPALKSRLT ISKDTSSNQV
FLKIASVDTA DTATYYCAQI NPAWFAYWGQ GTLVTVSA

The amino acid sequence of a Variable Light Chain Domain of anti-CD16 antibody A9 is (SEQ ID NO:118) (CDR residues are shown underlined):

DIQAVVTQES ALTTSPGETV TLTCRSNTGT VTTSNYANWV
QEKPDHLFTG LIGHTNNRAP GVPARFSGSL IGDKAALTIT
GAQTEDEAIY FCALWYNNHW VFGGGTKLTVL

The amino acid sequence of the Variable Heavy Chain Domain of anti-CD16 antibody A9 is (SEQ ID NO:119) (CDR residues are shown underlined):

QVQLQQSGAE LVRPGTSVKI SCKASGYTFT NYWLGWVKQR
PGHGLEWIGD IYPGGGYTNY NEKFKGKATV TADTSSRTAY
VQVRSLTSED SAVYFCARSA SWYFDVWGAR TTVTVSS

4. CD19-Binding Domains

CD19 antigen is a type I transmembrane glycoprotein belonging to the immunoglobulin Ig superfamily. CD19 is expressed on follicular dendritic cells and B cells. It is considered a pan B cell marker expressed throughout B cell development but with threefold higher expression in mature cells as compared to immature B cells (Raufi A. et al. (2013) “Targeting CD19 In B-Cell Lymphoma: Emerging Role Of SAR3419,” Cancer Manag. Res. 5:225-233). Many CD19 antibodies have been described (e.g., M1D1342, MEDI-551, etc.) (Mei, H. E. et al. (2012) “Rationale Of Anti-CD19 Immunotherapy: An Option To Target Autoreactive Plasma Cells In Autoimmunity,” Arthritis Res. Ther. 14(Suppl 5):S1:1-16). The anti-CD19 binding molecule “blinatumomab” is disclosed in EP 2186527.

The amino acid sequence of the VL Domain of a preferred anti-CD19 antibody (HD37) is (SEQ ID NO:120) (CDR residues are shown underlined):

DILITQSPKS MSMSVGERVT LTCKASENVV TYVSWYQQKP
EQSPKLLIYG ASNRYTGVPD RFTGSGSATD FTLTISSVQA
EDLADYHCGQ GYSYPYTFGG GTKLEIKR

The amino acid sequence of the VH Domain of anti-CD19 antibody HD37 is (SEQ ID NO:121) (CDR residues are shown underlined):

QVQLQQSGAE LVRPGSSVKI SCKASGYAFS SYWMNWVKQR
PGQGLEWIGQ IWPGDGDTNY NGKFKGKATL TADESSSTAY
MQLSSLASED SAVYFCARRE TTTVGRYYYA MDYWGQGTSV
TVSS

5. CD20-Binding Domains

CD20 is a B cell-specific differentiation antigen that is expressed on mature B cells and in most B cell non-Hodgkin's lymphomas but not on early B cell progenitors or later mature plasma cells (Maloney, D. G. (2012) “Anti-CD20 Antibody Therapy for B-Cell Lymphomas,” N. Engl. J. Med. 366:2008-2016). Rituximab is an illustrative anti-human CD20 antibody. The amino acid sequence of the VL Domain of a chimeric anti-CD20 antibody (rituximab) is (SEQ ID NO:122) (CDR residues are shown underlined):

QIVLSQSPAI LSASPGEKVT MTCRASSSVS YIHWFQQKPG
SSPKPWIYAT SNLASGVPVR FSGSGSGTSY SLTISRVEAE
DAATYYCQQW TSNPPTFGGG TKLEIKR

The amino acid sequence of the VH Domain of anti-CD20 antibody (rituximab) is (SEQ ID NO:123) (CDR residues are shown underlined):

QVQLQQPGAE LVKPGASVKM SCKASGYTFT SYNMHWVKQT
PGRGLEWIGA IYPGNGDTSY NQKFKGKATL TADKSSSTAY
MQLSSLTSED SAVYYCARST YYGGDWYFNV WGAGTTVTVS
A

Alternative anti-CD20 antibodies that may be used in accordance with the present invention include the following commercially available antibodies: Ibritumomab (e.g., ZEVALIN®, Spectrum Pharmaceuticals, Inc.), Ofatumumab (e.g., ARZERRA®, SmithKlineGlaxo) and Tositumomab (e.g., BEXXAR®, GlaxoSmithKline). 6. CD22-Binding Domains

CD22 is a sugar binding transmembrane protein found on the surface of mature B cells and to a lesser extent on some immature B cells (WO 2011/032633; Poe, J. C. et al. (2012) “CD22 And Siglec-G In B Cell Function And Tolerance,” Trends Immunol. 33(8):413-420; Chen, W. C. et al. (2012) “Targeting B Lymphoma With Nanoparticles Bearing Glycan Ligands Of CD22,” Leuk. Lymphoma 53(2):208-210; Walker, J. A. (2008) “CD22: An Inhibitory Enigma,” Immunology 123(3):314-325; Coleman, M. et al. (2003) “Epratuzumab: Targeting B-Cell Malignancies Through CD22,” Clin. Cancer Res. 9(10 Pt 2):3991S-3994S).

The amino acid sequence of the VL Domain of anti-CD22 antibody (epratuzumab) is (SEQ ID NO:124) (CDR residues are shown underlined):

DIQLTQSPSS LSASVGDRVT MSCKSSQSVL YSANHKNYLA
WYQQKPGKAP KLLIYWASTR ESGVPSRFSG SGSGTDFTFT
ISSLQPEDIA TYYCHQYLSS WTFGGGTKVQ IKR

The amino acid sequence of the VH Domain of anti-CD22 antibody (epratuzumab) is (SEQ ID NO:125) (CDR residues are shown underlined):

QVQLVQSGAE VKKPGSSVKV SCKASGYTFT SYWLHWVRQA
PGQGLEWIGY INPRNDYTEY NQNFKDKATI TADESTNTAY
MELSSLRSED TAFYFCARRD ITTFYWGQGT TVTVSS

7. CD32B-Binding Domains

A preferred sequence for the VL domain of an antibody that binds to human CD32B is CD32B mAb 1 (SEQ ID NO:126) (CDR residues are shown underlined):

DIQMTQSPSS LLAALGERVS LTCRASQEIS GYLSWLQQKP
DGTIKRLIYA ASTLDSGVPK RFSGSESGSD YSLTISSLES
EDFADYYCLQ YFSYPLTFGA GTKLELK

A preferred sequence for the VH domain of the CD32B mAb 1 antibody that binds to human CD32B is (SEQ ID NO:127) (CDR residues are shown underlined):

EVKLEESGGG LVQPGGSMKL SCEASGFTFS DAWMDWVRQS
PEKGLEWVAE IRNKAKNHAT YYAESVIGRF TISRDDSKSS
VYLQMNSLRA EDTGIYYCGA LGLDYWGQGT TLTVSS

8. CD64-Binding Domains

CD64 is the FcγRI receptor and is expressed on monocytes/macrophages, dendritic cells, and activated granulocytes. The expression can be upregulated by IFN-7 stimulation. CD64 binds IgG immune complex. CD64 plays a role in antigen capture, phagocytosis of IgG/antigen complexes, and antibody-dependent cellular cytotoxicity (WO 2006/002438).

A preferred sequence for the VL domain of an antibody that binds to human CD64 is CD64 mAb 1 (SEQ ID NO:128) (CDR residues are shown underlined):

EIVLTQSPAT LSLSPGERAT LSCRASQSVS SYLAWYQQKP
GQAPRLLIYD ASSRATGIPA RFGGSGSGGT DFTLTISSLE
PEDFAVYYCQ LRSNWPPYTF GQGTKLEIK

A preferred sequence for the VH domain of an antibody that binds to human CD64 is (SEQ ID NO:129) (CDR residues are shown underlined):

QVQLVESGGG VVQPGRSLRL SCAASGFIFS GYGMHWVRQA
PGKGLEWVTV IWYDGSNKYY ADSVKGRFTI SRDNSKNTLY
LQMNSLRAED TAVYYCARDT GDRFFDYWGQ GTLVTVSS

9. BCR/CD79-Binding Domains

The BCR is composed of a membrane immunoglobulin which, together with non-covalently associated α and β subunits of CD79 (“CD79a” and “CD79b,” respectively), forms the BCR complex. CD79a and CD79b are signal transducing subunits that contain a conserved immunoreceptor tyrosine-based activation motif (“ITAM”) required for signal transduction (Dylke, J. et al. (2007) “Role Of The Extracellular And Transmembrane Domain Of Ig-Alpha Beta In Assembly Of The B Cell Antigen Receptor (BCR),” Immunol. Lett. 112(1):47-57; Cambier, J. C. (1995) “New Nomenclature For The Reth Motif (or ARH1/TAM/ARAM/YXXL),” Immunol. Today 16:110). Aggregation of the BCR complex by multivalent antigen initiates transphosphorylation of the CD79a and CD79b ITAMs and activation of receptor-associated kinases (DeFranco, A. L. (1997) “The Complexity Of Signaling Pathways Activated By The BCR,” Curr. Opin. Immunol. 9:296-308; Kurosaki, T. (1997) “Molecular Mechanisms In B Cell Antigen Receptor Signaling,” Curr. Opin. Immunol. 9:309-318; Kim, K. M. et al. (1993) “Signalling Function Of The B-Cell Antigen Receptors,” Immun. Rev. 132:125-146). Phosphorylated ITAMs recruit additional effectors such as PI₃K, PLC-7 and members of the Ras/MAPK pathway. These signaling events are responsible for both the B cell proliferation and increased expression of activation markers (such as MHCII and CD86) that are required to prime B cells for their subsequent interactions with T helper (“Tₕ”) cells.

A preferred sequence for the VL domain of an antibody that binds to the human B Cell Receptor (CD79) is CD79 mAb 1 (SEQ ID NO:130) (CDR residues are shown underlined):

DVVMTQTPLT LSVNIGQPAS ISCKSSQSLL DTDGKTYLNW
LLQRPQGSPN RLIYLVSKLD SGVPDRFTGS GSGTDFTLKI
SRVEAEDLGI YYCWQGTHFP LTFGAGTKLE LK

A preferred sequence for the VH domain of the CD79 mAb 1 antibody that binds to the human B Cell Receptor (CD79) is (SEQ ID NO:131) (CDR residues are shown underlined):

QVQLQQPGAE LVRPGASVKL SCKASGYTFT SYWMNWVKQR
PGQGLEWIGM VDPSDSETHY NQMFKDKATL TVDKSSSTAY
MQLSSLTSED SAVYYCARAM GYWGQGTSVT VSS

10. T Cell Receptor-Binding Domains

In an alternate embodiment, the second epitope that is bound by the Tri-Specific Binding Molecules of the present invention will be an epitope of the T cell Receptor (TCR). The T cell Receptor is natively expressed by CD4+ or CD8+ T cells, and permits such cells to recognize antigenic peptides that are bound and presented by class I or class II MHC proteins of antigen presenting cells. Recognition of a pMHC (peptide-MHC) complex by a TCR initiates the propagation of a cellular immune response that leads to the production of cytokines and the lysis of the antigen presenting cell (see, e.g., Armstrong, K. M. et al. (2008) “Conformational Changes And Flexibility In T-Cell Receptor Recognition Of Peptide-MHC Complexes,” Biochem. J. 415(Pt 2):183-196; Willemsen, R. (2008) “Selection Of Human Antibody Fragments Directed Against Tumor T-Cell Epitopes For Adoptive T-Cell Therapy,” Cytometry A. 73(11):1093-1099; Beier, K. C. et al. (2007) “Master Switches Of T-Cell Activation And Differentiation,” Eur. Respir. J. 29:804-812; Mallone, R. et al. (2005) “Targeting T Lymphocytes For Immune Monitoring And Intervention In Autoimmune Diabetes,” Am. J. Ther. 12(6):534-550). CD3 is the receptor that binds to the TCR (Thomas, S. et al. (2010) “Molecular Immunology Lessons From Therapeutic T-Cell Receptor Gene Transfer,” Immunology 129(2):170-177; Guy, C. S. et al. (2009) “Organization Of Proximal Signal Initiation At The TCR:CD3 Complex,” Immunol. Rev. 232(1):7-21; St. Clair, E. W. (Epub 2009 Oct. 12) “Novel Targeted Therapies For Autoimmunity,” Curr. Opin. Immunol. 21(6):648-657; Baeuerle, P. A. et al. (Epub 2009 Jun. 9) “Bi-specific T-Cell Engaging Antibodies For Cancer Therapy,” Cancer Res. 69(12):4941-4944; Smith-Garvin, J. E. et al. (2009) “T Cell Activation,” Annu. Rev. Immunol. 27:591-619; Renders, L. et al. (2003) “Engineered CD3 Antibodies For Immunosuppression,” Clin. Exp. Immunol. 133(3):307-309).

Antibodies that specifically bind to the T cell Receptor include the anti-TCR antibody BMA 031 (EP 0403156; Kurrle, R. et al. (1989) “BMA 031-A TCR-Specific Monoclonal Antibody For Clinical Application,” Transplant Proc. 21(1 Pt 1):1017-1019; Nashan, B. et al. (1987) “Fine Specificity Of A Panel Of Antibodies Against The TCR/CD3 Complex,” Transplant Proc. 19(5):4270-4272; Shearman, C. W. et al. (1991) “Construction, Expression, And Biologic Activity Of Murine/Human Chimeric Antibodies With Specificity For The Human α/β T Cell,” J. Immunol. 146(3):928-935; Shearman, C. W. et al. (1991) “Construction, Expression And Characterization of Humanized Antibodies Directed Against The Human α/β T Cell Receptor,” J. Immunol. 147(12):4366-4373).

The amino acid sequence of the VL Domain of anti-TCR antibody BMA 031 is (SEQ ID NO:132) (CDR residues are shown underlined):

EIVLTQSPAT LSLSPGERAT LSCSATSSVS YMHWYQQKPG
KAPKRWIYDT SKLASGVPSR FSGSGSGTEF TLTISSLQPE
DFATYYCQQW SSNPLTFGQG TKLEIK

The amino acid sequence of a VH Domain of anti-TCR antibody BMA 031 is (SEQ ID NO:133) (CDR residues are shown underlined):

QVQLVQSGAE VKKPGASVKV SCKASGYKFT SYVMHWVRQA
PGQGLEWIGY INPYNDVTKY NEKFKGRVTI TADKSTSTAY
LQMNSLRSED TAVHYCARGS YYDYDGFVYW GQGTLVTVSS

11. NKG2D Receptor-Binding Domains

In an alternate embodiment, the second epitope that is bound by the Tri-Specific Binding Molecules of the present invention will be an epitope of the NKG2D receptor. The NKG2D receptor is expressed on all human (and other mammalian) Natural Killer cells (Bauer, S. et al. (1999) “Activation Of NK Cells And T Cells By NKG2D, A Receptor For Stress-Inducible MICA,” Science 285(5428):727-729; Jamieson, A. M. et al. (2002) “The Role Of The NKG2D Immunoreceptor In Immune Cell Activation And Natural Killing,” Immunity 17(1):19-29) as well as on all CD8⁺ T cells (Groh, V. et al. (2001) “Costimulation Of CD8αβ T Cells By NKG2D Via Engagement By MIC Induced On Virus-Infected Cells,” Nat. Immunol. 2(3):255-260; Jamieson, A. M. et al. (2002) “The Role Of The NKG2D Immunoreceptor In Immune Cell Activation And Natural Killing,” Immunity 17(1):19-29). Such binding ligands, and particularly those which are not expressed on normal cells, include the histocompatibility 60 (H60) molecule, the product of the retinoic acid early inducible gene-1 (RAE-1), and the murine UL16-binding protein like transcript 1 (MULT1) (Raulet D. H. (2003) “Roles Of The NKG2D Immunoreceptor And Its Ligands,” Nature Rev. Immunol. 3:781-790; Coudert, J. D. et al. (2005) “Altered NKG2D Function In NK Cells Induced By Chronic Exposure To Altered NKG2D Ligand-Expressing Tumor Cells,” Blood 106:1711-1717). Antibodies that specifically bind to the NKG2D Receptor include KYK-2.0 (Kwong, K Y et al. (2008) “Generation, Affinity Maturation, And Characterization Of A Human Anti-Human NKG2D Monoclonal Antibody With Dual Antagonistic And Agonistic Activity,” J. Mol. Biol. 384:1143-1156; and PCT/US09/54911).

The amino acid sequence of the VL Domain of anti-NKG2D antibody KYK-1.0 is (SEQ ID NO:134) (CDR residues are shown underlined):

QPVLTQPSSV SVAPGETARI PCGGDDIETK SVHWYQQKPG
QAPVLVIYDD DDRPSGIPER FFGSNSGNTA TLSISRVEAG
DEADYYCQVW DDNNDEWVFG GGTQLTVL

The amino acid sequence of the VH Domain of anti-NKG2D antibody KYK-1.0 is (SEQ ID NO:135) (CDR) residues are shown underlined):

EVQLVESGGG VVQPGGSLRL SCAASGFTFS SYGMHWVRQA
PGKGLEWVAF IRYDGSNKYY ADSVKGRFTI SRDNSKNTKY
LQMNSLRAED TAVYYCAKDR FGYYLDYWGQ GTLVTVSS

The amino acid sequence of a VL Domain of anti-NKG2D antibody KYK-2.0 is (SEQ ID NO:136) (CDR residues are shown underlined):

QSALTQPASV SGSPGQSITI SCSGSSSNIG NNAVNWYQQL
PGKAPKLLIY YDDLLPSGVS DRFSGSKSGT SAFLAISGLQ
SEDEADYYCA AWDDSLNGPV FGGGTKLTVL

The amino acid sequence of a VH Domain of anti-NKG2D antibody KYK-2.0 is (SEQ ID NO:137) (CDR residues are shown underlined):

QVQLVESGGG LVKPGGSLRL SCAASGFTFS SYGMHWVRQA
PGKGLEWVAF IRYDGSNKYY ADSVKGRFTI SRDNSKNTLY
LQMNSLRAED TAVYYCAKDR GLGDGTYFDY WGQGTTVTVS
S

### D. Preferred Trispecific Binding Molecules of the Present Invention

1. Preferred Fc Domains

The CH2 and CH3 Domains of the two heavy chains interact to form the Fc Domain, which is a domain that is recognized by cellular Fc Receptors (FcγRs). As used herein, the term “Fc Domain” is used to define a C-terminal region of an IgG heavy chain. The amino acid sequence of the CH2-CH3 domain of an exemplary human IgG1 is (SEQ ID NO:1):

```
|CH2→
APELLGGPS VFLFPPKPKD TLMISRTPEV TCVVVDVSHE DPEVKFNWYV DGVEVHNAKT
231 240 250 260 270 280
                                                                                                      →CH2 | CH3→
KPREEQYNST YRVVSVLTVL HQDWLNGKEY KCKVSNKALP APIEKTISKA K GQPREPQVY
290 300 310 320 330 340
TLPPSREEMT KNQVSLTCLV KGFYPSDIAV EWESNGQPEN NYKTTPPVLD SDGSFFLYSK
350 360 370 380 390 400
                                                                          ←CH3|
LTVDKSRWQQ GNVFSCSVMH EALHNHYTQK SLSLSPGK
410 420 430 440
```

Throughout the present specification, the numbering of the residues in an IgG heavy chain is that of the EU index as in Kabat et al., Sequences of Proteins of Immunological Interest, 5^{th} Ed. Public Health Service, NH1, MD (1991), expressly incorporated herein by references. The “EU index as in Kabat” refers to the numbering of the human IgG1 EU antibody. Amino acids from the variable regions of the mature heavy and light chains of immunoglobulins are designated by the position of an amino acid in the chain. Kabat described numerous amino acid sequences for antibodies, identified an amino acid consensus sequence for each subgroup, and assigned a residue number to each amino acid. Kabat's numbering scheme is extendible to antibodies not included in his compendium by aligning the antibody in question with one of the consensus sequences in Kabat by reference to conserved amino acids. This method for assigning residue numbers has become standard in the field and readily identifies amino acids at equivalent positions in different antibodies, including chimeric or humanized variants. For example, an amino acid at position 50 of a human antibody light chain occupies the equivalent position to an amino acid at position 50 of a mouse antibody light chain.

Although boundaries may vary slightly, the CH2 domain of a human IgG Fc Domain usually extends from amino acids 231 to amino acid 341 of a human IgG according to the numbering system of Kabat. The CH3 domain of a human IgG usually extends from amino acids 342 to 447 according to the numbering system of Kabat. The “hinge region” or “hinge domain” is generally defined as stretching from Glu216 to Pro230 of human IgG1.

Polymorphisms have been observed at a number of different positions within antibody constant regions (e.g., Fc positions, including but not limited to positions 270, 272, 312, 315, 356, and 358 as numbered by the EU index as set forth in Kabat), and thus slight differences between the presented sequence and sequences in the prior art can exist. Polymorphic forms of human immunoglobulins have been well-characterized. At present, 18 Gm allotypes are known: G1m (1, 2, 3, 17) or G1m (a, x, f, z), G2m (23) or G2m (n), G3m (5, 6, 10, 11, 13, 14, 15, 16, 21, 24, 26, 27, 28) or G3m (b1, c3, b3, b0, b3, b4, s, t, g1, c5, u, v, g5) (Lefranc, et al., The human IgG subclasses: molecular analysis of structure, function and regulation. Pergamon, Oxford, pp. 43-78 (1990); Lefranc, G. et al., 1979, Hum. Genet.: 50, 199-211). It is specifically contemplated that the antibodies of the present invention may be incorporate any allotype, isoallotype, or haplotype of any immunoglobulin gene, and are not limited to the allotype, isoallotype or haplotype of the sequences provided herein.

Activating and inhibitory signals are transduced through the Fc Receptors (FcγRs) following their ligation to an Fc Domain. These diametrically opposing functions result from structural differences among the different receptor isoforms. Two distinct domains within the cytoplasmic signaling domains of the receptor called immunoreceptor tyrosine-based activation motifs (ITAMs) or immunoreceptor tyrosine-based inhibitory motifs (ITIMS) account for the different responses. The recruitment of different cytoplasmic enzymes to these structures dictates the outcome of the FcγR-mediated cellular responses. ITAM-containing FcγR complexes include FcγRI, FcγRIIA, FcγRIIIA, whereas ITIM-containing complexes only include FcγRIIB. Human neutrophils express the FcγRIIA gene. FcγRIIA clustering via immune complexes or specific antibody cross-linking serves to aggregate ITAMs along with receptor-associated kinases which facilitate ITAM phosphorylation. ITAM phosphorylation serves as a docking site for Syk kinase, activation of which results in activation of downstream substrates (e.g., PI₃K). Cellular activation leads to release of proinflammatory mediators. The FcγRIIB gene is expressed on B lymphocytes; its extracellular domain is 96% identical to FcγRIIA and binds IgG complexes in an indistinguishable manner. The presence of an ITIM in the cytoplasmic domain of FcγRIIB defines this inhibitory subclass of FcγR. Recently the molecular basis of this inhibition was established. When co-ligated along with an activating FcγR, the ITIM in FcγRIIB becomes phosphorylated and attracts the SH2 domain of the inositol polyphosphate 5′-phosphatase (SHIP), which hydrolyzes phophoinositol messengers released as a consequence of ITAM-containing FcγR-mediated tyrosine kinase activation, consequently preventing the influx of intracellular Ca⁺⁺. Thus cross-linking of FcγRIIB dampens the activating response to FcγR ligation and inhibits cellular responsiveness. B cell activation, B cell proliferation and antibody secretion is thus aborted.

The Fc Domain of the binding molecules of the present invention may be either a complete Fc Domain (e.g., a complete IgG Fc Domain) or only a fragment of a complete Fc Domain. Although the Fc Domain of the bi-specific monovalent Fc diabodies of the present invention may possess the ability to bind to one or more Fc receptors (e.g., FcγR(s)), more preferably such Fc Domain will cause altered binding to FcγRIA (CD64), FcγRIIA (CD32A), FcγRIIB (CD32B), FcγRIIIA (CD16a) or FcγRIIIB (CD16b) (relative to the binding exhibited by a wild-type Fc Domain) or will substantially eliminate the ability of such Fc Domain to bind to inhibitory receptor(s). Thus, the Fc Domain of the Fc Domain-containing diabodies of the present invention may include some or all of the CH2 Domain and/or some or all of the CH3 Domain of a complete Fc Domain, or may comprise a variant CH2 and/or a variant CH3 sequence (that may include, for example, one or more insertions and/or one or more deletions with respect to the CH2 or CH3 domains of a complete Fc Domain). Such Fc Domains may comprise non-Fc polypeptide portions, or may comprise portions of non-naturally complete Fc Domains, or may comprise non-naturally occurring orientations of CH2 and/or CH3 domains (such as, for example, two CH2 domains or two CH3 Domains, or in the N-terminal to C-terminal direction, a CH3 Domain linked to a CH2 Domain, etc.).

Fc Domain modifications identified as altering effector function are known in the art, including modifications that increase binding to activating receptors (e.g., FcγRIIA (CD16A) and reduce binding to inhibitory receptors (e.g., FcγRIIB (CD32B) (see, e.g., Stavenhagen, J. B. et al. (2007) “Fc Optimization Of Therapeutic Antibodies Enhances Their Ability To Kill Tumor Cells In Vitro And Controls Tumor Expansion In Vivo Via Low-Affinity Activating Fcgamma Receptors,” Cancer Res. 57(18):8882-8890).

In particular, it is preferred for the CH2-CH3 domains of the polypeptide chains of the Fc Domain-containing diabodies of the present invention to exhibit decreased (or substantially no) binding to FcγRIA (CD64), FcγRIIA (CD32A), FcγRIIB (CD32B), FcγRIIIA (CD16a) or FcγRIIIB (CD16b) (relative to the binding exhibited by the wild-type Fc Domain (SEQ ID NO:1). Fc variants and mutant forms capable of mediating such altered binding are described above. In a preferred embodiment the CH2-CH3 Domain of the first and/or third polypeptide chains of such diabodies include any 1, 2, 3, 4, 5, 6, or 7 of the substitutions: L234A, L235A, F243L, R292P, Y300L, V305I and P396L. Exemplary variants of human IgG1 Fc Domains with reduced binding to CD32B and/or increased binding to CD16A contain F243L, R292P, Y300L, V305I or P296L substitutions. These amino acid substitutions may be present in a human IgG1 Fc Domain in any combination. In one embodiment, the human IgG1 Fc Domain variant contains a F243L, R292P and Y300L substitution. In another embodiment, the human IgG1 Fc Domain variant contains a F243L, R292P, Y300L, V305I and P296L substitution. In one embodiment the CH2-CH3 Domain of the first and/or third polypeptide chains of such diabodies include any 1, 2, or 3, of the substitutions: L234A, L235A, N297G, N297Q. In another embodiment, the human IgG1 Fc Domain variant contains an N297Q substitution, L234A and L235A substitutions or a D265A substitution, as these mutations abolish FcR binding. Alternatively, a CH2-CH3 domain which inherently exhibits decreased (or substantially no) binding to FcγRIIIA (CD16a) and/or reduced effector function (relative to the binding exhibited by the wild-type IgG1 Fc Domain (SEQ ID NO:1)) is utilized. In a specific embodiment, the Fc Domain-containing diabodies of the present invention comprise an IgG2 Fc Domain or an IgG4 Fc Domain. Where an IgG4 Fc Domain in utilized the instant invention also encompasses the introduction of a stabilizing mutation such as S228P, as numbered by the EU index as set forth in Kabat (Lu et al., (2008) “The Effect Of A Point Mutation On The Stability Of Igg4 As Monitored By Analytical Ultracentrifugation,” J Pharmaceutical Sciences 97:960-969) to reduce the incidence of strand exchange. Other stabilizing mutations known in the art may be introduced into an IgG4 Fc Domain (Peters, P et al., (2012) “Engineering an Improved IgG4 Molecule with Reduced Disulfide Bond Heterogeneity and Increased Fab Domain Thermal Stability,” J. Biol. Chem., 287:24525-24533; PCT Patent Publication No: WO 2008/145142). Since the N297A, L234A, L235A and D265A substitutions abolish effector function, in circumstances in which effector function is desired, these substitutions would preferably not be employed.

The CH2 and/or CH3 Domains of such polypeptide chains need not be identical in sequence, and advantageously are modified to foster complexing between the two polypeptide chains. For example, an amino acid substitution (preferably a substitution with an amino acid comprising a bulky side group forming a “knob”, e.g., tryptophan) can be introduced into the CH2 or CH3 Domain such that steric interference will prevent interaction with a similarly mutated domain and will obligate the mutated domain to pair with a domain into which a complementary, or accommodating mutation has been engineered, i.e., “the hole” (e.g., a substitution with glycine). Such sets of mutations can be engineered into any two of the polypeptides of the Tri-Specific Binding Molecule. Methods of protein engineering to favor heterodimerization over homodimerization are well-known in the art, in particular with respect to the engineering of immunoglobulin-like molecules, and are encompassed herein (see e.g., Ridgway et al. (1996) “Knobs-Into-Holes' Engineering Of Antibody CH3 Domains For Heavy Chain Heterodimerization,” Protein Engr. 9:617-621, Atwell et al. (1997) “Stable Heterodimers From Remodeling The Domain Interface Of A Homodimer Using A Phage Display Library,” J. Mol. Biol. 270: 26-35, and Xie et al. (2005) “A New Format Of Bi-specific Antibody: Highly Efficient Heterodimerization, Expression And Tumor Cell Lysis,” J. Immunol. Methods 296:95-101; each of which is hereby incorporated herein by reference in its entirety). Preferably the “knob” is engineered into the CH2-CH3 Domains of the first polypeptide chain and the “hole” is engineered into the CH2-CH3 Domains of the other CH2-CH3-containing polypeptide chain. Thus, the “knob” will help in preventing the first polypeptide chain from homodimerizing via its CH2 and/or CH3 Domains. The CH2-CH3 “hole-bearing” polypeptide chain will heterodimerize with the CH2-CH3 “knob-bearing” polypeptide chain, and will also homodimerize with itself. A preferred knob is created by modifying a native IgG Fc Domain to contain the modification T366W. A preferred hole is created by modifying a native IgG Fc Domain to contain the modification T366S, L368A and Y407V. To aid in purifying the “hole-bearing” polypeptide chain homodimer from the final Tri-Specific Binding Molecule, the protein A binding site of the CH2 and CH3 Domains of the “hole-bearing” Fc Domain is preferably mutated by amino acid substitution at position 435 (H435R). Thus, the “hole-bearing” Fc Domain homodimer will not bind to protein A, whereas the desired Tri-Specific Binding Molecule will retain its ability to bind protein A via the protein A binding site on the first polypeptide chain.

A preferred sequence for the CH2 and CH3 Domains of the first polypeptide chain of an Fc Domain-containing diabody of the present invention will have the “knob-bearing” sequence (SEQ ID NO:52):

APEAAGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLWCLVK GFYPSDIAVE WESNGQPENN
YKTTPPVLDS DGSFFLYSKL TVDKSRWQQG NVFSCSVMHE
ALHNHYTQKS LSLSPGK

A preferred sequence for the CH2 and CH3 Domains of the second polypeptide chain of an Fc Domain-containing diabody of the present invention having two polypeptide chains (or the third polypeptide chain of an Fc Domain-containing diabody having three polypeptide chains) will have the “hole-bearing” sequence (SEQ ID NO:53):

APEAAGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLSCAVK GFYPSDIAVE WESNGQPENN
YKTTPPVLDS DGSFFLVSKL TVDKSRWQQG NVFSCSVMHE
ALHNRYTQKS LSLSPGK

As will be noted, the CH2-CH3 Domains of SEQ ID NO:52 and SEQ ID NO:53 include a substitution at position 234 with alanine and 235 with alanine, and thus form an Fc Domain exhibit decreased (or substantially no) binding to FcγRIA (CD64), FcγRIIA (CD32A), FcγRIIB (CD32B), FcγRIIIA (CD16a) or FcγRIIIB (CD16b) (relative to the binding exhibited by the wild-type Fc Domain (SEQ ID NO:1).

It is preferred that the first polypeptide chain will have a “knob-bearing” CH2-CH3 sequence, such as that of SEQ ID NO:52. However, as will be recognized, a “hole-bearing” CH2-CH3 Domain (e.g., SEQ ID NO:53) could be employed in the first polypeptide chain, in which case, a “knob-bearing” CH2-CH3 Domain (e.g., SEQ ID NO:52) would be employed in the second polypeptide chain of an Fc Domain-containing diabody of the present invention having two polypeptide chains (or the third polypeptide chain of an Fc Domain-containing diabody having three polypeptide chains).

2. Preferred First Polypeptide Chain

A first polypeptide chain of a preferred binding molecule of the present invention will comprise a Variable Light Chain Domain capable of binding to Epitope I (VL_{I}), a Variable Heavy Chain Domain capable of binding to Epitope II (VH_{II}), a Heterodimer-Promoting Domain and a CH2-CH3 Domain.

Since the Variable Light Chain and Variable Heavy Chain Domains of the first polypeptide are directed toward different epitopes, they cannot associate together to form a Binding Domain that is able to bind either Epitope I or Epitope II. The Variable Light Chain and Variable Heavy Chain Domains of the first polypeptide are spaced apart from one another by an intervening linker peptide that is sufficiently short as to substantially prevent the association of these Domains. An exemplary linker, termed “Linker 1,” has the sequence (SEQ ID NO:33): GGGSGGGG.

The Variable Heavy Chain Domain of the first polypeptide and the Heterodimer-Promoting Domain of that polypeptide are preferably spaced apart from one another by an intervening linker peptide that contains 1, 2, 3 or more cysteine residues. A preferred cysteine-containing spacer peptide (“Linker 2”) has the sequence is SEQ ID NO:34: GGCGGG.

Linkers that may be employed to link a CH2-CH3 Domain to a polypeptide chain of the molecules of the present invention include: ASTKG (SEQ ID NO:47), DKTHTCPPCP (SEQ ID NO:48), LEPKSS (SEQ ID NO:49), and APSSSPME (SEQ ID NO:50), APSSS (SEQ ID NO:152) and GGG or GCG. SEQ ID NO:49 may be used in lieu of GGG or GCG for ease of cloning. Additionally, SEQ ID NO:49 may be immediately followed by SEQ ID NO:47 to form an alternate linker (LEPKSSDKTHTCPPCP; SEQ ID NO:51).

The Heterodimer-Promoting Domain of the first polypeptide and the Heterodimer-Promoting Domain of the second polypeptide are coordinately selected. The Domains differ from one another and are designed to associate with one another so as to promote the association of the first and second polypeptide chains. For example, one of the Heterodimer-Promoting Domains will be engineered to have a negative charge at pH 7, while the other of the two polypeptide chains will be engineered to have a positive charge at pH 7. The presence of such charged Domains promotes association between the first and second polypeptides, and thus fosters heterodimerization. It is immaterial which Heterodimer-Promoting Domains is provided to which chain, as long as the Domains employed on the first and second polypeptide chains differ so as to foster heterodimerization between such chains.

The Heterodimer-Promoting Domains may be the IgG CL and CH1 domains or may be a peptide having the amino acid sequence GVEPKSC (SEQ ID NO:35) or VEPKSC (SEQ ID NO:36), derived from the hinge domain of a human IgG, and in lieu of the CL domain, one may employ the C-terminal 6 amino acids of the human kappa light chain, GFNRGEC (SEQ ID NO:37) or FNRGEC (SEQ ID NO:38).

More preferably, however, the Heterodimer-Promoting Domains of such diabodies are formed from one, two, three or four tandemly repeated coil domains of opposing charge that comprise a sequence of at least six, at least seven or at least eight charged amino acid residues (Apostolovic, B. et al. (2008) “pH-Sensitivity of the E3 K3 Heterodimeric Coiled Coil,” Biomacromolecules 9:3173-3180; Arndt, K. M. et al. (2001) “Helix-stabilized Fv (hsFv) Antibody Fragments: Substituting the Constant Domains of a Fab Fragment for a Heterodimeric Coiled-coil Domain,” J. Molec. Biol. 312:221-228; Arndt, K. M. et al. (2002) “Comparison of In Vivo Selection and Rational Design of Heterodimeric Coiled Coils,” Structure 10:1235-1248; Boucher, C. et al. (2010) “Protein Detection By Western Blot Via Coiled-Coil Interactions,” Analytical Biochemistry 399:138-140; Cachia, P. J. et al. (2004) “Synthetic Peptide Vaccine Development: Measurement Of Polyclonal Antibody Affinity And Cross-Reactivity Using A New Peptide Capture And Release System For Surface Plasmon Resonance Spectroscopy,” J. Mol. Recognit. 17:540-557; De Crescenzo, G. D. et al. (2003) “Real-Time Monitoring of the Interactions of Two-Stranded de novo Designed Coiled-Coils: Effect of Chain Length on the Kinetic and Thermodynamic Constants of Binding,” Biochemistry 42:1754-1763; Fernandez-Rodriquez, J. et al. (2012) “Induced Heterodimerization And Purification Of Two Target Proteins By A Synthetic Coiled-Coil Tag,” Protein Science 21:511-519; Ghosh, T. S. et al. (2009) “End-To-End And End-To-Middle Interhelical Interactions: New Classes Of Interacting Helix Pairs In Protein Structures,” Acta Crystallographica D65:1032-1041; Grigoryan, G. et al. (2008) “Structural Specificity In Coiled-Coil Interactions,” Curr. Opin. Struc. Biol. 18:477-483; Litowski, J. R. et al. (2002) “Designing Heterodimeric Two-Stranded a-Helical Coiled-Coils: The Effects Of Hydrophobicity And a-Helical Propensity On Protein Folding, Stability, And Specificity,” J. Biol. Chem. 277:37272-37279; Steinkruger, J. D. et al. (2012) “The d′--d--d′ Vertical Triad is Less Discriminating Than the a′--a--a′ Vertical Triad in the Antiparallel Coiled-coil Dimer Motif,” J. Amer. Chem. Soc. 134(5):2626-2633; Straussman, R. et al. (2007) “Kinking the Coiled Coil-Negatively Charged Residues at the Coiled-coil Interface,” J. Molec. Biol. 366:1232-1242; Tripet, B. et al. (2002) “Kinetic Analysis of the Interactions between Troponin C and the C-terminal Troponin I Regulatory Region and Validation of a New Peptide Delivery Capture System used for Surface Plasmon Resonance,” J. Molec. Biol. 323:345-362; Woolfson, D. N. (2005) “The Design Of Coiled-Coil Structures And Assemblies,” Adv. Prot. Chem. 70:79-112; Zeng, Y. et al. (2008) “A Ligand-Pseudoreceptor System Based On de novo Designed Peptides For The Generation Of Adenoviral Vectors With Altered Tropism,” J. Gene Med. 10:355-367).

Such repeated coil domains may be exact repeats or may have substitutions. For example, the Heterodimer-Promoting Domain of the first polypeptide chain may comprise a sequence of eight negatively charged amino acid residues and the Heterodimer-Promoting Domain of the second polypeptide chain may comprise a sequence of eight negatively charged amino acid residues. It is immaterial which coil is provided to the first or second polypeptide chains, provided that a coil of opposite charge is used for the other polypeptide chain. The positively charged amino acid may be lysine, arginine, histidine, etc. and/or the negatively charged amino acid may be glutamic acid, aspartic acid, etc. The positively charged amino acid is preferably lysine and/or the negatively charged amino acid is preferably glutamic acid. It is possible for only a single Heterodimer-Promoting Domain to be employed (since such domain will inhibit homodimerization and thereby promote heterodimerization), however, it is preferred for both the first and second polypeptide chains of the diabodies of the present invention to contain Heterodimer-Promoting Domains.

In a preferred embodiment, one of the Heterodimer-Promoting Domains will comprise four tandem “E-coil” helical domains (SEQ ID NO:39: EVAALEK-EVAALEK-EVAALEK-EVAALEK), whose glutamate residues will form a negative charge at pH 7, while the other of the Heterodimer-Promoting Domains will comprise four tandem “K-coil” domains (SEQ ID NO:40: KVAALKE-KVAALKE-KVAALKE-KVAALKE), whose lysine residues will form a positive charge at pH 7. The presence of such charged domains promotes association between the first and second polypeptides, and thus fosters heterodimerization. Especially preferred is a Heterodimer-Promoting Domain in which one of the four tandem “E-coil” helical domains of SEQ ID NO:39 has been modified to contain a cysteine residue: EVAACEK-EVAALEK-EVAALEK-EVAALEK (SEQ ID NO:41). Likewise, especially preferred is a Heterodimer-Promoting Domain in which one of the four tandem “K-coil” helical domains of SEQ ID NO:40 has been modified to contain a cysteine residue: KVAACKE-KVAALKE-KVAALKE-KVAALKE (SEQ ID NO:42).

As disclosed in WO 2012/018687, in order to improve the in vivo pharmacokinetic properties of diabodies, a diabody may be modified to contain a polypeptide portion of a serum-binding protein at one or more of the termini of the diabody. Most preferably, such polypeptide portion of a serum-binding protein will be installed at the C-terminus of the diabody. Albumin is the most abundant protein in plasma and has a half-life of 19 days in humans. Albumin possesses several small molecule binding sites that permit it to non-covalently bind to other proteins and thereby extend their serum half-lives. The Albumin-Binding Domain 3 (ABD3) of protein G of Streptococcus strain G148 consists of 46 amino acid residues forming a stable three-helix bundle and has broad albumin-binding specificity (Johansson, M. U. et al. (2002) “Structure, Specificity, And Mode Of Interaction For Bacterial Albumin-Binding Modules,” J. Biol. Chem. 277(10):8114-8120. Thus, a particularly preferred polypeptide portion of a serum-binding protein for improving the in vivo pharmacokinetic properties of a diabody is the Albumin-Binding Domain (ABD) from streptococcal protein G, and more preferably, the Albumin-Binding Domain 3 (ABD3) of protein G of Streptococcus strain G148 (SEQ ID NO:43): LAEAKVLANR ELDKYGVSDY YKNLIDNAKS AEGVKALIDE ILAALP.

As disclosed in WO 2012/162068 (herein incorporated by reference), “deimmunized” variants of SEQ ID NO:43 have the ability to attenuate or eliminate MHC class II binding. Based on combinational mutation results, the following combinations of substitutions are considered to be preferred substitutions for forming such a deimmunized albumin-binding domain: 66S/70S+71A; 66S/70S+79A; 64A/65A/71A+66S; 64A/65A/71A+66D; 64A/65A/71A+66E; 64A/65A/79A+66S; 64A/65A/79A+66D; 64A/65A/79A+66E. Variant ABDs having the modifications L64A, I65A and D79A or the modifications N66S, T70S and D79A. Variant deimmunized ABD having the amino acid sequence:

(SEQ ID NO: 44)
LAEAKVLANR ELDKYGVSDY YKNA₆₄A₆₅NNAKT VEGVKALIA₇₉E
ILAALP,

or the amino acid sequence:

(SEQ ID NO: 45)
LAEAKVLANR ELDKYGVSDY YKNLIS₆₆NAKS₇₀ VEGVKALIA₇₉E
ILAALP,

are particularly preferred as such deimmunized Albumin-Binding Domains exhibit substantially wild-type binding while providing attenuated MHC class II binding. Thus, the first polypeptide chain of such a diabody having an Albumin-Binding Domain contains a third linker (Linker 3) preferably positioned C-terminally to the E-coil (or K-coil) Domain of such polypeptide chain so as to intervene between the E-coil (or K-coil) Domain and the Albumin-Binding Domain (which is preferably a deimmunized Albumin-Binding Domain). A preferred sequence for such Linker 3 is SEQ ID NO:46: GGGS.

Thus, in sum, a preferred first polypeptide chain of a preferred Tri-Specific Binding Molecule of the present invention will comprise the Domains and linkers: (VL_{I} Domain)-(Linker 1)-(VH_{II} Domain)-(Linker 2)-(E-coil Heterodimer-Promoting Domain)-(Linker 3)-(Knob-Bearing CH2-CH3 Domain).

3. Alternative First Polypeptide Chain

In one embodiment, the orientations of the above-described Domains will be in the N-terminal to C-terminal direction. The present invention, however, also contemplates a variation thereof, wherein the orientations of the Domains of the first polypeptide chain are: NH₂-(Knob-Bearing CH3-CH2 Domain)-(VL_{I} Domain)-(Linker 1)-(VH_{II} Domain)-(Linker 2)-(E-coil Heterodimer-Promoting Domain). Preferably, a cysteine-containing peptide is present, N-terminal to such CH2-CH3 Domain. The sequence of an exemplary peptide is sequence (SEQ ID NO:48): DKTHTCPPCP. Preferably in this embodiment, the CH3 Domain is spaced apart from the VL_{I} Domain by an intervening peptide linker (Linker 4), such as one having the amino acid sequence of (SEQ ID NO:152): APSSS, and more preferably, the amino acid sequence (SEQ ID NO:50): APSSSPME.

4. Preferred Second Polypeptide Chain

A second polypeptide chain of such preferred Tri-Specific Binding Molecules will comprise, in the N-terminal to C-terminal direction, a Variable Light Chain Domain capable of binding to Epitope II (VL_{II}), a Variable Heavy Chain Domain capable of binding to Epitope I (VH_{I}), and a Heterodimer-Promoting Domain.

Since the Variable Light Chain and Variable Heavy Chain Domains of the second polypeptide are directed toward different epitopes, they cannot associate together to form a Binding Domain that is able to bind either Epitope I or Epitope II. The Variable Light Chain and Variable Heavy Chain Domains of the second polypeptide are spaced apart from one another by an intervening linker peptide that is sufficiently short as to substantially prevent the association of these Domains. “Linker 1,” having the sequence (SEQ ID NO:33): GGGSGGGG is an exemplary linker for this purpose.

As in the case of the first polypeptide chain, the Variable Heavy Chain Domain of the second polypeptide and the Heterodimer-Promoting Domain of that polypeptide are preferably spaced apart from one another by an intervening linker peptide that contains 1, 2, 3 or more cysteine residues. “Linker 2,” having the sequence (SEQ ID NO:34) GGCGGG is an exemplary linker for this purpose. Such cysteine residues can form disulfide bonds with cysteine residues in the cysteine-containing spacer peptide that separates the Variable Heavy Chain Domain of the first polypeptide and the Heterodimer-Promoting Domain of that polypeptide. Thus, the first and second polypeptides of the Binding Molecules of the present invention are covalently bonded to one another.

As discussed above, the Heterodimer-Promoting Domain of the second polypeptide chain is selected so as coordinate with the Heterodimer-Promoting Domain of the first polypeptide chain. Thus, in a preferred embodiment, the Heterodimer-Promoting Domain of the first polypeptide chain is either a “K-coil” Domain (SEQ ID NO:40) or an “E-coil” Domain (SEQ ID NO:39). If the cysteine-containing E-coil (SEQ ID NO:41) is employed in the first polypeptide chain, then the cysteine-containing K-coil (SEQ ID NO:42) is preferably employed in the second polypeptide chain. Conversely, if the cysteine-containing K-coil (SEQ ID NO:42) is employed in the first polypeptide chain, then the cysteine-containing E-coil (SEQ ID NO:41) is preferably employed in the second polypeptide chain. Since the first polypeptide chain will preferably possess an “E-coil” Domain, the second polypeptide chain will preferably contain a “K-coil” Domain.

As the first and second polypeptide chains are polypeptide chains of a diabody, they are able to associate together to form a Domain I Binding Domain (VL_{A}/VH_{A}) that recognizes and immunospecifically binds to Epitope I, and a Domain II Binding Domain (VL_{B}/VH_{B}) that recognizes and immunospecifically binds to Epitope II.

Thus, in sum, a preferred second polypeptide chain of a preferred Binding Molecule of the present invention will comprise the Domains and linkers: (VL_{II} Domain)-(Linker 1)-(VH_{I} Domain)-(Linker 2)-(K-coil Heterodimer-Promoting Domain).

5. Preferred Third Polypeptide Chain

A third polypeptide chain of a preferred Binding Molecule of the present invention is a polypeptide that comprises, in the N-terminal to C-terminal direction, a Binding Domain, an optional CH1-Hinge Domain, and a CH2-CH3 Domain. The Binding Domain of the third polypeptide chain of a preferred Binding Molecule of the present invention may be a Variable Heavy Chain Domain capable of binding to Epitope III (VH_{III}), in which case, the fourth polypeptide chain of the preferred Binding Molecules of the present invention (discussed below) is a polypeptide that comprises a Variable Light Chain Domain capable of binding to Epitope III (VL_{III}), such that the Binding Domain is capable of immunospecific binding to an antigen possessing Epitope III. Alternatively, the Binding Domain of the third polypeptide chain of the preferred Binding Molecules of the present invention may comprise an Effector Cell Receptor-Type Binding Domain, in which case, the fourth polypeptide chain of the preferred Binding Molecules of the present invention (discussed below) is a polypeptide that comprises a complementary Effector Cell Receptor-Type Binding Domain, such that the interaction of two polypeptide chains forms a Binding Domain that is capable of physiospecific binding to molecule present on the surface of the effector cell. The third polypeptide chain may be isolated from naturally occurring antibodies. Alternatively, it may be constructed recombinantly. An exemplary CH1 Domain is a human IgG1 CH1 Domain having the amino acid sequence (SEQ ID NO:207):

ASTKGPSVFP LAPSSKSTSG GTAALGCLVK DYFPEPVTVS
WNSGALTSGV HTFPAVLQSS GLYSLSSVVT VPSSSLGTQT
YICNVNHKPS NTKVDKKV

A variant of the human IgG1 CH1 Domain of SEQ ID NO:207 is (SEQ ID NO:208):

ASTKGPSVFP LAPSSKSTSG GTAALGCLVK DYFPEPVTVS
WNSGALTSGV HTFPAVLQSS GLYSLSSVVT VPSSSLGTQT
YICNVNHKPS NTKVDKRV

An exemplary Hinge Domain is a human IgG1 Hinge Domain having the amino acid sequence (SEQ ID NO:209): EPKSCDKTHTCPPCP. As will be recognized, the exemplary Hinge Domain comprises multiple cysteine residues (Elkabetz et al. (2005) “Cysteines In CH1 Underlie Retention Of Unassembled Ig Heavy Chains,” J. Biol. Chem. 280:14402-14412) that may participate in interchain covalent bonding.

Although a wild-type CH2-CH3 Domain may be employed, it is preferred, as described above, to employ a modified CH2-CH3 Domain that promotes heterodimerization with the CH2-CH3 Domain of the first polypeptide chain.

Preferably, therefore the CH2-CH3 Domain of the third polypeptide chain will be a “hole-bearing” CH2-CH3 Domain whose amino acid sequence is complementary to the “knob-bearing” CH2-CH3 Domain (SEQ ID NO:52) employed in the first polypeptide. As discussed above, the “hole-bearing” CH2-CH3 domain preferably should comprise a substitution at position 435 (H435R) to remove the Protein A binding site. An exemplary “hole-bearing” CH2-CH3 Domain with the H435R substitution for the third polypeptide is SEQ ID NO:53.

As will be recognized, a “knob-bearing” CH2-CH3 Domain (e.g., SEQ ID NO:52) could be employed in the third polypeptide chain, in which case, a “hole-bearing” CH2-CH3 Domain (e.g., SEQ ID NO:53) would be employed in the first polypeptide chain.

In the embodiment in which the third (and fourth) polypeptide chains of the preferred Tri-Specific Binding Molecules of the present invention each comprise a polypeptide chain of an Effector Cell Receptor-Type Binding Domain, methods for producing such Effector Cell Receptor-Type Binding Domains are well-known (e.g., US2012/0294874A1).

Thus, in sum, a third polypeptide chain of the preferred Binding Molecules of the present invention will comprise the Domains and linkers: (VH_{III} Domain)-(Optional CH1 Domain)-(Optional Hinge Domain)-(“Hole-Bearing” CH2-CH3 Domain), or (T Cell Receptor-Type Binding Domain; first or second polypeptide thereof)-(Optional CH1 Domain)-(Optional Hinge Domain)-(“Hole-Bearing” CH2-CH3 Domain).

6. Preferred Fourth Polypeptide Chain

A fourth polypeptide chain of the preferred Tri-Specific Binding Molecules of the present invention is either a polypeptide of an Effector Cell Receptor-Type Binding Domain (wherein the third and fourth polypeptides form a ligand for a receptor found on the surface of an effector cell, or more preferably, a light chain of the above-indicated antibody that immunospecifically binds to Epitope III or which are complementary to the binding domain of the third polypeptide chain.

Thus, wherein the third and fourth polypeptides form a Fab-Type Binding Domain such fourth polypeptide chain comprises, in the N-terminal to C-terminal direction, a Variable Light Chain Domain capable of binding to Epitope III (VL_{III}), and a Domain for promoting covalent bonding to the third polypeptide chain or a Binding Domain and such Domain for promoting covalent bonding to the third polypeptide chain. Such Domain may be a CL Domain, or a cysteine-containing portion thereof, such as (SEQ ID NO:38) FNRGEC or a linker such as Linker 2 (having the sequence (SEQ ID NO:34) GGCGGG. An exemplary a cysteine-containing peptide that forms disulfide bonds with such Linker 2 comprises the amino acid sequence VEPKSC (SEQ ID NO:36) or a Hinge Domain.

The fourth polypeptide chain may be isolated from naturally occurring antibodies. Alternatively, it may be constructed recombinantly. A preferred CL Domain is a human IgG1 CL Kappa Domain having the amino acid sequence (SEQ ID NO:210):

RTVAAPSVFI FPPSDEQLKS GTASVVCLLN NFYPREAKVQ
WKVDNALQSG NSQESVTEQD SKDSTYSLSS TLTLSKADYE
KHKVYACEVT HQGLSSPVTK SFNRGEC

Alternatively, an exemplary CL Domain is a human IgG1 CL Lambda2 Domain having the amino acid sequence (SEQ ID NO:211):

QPKAAPSVTL FPPSSEELQA NKATLVCLIS DFYPGAVTVA
WKADSSPVKA GVETTPSKQS NNKYAASSYL SLTPEQWKSH
RSYSCQVTHE GSTVEKTVAP TECS

As will be noticed, the CL Domain, or other Cysteine-Containing Domain, of the fourth polypeptide chain comprises cysteine residues. Such cysteine residues are able to covalently bond to cysteine residues of the CH1 Domain of the third polypeptide chain to thereby covalently complex the third and fourth polypeptide chains of the binding molecules of the present invention to one another. Thus the third and fourth polypeptide chains are covalently bonded to one another.

Additionally, cysteine residues of the CH2-CH3 Domain of the first polypeptide chain can form disulfide bonds with cysteine residues of the CH2-CH3 Domain of the third polypeptide chain. Thus the first and third polypeptide chains are covalently bonded to one another.

### E. Variant Fc Domains

In traditional immune function, the interaction of antibody-antigen complexes with cells of the immune system results in a wide array of responses, ranging from effector functions such as antibody-dependent cytotoxicity, mast cell degranulation, and phagocytosis to immunomodulatory signals such as regulating lymphocyte proliferation and antibody secretion. All of these interactions are initiated through the binding of the Fc Domain of antibodies or immune complexes to specialized cell surface receptors on hematopoietic cells. The diversity of cellular responses triggered by antibodies and immune complexes results from the structural heterogeneity of the three Fc receptors: FcγRI (CD64), FcγRII (CD32), and FcγRIII (CD16). FcγRI (CD64), FcγRIIA (CD32A) and FcγRIII (CD16) are activating (i.e., immune system enhancing) receptors; FcγRIIB (CD32B) is an inhibiting (i.e., immune system dampening) receptor. The amino acid sequence of an exemplary IgG1 Fc Domain (SEQ ID NO:1) is presented above.

Modification of the Fc Domain normally leads to an altered phenotype, for example altered serum half-life, altered stability, altered susceptibility to cellular enzymes or altered effector function. It may be desirable to modify the antibody of the invention with respect to effector function, so as to enhance the effectiveness of the antibody in treating cancer, for example. Reduction or elimination of effector function is desirable in certain cases, for example in the case of antibodies whose mechanism of action involves blocking or antagonism, but not killing of the cells bearing a target antigen. Increased effector function is generally desirable when directed to undesirable cells, such as tumor and foreign cells, where the FcγRs are expressed at low levels, for example, tumor-specific B cells with low levels of FcγRIIB (e.g., non-Hodgkins lymphoma, CLL, and Burkitt's lymphoma). In said embodiments, molecules of the invention with conferred or altered effector function activity are useful for the treatment and/or prevention of a disease, disorder or infection where an enhanced efficacy of effector function activity is desired.

In certain embodiments, the Tri-Specific Binding Molecules of the present invention comprise an Fc Domain that possesses one or more modifications (e.g., substitutions, deletions, or insertions) to the sequence of amino acids of a wild-type Fc Domain (SEQ ID NO:1), which reduce the affinity and avidity of the Fc Domain and, thus, the molecule of the invention, for one or more FcγR receptors. In other embodiments, the molecules of the invention comprise an Fc Domain that possesses one or more modifications to the amino acids of the wild-type Fc Domain, which increase the affinity and avidity of the Fc Domain and, thus, the molecule of the invention, for one or more FcγR receptors. In other embodiments, the molecules comprise a variant Fc Domain wherein said variant confers or mediates increased ADCC activity and/or an increased binding to FcγRIIA, relative to a molecule comprising no Fc Domain or comprising a wild-type Fc Domain. In alternate embodiments, the molecules comprise a variant Fc Domain wherein said variant confers or mediates decreased ADCC activity (or other effector function) and/or an increased binding to FcγRIIB, relative to a molecule comprising no Fc Domain or comprising a wild-type Fc Domain. In some embodiments, the invention encompasses Tri-Specific Binding Molecules comprising a variant Fc Domain, which variant Fc Domain does not show a detectable binding to any FcγR, relative to a comparable molecule comprising the wild-type Fc Domain. In other embodiments, the invention encompasses Tri-Specific Binding Molecules comprising a variant Fc Domain, which variant Fc Domain only binds a single FcγR, preferably one of FcγRIIA, FcγRIIB, or FcγRIIIA. Any such increased affinity and/or avidity is preferably assessed by measuring in vitro the extent of detectable binding to the FcγR or FcγR-related activity in cells that express low levels of the FcγR when binding activity of the parent molecule (without the modified Fc Domain) cannot be detected in the cells, or in cells which express non-FcγR receptor target antigens at a density of 30,000 to 20,000 molecules/cell, at a density of 20,000 to 10,000 molecules/cell, at a density of 10,000 to 5,000 molecules/cell, at a density of 5,000 to 1,000 molecules/cell, at a density of 1,000 to 200 molecules/cell or at a density of 200 molecules/cell or less (but at least 10, 50, 100 or 150 molecules/cell).

The Tri-Specific Binding Molecules of the present invention may comprise altered affinities for an activating and/or inhibitory Fcγ receptor. In one embodiment, the Tri-Specific Binding Molecule comprises a variant Fc Domain that has increased affinity for FcγRIIB and decreased affinity for FcγRIIIA and/or FcγRIIA, relative to a comparable molecule with a wild-type Fc Domain. In another embodiment, the Tri-Specific Binding Molecule of the present invention comprise a variant Fc Domain, which has decreased affinity for FcγRIIB and increased affinity for FcγRIIIA and/or FcγRIIA, relative to a comparable molecule with a wild-type Fc Domain. In yet another embodiment, the Tri-Specific Binding Molecules of the present invention comprise a variant Fc Domain that has decreased affinity for FcγRIIB and decreased affinity for FcγRIIIA and/or FcγRIIA, relative to a comparable molecule with a wild-type Fc Domain. In still another embodiment, the Tri-Specific Binding Molecules of the present invention comprise a variant Fc Domain, which has unchanged affinity for FcγRIIB and decreased (or increased) affinity for FcγRIIIA and/or FcγRIIA, relative to a comparable molecule with a wild-type Fc Domain.

In certain embodiments, the Tri-Specific Binding Molecules of the present invention comprise a variant Fc Domain having an altered affinity for FcγRIIIA and/or FcγRIIA such that the immunoglobulin has an enhanced effector function, e.g., antibody-dependent cell-mediated cytotoxicity. Non-limiting examples of effector cell functions include antibody-dependent cell-mediated cytotoxicity (ADCC), antibody-dependent phagocytosis, phagocytosis, opsonization, opsonophagocytosis, cell binding, rosetting, C1q binding, and complement dependent cell-mediated cytotoxicity.

In a preferred embodiment, the alteration in affinity or effector function is at least 2-fold, preferably at least 4-fold, at least 5-fold, at least 6-fold, at least 7-fold, at least 8-fold, at least 9-fold, at least 10-fold, at least 50-fold, or at least 100-fold, relative to a comparable molecule comprising a wild-type Fc Domain. In other embodiments of the invention, the variant Fc Domain immunospecifically binds one or more FcRs with at least 65%, preferably at least 70%, 75%, 80%, 85%, 90%, 95%, 100%, 125%, 150%, 175%, 200%, 225%, or 250% greater affinity relative to a molecule comprising a wild-type Fc Domain. Such measurements can be in vivo or in vitro assays, and in a preferred embodiment are in vitro assays such as ELISA or surface plasmon resonance assays.

In different embodiments, the Tri-Specific Binding Molecules of the present invention comprise a variant Fc Domain wherein said variant agonizes at least one activity of an FcγR receptor, or antagonizes at least one activity of an FcγR receptor. In a preferred embodiment, the molecules comprise a variant that antagonizes one or more activities of FcγRIIB, for example, B cell receptor-mediated signaling, activation of B cells, B cell proliferation, antibody production, intracellular calcium influx of B cells, cell cycle progression, FcγRIIB-mediated inhibition of FcεRI signaling, phosphorylation of FcγRIIB, SHIP recruitment, SHIP phosphorylation and association with Shc, or activity of one or more downstream molecules (e.g., MAP kinase, INK, p38, or Akt) in the FcγRIIB signal transduction pathway. In another embodiment, the Tri-Specific Binding Molecules of the present invention comprise a variant that agonizes one or more activities of FcεRI, for example, mast cell activation, calcium mobilization, degranulation, cytokine production, or serotonin release.

In certain embodiments, the molecules comprise an Fc Domain comprising regions from two or more IgG isotypes (e.g., IgG1, IgG2, IgG3 and IgG4). The various IgG isotypes exhibit differing physical and functional properties including serum half-life, complement fixation, FcγR binding affinities and effector function activities (e.g., ADCC, CDC, etc.) due to differences in the amino acid sequences of their hinge and/or Fc Domains, for example as described in Flesch and Neppert (1999) J. Clin. Lab. Anal. 14:141-156; Chappel et al. (1993) J. Biol. Chem. 33:25124-25131; Chappel et al. (1991) Proc. Natl. Acad. Sci. (U.S.A.) 88:9036-9040; or Bruggemann et al. (1987) J. Exp. Med 166:1351-1361. This type of variant Fc Domain may be used alone, or in combination with an amino acid modification, to affect Fc-mediated effector function and/or binding activity. In combination, the amino acid modification and IgG hinge/Fc Domain may display similar functionality (e.g., increased affinity for FcγRIIA) and may act additively or, more preferably, synergistically to modify the effector functionality in the molecule of the invention, relative to a molecule of the invention comprising a wild-type Fc Domain. In other embodiments, the amino acid modification and IgG Fc Domain may display opposite functionality (e.g., increased and decreased affinity for FcγRIIA, respectively) and may act to selectively temper or reduce a specific functionality in the molecule of the invention, relative to a molecule of the invention not comprising an Fc Domain or comprising a wild-type Fc Domain of the same isotype.

In a preferred specific embodiment, the Tri-Specific Binding Molecules of the present invention comprise a variant Fc Domain, wherein said variant Fc Domain comprises at least one amino acid modification relative to a wild-type Fc Domain, such that said molecule has an altered affinity for an FcR, provided that said variant Fc Domain does not have a substitution at positions that make a direct contact with FcγR based on crystallographic and structural analysis of Fc-FcR interactions such as those disclosed by Sondermann et al. (2000) Nature 406:267-73. Examples of positions within the Fc Domain that make a direct contact with FcγR are amino acid residues 234-239 (hinge region), amino acid residues 265-269 (B/C loop), amino acid residues 297-299 (C′/E loop), and amino acid residues 327-332 (F/G loop). In some embodiments, the molecules of the invention comprise variant Fc Domains comprise modification of at least one residue that does not make a direct contact with an FcγR based on structural and crystallographic analysis, e.g., is not within the Fc-FcγR binding site.

Variant Fc Domains are well-known in the art, and any known Fc variant may be used in the present invention to confer or modify the effector function exhibited by a molecule of the invention comprising an Fc Domain (or portion thereof) as functionally assayed, e.g., in an NK dependent or macrophage dependent assay. For example, Fc Domain variants identified as altering effector function are disclosed in the Antibody Engineering Technology Art, and any suitable variant disclosed therein may be used in the present molecules.

In certain embodiments, the Tri-Specific Binding Molecules of the present invention comprise a variant Fc Domain, having one or more amino acid modifications in one or more sites, which modification(s) alter (relative to a wild-type Fc Domain) the Ratio of Affinities of the variant Fc Domain to an activating FcγR (such as FcγRIIA or FcγRIIIA) relative to an inhibiting FcγR (such as FcγRIIB):

$$Ratio of Affinities = \frac{Wild - Type to Variant Change in Affinity to Fc γ {R}_{Activating}}{Wild - Type to Variant Change in Affinity to Fc γ {R}_{Inhibiting}}$$

Particularly preferred are Tri-Specific Binding Molecules of the present invention that possess a variant Fc Domain (relative to the wild-type Fc Domain) in which the Fc variant has a Ratio of Affinities greater than 1. Such molecules have particular use in providing a therapeutic or prophylactic treatment of a disease, disorder, or infection, or the amelioration of a symptom thereof, where an enhanced efficacy of effector cell function (e.g., ADCC) mediated by FcγR is desired, e.g., cancer or infectious disease. In contrast, an Fc variant having a Ratio of Affinities less than 1 mediates decreased efficacy of effector cell function. Table 1 lists exemplary single, double, triple, quadruple and quintuple mutations by whether their Ratio of Affinities is greater than or less than 1.

TABLE 1
Exemplary Single and Multiple Mutations Listed by
Ratio of Affinities

| | | | | |
|---|---|---|---|---|
| Single | Double | Triple | Quadruple | Quintuple |

Ratio of Affinities > 1

| | | | | |
|---|---|---|---|---|
| F243L | F243L & | F243L, P247L & | L234F, F243L, | L235V, F243L, |
| D270E | R292P | N421K | R292P & Y300L | R292P, Y300L |
| R292G | F243L & | F243L, R292P & | L2351, F243L, | & P396L |
| R292P | Y300L | Y300L | R292P & Y300L | L235P, F243L, |
| | F243L & | F243L, R292P & | L235Q, F243L, | R292P, Y300L |
| | P396L | V305I | R292P & Y300L | & P396L |
| | D270E & | F243L, R292P & | F243L, P247L, | F243L, R292P, |
| | P396L | P396L | D270E & N421K | V3051, Y300L |
| | R292P & | F243L, Y300L & | F243L, R255L, | & P396L |
| | Y300L | P396L | D270E & P396L | |
| | R292P & | P247L, D270E & | F243L, D270E, | |
| | V3051 | N421K | G316D & R416G | |
| | R292P & | R255L, D270E & | F243L, D270E, | |
| | P396L | P396L | K392T & P396L | |
| | Y300L & | D270E, G316D & | F243L, D270E, | |
| | P396L | R416G | P396L & Q419H | |
| | P396L & | D270E, K392T & | F243L, R292P, | |
| | Q419H | P396L | Y300L, & P396L | |
| | | D270E, P396L & | F243L, R292P, | |
| | | Q419H | V3051 & P396L | |
| | | V284M, R292L & | P247L, D270E, | |
| | | K370N | Y300L & N421K | |
| | | R292P, Y300L & | R255L, D270E, | |
| | | P396L | R292G & P396L | |
| | | | R255L, D270E, | |
| | | | Y300L & P396L | |
| | | | D270E, G316D, | |
| | | | P396L & R416G | |

Ratio of Affinities < 1

| | | |
|---|---|---|
| Y300L | F243L & | F243L, R292P & |
| P396L | P396L | V305I |
| | P247L & | |
| | N421K | |
| | R255L & | |
| | P396L | |
| | R292P & | |
| | V305I | |
| | K392T & | |
| | P396L | |
| | P396L & | |
| | Q419H | |

In a specific embodiment, in variant Fc Domains, any amino acid modifications (e.g., substitutions) at any of positions 235, 240, 241, 243, 244, 247, 262, 263, 269, 298, 328, or 330 and preferably one or more of the following residues: A240, I240, L241, L243, H244, N298, I328 or V330. In a different specific embodiment, in variant Fc Domains, any amino acid modifications (e.g., substitutions) at any of positions 268, 269, 270, 272, 276, 278, 283, 285, 286, 289, 292, 293, 301, 303, 305, 307, 309, 331, 333, 334, 335, 337, 338, 340, 360, 373, 376, 416, 419, 430, 434, 435, 437, 438 or 439 and preferably one or more of the following residues: H280, Q280, Y280, G290, S290, T290, Y290, N294, K295, P296, D298, N298, P298, V298, I300 or L300.

In a preferred embodiment, in variant Fc Domains that bind an FcγR with an altered affinity, any amino acid modifications (e.g., substitutions) at any of positions 255, 256, 258, 267, 268, 269, 270, 272, 276, 278, 280, 283, 285, 286, 289, 290, 292, 293, 294, 295, 296, 298, 300, 301, 303, 305, 307, 309, 312, 320, 322, 326, 329, 330, 332, 331, 333, 334, 335, 337, 338, 339, 340, 359, 360, 373, 376, 416, 419, 430, 434, 435, 437, 438 or 439. Preferably, the variant Fc Domain has any of the following residues: A256, N268, Q272, D286, Q286, S286, A290, S290, A298, M301, A312, E320, M320, Q320, R320, E322, A326, D326, E326, N326, S326, K330, T339, A333, A334, E334, H334, L334, M334, Q334, V334, K335, Q335, A359, A360 or A430.

In a different embodiment, in variant Fc Domains that bind an FcγR (via its Fc Domain) with a reduced affinity, any amino acid modifications (e.g., substitutions) at any of positions 252, 254, 265, 268, 269, 270, 278, 289, 292, 293, 294, 295, 296, 298, 300, 301, 303, 322, 324, 327, 329, 333, 335, 338, 340, 373, 376, 382, 388, 389, 414, 416, 419, 434, 435, 437, 438 or 439.

In a different embodiment, in variant Fc Domains that bind an FcγR (via its Fc Domain) with an enhanced affinity, any amino acid modifications (e.g., substitutions) at any of positions 280, 283, 285, 286, 290, 294, 295, 298, 300, 301, 305, 307, 309, 312, 315, 331, 333, 334, 337, 340, 360, 378, 398 or 430. In a different embodiment, in variant Fc Domains that binds FcγRIIA with an enhanced affinity, any of the following residues: A255, A256, A258, A267, A268, N268, A272, Q272, A276, A280, A283, A285, A286, D286, Q286, S286, A290, S290, M301, E320, M320, Q320, R320, E322, A326, D326, E326, S326, K330, A331, Q335, A337 or A430.

Preferred variants include one or more modifications at any of positions: 228, 230, 231, 232, 233, 234, 235, 239, 240, 241, 243, 244, 245, 247, 262, 263, 264, 265, 266, 271, 273, 275, 281, 284, 291, 296, 297, 298, 299, 302, 304, 305, 313, 323, 325, 326, 328, 330 or 332.

Particularly preferred variants include one or more modifications selected from groups A-AI:

| | |
|---|---|
| A | 228E, 228K, 228Y or 228G; |
| B | 230A, 230E, 230Y or 230G; |
| C | 231E, 231K, 231Y, 231P or 231G; |
| D | 232E, 232K, 232Y, 232G; |
| E | 233D; |
| F | 234I or 234F; |
| G | 235D, 235Q, 235P, 235I or 235V; |
| H | 239D, 239E, 239N or 239Q; |
| I | 240A, 240I, 240M or 240T; |
| J | 243R, 243, 243Y, 243L, 243Q, 243W, 243H or 243I; |
| K | 244H; |
| L | 245A; |
| M | 247G, 247V or 247L; |
| N | 262A, 262E, 262I, 262T, 262E or 262F; |
| O | 263A, 263I, 263M or 263T; |
| P | 264F, 264E, 264R, 264I, 264A, 264T or 264W; |
| Q | 265F, 265Y, 265H, 265I, 265L, 265T, 265V, 265N or 265Q; |
| R | 266A, 266I, 266M or 266T; |
| S | 271D, 271E, 271N, 271Q, 271K, 271R, 271S, 271T, 271H, |
| | 271A, 271V, 271L, 271I, 271F, 271M, 271Y, 271W or 271G; |
| T | 273I; |
| U | 275L or 275W; |
| V | 281D, 281K, 281Y or 281P; |
| W | 284E, 284N, 284T, 284L, 284Y or284M; |
| X | 291D, 291E, 291Q, 291T, 291H, 291I or 291G; |
| Y | 299A, 299D, 299E, 299F, 299G, 299H, 299I, 299K, 299L, |
| | 299M, 299N, 299P, 299Q, 299R, 299S, 299V, 299W or 299Y; |
| Z | 302I; |
| AA | 304D, 304N, 304T, 304H or 304L |
| AB | 305I; |
| AC | 313F; |
| AD | 323I; |
| AE | 325A, 325D, 325E, 325G, 325H, 325I, 325L, |
| | 325K, 325R, 325S, 325F, 325M, 325T, |
| | 325V, 325Y, 325W or 325P; |
| AF | 328D, 328Q, 328K, 328R, 328S, 328T, 328V, |
| | 328I, 328Y, 328W, 328P, 328G, 328A, |
| | 328E, 328F, 328H, 328M or 328N; |
| AG | 330L, 330Y, 330I or 330V; |
| AH | 332A, 332D, 332E, 332H, 332N, 332Q, 332T, 332K, 332R, 332S, |
| | 332V, 332L, 332F, 332M, 332W, 332P, 332G or 332Y; and |
| AI | 336E, 336K or 336Y |

Still more particularly preferred variants include one or more modifications selected from Groups 1-105:

| Group | Variant |
|---|---|
| 1 | A330L/I332E |
| 2 | D265F/N297E/I332E |
| 3 | D265Y/N297D/I332E |
| 4 | D265Y/N297D/T299L/I332E |
| 5 | F241E/F243Q/V262T/V264F |
| 6 | F241E/F243Q/V262T/V264E/I332E |
| 7 | F241E/F243R/V262E/V264R |
| 8 | F241E/F243R/V262E/V264R/I332E |
| 9 | F241E/F243Y/V262T/V264R |
| 10 | F241E/F243Y/V262T/V264R/I332E |
| 11 | F241L/F243L/V262I/V264I |
| 12 | F241L/V262I |
| 13 | F241R/F243Q/V262T/V264R |
| 14 | F241R/F243Q/V262T/V264R/I332E |
| 15 | F241W/F243W/V262A/V264A |
| 16 | F241Y/F243Y/V262T/V264T |
| 17 | F241Y/F243Y/V262T/V264T/N297D/I332E |
| 18 | F243L/V262I/V264W |
| 19 | P243L/V264I |
| 20 | L328D/I332E |
| 21 | L328E/I332E |
| 22 | L328H/I332E |
| 23 | L328I/I332E |
| 24 | L328M/I332E |
| 25 | L328N/I332E |
| 26 | L328Q/I332E |
| 27 | L328T/I332E |
| 28 | L328V/I332E |
| 29 | N297D/A330Y/I332E |
| 30 | N297D/I332E |
| 31 | N297D/I332E/S239D/A330L |
| 32 | N297D/S298A/A330Y/I332E |
| 33 | N297D/T299L/I332E |
| 34 | N297D/T299F/I332E/N297D/T299H/I332E |
| 35 | N297D/T299I/I332E |
| 36 | N297D/T299L/I332E |
| 37 | N297D/T299V/I332E |
| 38 | N297E/I332E |
| 39 | N297S/I332E |
| 40 | P230A/E233D/I332E |
| 41 | P244H/P245A/P247V |
| 42 | S239D/A330L/I332E |
| 43 | S239D/A330Y/I332E |
| 44 | S239D/A330Y/I332E/K326E |
| 45 | S239D/A330Y/I332E/K326T |
| 46 | S239D/A330Y/I332E/L234I |
| 47 | S239D/A330Y/I332E/L235D |
| 48 | S239D/A330Y/I332E/V240I |
| 49 | S239D/A330Y/I332E/V264T |
| 50 | S239D/A330Y/I332E/V266I |
| 51 | S239D/D265F/N297D/I332E |
| 52 | S239D/D265H/N297D/I332E |
| 53 | S239D/D265I/N297D/I332E |
| 54 | S239D/D265L/N297D/I332E |
| 55 | S239D/D265T/N297D/I332E |
| 56 | S239D/D265V/N297D/I332E |
| 57 | S239D/D265Y/N297D/I332E |
| 58 | S239D/I332D |
| 59 | S239D/I332E |
| 60 | S239D/I332E/A330I |
| 61 | S239D/I332N |
| 62 | S239D/I332Q |
| 63 | S239D/N297D/I332E |
| 64 | S239D/N297D/I332E/A330Y |
| 65 | S239D/N297D/I332E/A330Y/F241S/F243H/V262T/V264T |
| 66 | S239D/N297D/I332E/K326E |
| 67 | S239D/N297D/I332E/ L235D |
| 68 | S239D/S298A/I332E |
| 69 | S239D/V264I/A330L/I332E |
| 70 | S239D/V264I/I332E |
| 71 | S239D/V264I/S298A/I332E |
| 72 | S239E/D265N |
| 73 | S239E/D265Q |
| 74 | S239E/I332D |
| 75 | S239E/I332E |
| 76 | S239E/I332N |
| 77 | S239E/I332Q |
| 78 | S239E/N297D/I332E |
| 79 | S239E/V264I/A330Y/I332E |
| 80 | S239E/V264I/I332E |
| 81 | S239E/V264I/S298A/A330Y/I332E |
| 82 | S239N/A330L/I332E |
| 83 | S239N/A330Y/I332E |
| 84 | S239N/I332D |
| 85 | S239N/I332E |
| 86 | S239N/I332N |
| 87 | S239N/I332Q |
| 88 | S239N1S298A/I332E |
| 89 | S239Q/I332D |
| 90 | S239Q/I332E |
| 91 | S239Q/I332N |
| 92 | S239Q/I332Q |
| 93 | S239Q/V264I/I332E |
| 94 | S298A/I332E |
| 95 | V264E/N297D/I332E |
| 96 | V264I/A330L/I332E |
| 97 | V264I/A330Y/I332E |
| 98 | V264I/I332E |
| 99 | V264I/S298A/I332E |
| 100 | Y296D/N297D/I332E |
| 101 | Y296E/N297D/I332E |
| 102 | Y296H/N297D/I332E |
| 103 | Y296N/N297D/I332E |
| 104 | Y296Q/N297I/I332E |
| 105 | Y296T/N297D/I332E. |

In one embodiment, a multivalent DR5 binding molecule of the invention will comprise a variant Fe Domain having at least one modification in the Fc Domain. In certain embodiments, the variant Fc Domain comprises at least one substitution selected from the group consisting of L235V, F243L, R292P, Y300L, V305I, and P396L, wherein said numbering is that of the EU index as in Kabat.

In a specific embodiment, the variant Fc Domain comprises:
(A) at least one substitution selected from the group consisting of F243L, R292P, Y300L, V305I, and P396L;
(B) at least two substitutions selected from the group consisting of:
   (1) F243L and P396L;
   (2) F243L and R292P; and
   (3) R292P and V305I;
(C) at least three substitutions selected from the group consisting of:
   (1) F243L, R292P and Y300L;
   (2) F243L, R292P and V305I;
   (3) F243L, R292P and P396L; and
   (4) R292P, V305I and P396L;
(D) at least four substitutions selected from the group consisting of:
   (1) F243L, R292P, Y300L and P396L; and
   (2) F243L, R292P, V305I and P396L; or
(E) at least the five substitutions selected from the group consisting of:
   (1) F243L, R292P, Y300L, V305I and P396L; and
   (2) L235V, F243L, R292P, Y300L and P396L.

In another specific embodiment, the variant Fc Domain comprises substitutions of:
(A) F243L, R292P, and Y300L;
(B) L235V, F243L, R292P, Y300L, and P396L; or
(C) F243L, R292P, Y300L, V305I, and P396L.

In other embodiments, the invention encompasses the use of any Fc variant known in the art, such as those disclosed in Jefferis, B. J. et al. (2002) “Interaction Sites On Human IgG-Fc For FcgammaR: Current Models,” Immunol. Lett. 82:57-65; Presta, L. G. et al. (2002) “Engineering Therapeutic Antibodies For Improved Function,” Biochem. Soc. Trans. 30:487-90; Idusogie, E. E. et al. (2001) “Engineered Antibodies With Increased Activity To Recruit Complement,” J. Immunol. 166:2571-75; Shields, R. L. et al. (2001) “High Resolution Mapping Of The Binding Site On Human IgG1 For Fc Gamma RI, Fc Gamma RII, Fc Gamma RIII, And FcRn And Design Of IgG1 Variants With Improved Binding To The Fc gamma R,” J. Biol. Chem. 276:6591-6604; Idusogie, E. E. et al. (2000) “Mapping Of The C1q Binding Site On Rituxan, A Chimeric Antibody With A Human IgG Fc,” J. Immunol. 164:4178-84; Reddy, M. P. et al. (2000) “Elimination Of Fc Receptor-Dependent Effector Functions Of A Modified IgG4 Monoclonal Antibody To Human CD4,” J. Immunol. 164:1925-1933; Xu, D. et al. (2000) “In Vitro Characterization of Five Humanized OKT3 Effector Function Variant Antibodies,” Cell. Immunol. 200:16-26; Armour, K. L. et al. (1999) “Recombinant human IgG Molecules Lacking Fcgamma Receptor I Binding And Monocyte Triggering Activities,” Eur. J. Immunol. 29:2613-24; Jefferis, R. et al. (1996) “Modulation Of Fc(Gamma)R And Human Complement Activation By IgG3-Core Oligosaccharide Interactions,” Immunol. Lett. 54:101-04; Lund, J. et al. (1996) “Multiple Interactions Of IgG With Its Core Oligosaccharide Can Modulate Recognition By Complement And Human Fc Gamma Receptor I And Influence The Synthesis Of Its Oligosaccharide Chains,” J. Immunol. 157:4963-4969; Hutchins et al. (1995) “Improved Biodistribution, Tumor Targeting, And Reduced Immunogenicity In Mice With A Gamma 4 Variant Of Campath-1H,” Proc. Natl. Acad. Sci. (U.S.A.) 92:11980-84; Jefferis, R. et al. (1995) “Recognition Sites On Human IgG For Fc Gamma Receptors: The Role Of Glycosylation,” Immunol. Lett. 44:111-17; Lund, J. et al. (1995) “Oligosaccharide-Protein Interactions In IgG Can Modulate Recognition By Fc Gamma Receptors,” FASEB J. 9:115-19; Alegre, M. L. et al. (1994) “A Non-Activating “Humanized” Anti-CD3 Monoclonal Antibody Retains Immunosuppressive Properties In Vivo,” Transplantation 57:1537-1543; Lund et al. (1992) “Multiple Binding Sites On The CH2 Domain Of IgG For Mouse Fc Gamma R11,” Mol. Immunol. 29:53-59; Lund et al. (1991) “Human Fc Gamma RI And Fc Gamma RII Interact With Distinct But Overlapping Sites On Human IgG,” J. Immunol. 147:2657-2662; Duncan, A. R. et al. (1988) “Localization Of The Binding Site For The Human High-Affinity Fc Receptor On IgG,” Nature 332:563-564; U.S. Pat. Nos. 5,624,821; 5,885,573; 6,194,551; 7,276,586; and 7,317,091; and PCT Publications WO 00/42072 and PCT WO 99/58572.

In some embodiments, the molecules of the invention further comprise one or more glycosylation sites, so that one or more carbohydrate moieties are covalently attached to the molecule. Preferably, the molecules of the invention with one or more glycosylation sites and/or one or more modifications in the Fc Domain confer or have an enhanced antibody-mediated effector function, e.g., enhanced ADCC activity, compared to a parent antibody. In some embodiments, the invention further comprises molecules comprising one or more modifications of amino acids that are directly or indirectly known to interact with a carbohydrate moiety of the antibody, including but not limited to amino acids at positions 241, 243, 244, 245, 245, 249, 256, 258, 260, 262, 264, 265, 296, 299, and 301. Amino acids that directly or indirectly interact with a carbohydrate moiety of an antibody are known in the art, see, e.g., Jefferis et al., 1995 Immunology Letters, 44: 111-7, which is incorporated herein by reference in its entirety.

In another embodiment, the invention encompasses molecules that have been modified by introducing one or more glycosylation sites into one or more sites of the molecules, preferably without altering the functionality of the molecules, e.g., binding activity to target antigen or FcγR. Glycosylation sites may be introduced into the variable and/or constant region of the molecules of the invention. As used herein, “glycosylation sites” include any specific amino acid sequence in an antibody to which an oligosaccharide (i.e., carbohydrates containing two or more simple sugars linked together) will specifically and covalently attach. Oligosaccharide side chains are typically linked to the backbone of an antibody via either N- or O-linkages. N-linked glycosylation refers to the attachment of an oligosaccharide moiety to the side chain of an asparagine residue. O-linked glycosylation refers to the attachment of an oligosaccharide moiety to a hydroxyamino acid, e.g., serine, threonine. The molecules of the invention may comprise one or more glycosylation sites, including N-linked and O-linked glycosylation sites. Any glycosylation site for N-linked or O-linked glycosylation known in the art may be used in accordance with the instant invention. An exemplary N-linked glycosylation site that is useful in accordance with the methods of the present invention is the amino acid sequence: Asn-X-Thr/Ser, wherein X may be any amino acid and Thr/Ser indicates a threonine or a serine. Such a site or sites may be introduced into a molecule of the invention using methods well-known in the art to which this invention pertains (see for example, IN VITRO MUTAGENESIS, RECOMBINANT DNA: A SHORT COURSE, J. D. Watson, et al. W. H. Freeman and Company, New York, 1983, chapter 8, pp. 106-116, which is incorporated herein by reference in its entirety. An exemplary method for introducing a glycosylation site into a molecule of the invention may comprise: modifying or mutating an amino acid sequence of the molecule so that the desired Asn-X-Thr/Ser sequence is obtained.

In some embodiments, the invention encompasses methods of modifying the carbohydrate content of a molecule of the invention by adding or deleting a glycosylation site. Methods for modifying the carbohydrate content of antibodies (and molecules comprising antibody domains) are well-known in the art and encompassed within the invention, see, e.g., U.S. Pat. No. 6,218,149; EP 0 359 096 Bi; U.S. Publication No. US 2002/0028486; WO 03/035835; U.S. Publication No. 2003/0115614; U.S. Pat. Nos. 6,218,149; 6,472,511; all of which are incorporated herein by reference in their entirety. In other embodiments, the invention encompasses methods of modifying the carbohydrate content of a molecule of the invention by deleting one or more endogenous carbohydrate moieties of the molecule. In a specific embodiment, the invention encompasses shifting the glycosylation site of the Fc Domain of an antibody, by modifying positions adjacent to 297. In a specific embodiment, the invention encompasses modifying position 296 so that position 296 and not position 297 is glycosylated.

Effector function can also be modified by techniques such as by introducing one or more cysteine residues into the Fc Domain, thereby allowing interchain disulfide bond formation in this region to occur, resulting in the generation of a homodimeric antibody that may have improved internalization capability and/or increased complement-mediated cell killing and ADCC (Caron, P. C. et al. (1992) “Engineered Humanized Dimeric Forms Of IgG Are More Effective Antibodies,” J. Exp. Med. 176:1191-1195; Shopes, B. (1992) “A Genetically Engineered Human IgG Mutant With Enhanced Cytolytic Activity,” J. Immunol. 148(9):2918-2922. Homodimeric antibodies with enhanced antitumor activity may also be prepared using heterobifunctional cross-linkers as described in Wolff, E. A. et al. (1993) “Monoclonal Antibody Homodimers: Enhanced Antitumor Activity In Nude Mice,” Cancer Research 53:2560-2565. Alternatively, an antibody can be engineered which has dual Fc Domains and may thereby have enhanced complement lysis and ADCC capabilities (Stevenson, G. T. et al. (1989) “A Chimeric Antibody With Dual Fc Domains (bisFabFc) Prepared By Manipulations At The IgG Hinge,” Anti-Cancer Drug Design 3:219-230).

### III. Exemplary Trispecific Binding Molecules

### F. gpA33 mAb 1×CD3 mAb 2×DR5 mAb 1

An exemplary Tri-Specific Binding Molecule composed of four polypeptide chains was constructed. The Tri-Specific Binding Molecule comprises the VL and VH domains of gpA33 mAb 1, the VL and VH domains of antibody CD3 mAb 2 and the VL and VH domains of DR5 mAb 1, and was accordingly designated “gpA33 mAb 1×CD3 mAb 2×DR5 mAb 1.” The amino acid sequence of the first polypeptide chain of this Tri-Specific Binding Molecule is (SEQ ID NO:212):

DIQLTQSPSF LSASVGDRVT ITCSARSSIS FMYWYQQKPG
KAPKLLIYDT SNLASGVPSR FSGSGSGTEF TLTISSLEAE
DAATYYCQQW SSYPLTFGQG TKLEIKGGGS GGGGEVQLVE
SGGGLVQPGG SLRLSCAASG FTFSTYAMNW VRQAPGKGLE
WVGRIRSKYN NYATYYADSV KGRFTISRDD SKNSLYLQMN
SLKTEDTAVY YCVRHGNFGN SYVSWFAYWG QGTLVTVSSG
GCGGGEVAAL EKEVAALEKE VAALEKEVAA LEKGGGDKTH
TCPPCPAPEA AGGPSVFLFP PKPKDTLMIS RTPEVTCVVV
DVSHEDPEVK FNWYVDGVEV HNAKTKPREE QYNSTYRVVS
VLTVLHQDWL NGKEYKCKVS NKALPAPIEK TISKAKGQPR
EPQVYTLPPS REEMTKNQVS LWCLVKGFYP SDIAVEWESN
GQPENNYKTT PPVLDSDGSF FLYSKLTVDK SRWQQGNVFS
CSVMHEALHN HYTQKSLSLS PGK

In SEQ ID NO:212, amino acid residues 1-106 correspond to the amino acid sequence of the VL Domain of gpA33 mAb 1 (SEQ ID NO:181), residues 107-114 correspond to the intervening spacer peptide GGGSGGGG (Linker 1) (SEQ ID NO:33), residues 115-239 correspond to the amino acid sequence of the VH Domain of CD3 mAb 2 having the D65G substitution (SEQ ID NO:112), residues 240-245 correspond to the GGCGGG linker (SEQ ID NO:34), residues 246-273 correspond to an E-coil Domain (SEQ ID NO:39), residues 274-276 are the linker GGG, residues 277-286 are the linker DKTHTCPPCP (SEQ ID NO:48), and residues 287-503 are the “knob-bearing” CH2-CH3 Domain (SEQ ID NO:52).

A polynucleotide that encodes SEQ ID NO:212 is SEQ ID NO:213:

gacattcagc tgactcagtc cccctctttt ctgtccgcat
ccgtcggaga tcgagtgact attacttgct ctgctaggtc
ctcaatcagc ttcatgtact ggtatcagca gaagcccggc
aaagcaccta agctgctgat ctacgacaca agcaacctgg
cctccggggt gccatctcgg ttctctggca gtgggtcagg
aactgagttt accctgacaa ttagctccct ggaggctgaa
gatgccgcta cctactattg ccagcagtgg agcagctatc
ctctgacctt cggacagggg actaaactgg aaatcaaggg
tggaggatcc ggcggcggag gcgaggtgca gctggtggag
tctgggggag gcttggtcca gcctggaggg tccctgagac
tctcctgtgc agcctctgga ttcaccttca gcacatacgc
tatgaattgg gtccgccagg ctccagggaa ggggctggag
tgggttggaa ggatcaggtc caagtacaac aattatgcaa
cctactatgc cgactctgtg aagggtagat tcaccatctc
aagagatgat tcaaagaact cactgtatct gcaaatgaac
agcctgaaaa ccgaggacac ggccgtgtat tactgtgtga
gacacggtaa cttcggcaat tcttacgtgt cttggtttgc
ttattgggga caggggacac tggtgactgt gtcttccgga
ggatgtggcg gtggagaagt ggccgcactg gagaaagagg
ttgctgcttt ggagaaggag gtcgctgcac ttgaaaagga
ggtcgcagcc ctggagaaag gcggcgggga caaaactcac
acatgcccac cgtgcccagc acctgaagcc gcggggggac
cgtcagtctt cctcttcccc ccaaaaccca aggacaccct
catgatctcc cggacccctg aggtcacatg cgtggtggtg
gacgtgagcc acgaagaccc tgaggtcaag ttcaactggt
acgtggacgg cgtggaggtg cataatgcca agacaaagcc
gcgggaggag cagtacaaca gcacgtaccg tgtggtcagc
gtcctcaccg tcctgcacca ggactggctg aatggcaagg
agtacaagtg caaggtctcc aacaaagccc tcccagcccc
catcgagaaa accatctcca aagccaaagg gcagccccga
gaaccacagg tgtacaccct gcccccatcc cgggaggaga
tgaccaagaa ccaggtcagc ctgtggtgcc tggtcaaagg
cttctatccc agcgacatcg ccgtggagtg ggagagcaat
gggcagccgg agaacaacta caagaccacg cctcccgtgc
tggactccga cggctccttc ttcctctaca gcaagctcac
cgtggacaag agcaggtggc agcaggggaa cgtcttctca
tgctccgtga tgcatgaggc tctgcacaac cactacacgc
agaagagcct ctccctgtct ccgggtaaa

The amino acid sequence of the second polypeptide chain of gpA33 mAb 1×CD3 mAb 2×DR5 mAb 1 is (SEQ ID NO:214):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGQV
QLVQSGAEVK KPGASVKVSC KASGYTFTGS WMNWVRQAPG
QGLEWIGRIY PGDGETNYNG KFKDRVTITA DKSTSTAYME
LSSLRSEDTA VYYCARIYGN NVYFDVWGQG TTVTVSSGGC
GGGKVAALKE KVAALKEKVA ALKEKVAALK E

In SEQ ID NO:214, amino acid residues 1-110 correspond to the amino acid sequence of the VL Domain of CD3 mAb 2 (SEQ ID NO:104), residues 111-118 correspond to the intervening spacer peptide GGGSGGGG (Linker 1) (SEQ ID NO:33), residues 119-237 correspond to the amino acid sequence of the VH Domain of gpA33 mAb 1 (SEQ ID NO:186), residues 238-243 correspond to the linker GGCGGG (SEQ ID NO:34), and residues 244-271 are a K-coil Domain (SEQ ID NO:40).

A polynucleotide that encodes SEQ ID NO:214 is (SEQ ID NO:215):

caggctgtgg tgactcagga gccttcactg accgtgtccc
caggcggaac tgtgaccctg acatgcagat ccagcacagg
cgcagtgacc acatctaact acgccaattg ggtgcagcag
aagccaggac aggcaccaag gggcctgatc gggggtacaa
acaaaagggc tccctggacc cctgcacggt tttctggaag
tctgctgggc ggaaaggccg ctctgactat taccggggca
caggccgagg acgaagccga ttactattgt gctctgtggt
atagcaatct gtgggtgttc gggggtggca caaaactgac
tgtgctggga gggggtggat ccggcggagg tggacaggtc
cagctggtcc agagcggggc cgaagtcaaa aaacccggag
caagcgtgaa ggtctcctgc aaagcatcag gctatacatt
tacaggcagc tggatgaact gggtgaggca ggctccagga
cagggactgg agtggatcgg gcgcatctac cctggagacg
gcgaaactaa ctataatgga aagttcaaag accgagtgac
catcacagcc gataagtcta ctagtaccgc ctacatggag
ctgagctccc tgcggtctga agataccgcc gtctactatt
gcgctagaat ttacggaaac aatgtctatt ttgacgtgtg
ggggcaggga acaactgtga ctgtctcctc cggaggatgt
ggcggtggaa aagtggccgc actgaaggag aaagttgctg
ctttgaaaga gaaggtcgcc gcacttaagg aaaaggtcgc
agccctgaaa gag

The amino acid sequence of the third polypeptide chain of gpA33 mAb 1×CD3 mAb 2×DR5 mAb 1 is (SEQ ID NO:216):

EVKFLESGGG LVQPGGSLKL SCVASGFDFS RYWMSWVRQA
PGKGLEWIGE INPDSNTINY TPSLKDKFII SRDNAKNTLY
LQMTKVRSED TALYYCTRRA YYGNPAWFAY WGQGTLVTVS
SASTKGPSVF PLAPSSKSTS GGTAALGCLV KDYFPEPVTV
SWNSGALTSG VHTFPAVLQS SGLYSLSSVV TVPSSSLGTQ
TYICNVNHKP SNTKVDKRVE PKSCDKTHTC PPCPAPEAAG
GPSVFLFPPK PKDTLMISRT PEVTCVVVDV SHEDPEVKFN
WYVDGVEVHN AKTKPREEQY NSTYRVVSVL TVLHQDWLNG
KEYKCKVSNK ALPAPIEKTI SKAKGQPREP QVYTLPPSRE
EMTKNQVSLS CAVKGFYPSD IAVEWESNGQ PENNYKTTPP
VLDSDGSFFL VSKLTVDKSR WQQGNVFSCS VMHEALHNRY
TQKSLSLSPG K

In SEQ ID NO:216, amino acid residues 1-121 correspond to the amino acid sequence of the VH Domain of DR5 mAb 1 (SEQ ID NO:8), residues 122-219 correspond to a modified CH1 Domain (SEQ ID NO:208), residues 220-234 correspond to a linker (SEQ ID NO:209), and residues 235-451 correspond to the “hole-bearing” CH2-CH3 Domain (SEQ ID NO:53).

A polynucleotide that encodes SEQ ID NO:216 is (SEQ ID NO:217):

gaggtgaagt ttctcgagtc tggaggtggc ctggtgcagc
ctggaggatc cctgaaactc tcctgtgtag cctcaggatt
cgattttagt agatactgga tgagttgggt ccggcaggct
ccagggaaag ggctagaatg gattggagaa attaatccag
atagcaatac gataaactat acgccatctc taaaggataa
attcatcatc tccagagaca acgccaaaaa tacgctgtat
ctgcaaatga ccaaagtgag atctgaggac acagcccttt
attattgtac aagaagggcc tactatggta acccggcctg
gtttgcttac tggggccaag ggactctggt cactgtctct
tccgcctcca ccaagggccc atcggtcttc cccctggcac
cctcctccaa gagcacctct gggggcacag cggccctggg
ctgcctggtc aaggactact tccccgaacc ggtgacggtg
tcgtggaact caggcgccct gaccagcggc gtgcacacct
tcccggctgt cctacagtcc tcaggactct actccctcag
cagcgtggtg accgtgccct ccagcagctt gggcacccag
acctacatct gcaacgtgaa tcacaagccc agcaacacca
aggtggacaa gagagttgag cccaaatctt gtgacaaaac
tcacacatgc ccaccgtgcc cagcacctga agccgcgggg
ggaccgtcag tcttcctctt ccccccaaaa cccaaggaca
ccctcatgat ctcccggacc cctgaggtca catgcgtggt
ggtggacgtg agccacgaag accctgaggt caagttcaac
tggtacgtgg acggcgtgga ggtgcataat gccaagacaa
agccgcggga ggagcagtac aacagcacgt accgtgtggt
cagcgtcctc accgtcctgc accaggactg gctgaatggc
aaggagtaca agtgcaaggt ctccaacaaa gccctcccag
cccccatcga gaaaaccatc tccaaagcca aagggcagcc
ccgagaacca caggtgtaca ccctgccccc atcccgggag
gagatgacca agaaccaggt cagcctgagt tgcgcagtca
aaggcttcta tcccagcgac atcgccgtgg agtgggagag
caatgggcag ccggagaaca actacaagac cacgcctccc
gtgctggact ccgacggctc cttcttcctc gtcagcaagc
tcaccgtgga caagagcagg tggcagcagg ggaacgtctt
ctcatgctcc gtgatgcatg aggctctgca caaccgctac
acgcagaaga gcctctccct gtctccgggt aaa

The amino acid sequence of the fourth polypeptide chain of gpA33 mAb 1×CD3 mAb 2×DR5 mAb 1 is (SEQ ID NO:218):

DIVLTQSPAS LAVSLGQRAT ISCRASKSVS SSGYSYMHWY
QQKPGQPPKV LIFLSSNLDS GVPARFSGSG SGTDFTLNIH
PVEDGDAATY YCQHSRDLPP TFGGGTKLEI KRTVAAPSVF
IFPPSDEQLK SGTASVVCLL NNFYPREAKV QWKVDNALQS
GNSQESVTEQ DSKDSTYSLS STLTLSKADY EKHKVYACEV
THQGLSSPVT KSFNRGEC

In SEQ ID NO:218, amino acid residues 1-111 correspond to the amino acid sequence of the VL Domain of DR5 mAb 1 (SEQ ID NO:3), and residues 112-218 correspond to the CL Kappa Domain (SEQ ID NO:210).

A polynucleotide that encodes SEQ ID NO:218 is (SEQ ID NO:219):

gacattgtgc tgacacagtc tcctgcttcc ttagctgtat
ctctcgggca gagggccacc atctcatgca gggccagcaa
aagtgtcagt tcctctggct atagttatat gcactggtac
caacagaaac caggacagcc acccaaagtc ctcatctttc
tttcatccaa cctagattct ggggtccctg ccaggttcag
tggcagtggg tctgggacag acttcaccct caacatccat
cctgtggagg atggggatgc tgcaacctat tactgtcagc
acagtaggga tcttcctccg acgttcggtg gaggcaccaa
gctggaaatc aaacgtacgg tggctgcacc atcggtcttc
atcttcccgc catctgatga gcagttgaaa tctggaactg
cctctgttgt gtgcctgctg aataacttct atcccagaga
ggccaaagta cagtggaagg tggataacgc cctccaatcg
ggtaactccc aggagagtgt cacagagcag gacagcaagg
acagcaccta cagcctcagc agcaccctga cgctgagcaa
agcagactac gagaaacaca aagtctacgc ctgcgaagtc
acccatcagg gcctgagctc gcccgtcaca aagagcttca
acaggggaga gtgt

### G. Gpa33 mAB 1×CD3 mAB 2×DR5 mAB 2

A second exemplary Tri-Specific Binding Molecule composed of four polypeptide chains was constructed. The Tri-Specific Binding Molecule comprises the VL and VH domains of gpA33 mAb 1, the VL and VH domains of antibody CD3 mAb 2 and the VL and VH domains of DR5 mAb 2, and was accordingly designated “gpA33 mAb 1×CD3 mAb 2×DR5 mAb 2.” The amino acid sequence of the first polypeptide chain of this Tri-Specific Binding Molecule is (SEQ ID NO:220):

DIQLTQSPSF LSASVGDRVT ITCSARSSIS FMYWYQQKPG
KAPKLLIYDT SNLASGVPSR FSGSGSGTEF TLTISSLEAE
DAATYYCQQW SSYPLTFGQG TKLEIKGGGS GGGGEVQLVE
SGGGLVQPGG SLRLSCAASG FTFSTYAMNW VRQAPGKGLE
WVGRIRSKYN NYATYYADSV KGRFTISRDD SKNSLYLQMN
SLKTEDTAVY YCVRHGNFGN SYVSWFAYWG QGTLVTVSSG
GCGGGEVAAL EKEVAALEKE VAALEKEVAA LEKGGGDKTH
TCPPCPAPEA AGGPSVFLFP PKPKDTLMIS RTPEVTCVVV
DVSHEDPEVK FNWYVDGVEV HNAKTKPREE QYNSTYRVVS
VLTVLHQDWL NGKEYKCKVS NKALPAPIEK TISKAKGQPR
EPQVYTLPPS REEMTKNQVS LWCLVKGFYP SDIAVEWESN
GQPENNYKTT PPVLDSDGSF FLYSKLTVDK SRWQQGNVFS
CSVMHEALHN HYTQKSLSLS PGK

In SEQ ID NO:220, amino acid residues 1-106 correspond to the amino acid sequence of the VL Domain of gpA33 mAb 1 (SEQ ID NO:181), residues 107-114 correspond to the intervening spacer peptide GGGSGGGG (Linker 1) (SEQ ID NO:33), residues 115-239 correspond to the amino acid sequence of the VH Domain of CD3 mAb 2 having the D65G substitution (SEQ ID NO:112), residues 240-245 correspond to the GGCGGG linker (SEQ ID NO:34), residues 246-273 correspond to an E-coil Domain (SEQ ID NO:39), residues 274-276 are the linker GGG, residues 277-286 are the linker DKTHTCPPCP (SEQ ID NO:48), and residues 287-503 are the “knob-bearing” CH2-CH3 Domain (SEQ ID NO:52).

A polynucleotide that encodes SEQ ID NO:220 is (SEQ ID NO:221):

gacattcagc tgactcagtc cccctctttt ctgtccgcat
ccgtcggaga tcgagtgact attacttgct ctgctaggtc
ctcaatcagc ttcatgtact ggtatcagca gaagcccggc
aaagcaccta agctgctgat ctacgacaca agcaacctgg
cctccggggt gccatctcgg ttctctggca gtgggtcagg
aactgagttt accctgacaa ttagctccct ggaggctgaa
gatgccgcta cctactattg ccagcagtgg agcagctatc
ctctgacctt cggacagggg actaaactgg aaatcaaggg
tggaggatcc ggcggcggag gcgaggtgca gctggtggag
tctgggggag gcttggtcca gcctggaggg tccctgagac
tctcctgtgc agcctctgga ttcaccttca gcacatacgc
tatgaattgg gtccgccagg ctccagggaa ggggctggag
tgggttggaa ggatcaggtc caagtacaac aattatgcaa
cctactatgc cgactctgtg aagggtagat tcaccatctc
aagagatgat tcaaagaact cactgtatct gcaaatgaac
agcctgaaaa ccgaggacac ggccgtgtat tactgtgtga
gacacggtaa cttcggcaat tcttacgtgt cttggtttgc
ttattgggga caggggacac tggtgactgt gtcttccgga
ggatgtggcg gtggagaagt ggccgcactg gagaaagagg
ttgctgcttt ggagaaggag gtcgctgcac ttgaaaagga
ggtcgcagcc ctggagaaag gcggcgggga caaaactcac
acatgcccac cgtgcccagc acctgaagcc gcggggggac
cgtcagtctt cctcttcccc ccaaaaccca aggacaccct
catgatctcc cggacccctg aggtcacatg cgtggtggtg
gacgtgagcc acgaagaccc tgaggtcaag ttcaactggt
acgtggacgg cgtggaggtg cataatgcca agacaaagcc
gcgggaggag cagtacaaca gcacgtaccg tgtggtcagc
gtcctcaccg tcctgcacca ggactggctg aatggcaagg
agtacaagtg caaggtctcc aacaaagccc tcccagcccc
catcgagaaa accatctcca aagccaaagg gcagccccga
gaaccacagg tgtacaccct gcccccatcc cgggaggaga
tgaccaagaa ccaggtcagc ctgtggtgcc tggtcaaagg
cttctatccc agcgacatcg ccgtggagtg ggagagcaat
gggcagccgg agaacaacta caagaccacg cctcccgtgc
tggactccga cggctccttc ttcctctaca gcaagctcac
cgtggacaag agcaggtggc agcaggggaa cgtcttctca
tgctccgtga tgcatgaggc tctgcacaac cactacacgc
agaagagcct ctccctgtct ccgggtaaa

The amino acid sequence of the second polypeptide chain of gpA33 mAb 1×CD3 mAb 2×DR5 mAb 2 is (SEQ ID NO:222):

QAVVTQEPSL TVSPGGTVTL TCRSSTGAVT TSNYANWVQQ
KPGQAPRGLI GGINKRAPWT PARFSGSLLG GKAALTITGA
QAEDEADYYC ALWYSNLWVF GGGTKLTVLG GGGSGGGGQV
QLVQSGAEVK KPGASVKVSC KASGYTFTGS WMNWVRQAPG
QGLEWIGRIY PGDGETNYNG KFKDRVTITA DKSTSTAYME
LSSLRSEDTA VYYCARIYGN NVYFDVWGQG TTVTVSSGGC
GGGKVAALKE KVAALKEKVA ALKEKVAALK E

In SEQ ID NO:222, amino acid residues 1-110 correspond to the amino acid sequence of the VL Domain of CD3 mAb 2 (SEQ ID NO:104), residues 111-118 correspond to the intervening spacer peptide GGGSGGGG (Linker 1) (SEQ ID NO:33), residues 119-237 correspond to the amino acid sequence of the VH Domain of gpA33 mAb 1 (SEQ ID NO:186), residues 238-243 correspond to the linker GGCGGG (SEQ ID NO:34), and residues 244-271 are a K-coil Domain (SEQ ID NO:40).

A polynucleotide that encodes SEQ ID NO:222 is (SEQ ID NO:223):

caggctgtgg tgactcagga gccttcactg accgtgtccc
caggcggaac tgtgaccctg acatgcagat ccagcacagg
cgcagtgacc acatctaact acgccaattg ggtgcagcag
aagccaggac aggcaccaag gggcctgatc gggggtacaa
acaaaagggc tccctggacc cctgcacggt tttctggaag
tctgctgggc ggaaaggccg ctctgactat taccggggca
caggccgagg acgaagccga ttactattgt gctctgtggt
atagcaatct gtgggtgttc gggggtggca caaaactgac
tgtgctggga gggggtggat ccggcggagg tggacaggtc
cagctggtcc agagcggggc cgaagtcaaa aaacccggag
caagcgtgaa ggtctcctgc aaagcatcag gctatacatt
tacaggcagc tggatgaact gggtgaggca ggctccagga
cagggactgg agtggatcgg gcgcatctac cctggagacg
gcgaaactaa ctataatgga aagttcaaag accgagtgac
catcacagcc gataagtcta ctagtaccgc ctacatggag
ctgagctccc tgcggtctga agataccgcc gtctactatt
gcgctagaat ttacggaaac aatgtctatt ttgacgtgtg
ggggcaggga acaactgtga ctgtctcctc cggaggatgt
ggcggtggaa aagtggccgc actgaaggag aaagttgctg
ctttgaaaga gaaggtcgcc gcacttaagg aaaaggtcgc
agccctgaaa gag

The amino acid sequence of the third polypeptide chain of gpA33 mAb 1×CD3 mAb 2×DR5 mAb 2 is (SEQ ID NO:224):

KVQLQQSGAE LVKPGASVKL SCKASGYTFT EYILHWVKQK
SGQGLEWIGW FYPGNNNIKY NEKFKDKATL TADKSSSTVY
MELSRLTSED SAVYFCARHE QGPGYFDYWG QGTTLTVSSA
STKGPSVFPL APSSKSTSGG TAALGCLVKD YFPEPVTVSW
NSGALTSGVH TFPAVLQSSG LYSLSSVVTV PSSSLGTQTY
ICNVNHKPSN TKVDKRVEPK SCDKTHTCPP CPAPEAAGGP
SVFLFPPKPK DTLMISRTPE VTCVVVDVSH EDPEVKFNWY
VDGVEVHNAK TKPREEQYNS TYRVVSVLTV LHQDWLNGKE
YKCKVSNKAL PAPIEKTISK AKGQPREPQV YTLPPSREEM
TKNQVSLSCA VKGFYPSDIA VEWESNGQPE NNYKTTPPVL
DSDGSFFLVS KLTVDKSRWQ QGNVFSCSVM HEALHNRYTQ
KSLSLSPGK

In SEQ ID NO:224, amino acid residues 1-119 correspond to the amino acid sequence of the VH Domain of DR5 mAb 2 (SEQ ID NO:18), residues 120-217 correspond to a modified CH1 Domain (SEQ ID NO:208), residues 218-232 correspond to a linker (SEQ ID NO:209), and residues 233-449 correspond to the “hole-bearing” CH2-CH3 Domain (SEQ ID NO:53).

A polynucleotide that encodes SEQ ID NO:224 is (SEQ ID NO:225):

aaggtccagc tgcagcagtc tggagctgaa ctggtgaaac
ccggggcatc agtgaagctg tcctgcaagg cttctgggta
caccttcact gagtatattt tacactgggt aaagcagaag
tctggacagg gtcttgagtg gattgggtgg ttttatcctg
gaaataataa tataaagtac aatgagaaat tcaaggacaa
ggccacactg actgcggaca aatcctccag cacagtctat
atggaactta gtagattgac atctgaagac tctgcggtct
atttctgtgc aagacacgaa caaggaccag gttactttga
ctactggggc caaggcacca ctctcacagt ctcctccgcc
tccaccaagg gcccatcggt cttccccctg gcaccctcct
ccaagagcac ctctgggggc acagcggccc tgggctgcct
ggtcaaggac tacttccccg aaccggtgac ggtgtcgtgg
aactcaggcg ccctgaccag cggcgtgcac accttcccgg
ctgtcctaca gtcctcagga ctctactccc tcagcagcgt
ggtgaccgtg ccctccagca gcttgggcac ccagacctac
atctgcaacg tgaatcacaa gcccagcaac accaaggtgg
acaagagagt tgagcccaaa tcttgtgaca aaactcacac
atgcccaccg tgcccagcac ctgaagccgc ggggggaccg
tcagtcttcc tcttcccccc aaaacccaag gacaccctca
tgatctcccg gacccctgag gtcacatgcg tggtggtgga
cgtgagccac gaagaccctg aggtcaagtt caactggtac
gtggacggcg tggaggtgca taatgccaag acaaagccgc
gggaggagca gtacaacagc acgtaccgtg tggtcagcgt
cctcaccgtc ctgcaccagg actggctgaa tggcaaggag
tacaagtgca aggtctccaa caaagccctc ccagccccca
tcgagaaaac catctccaaa gccaaagggc agccccgaga
accacaggtg tacaccctgc ccccatcccg ggaggagatg
accaagaacc aggtcagcct gagttgcgca gtcaaaggct
tctatcccag cgacatcgcc gtggagtggg agagcaatgg
gcagccggag aacaactaca agaccacgcc tcccgtgctg
gactccgacg gctccttctt cctcgtcagc aagctcaccg
tggacaagag caggtggcag caggggaacg tcttctcatg
ctccgtgatg catgaggctc tgcacaaccg ctacacgcag
aagagcctct ccctgtctcc gggtaaa

The amino acid sequence of the fourth polypeptide chain of gpA33 mAb 1×CD3 mAb 2×DR5 mAb 2 is (SEQ ID NO:226):

DIVMTQSHKF MSTSVGDRVS ITCKASQDVN TAVAWYQQKP
GQSPKLLIYW ASTRHTGVPD RFTGSGSGTD YTLTIKSVQA
EDLTLYYCQQ HYITPWTFGG GTKLEIKRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC

In SEQ ID NO:226, amino acid residues 1-107 correspond to the amino acid sequence of the VL Domain of DR5 mAb 2 (SEQ ID NO:13), and residues 108-214 correspond to the CL Kappa Domain (SEQ ID NO:210).

A polynucleotide that encodes SEQ ID NO:226 is (SEQ ID NO:227):

gacattgtga tgacccagtc tcacaaattc atgtccactt
cagtaggaga cagggtcagc atcacctgca aggccagtca
ggatgtgaat actgctgtag cctggtatca acaaaaacca
gggcaatctc ctaaactact gatttactgg gcatccaccc
ggcacactgg agtccctgat cgcttcacag gcagtggatc
tgggacagat tatacactca ccatcaaaag tgtgcaggct
gaagacctga cactttatta ctgtcagcaa cactatatca
ctccgtggac gttcggtgga ggcaccaagc tggaaatcaa
acgtacggtg gctgcaccat cggtcttcat cttcccgcca
tctgatgagc agttgaaatc tggaactgcc tctgttgtgt
gcctgctgaa taacttctat cccagagagg ccaaagtaca
gtggaaggtg gataacgccc tccaatcggg taactcccag
gagagtgtca cagagcagga cagcaaggac agcacctaca
gcctcagcag caccctgacg ctgagcaaag cagactacga
gaaacacaaa gtctacgcct gcgaagtcac ccatcagggc
ctgagctcgc ccgtcacaaa gagcttcaac aggggagagt gt

### H. Epha2 mAB 1×CD3 mAB 2×DR5 mAB 1

A further exemplary Tri-Specific Binding Molecule composed of four polypeptide chains was constructed. The Tri-Specific Binding Molecule comprises the VL and VH domains of EphA2 mAb 1, the VL and VH domains of antibody CD3 mAb 2 and the VL and VH domains of DR5 mAb 1, and was accordingly designated “EphA2 mAb 1×CD3 mAb 2×DR5 mAb 1.” The amino acid sequence of the first polypeptide chain of this Tri-Specific Binding Molecule is (SEQ ID NO:228):

DIQMTQTTSS LSASLGDRIT ISCRASQDIS NYLNWYQQKP
DGTVKLLIYY TSRLHSGVPS RFSGSGSGTD YSLTISNLEQ
EDIATYFCQQ GYTLYTFGGG TKLEIKGGGS GGGGEVQLVE
SGGGLVQPGG SLRLSCAASG FTFSTYAMNW VRQAPGKGLE
WVGRIRSKYN NYATYYADSV KGRFTISRDD SKNSLYLQMN
SLKTEDTAVY YCVRHGNFGN SYVSWFAYWG QGTLVTVSSG
GCGGGEVAAL EKEVAALEKE VAALEKEVAA LEKGGGDKTH
TCPPCPAPEA AGGPSVFLFP PKPKDTLMIS RTPEVTCVVV
DVSHEDPEVK FNWYVDGVEV HNAKTKPREE QYNSTYRVVS
VLTVLHQDWL NGKEYKCKVS NKALPAPIEK TISKAKGQPR
EPQVYTLPPS REEMTKNQVS LWCLVKGFYP SDIAVEWESN
GQPENNYKTT PPVLDSDGSF FLYSKLTVDK SRWQQGNVFS
CSVMHEALHN HYTQKSLSLS PGK

In SEQ ID NO:228, amino acid residues 1-106 correspond to the amino acid sequence of the VL Domain of EphA2 mAb 1 (SEQ ID NO:153), residues 107-114 correspond to the intervening spacer peptide GGGSGGGG (Linker 1) (SEQ ID NO:33), residues 115-239 correspond to the amino acid sequence of the VH Domain of CD3 mAb 2 having the D65G substitution (SEQ ID NO:112), residues 240-245 correspond to the GGCGGG linker (SEQ ID NO:34), residues 246-273 correspond to an E-coil Domain (SEQ ID NO:39), residues 274-276 are the linker GGG, residues 277-286 are the linker DKTHTCPPCP (SEQ ID NO:48), and residues 287-503 are the “knob-bearing” CH2-CH3 Domain (SEQ ID NO:52).

A polynucleotide that encodes SEQ ID NO:228 is (SEQ ID NO:229):

gatatccaga tgacacagac tacatcctcc ctgtctgcct
ctctgggaga cagaatcacc atcagttgca gggcaagtca
ggacattagc aattatttaa actggtatca gcagaaacca
gatggaactg ttaaactcct gatctactac acatcaagat
tacactcagg agtcccatca aggttcagtg gcagtgggtc
tggaacagat tattctctca ccattagcaa cctggagcaa
gaagatattg ccacttactt ttgccaacag ggttatacgc
tgtacacgtt cggagggggg accaagctgg aaataaaagg
tggaggatcc ggcggcggag gcgaggtgca gctggtggag
tctgggggag gcttggtcca gcctggaggg tccctgagac
tctcctgtgc agcctctgga ttcaccttca gcacatacgc
tatgaattgg gtccgccagg ctccagggaa ggggctggag
tgggttggaa ggatcaggtc caagtacaac aattatgcaa
cctactatgc cgactctgtg aagggtagat tcaccatctc
aagagatgat tcaaagaact cactgtatct gcaaatgaac
agcctgaaaa ccgaggacac ggccgtgtat tactgtgtga
gacacggtaa cttcggcaat tcttacgtgt cttggtttgc
ttattgggga caggggacac tggtgactgt gtcttccgga
ggatgtggcg gtggagaagt ggccgcactg gagaaagagg
ttgctgcttt ggagaaggag gtcgctgcac ttgaaaagga
ggtcgcagcc ctggagaaag gcggcgggga caaaactcac
acatgcccac cgtgcccagc acctgaagcc gcggggggac
cgtcagtctt cctcttcccc ccaaaaccca aggacaccct
catgatctcc cggacccctg aggtcacatg cgtggtggtg
gacgtgagcc acgaagaccc tgaggtcaag ttcaactggt
acgtggacgg cgtggaggtg cataatgcca agacaaagcc
gcgggaggag cagtacaaca gcacgtaccg tgtggtcagc
gtcctcaccg tcctgcacca ggactggctg aatggcaagg
agtacaagtg caaggtctcc aacaaagccc tcccagcccc
catcgagaaa accatctcca aagccaaagg gcagccccga
gaaccacagg tgtacaccct gcccccatcc cgggaggaga
tgaccaagaa ccaggtcagc ctgtggtgcc tggtcaaagg
cttctatccc agcgacatcg ccgtggagtg ggagagcaat
gggcagccgg agaacaacta caagaccacg cctcccgtgc
tggactccga cggctccttc ttcctctaca gcaagctcac
cgtggacaag agcaggtggc agcaggggaa cgtcttctca
tgctccgtga tgcatgaggc tctgcacaac cactacacgc
agaagagcct ctccctgtct ccgggtaa

The amino acid sequence of the second polypeptide chain of EphA2 mAb 1×CD3 mAb 2×DR5 mAb 1 is (SEQ ID NO:230):

QAVVTQEPSL TVSPGGTVTL TCRSSTGAVT TSNYANWVQQ
KPGQAPRGLI GGINKRAPWT PARFSGSLLG GKAALTITGA
QAEDEADYYC ALWYSNLWVF GGGTKLTVLG GGGSGGGGQV
QLKESGPGLV APSQSLSITC TVSGFSLSRY SVHWVRQPPG
KGLEWLGMIW GGGSTDYNSA LKSRLSISKD NSKSQVFLKM
NSLQTDDTAM YYCARKHGNY YTMDYWGQGT SVTVSSGGCG
GGKVAALKEK VAALKEKVAA LKEKVAALKE

In SEQ ID NO:230, amino acid residues 1-110 correspond to the amino acid sequence of the VL Domain of CD3 mAb 2 (SEQ ID NO:104), residues 111-118 correspond to the intervening spacer peptide GGGSGGGG (Linker 1) (SEQ ID NO:33), residues 119-236 correspond to the amino acid sequence of the VH Domain of EphA2 mAb 1 (SEQ ID NO:158), residues 237-242 correspond to the linker GGCGGG (SEQ ID NO:34), and residues 243-270 are a K-coil Domain (SEQ ID NO:40).

A polynucleotide that encodes SEQ ID NO:230 is (SEQ ID NO:231):

caggctgtgg tgactcagga gccttcactg accgtgtccc
caggcggaac tgtgaccctg acatgcagat ccagcacagg
cgcagtgacc acatctaact acgccaattg ggtgcagcag
aagccaggac aggcaccaag gggcctgatc gggggtacaa
acaaaagggc tccctggacc cctgcacggt tttctggaag
tctgctgggc ggaaaggccg ctctgactat taccggggca
caggccgagg acgaagccga ttactattgt gctctgtggt
atagcaatct gtgggtgttc gggggtggca caaaactgac
tgtgctggga gggggtggat ccggcggagg tggacaggtg
cagctgaagg agtcaggacc tggcctggtg gcaccctcac
agagcctgtc catcacatgc actgtctctg ggttctcatt
atccagatat agtgtacact gggttcgcca gcctccagga
aagggtctgg agtggctggg aatgatatgg ggtggtggaa
gcacagacta taattcagct ctcaaatcca gactgagtat
cagcaaggac aactccaaga gccaagtttt cttaaaaatg
aacagtctgc aaactgatga cacagccatg tactactgtg
ccagaaaaca tggtaactac tatactatgg actactgggg
tcaaggaacc tcagtcaccg tctcctccgg aggatgtggc
ggtggaaaag tggccgcact gaaggagaaa gttgctgctt
tgaaagagaa ggtcgccgca cttaaggaaa aggtcgcagc
cctgaaagag

The amino acid sequence of the third polypeptide chain of EphA2 mAb 1×CD3 mAb 2×DR5 mAb 1 is (SEQ ID NO:232):

EVKFLESGGG LVQPGGSLKL SCVASGFDFS RYWMSWVRQA
PGKGLEWIGE INPDSNTINY TPSLKDKFII SRDNAKNTLY
LQMTKVRSED TALYYCTRRA YYGNPAWFAY WGQGTLVTVS
SASTKGPSVF PLAPSSKSTS GGTAALGCLV KDYFPEPVTV
SWNSGALTSG VHTFPAVLQS SGLYSLSSVV TVPSSSLGTQ
TYICNVNHKP SNTKVDKRVE PKSCDKTHTC PPCPAPEAAG
GPSVFLFPPK PKDTLMISRT PEVTCVVVDV SHEDPEVKFN
WYVDGVEVHN AKTKPREEQY NSTYRVVSVL TVLHQDWLNG
KEYKCKVSNK ALPAPIEKTI SKAKGQPREP QVYTLPPSRE
EMTKNQVSLS CAVKGFYPSD IAVEWESNGQ PENNYKTTPP
VLDSDGSFFL VSKLTVDKSR WQQGNVFSCS VMHEALHNRY
TQKSLSLSPG K

In SEQ ID NO:232, amino acid residues 1-121 correspond to the amino acid sequence of the VH Domain of DR5 mAb 1 (SEQ ID NO:8), residues 122-219 correspond to a modified CH1 Domain (SEQ ID NO:208), residues 220-234 correspond to a linker (SEQ ID NO:209), and residues 235-451 correspond to the “hole-bearing” CH2-CH3 Domain (SEQ ID NO:53).

A polynucleotide that encodes SEQ ID NO:232 is (SEQ ID NO:233):

gaggtgaagt ttctcgagtc tggaggtggc ctggtgcagc
ctggaggatc cctgaaactc tcctgtgtag cctcaggatt
cgattttagt agatactgga tgagttgggt ccggcaggct
ccagggaaag ggctagaatg gattggagaa attaatccag
atagcaatac gataaactat acgccatctc taaaggataa
attcatcatc tccagagaca acgccaaaaa tacgctgtat
ctgcaaatga ccaaagtgag atctgaggac acagcccttt
attattgtac aagaagggcc tactatggta acccggcctg
gtttgcttac tggggccaag ggactctggt cactgtctct
tccgcctcca ccaagggccc atcggtcttc cccctggcac
cctcctccaa gagcacctct gggggcacag cggccctggg
ctgcctggtc aaggactact tccccgaacc ggtgacggtg
tcgtggaact caggcgccct gaccagcggc gtgcacacct
tcccggctgt cctacagtcc tcaggactct actccctcag
cagcgtggtg accgtgccct ccagcagctt gggcacccag
acctacatct gcaacgtgaa tcacaagccc agcaacacca
aggtggacaa gagagttgag cccaaatctt gtgacaaaac
tcacacatgc ccaccgtgcc cagcacctga agccgcgggg
ggaccgtcag tcttcctctt ccccccaaaa cccaaggaca
ccctcatgat ctcccggacc cctgaggtca catgcgtggt
ggtggacgtg agccacgaag accctgaggt caagttcaac
tggtacgtgg acggcgtgga ggtgcataat gccaagacaa
agccgcggga ggagcagtac aacagcacgt accgtgtggt
cagcgtcctc accgtcctgc accaggactg gctgaatggc
aaggagtaca agtgcaaggt ctccaacaaa gccctcccag
cccccatcga gaaaaccatc tccaaagcca aagggcagcc
ccgagaacca caggtgtaca ccctgccccc atcccgggag
gagatgacca agaaccaggt cagcctgagt tgcgcagtca
aaggcttcta tcccagcgac atcgccgtgg agtgggagag
caatgggcag ccggagaaca actacaagac cacgcctccc
gtgctggact ccgacggctc cttcttcctc gtcagcaagc
tcaccgtgga caagagcagg tggcagcagg ggaacgtctt
ctcatgctcc gtgatgcatg aggctctgca caaccgctac
acgcagaaga gcctctccct gtctccgggt aaa

The amino acid sequence of the fourth polypeptide chain of EphA2 mAb 1×CD3 mAb 2×DR5 mAb 1 is (SEQ ID NO:234):

DIVLTQSPAS LAVSLGQRAT ISCRASKSVS SSGYSYMHWY
QQKPGQPPKV LIFLSSNLDS GVPARFSGSG SGTDFTLNIH
PVEDGDAATY YCQHSRDLPP TFGGGTKLEI KRTVAAPSVF
IFPPSDEQLK SGTASVVCLL NNFYPREAKV QWKVDNALQS
GNSQESVTEQ DSKDSTYSLS STLTLSKADY EKHKVYACEV
THQGLSSPVT KSFNRGEC

In SEQ ID NO:234, amino acid residues 1-111 correspond to the amino acid sequence of the VL Domain of DR5 mAb 1 (SEQ ID NO:3), and residues 112-218 correspond to the CL Kappa Domain (SEQ ID NO:210).

A polynucleotide that encodes SEQ ID NO:234 is (SEQ ID NO:235):

gacattgtgc tgacacagtc tcctgcttcc ttagctgtat
ctctcgggca gagggccacc atctcatgca gggccagcaa
aagtgtcagt tcctctggct atagttatat gcactggtac
caacagaaac caggacagcc acccaaagtc ctcatctttc
tttcatccaa cctagattct ggggtccctg ccaggttcag
tggcagtggg tctgggacag acttcaccct caacatccat
cctgtggagg atggggatgc tgcaacctat tactgtcagc
acagtaggga tcttcctccg acgttcggtg gaggcaccaa
gctggaaatc aaacgtacgg tggctgcacc atcggtcttc
atcttcccgc catctgatga gcagttgaaa tctggaactg
cctctgttgt gtgcctgctg aataacttct atcccagaga
ggccaaagta cagtggaagg tggataacgc cctccaatcg
ggtaactccc aggagagtgt cacagagcag gacagcaagg
acagcaccta cagcctcagc agcaccctga cgctgagcaa
agcagactac gagaaacaca aagtctacgc ctgcgaagtc
acccatcagg gcctgagctc gcccgtcaca aagagcttca
acaggggaga gtgt

### I. EphA2 mAb 2×CD3 mAb 2×DR5 mAb 1

A further exemplary Tri-Specific Binding Molecule composed of four polypeptide chains was constructed. The Tri-Specific Binding Molecule comprises the VL and VH domains of EphA2 mAb 2, the VL and VH domains of antibody CD3 mAb 2 and the VL and VH domains of DR5 mAb 1, and was accordingly designated “EphA2 mAb 2×CD3 mAb 2×DR5 mAb 1.” The amino acid sequence of the first polypeptide chain of this Tri-Specific Binding Molecule is (SEQ ID NO:236):

DVVMTQTPLS LPVSLGDQAS ISCRSSQSLV HSSGNTYLHW
YLQKPGQSPK LLIYKVSNRF SGVPDRFSGS GSGTDFTLKI
SRVEAEDLGV YFCSQSTHVP TFGSGTKLEI KGGGSGGGGE
VQLVESGGGL VQPGGSLRLS CAASGFTFST YAMNWVRQAP
GKGLEWVGRI RSKYNNYATY YADSVKGRFT ISRDDSKNSL
YLQMNSLKTE DTAVYYCVRH GNFGNSYVSW FAYWGQGTLV
TVSSGGCGGG EVAALEKEVA ALEKEVAALE KEVAALEKGG
GDKTHTCPPC PAPEAAGGPS VFLFPPKPKD TLMISRTPEV
TCVVVDVSHE DPEVKFNWYV DGVEVHNAKT KPREEQYNST
YRVVSVLTVL HQDWLNGKEY KCKVSNKALP APIEKTISKA
KGQPREPQVY TLPPSREEMT KNQVSLWCLV KGFYPSDIAV
EWESNGQPEN NYKTTPPVLD SDGSFFLYSK LTVDKSRWQQ
GNVFSCSVMH EALHNHYTQK SLSLSPGK

In SEQ ID NO:236, amino acid residues 1-111 correspond to the amino acid sequence of the VL Domain of EphA2 mAb 2 (SEQ ID NO:163), residues 112-119 correspond to the intervening spacer peptide GGGSGGGG (Linker 1) (SEQ ID NO:33), residues 120-244 correspond to the amino acid sequence of the VH Domain of CD3 mAb 2 having the D65G substitution (SEQ ID NO:112), residues 245-250 correspond to the GGCGGG linker (SEQ ID NO:34), residues 251-278 correspond to an E-coil Domain (SEQ ID NO:39), residues 279-281 are the linker GGG, residues 282-291 are the linker DKTHTCPPCP (SEQ ID NO:48), and residues 292-508 are the “knob-bearing” CH2-CH3 Domain (SEQ ID NO:52).

A polynucleotide that encodes SEQ ID NO:236 is (SEQ ID NO:237):

gatgttgtga tgacccaaac tccactctcc ctgcctgtca
gtcttggaga tcaagcctcc atctcttgca gatctagtca
gagccttgta cacagtagtg gaaacaccta tttacattgg
tacctgcaga agccaggcca gtctccaaag ctcctgatct
acaaagtttc caaccgattt tctggggtcc cagacaggtt
cagtggcagt ggatcaggga cagatttcac actcaagatc
agcagagtgg aggctgagga tctgggagtt tatttctgct
ctcaaagtac acatgttccc acgttcggct cggggacaaa
gttggaaata aaaggtggag gatccggcgg cggaggcgag
gtgcagctgg tggagtctgg gggaggcttg gtccagcctg
gagggtccct gagactctcc tgtgcagcct ctggattcac
cttcagcaca tacgctatga attgggtccg ccaggctcca
gggaaggggc tggagtgggt tggaaggatc aggtccaagt
acaacaatta tgcaacctac tatgccgact ctgtgaaggg
tagattcacc atctcaagag atgattcaaa gaactcactg
tatctgcaaa tgaacagcct gaaaaccgag gacacggccg
tgtattactg tgtgagacac ggtaacttcg gcaattctta
cgtgtcttgg tttgcttatt ggggacaggg gacactggtg
actgtgtctt ccggaggatg tggcggtgga gaagtggccg
cactggagaa agaggttgct gctttggaga aggaggtcgc
tgcacttgaa aaggaggtcg cagccctgga gaaaggcggc
ggggacaaaa ctcacacatg cccaccgtgc ccagcacctg
aagccgcggg gggaccgtca gtcttcctct tccccccaaa
acccaaggac accctcatga tctcccggac ccctgaggtc
acatgcgtgg tggtggacgt gagccacgaa gaccctgagg
tcaagttcaa ctggtacgtg gacggcgtgg aggtgcataa
tgccaagaca aagccgcggg aggagcagta caacagcacg
taccgtgtgg tcagcgtcct caccgtcctg caccaggact
ggctgaatgg caaggagtac aagtgcaagg tctccaacaa
agccctccca gcccccatcg agaaaaccat ctccaaagcc
aaagggcagc cccgagaacc acaggtgtac accctgcccc
catcccggga ggagatgacc aagaaccagg tcagcctgtg
gtgcctggtc aaaggcttct atcccagcga catcgccgtg
gagtgggaga gcaatgggca gccggagaac aactacaaga
ccacgcctcc cgtgctggac tccgacggct ccttcttcct
ctacagcaag ctcaccgtgg acaagagcag gtggcagcag
gggaacgtct tctcatgctc cgtgatgcat gaggctctgc
acaaccacta cacgcagaag agcctctccc tgtctccggg
taaa

The amino acid sequence of the second polypeptide chain of EphA2 mAb 2×CD3 mAb 2×DR5 mAb 1 is (SEQ ID NO:238):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGQI
QLVQSGPELK KPGETVKISC KASGFTFTNY GMNWVKQAPG
KGLKWMGWIN TYIGEPTYAD DFKGRFVFSL ETSASTAYLQ
INNLKNEDMA TYFCARELGP YYFDYWGQGT TLTVSSGGCG
GGKVAALKEK VAALKEKVAA LKEKVAALKE

In SEQ ID NO:238, amino acid residues 1-110 correspond to the amino acid sequence of the VL Domain of CD3 mAb 2 (SEQ ID NO:104), residues 111-118 correspond to the intervening spacer peptide GGGSGGGG (Linker 1) (SEQ ID NO:33), residues 119-236 correspond to the amino acid sequence of the VH Domain of EphA2 mAb 2 (SEQ ID NO:167), residues 237-242 correspond to the linker GGCGGG (SEQ ID NO:34), and residues 243-270 are a K-coil Domain (SEQ ID NO:40).

A polynucleotide that encodes SEQ ID NO:238 is (SEQ ID NO:239):

caggctgtgg tgactcagga gccttcactg accgtgtccc
caggcggaac tgtgaccctg acatgcagat ccagcacagg
cgcagtgacc acatctaact acgccaattg ggtgcagcag
aagccaggac aggcaccaag gggcctgatc gggggtacaa
acaaaagggc tccctggacc cctgcacggt tttctggaag
tctgctgggc ggaaaggccg ctctgactat taccggggca
caggccgagg acgaagccga ttactattgt gctctgtggt
atagcaatct gtgggtgttc gggggtggca caaaactgac
tgtgctggga gggggtggat ccggcggagg tggacagatc
cagttggtgc agtctggacc tgagctgaag aagcctggag
agacagtcaa gatctcctgc aaggcttctg ggtttacctt
cacaaactat ggaatgaact gggtgaagca ggctccagga
aagggtttaa agtggatggg ctggataaac acctatattg
gagagccgac atatgctgat gacttcaagg gacggtttgt
cttctctttg gaaacctctg ccagcactgc ctatttgcag
atcaacaacc tcaaaaatga ggacatggcc acatatttct
gtgcaagaga actgggacca tactactttg actactgggg
ccaaggcacc actctcacag tctcctccgg aggatgtggc
ggtggaaaag tggccgcact gaaggagaaa gttgctgctt
tgaaagagaa ggtcgccgca cttaaggaaa aggtcgcagc
cctgaaagag

The amino acid sequence of the third polypeptide chain of EphA2 mAb 2×CD3 mAb 2×DR5 mAb 1 is (SEQ ID NO:240):

EVKFLESGGG LVQPGGSLKL SCVASGFDFS RYWMSWVRQA
PGKGLEWIGE INPDSNTINY TPSLKDKFII SRDNAKNTLY
LQMTKVRSED TALYYCTRRA YYGNPAWFAY WGQGTLVTVS
SASTKGPSVF PLAPSSKSTS GGTAALGCLV KDYFPEPVTV
SWNSGALTSG VHTFPAVLQS SGLYSLSSVV TVPSSSLGTQ
TYICNVNHKP SNTKVDKRVE PKSCDKTHTC PPCPAPEAAG
GPSVFLFPPK PKDTLMISRT PEVTCVVVDV SHEDPEVKFN
WYVDGVEVHN AKTKPREEQY NSTYRVVSVL TVLHQDWLNG
KEYKCKVSNK ALPAPIEKTI SKAKGQPREP QVYTLPPSRE
EMTKNQVSLS CAVKGFYPSD IAVEWESNGQ PENNYKTTPP
VLDSDGSFFL VSKLTVDKSR WQQGNVFSCS VMHEALHNRY
TQKSLSLSPG K

In SEQ ID NO:240, amino acid residues 1-121 correspond to the amino acid sequence of the VH Domain of DR5 mAb 1 (SEQ ID NO:8), residues 122-219 correspond to a modified CH1 Domain (SEQ ID NO:208), residues 220-234 correspond to a linker (SEQ ID NO:209), and residues 235-451 correspond to the “hole-bearing” CH2-CH3 Domain (SEQ ID NO:53).

A polynucleotide that encodes SEQ ID NO:240 is (SEQ ID NO:241):

gaggtgaagt ttctcgagtc tggaggtggc ctggtgcagc
ctggaggatc cctgaaactc tcctgtgtag cctcaggatt
cgattttagt agatactgga tgagttgggt ccggcaggct
ccagggaaag ggctagaatg gattggagaa attaatccag
atagcaatac gataaactat acgccatctc taaaggataa
attcatcatc tccagagaca acgccaaaaa tacgctgtat
ctgcaaatga ccaaagtgag atctgaggac acagcccttt
attattgtac aagaagggcc tactatggta acccggcctg
gtttgcttac tggggccaag ggactctggt cactgtctct
tccgcctcca ccaagggccc atcggtcttc cccctggcac
cctcctccaa gagcacctct gggggcacag cggccctggg
ctgcctggtc aaggactact tccccgaacc ggtgacggtg
tcgtggaact caggcgccct gaccagcggc gtgcacacct
tcccggctgt cctacagtcc tcaggactct actccctcag
cagcgtggtg accgtgccct ccagcagctt gggcacccag
acctacatct gcaacgtgaa tcacaagccc agcaacacca
aggtggacaa gagagttgag cccaaatctt gtgacaaaac
tcacacatgc ccaccgtgcc cagcacctga agccgcgggg
ggaccgtcag tcttcctctt ccccccaaaa cccaaggaca
ccctcatgat ctcccggacc cctgaggtca catgcgtggt
ggtggacgtg agccacgaag accctgaggt caagttcaac
tggtacgtgg acggcgtgga ggtgcataat gccaagacaa
agccgcggga ggagcagtac aacagcacgt accgtgtggt
cagcgtcctc accgtcctgc accaggactg gctgaatggc
aaggagtaca agtgcaaggt ctccaacaaa gccctcccag
cccccatcga gaaaaccatc tccaaagcca aagggcagcc
ccgagaacca caggtgtaca ccctgccccc atcccgggag
gagatgacca agaaccaggt cagcctgagt tgcgcagtca
aaggcttcta tcccagcgac atcgccgtgg agtgggagag
caatgggcag ccggagaaca actacaagac cacgcctccc
gtgctggact ccgacggctc cttcttcctc gtcagcaagc
tcaccgtgga caagagcagg tggcagcagg ggaacgtctt
ctcatgctcc gtgatgcatg aggctctgca caaccgctac
acgcagaaga gcctctccct gtctccgggt aaa

The amino acid sequence of the fourth polypeptide chain of EphA2 mAb 2×CD3 mAb 2×DR5 mAb 1 is (SEQ ID NO:242):

DIVLTQSPAS LAVSLGQRAT ISCRASKSVS SSGYSYMHWY
QQKPGQPPKV LIFLSSNLDS GVPARFSGSG SGTDFTLNIH
PVEDGDAATY YCQHSRDLPP TFGGGTKLEI KRTVAAPSVF
IFPPSDEQLK SGTASVVCLL NNFYPREAKV QWKVDNALQS
GNSQESVTEQ DSKDSTYSLS STLTLSKADY EKHKVYACEV
THQGLSSPVT KSFNRGEC

In SEQ ID NO:242, amino acid residues 1-111 correspond to the amino acid sequence of the VL Domain of DR5 mAb 1 (SEQ ID NO:3), and residues 112-218 correspond to the CL Kappa Domain (SEQ ID NO:210).

A polynucleotide that encodes SEQ ID NO:242 is (SEQ ID NO:243):

| | | | | |
|---|---|---|---|---|
| gacattgtgc | tgacacagtc | tcctgcttcc | ttagctgtat | ctctcgggca |
| gagggccacc | atctcatgca | gggccagcaa | aagtgtcagt | tcctctggct |
| atagttatat | gcactggtac | caacagaaac | caggacagcc | acccaaagtc |
| ctcatctttc | tttcatccaa | cctagattct | ggggtccctg | ccaggttcag |
| tggcagtggg | tctgggacag | acttcaccct | caacatccat | cctgtggagg |
| atggggatgc | tgcaacctat | tactgtcagc | acagtaggga | tcttcctccg |
| acgttcggtg | gaggcaccaa | gctggaaatc | aaacgtacgg | tggctgcacc |
| atcggtcttc | atcttcccgc | catctgatga | gcagttgaaa | tctggaactg |
| cctctgttgt | gtgcctgctg | aataacttct | atcccagaga | ggccaaagta |
| cagtggaagg | tggataacgc | cctccaatcg | ggtaactccc | aggagagtgt |
| cacagagcag | gacagcaagg | acagcaccta | cagcctcagc | agcaccctga |
| cgctgagcaa | agcagactac | gagaaacaca | aagtctacgc | ctgcgaagtc |
| acccatcagg | gcctgagctc | gcccgtcaca | aagagcttca | acaggggaga |
| gtgt | | | | |

### J. EphA2 mAb 3×CD3 mAb 2×DR5 mAb 1

A further exemplary Tri-Specific Binding Molecule composed of four polypeptide chains was constructed. The Tri-Specific Binding Molecule comprises the VL and VH domains of EphA2 mAb 3, the VL and VH domains of antibody CD3 mAb 2 and the VL and VH domains of DR5 mAb 1, and was accordingly designated “EphA2 mAb 3×CD3 mAb 2×DR5 mAb 1.” The amino acid sequence of the first polypeptide chain of this Tri-Specific Binding Molecule is (SEQ ID NO:244):

| | | | | |
|---|---|---|---|---|
| DIVLTQSHRS | MSTSVGDRVN | ITCKASQDVT | TAVAWYQQKP | GQSPKLLIFW |
| ASTRHAGVPD | RFTGSGSGTD | FTLTISSVQA | GDLALYYCQQ | HYSTPYTFGG |
| GTKLEIKGGG | SGGGGEVQLV | ESGGGLVQPG | GSLRLSCAAS | GFTFSTYAMN |
| WVRQAPGKGL | EWVGRIRSKY | NNYATYYADS | VKGRFTISRD | DSKNSLYLQM |
| NSLKTEDTAV | YYCVRHGNFG | NSYVSWFAYW | GQGTLVTVSS | GGCGGGEVAA |
| LEKEVAALEK | EVAALEKEVA | ALEKGGGDKT | HTCPPCPAPE | AAGGPSVFLF |
| PPKPKDTLMI | SRIPEVICVV | VDVSHEDPEV | KFNWYVDGVE | VHNAKTKPRE |
| EQYNSTYRVV | SVLTVLHQDW | LNGKEYKCKV | SNKALPAPIE | KTISKAKGQP |
| REPQVYTLPP | SREEMTKNQV | SLWCLVKGFY | PSDIAVEWES | NGQPENNYKT |
| TPPVLDSDGS | FFLYSKLTVD | KSRWQQGNVF | SCSVMHEALH | NHYTQKSLSL |
| SPGK | | | | |

In SEQ ID NO:244, amino acid residues 1-107 correspond to the amino acid sequence of the VL Domain of EphA2 mAb 3 (SEQ ID NO:172), residues 108-115 correspond to the intervening spacer peptide GGGSGGGG (Linker 1) (SEQ ID NO:33), residues 116-240 correspond to the amino acid sequence of the VH Domain of CD3 mAb 2 having the D65G substitution (SEQ ID NO:112), residues 241-246 correspond to the GGCGGG linker (SEQ ID NO:34), residues 247-274 correspond to an E-coil Domain (SEQ ID NO:39), residues 275-277 are the linker GGG, residues 278-287 are the linker DKTHTCPPCP (SEQ ID NO:48), and residues 288-504 are the “knob-bearing” CH2-CH3 Domain (SEQ ID NO:52).

A polynucleotide that encodes SEQ ID NO:244 is (SEQ ID NO:245):

| | | | | |
|---|---|---|---|---|
| gacattgtgc | tgacccagtc | tcacagatcc | atgtccacat | cagtaggaga |
| cagggtcaac | atcacctgca | aggccagtca | ggatgtgact | actgctgtag |
| cctggtatca | acaaaaacca | gggcaatctc | ctaaattact | gattttctgg |
| gcatccaccc | ggcacgctgg | agtccctgat | cgcttcacag | gcagtggatc |
| tgggacagat | tttactctca | ccatcagcag | tgtgcaggct | ggagacctgg |
| cactttatta | ctgtcaacaa | cattatagca | caccgtacac | attcggaggg |
| gggaccaagc | tggaaataaa | aggtggagga | tccggcggcg | gaggcgaggt |
| gcagctggtg | gagtctgggg | gaggcttggt | ccagcctgga | gggtccctga |
| gactctcctg | tgcagcctct | ggattcacct | tcagcacata | cgctatgaat |
| tgggtccgcc | aggctccagg | gaaggggctg | gagtgggttg | gaaggatcag |
| gtccaagtac | aacaattatg | caacctacta | tgccgactct | gtgaagggta |
| gattcaccat | ctcaagagat | gattcaaaga | actcactgta | tctgcaaatg |
| aacagcctga | aaaccgagga | cacggccgtg | tattactgtg | tgagacacgg |
| taacttcggc | aattcttacg | tgtcttggtt | tgcttattgg | ggacagggga |
| cactggtgac | tgtgtcttcc | ggaggatgtg | gcggtggaga | agtggccgca |
| ctggagaaag | aggttgctgc | tttggagaag | gaggtcgctg | cacttgaaaa |
| ggaggtcgca | gccctggaga | aaggcggcgg | ggacaaaact | cacacatgcc |
| caccgtgccc | agcacctgaa | gccgcggggg | gaccgtcagt | cttcctcttc |
| cccccaaaac | ccaaggacac | cctcatgatc | tcccggaccc | ctgaggtcac |
| atgcgtggtg | gtggacgtga | gccacgaaga | ccctgaggtc | aagttcaact |
| ggtacgtgga | cggcgtggag | gtgcataatg | ccaagacaaa | gccgcgggag |
| gagcagtaca | acagcacgta | ccgtgtggtc | agcgtcctca | ccgtcctgca |
| ccaggactgg | ctgaatggca | aggagtacaa | gtgcaaggtc | tccaacaaag |
| ccctcccagc | ccccatcgag | aaaaccatct | ccaaagccaa | agggcagccc |
| cgagaaccac | aggtgtacac | cctgccccca | tcccgggagg | agatgaccaa |
| gaaccaggtc | agcctgtggt | gcctggtcaa | aggcttctat | cccagcgaca |
| tcgccgtgga | gtgggagagc | aatgggcagc | cggagaacaa | ctacaagacc |
| acgcctcccg | tgctggactc | cgacggctcc | ttcttcctct | acagcaagct |
| caccgtggac | aagagcaggt | ggcagcaggg | gaacgtcttc | tcatgctccg |
| tgatgcatga | ggctctgcac | aaccactaca | cgcagaagag | cctctccctg |
| tctccgggta | aa | | | |

The amino acid sequence of the second polypeptide chain of EphA2 mAb 3×CD3 mAb 2×DR5 mAb 1 is (SEQ ID NO:246):

| | | | | |
|---|---|---|---|---|
| QAVVTQEPSL | TVSPGGTVTL | TCRSSTGAVT | TSNYANWVQQ | KPGQAPRGLI |
| GGINKRAPWT | PARFSGSLLG | GKAALTITGA | QAEDEADYYC | ALWYSNLWVF |
| GGGTKLTVLG | GGGSGGGGEV | QLVESGGGSV | KPGGSLKLSC | AASGFTFTDH |
| YMYWVRQTPE | KRLEWVATIS | DGGSFTSYPD | SVKGRFTISR | DIAKNNLYLQ |
| MSSLKSEDTA | MYYCTRDESD | RPFPYWGQGT | LVTVSSGGCG | GGKVAALKEK |
| VAALKEKVAA | LKEKVAALKE | | | |

In SEQ ID NO:246, amino acid residues 1-110 correspond to the amino acid sequence of the VL Domain of CD3 mAb 2 (SEQ ID NO:104), residues 111-118 correspond to the intervening spacer peptide GGGSGGGG (Linker 1) (SEQ ID NO:33), residues 119-236 correspond to the amino acid sequence of the VH Domain of EphA2 mAb 3 (SEQ ID NO:177), residues 237-242 correspond to the linker GGCGGG (SEQ ID NO:34), and residues 243-270 are a K-coil Domain (SEQ ID NO:40).

A polynucleotide that encodes SEQ ID NO:246 is (SEQ ID NO:247):

| | | | | |
|---|---|---|---|---|
| caggctgtgg | tgactcagga | gccttcactg | accgtgtccc | caggcggaac |
| tgtgaccctg | acatgcagat | ccagcacagg | cgcagtgacc | acatctaact |
| acgccaattg | ggtgcagcag | aagccaggac | aggcaccaag | gggcctgatc |
| gggggtacaa | acaaaagggc | tccctggacc | cctgcacggt | tttctggaag |
| tctgctgggc | ggaaaggccg | ctctgactat | taccggggca | caggccgagg |
| acgaagccga | ttactattgt | gctctgtggt | atagcaatct | gtgggtgttc |
| gggggtggca | caaaactgac | tgtgctggga | gggggtggat | ccggcggagg |
| tggagaagtg | cagctggtgg | agtctggggg | aggctcagtg | aagcctggag |
| ggtccctgaa | actctcctgt | gcagcctctg | gattcacttt | cactgaccat |
| tacatgtatt | gggttcgcca | gactccggaa | aagaggctgg | agtgggtcgc |
| aaccattagt | gatggcggta | gtttcacctc | ctatccagac | agtgtgaagg |
| ggcgattcac | catctccaga | gacattgcca | agaacaacct | gtacctccaa |
| atgagcagtc | tgaagtctga | ggacacagcc | atgtattact | gtacaagaga |
| tgagagcgat | aggccgtttc | cttactgggg | ccaagggact | ctggtcactg |
| tctcctccgg | aggatgtggc | ggtggaaaag | tggccgcact | gaaggagaaa |
| gttgctgctt | tgaaagagaa | ggtcgccgca | cttaaggaaa | aggtcgcagc |
| cctgaaagag | | | | |

The amino acid sequence of the third polypeptide chain of EphA2 mAb 3×CD3 mAb 2×DR5 mAb 1 is (SEQ ID NO:248):

| | | | | |
|---|---|---|---|---|
| EVKFLESGGG | LVQPGGSLKL | SCVASGFDFS | RYWMSWVRQA | PGKGLEWIGE |
| INPDSNTINY | TPSLKDKFII | SRDNAKNTLY | LQMTKVRSED | TALYYCTRRA |
| YYGNPAWFAY | WGQGTLVTVS | SASTKGPSVF | PLAPSSKSTS | GGTAALGCLV |
| KDYFPEPVTV | SWNSGALTSG | VHTFPAVLQS | SGLYSLSSVV | TVPSSSLGTQ |
| TYICNVNHKP | SNTKVDKRVE | PKSCDKTHTC | PPCPAPEAAG | GPSVFLFPPK |
| PKDTLMISRT | PEVTCVVVDV | SHEDPEVKFN | WYVDGVEVHN | AKTKPREEQY |
| NSTYRVVSVL | TVLHQDWLNG | KEYKCKVSNK | ALPAPIEKTI | SKAKGQPREP |
| QVYTLPPSRE | EMTKNQVSLS | CAVKGFYPSD | IAVEWESNGQ | PENNYKTTPP |
| VLDSDGSFFL | VSKLTVDKSR | WQQGNVFSCS | VMHEALHNRY | TQKSLSLSPG |
| K | | | | |

In SEQ ID NO:248, amino acid residues 1-121 correspond to the amino acid sequence of the VH Domain of DR5 mAb 1 (SEQ ID NO:8), residues 122-219 correspond to a modified CH1 Domain (SEQ ID NO:208), residues 220-234 correspond to a linker (SEQ ID NO:209), and residues 235-451 correspond to the “hole-bearing” CH2-CH3 Domain (SEQ ID NO:53).

A polynucleotide that encodes SEQ ID NO:248 is (SEQ ID NO:249):

| | | | | |
|---|---|---|---|---|
| gaggtgaagt | ttctcgagtc | tggaggtggc | ctggtgcagc | ctggaggatc |
| cctgaaactc | tcctgtgtag | cctcaggatt | cgattttagt | agatactgga |
| tgagttgggt | ccggcaggct | ccagggaaag | ggctagaatg | gattggagaa |
| attaatccag | atagcaatac | gataaactat | acgccatctc | taaaggataa |
| attcatcatc | tccagagaca | acgccaaaaa | tacgctgtat | ctgcaaatga |
| ccaaagtgag | atctgaggac | acagcccttt | attattgtac | aagaagggcc |
| tactatggta | acccggcctg | gtttgcttac | tggggccaag | ggactctggt |
| cactgtctct | tccgcctcca | ccaagggccc | atcggtcttc | cccctggcac |
| cctcctccaa | gagcacctct | gggggcacag | cggccctggg | ctgcctggtc |
| aaggactact | tccccgaacc | ggtgacggtg | tcgtggaact | caggcgccct |
| gaccagcggc | gtgcacacct | tcccggctgt | cctacagtcc | tcaggactct |
| actccctcag | cagcgtggtg | accgtgccct | ccagcagctt | gggcacccag |
| acctacatct | gcaacgtgaa | tcacaagccc | agcaacacca | aggtggacaa |
| gagagttgag | cccaaatctt | gtgacaaaac | tcacacatgc | ccaccgtgcc |
| cagcacctga | agccgcgggg | ggaccgtcag | tcttcctctt | ccccccaaaa |
| cccaaggaca | ccctcatgat | ctcccggacc | cctgaggtca | catgcgtggt |
| ggtggacgtg | agccacgaag | accctgaggt | caagttcaac | tggtacgtgg |
| acggcgtgga | ggtgcataat | gccaagacaa | agccgcggga | ggagcagtac |
| aacagcacgt | accgtgtggt | cagcgtcctc | accgtcctgc | accaggactg |
| gctgaatggc | aaggagtaca | agtgcaaggt | ctccaacaaa | gccctcccag |
| cccccatcga | gaaaaccatc | tccaaagcca | aagggcagcc | ccgagaacca |
| caggtgtaca | ccctgccccc | atcccgggag | gagatgacca | agaaccaggt |
| cagcctgagt | tgcgcagtca | aaggcttcta | tcccagcgac | atcgccgtgg |
| agtgggagag | caatgggcag | ccggagaaca | actacaagac | cacgcctccc |
| gtgctggact | ccgacggctc | cttcttcctc | gtcagcaagc | tcaccgtgga |
| caagagcagg | tggcagcagg | ggaacgtctt | ctcatgctcc | gtgatgcatg |
| aggctctgca | caaccgctac | acgcagaaga | gcctctccct | gtctccgggt |
| aaa | | | | |

The amino acid sequence of the fourth polypeptide chain of EphA2 mAb 3×CD3 mAb 2×DR5 mAb 1 is (SEQ ID NO:250):

| | | | | |
|---|---|---|---|---|
| DIVLTQSPAS | LAVSLGQRAT | ISCRASKSVS | SSGYSYMHWY | QQKPGQPPKV |
| LIFLSSNLDS | GVPARFSGSG | SGTDFTLNIH | PVEDGDAATY | YCQHSRDLPP |
| TFGGGTKLEI | KRTVAAPSVF | IFPPSDEQLK | SGTASVVCLL | NNFYPREAKV |
| QWKVDNALQS | GNSQESVTEQ | DSKDSTYSLS | STLTLSKADY | EKHKVYACEV |
| THQGLSSPVT | KSFNRGEC | | | |

In SEQ ID NO:250, amino acid residues 1-111 correspond to the amino acid sequence of the VL Domain of DR5 mAb 1 (SEQ ID NO:3), and residues 112-218 correspond to the CL Kappa Domain (SEQ ID NO:210).

A polynucleotide that encodes SEQ ID NO:250 is (SEQ ID NO:251):

| | | | | |
|---|---|---|---|---|
| gacattgtgc | tgacacagtc | tcctgcttcc | ttagctgtat | ctctcgggca |
| gagggccacc | atctcatgca | gggccagcaa | aagtgtcagt | tcctctggct |
| atagttatat | gcactggtac | caacagaaac | caggacagcc | acccaaagtc |
| ctcatctttc | tttcatccaa | cctagattct | ggggtccctg | ccaggttcag |
| tggcagtggg | tctgggacag | acttcaccct | caacatccat | cctgtggagg |
| atggggatgc | tgcaacctat | tactgtcagc | acagtaggga | tcttcctccg |
| acgttcggtg | gaggcaccaa | gctggaaatc | aaacgtacgg | tggctgcacc |
| atcggtcttc | atcttcccgc | catctgatga | gcagttgaaa | tctggaactg |
| cctctgttgt | gtgcctgctg | aataacttct | atcccagaga | ggccaaagta |
| cagtggaagg | tggataacgc | cctccaatcg | ggtaactccc | aggagagtgt |
| cacagagcag | gacagcaagg | acagcaccta | cagcctcagc | agcaccctga |
| cgctgagcaa | agcagactac | gagaaacaca | aagtctacgc | ctgcgaagtc |
| acccatcagg | gcctgagctc | gcccgtcaca | aagagcttca | acaggggaga |
| gtgt | | | | |

Although the exemplary Tri-Specific Binding Molecules described above comprise three Light Chain (VL) CDRs and three Heavy Chain (VH) CDRs for each binding domain, it will be recognized that the invention also includes Tri-Specific Binding Molecules that possess:
(1) at least one of the CDRs of the VL Domain of any such binding domain;
(2) at least two of the CDRs of the VL Domain of any such binding domain;
(3) the three CDRs of the VL Domain of any such binding domain; (4) at least one of the CDRs of the VH Domain of any such binding domain;
(5) at least two of the CDRs of the VH Domain of any such binding domain;
(6) the three CDRs of the VH Domain of any such binding domain;
(7) at least one of the CDRs of the VL Domain of any such binding domain and at least one of the CDRs of the VH Domain of that binding domain;
(8) at least two of the CDRs of the VL Domain of any such binding domain and at least two of the CDRs of the VH Domain of that binding domain;
(9) the three CDRs of the VL Domain of any such binding domain and the three CDRs of the VH Domain of that binding domain;
(10) the VL Domain of any such binding domain;
(11) the VH Domain of any such binding domain; or
(12) the VL and VH Domains of any such binding domain.

### K. gpA33 mAb 1×CD3 mAb 2×EphA2 mAb 1

A Tri-Specific Binding Molecule composed of four polypeptide chains was constructed that comprises the VL and VH domains of gpA33 mAb 1, the VL and VH domains of antibody CD3 mAb 2 and the VL and VH domains of EphA2 mAb 1. The Tri-Specific Binding Molecule was accordingly designated “gpA33 mAb 1×CD3 mAb 2×EphA2 mAb 1.” The amino acid sequence of the first polypeptide chain of this Tri-Specific Binding Molecule is (SEQ ID NO:252):

| | | | | |
|---|---|---|---|---|
| DIQLTQSPSF | LSASVGDRVT | ITCSARSSIS | FMYWYQQKPG | KAPKLLIYDT |
| SNLASGVPSR | FSGSGSGTEF | TLTISSLEAE | DAATYYCQQW | SSYPLTFGQG |
| TKLEIKGGGS | GGGGEVQLVE | SGGGLVQPGG | SLRLSCAASG | FTFSTYAMNW |
| VRQAPGKGLE | WVGRIRSKYN | NYATYYADSV | KGRFTISRDD | SKNSLYLQMN |
| SLKTEDTAVY | YCVRHGNFGN | SYVSWFAYWG | QGTLVTVSSG | GCGGGEVAAL |
| EKEVAALEKE | VAALEKEVAA | LEKGGGDKTH | TCPPCPAPEA | AGGPSVFLFP |
| PKPKDTLMIS | RTPEVTCVVV | DVSHEDPEVK | FNWYVDGVEV | HNAKTKPREE |
| QYNSTYRVVS | VLTVLHQDWL | NGKEYKCKVS | NKALPAPIEK | TISKAKGQPR |
| EPQVYTLPPS | REEMTKNQVS | LWCLVKGFYP | SDIAVEWESN | GQPENNYKTT |
| PPVLDSDGSF | FLYSKLTVDK | SRWQQGNVFS | CSVMHEALHN | HYTQKSLSLS |
| PGK | | | | |

In SEQ ID NO:252, amino acid residues 1-106 correspond to the amino acid sequence of the VL Domain of gpA33 mAb 1 (SEQ ID NO:181), residues 107-114 correspond to the intervening spacer peptide GGGSGGGG (Linker 1) (SEQ ID NO:33), residues 115-239 correspond to the amino acid sequence of the VH Domain of CD3 mAb 2 having the D65G substitution (SEQ ID NO:112), residues 240-245 correspond to the GGCGGG linker (SEQ ID NO:34), residues 246-273 correspond to an E-coil Domain (SEQ ID NO:39), residues 274-276 are the linker GGG, residues 277-286 are the linker DKTHTCPPCP (SEQ ID NO:48), and residues 287-503 are the “knob-bearing” CH2-CH3 Domain (SEQ ID NO:52).

A polynucleotide that encodes SEQ ID NO:252 is (SEQ ID NO:253):

| | | | | |
|---|---|---|---|---|
| gacattcagc | tgactcagtc | cccctctttt | ctgtccgcat | ccgtcggaga |
| tcgagtgact | attacttgct | ctgctaggtc | ctcaatcagc | ttcatgtact |
| ggtatcagca | gaagcccggc | aaagcaccta | agctgctgat | ctacgacaca |
| agcaacctgg | cctccggggt | gccatctcgg | ttctctggca | gtgggtcagg |
| aactgagttt | accctgacaa | ttagctccct | ggaggctgaa | gatgccgcta |
| cctactattg | ccagcagtgg | agcagctatc | ctctgacctt | cggacagggg |
| actaaactgg | aaatcaaggg | tggaggatcc | ggcggcggag | gcgaggtgca |
| gctggtggag | tctgggggag | gcttggtcca | gcctggaggg | tccctgagac |
| tctcctgtgc | agcctctgga | ttcaccttca | gcacatacgc | tatgaattgg |
| gtccgccagg | ctccagggaa | ggggctggag | tgggttggaa | ggatcaggtc |
| caagtacaac | aattatgcaa | cctactatgc | cgactctgtg | aagggtagat |
| tcaccatctc | aagagatgat | tcaaagaact | cactgtatct | gcaaatgaac |
| agcctgaaaa | ccgaggacac | ggccgtgtat | tactgtgtga | gacacggtaa |
| cttcggcaat | tcttacgtgt | cttggtttgc | ttattgggga | caggggacac |
| tggtgactgt | gtcttccgga | ggatgtggcg | gtggagaagt | ggccgcactg |
| gagaaagagg | ttgctgcttt | ggagaaggag | gtcgctgcac | ttgaaaagga |
| ggtcgcagcc | ctggagaaag | gcggcgggga | caaaactcac | acatgcccac |
| cgtgcccagc | acctgaagcc | gcggggggac | cgtcagtctt | cctcttcccc |
| ccaaaaccca | aggacaccct | catgatctcc | cggacccctg | aggtcacatg |
| cgtggtggtg | gacgtgagcc | acgaagaccc | tgaggtcaag | ttcaactggt |
| acgtggacgg | cgtggaggtg | cataatgcca | agacaaagcc | gcgggaggag |
| cagtacaaca | gcacgtaccg | tgtggtcagc | gtcctcaccg | tcctgcacca |
| ggactggctg | aatggcaagg | agtacaagtg | caaggtctcc | aacaaagccc |
| tcccagcccc | catcgagaaa | accatctcca | aagccaaagg | gcagccccga |
| gaaccacagg | tgtacaccct | gcccccatcc | cgggaggaga | tgaccaagaa |
| ccaggtcagc | ctgtggtgcc | tggtcaaagg | cttctatccc | agcgacatcg |
| ccgtggagtg | ggagagcaat | gggcagccgg | agaacaacta | caagaccacg |
| cctcccgtgc | tggactccga | cggctccttc | ttcctctaca | gcaagctcac |
| cgtggacaag | agcaggtggc | agcaggggaa | cgtcttctca | tgctccgtga |
| tgcatgaggc | tctgcacaac | cactacacgc | agaagagcct | ctccctgtct |
| ccgggtaaa | | | | |

The amino acid sequence of the second polypeptide chain of gpA33 mAb 1×CD3 mAb 2×EphA2 mAb 1 is (SEQ ID NO:254):

| | | | | |
|---|---|---|---|---|
| QAVVTQEPSL | TVSPGGTVTL | TCRSSTGAVT | TSNYANWVQQ | KPGQAPRGLI |
| GGTNKRAPWT | PARFSGSLLG | GKAALTITGA | QAEDEADYYC | ALWYSNLWVF |
| GGGTKLTVLG | GGGSGGGGQV | QLVQSGAEVK | KPGASVKVSC | KASGYTFTGS |
| WMNWVRQAPG | QGLEWIGRIY | PGDGETNYNG | KFKDRVTITA | DKSTSTAYME |
| LSSLRSEDTA | VYYCARIYGN | NVYFDVWGQG | TTVTVSSGGC | GGGKVAALKE |
| KVAALKEKVA | ALKEKVAALK | E | | |

In SEQ ID NO:254, amino acid residues 1-110 correspond to the amino acid sequence of the VL Domain of CD3 mAb 2 (SEQ ID NO:104), residues 111-118 correspond to the intervening spacer peptide GGGSGGGG (Linker 1) (SEQ ID NO:33), residues 119-237 correspond to the amino acid sequence of the VH Domain of gpA33 mAb 1 (SEQ ID NO:186), residues 238-243 correspond to the linker GGCGGG (SEQ ID NO:34), and residues 244-271 are a K-coil Domain (SEQ ID NO:40).

A polynucleotide that encodes SEQ ID NO:254 is (SEQ ID NO:255):

| | | | | |
|---|---|---|---|---|
| caggctgtgg | tgactcagga | gccttcactg | accgtgtccc | caggcggaac |
| tgtgaccctg | acatgcagat | ccagcacagg | cgcagtgacc | acatctaact |
| acgccaattg | ggtgcagcag | aagccaggac | aggcaccaag | gggcctgatc |
| gggggtacaa | acaaaagggc | tccctggacc | cctgcacggt | tttctggaag |
| tctgctgggc | ggaaaggccg | ctctgactat | taccggggca | caggccgagg |
| acgaagccga | ttactattgt | gctctgtggt | atagcaatct | gtgggtgttc |
| gggggtggca | caaaactgac | tgtgctggga | gggggtggat | ccggcggagg |
| tggacaggtc | cagctggtcc | agagcggggc | cgaagtcaaa | aaacccggag |
| caagcgtgaa | ggtctcctgc | aaagcatcag | gctatacatt | tacaggcagc |
| tggatgaact | gggtgaggca | ggctccagga | cagggactgg | agtggatcgg |
| gcgcatctac | cctggagacg | gcgaaactaa | ctataatgga | aagttcaaag |
| accgagtgac | catcacagcc | gataagtcta | ctagtaccgc | ctacatggag |
| ctgagctccc | tgcggtctga | agataccgcc | gtctactatt | gcgctagaat |
| ttacggaaac | aatgtctatt | ttgacgtgtg | ggggcaggga | acaactgtga |
| ctgtctcctc | cggaggatgt | ggcggtggaa | aagtggccgc | actgaaggag |
| aaagttgctg | ctttgaaaga | gaaggtcgcc | gcacttaagg | aaaaggtcgc |
| agccctgaaa | gag | | | |

The amino acid sequence of the third polypeptide chain of gpA33 mAb 1×CD3 mAb 2×EphA2 mAb 1 is (SEQ ID NO:256):

| | | | | |
|---|---|---|---|---|
| QVQLKESGPG | LVAPSQSLSI | TCTVSGFSLS | RYSVHWVRQP | PGKGLEWLGM |
| IWGGGSTDYN | SALKSRLSIS | KDNSKSQVFL | KMNSLQTDDT | AMYYCARKHG |
| NYYTMDYWGQ | GTSVTVSSAS | TKGPSVFPLA | PSSKSTSGGT | AALGCLVKDY |
| FPEPVTVSWN | SGALTSGVHT | FPAVLQSSGL | YSLSSVVTVP | SSSLGTQTYI |
| CNVNHKPSNT | KVDKRVEPKS | CDKTHTCPPC | PAPEAAGGPS | VFLFPPKPKD |
| TLMISRTPEV | TCVVVDVSHE | DPEVKFNWYV | DGVEVHNAKT | KPREEQYNST |
| YRVVSVLTVL | HQDWLNGKEY | KCKVSNKALP | APIEKTISKA | KGQPREPQVY |
| TLPPSREEMT | KNQVSLSCAV | KGFYPSDIAV | EWESNGQPEN | NYKTTPPVLD |
| SDGSFFLVSK | LTVDKSRWQQ | GNVFSCSVMH | EALHNRYTQK | SLSLSPGK |

In SEQ ID NO:256, amino acid residues 1-118 correspond to the amino acid sequence of the VH Domain of EphA2 mAb 1 (SEQ ID NO:158), residues 119-216 correspond to a modified CH1 Domain (SEQ ID NO:208), residues 217-231 correspond to a linker (SEQ ID NO:209), and residues 232-448 correspond to the “hole-bearing” CH2-CH3 Domain (SEQ ID NO:53).

A polynucleotide that encodes SEQ ID NO:256 is (SEQ ID NO:257):

| | | | | |
|---|---|---|---|---|
| caggtgcagc | tgaaggagtc | aggacctggc | ctggtggcac | cctcacagag |
| cctgtccatc | acatgcactg | tctctgggtt | ctcattatcc | agatatagtg |
| tacactgggt | tcgccagcct | ccaggaaagg | gtctggagtg | gctgggaatg |
| atatggggtg | gtggaagcac | agactataat | tcagctctca | aatccagact |
| gagtatcagc | aaggacaact | ccaagagcca | agttttctta | aaaatgaaca |
| gtctgcaaac | tgatgacaca | gccatgtact | actgtgccag | aaaacatggt |
| aactactata | ctatggacta | ctggggtcaa | ggaacctcag | tcaccgtctc |
| ctccgcctcc | accaagggcc | catcggtctt | ccccctggca | ccctcctcca |
| agagcacctc | tgggggcaca | gcggccctgg | gctgcctggt | caaggactac |
| ttccccgaac | cggtgacggt | gtcgtggaac | tcaggcgccc | tgaccagcgg |
| cgtgcacacc | ttcccggctg | tcctacagtc | ctcaggactc | tactccctca |
| gcagcgtggt | gaccgtgccc | tccagcagct | tgggcaccca | gacctacatc |
| tgcaacgtga | atcacaagcc | cagcaacacc | aaggtggaca | agagagttga |
| gcccaaatct | tgtgacaaaa | ctcacacatg | cccaccgtgc | ccagcacctg |
| aagccgcggg | gggaccgtca | gtcttcctct | tccccccaaa | acccaaggac |
| accctcatga | tctcccggac | ccctgaggtc | acatgcgtgg | tggtggacgt |
| gagccacgaa | gaccctgagg | tcaagttcaa | ctggtacgtg | gacggcgtgg |
| aggtgcataa | tgccaagaca | aagccgcggg | aggagcagta | caacagcacg |
| taccgtgtgg | tcagcgtcct | caccgtcctg | caccaggact | ggctgaatgg |
| caaggagtac | aagtgcaagg | tctccaacaa | agccctccca | gcccccatcg |
| agaaaaccat | ctccaaagcc | aaagggcagc | cccgagaacc | acaggtgtac |
| accctgcccc | catcccggga | ggagatgacc | aagaaccagg | tcagcctgag |
| ttgcgcagtc | aaaggcttct | atcccagcga | catcgccgtg | gagtgggaga |
| gcaatgggca | gccggagaac | aactacaaga | ccacgcctcc | cgtgctggac |
| tccgacggct | ccttcttcct | cgtcagcaag | ctcaccgtgg | acaagagcag |
| gtggcagcag | gggaacgtct | tctcatgctc | cgtgatgcat | gaggctctgc |
| acaaccgcta | cacgcagaag | agcctctccc | tgtctccggg | taaa |

The amino acid sequence of the fourth polypeptide chain of gpA33 mAb 1×CD3 mAb 2×EphA2 mAb 1 is (SEQ ID NO:258):

DIQMTQTTSS LSASLGDRIT ISCRASQDIS NYLNWYQQKP
DGTVKLLIYY TSRLHSGVPS RFSGSGSGTD YSLTISNLEQ
EDIATYFCQQ GYTLYTFGGG TKLEIKRTVA APSVFIFPPS
DEQLKSGTAS VVCLLNNFYP REAKVQWKVD NALQSGNSQE
SVTEQDSKDS TYSLSSTLTL SKADYEKHKV YACEVTHQGL
SSPVTKSFNR GEC

In SEQ ID NO:258, amino acid residues 1-106 correspond to the amino acid sequence of the VL Domain of EphA2 mAb 1 (SEQ ID NO:153), and residues 107-213 correspond to the CL Kappa Domain (SEQ ID NO:210).

A polynucleotide that encodes SEQ ID NO:258 is (SEQ ID NO:259):

gatatccaga tgacacagac tacatcctcc ctgtctgcct
ctctgggaga cagaatcacc atcagttgca gggcaagtca
ggacattagc aattatttaa actggtatca gcagaaacca
gatggaactg ttaaactcct gatctactac acatcaagat
tacactcagg agtcccatca aggttcagtg gcagtgggtc
tggaacagat tattctctca ccattagcaa cctggagcaa
gaagatattg ccacttactt ttgccaacag ggttatacgc
tgtacacgtt cggagggggg accaagctgg aaataaaacg
tacggtggct gcaccatcgg tcttcatctt cccgccatct
gatgagcagt tgaaatctgg aactgcctct gttgtgtgcc
tgctgaataa cttctatccc agagaggcca aagtacagtg
gaaggtggat aacgccctcc aatcgggtaa ctcccaggag
agtgtcacag agcaggacag caaggacagc acctacagcc
tcagcagcac cctgacgctg agcaaagcag actacgagaa
acacaaagtc tacgcctgcg aagtcaccca tcagggcctg
agctcgcccg tcacaaagag cttcaacagg ggagagtgt

### L. gpA33 mAb 1×CD3 mAb 2×EphA2 mAb 2

A Tri-Specific Binding Molecule composed of four polypeptide chains was constructed that comprises the VL and VH domains of gpA33 mAb 1, the VL and VH domains of antibody CD3 mAb 2 and the VL and VH domains of EphA2 mAb 2. The Tri-Specific Binding Molecule was accordingly designated “gpA33 mAb 1×CD3 mAb 2×EphA2 mAb 2.” The amino acid sequence of the first polypeptide chain of this Tri-Specific Binding Molecule is (SEQ ID NO:260):

DIQLTQSPSF LSASVGDRVT ITCSARSSIS FMYWYQQKPG
KAPKLLIYDT SNLASGVPSR FSGSGSGTEF TLTISSLEAE
DAATYYCQQW SSYPLTFGQG TKLEIKGGGS GGGGEVQLVE
SGGGLVQPGG SLRLSCAASG FTFSTYAMNW VRQAPGKGLE
WVGRIRSKYN NYATYYADSV KGRFTISRDD SKNSLYLQMN
SLKTEDTAVY YCVRHGNFGN SYVSWFAYWG QGTLVTVSSG
GCGGGEVAAL EKEVAALEKE VAALEKEVAA LEKGGGDKTH
TCPPCPAPEA AGGPSVFLFP PKPKDTLMIS RTPEVTCVVV
DVSHEDPEVK FNWYVDGVEV HNAKTKPREE QYNSTYRVVS
VLTVLHQDWL NGKEYKCKVS NKALPAPIEK TISKAKGQPR
EPQVYTLPPS REEMTKNQVS LWCLVKGFYP SDIAVEWESN
GQPENNYKTT PPVLDSDGSF FLYSKLTVDK SRWQQGNVFS
CSVMHEALHN HYTQKSLSLS PGK

In SEQ ID NO:260, amino acid residues 1-106 correspond to the amino acid sequence of the VL Domain of gpA33 mAb 1 (SEQ ID NO:181), residues 107-114 correspond to the intervening spacer peptide GGGSGGGG (Linker 1) (SEQ ID NO:33), residues 115-239 correspond to the amino acid sequence of the VH Domain of CD3 mAb 2 having the D65G substitution (SEQ ID NO:112), residues 240-245 correspond to the GGCGGG linker (SEQ ID NO:34), residues 246-273 correspond to an E-coil Domain (SEQ ID NO:39), residues 274-276 are the linker GGG, residues 277-286 are the linker DKTHTCPPCP (SEQ ID NO:48), and residues 287-503 are the “knob-bearing” CH2-CH3 Domain (SEQ ID NO:52).

A polynucleotide that encodes SEQ ID NO:260 is (SEQ ID NO:261):

gacattcagc tgactcagtc cccctctttt ctgtccgcat
ccgtcggaga tcgagtgact attacttgct ctgctaggtc
ctcaatcagc ttcatgtact ggtatcagca gaagcccggc
aaagcaccta agctgctgat ctacgacaca agcaacctgg
cctccggggt gccatctcgg ttctctggca gtgggtcagg
aactgagttt accctgacaa ttagctccct ggaggctgaa
gatgccgcta cctactattg ccagcagtgg agcagctatc
ctctgacctt cggacagggg actaaactgg aaatcaaggg
tggaggatcc ggcggcggag gcgaggtgca gctggtggag
tctgggggag gcttggtcca gcctggaggg tccctgagac
tctcctgtgc agcctctgga ttcaccttca gcacatacgc
tatgaattgg gtccgccagg ctccagggaa ggggctggag
tgggttggaa ggatcaggtc caagtacaac aattatgcaa
cctactatgc cgactctgtg aagggtagat tcaccatctc
aagagatgat tcaaagaact cactgtatct gcaaatgaac
agcctgaaaa ccgaggacac ggccgtgtat tactgtgtga
gacacggtaa cttcggcaat tcttacgtgt cttggtttgc
ttattgggga caggggacac tggtgactgt gtcttccgga
ggatgtggcg gtggagaagt ggccgcactg gagaaagagg
ttgctgcttt ggagaaggag gtcgctgcac ttgaaaagga
ggtcgcagcc ctggagaaag gcggcgggga caaaactcac
acatgcccac cgtgcccagc acctgaagcc gcggggggac
cgtcagtctt cctcttcccc ccaaaaccca aggacaccct
catgatctcc cggacccctg aggtcacatg cgtggtggtg
gacgtgagcc acgaagaccc tgaggtcaag ttcaactggt
acgtggacgg cgtggaggtg cataatgcca agacaaagcc
gcgggaggag cagtacaaca gcacgtaccg tgtggtcagc
gtcctcaccg tcctgcacca ggactggctg aatggcaagg
agtacaagtg caaggtctcc aacaaagccc tcccagcccc
catcgagaaa accatctcca aagccaaagg gcagccccga
gaaccacagg tgtacaccct gcccccatcc cgggaggaga
tgaccaagaa ccaggtcagc ctgtggtgcc tggtcaaagg
cttctatccc agcgacatcg ccgtggagtg ggagagcaat
gggcagccgg agaacaacta caagaccacg cctcccgtgc
tggactccga cggctccttc ttcctctaca gcaagctcac
cgtggacaag agcaggtggc agcaggggaa cgtcttctca
tgctccgtga tgcatgaggc tctgcacaac cactacacgc
agaagagcct ctccctgtct ccgggtaaa

The amino acid sequence of the second polypeptide chain of gpA33 mAb 1×CD3 mAb 2×EphA2 mAb 2 is (SEQ ID NO:262):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGQV
QLVQSGAEVK KPGASVKVSC KASGYTFTGS WMNWVRQAPG
QGLEWIGRIY PGDGETNYNG KFKDRVTITA DKSTSTAYME
LSSLRSEDTA VYYCARIYGN NVYFDVWGQG TTVTVSSGGC
GGGKVAALKE KVAALKEKVA ALKEKVAALK E

In SEQ ID NO:262, amino acid residues 1-110 correspond to the amino acid sequence of the VL Domain of CD3 mAb 2 (SEQ ID NO:104), residues 111-118 correspond to the intervening spacer peptide GGGSGGGG (Linker 1) (SEQ ID NO:33), residues 119-237 correspond to the amino acid sequence of the VH Domain of gpA33 mAb 1 (SEQ ID NO:186), residues 238-243 correspond to the linker GGCGGG (SEQ ID NO:34), and residues 244-271 are a K-coil Domain (SEQ ID NO:40).

A polynucleotide that encodes SEQ ID NO:262 is (SEQ ID NO:263):

caggctgtgg tgactcagga gccttcactg accgtgtccc
caggcggaac tgtgaccctg acatgcagat ccagcacagg
cgcagtgacc acatctaact acgccaattg ggtgcagcag
aagccaggac aggcaccaag gggcctgatc gggggtacaa
acaaaagggc tccctggacc cctgcacggt tttctggaag
tctgctgggc ggaaaggccg ctctgactat taccggggca
caggccgagg acgaagccga ttactattgt gctctgtggt
atagcaatct gtgggtgttc gggggtggca caaaactgac
tgtgctggga gggggtggat ccggcggagg tggacaggtc
cagctggtcc agagcggggc cgaagtcaaa aaacccggag
caagcgtgaa ggtctcctgc aaagcatcag gctatacatt
tacaggcagc tggatgaact gggtgaggca ggctccagga
cagggactgg agtggatcgg gcgcatctac cctggagacg
gcgaaactaa ctataatgga aagttcaaag accgagtgac
catcacagcc gataagtcta ctagtaccgc ctacatggag
ctgagctccc tgcggtctga agataccgcc gtctactatt
gcgctagaat ttacggaaac aatgtctatt ttgacgtgtg
ggggcaggga acaactgtga ctgtctcctc cggaggatgt
ggcggtggaa aagtggccgc actgaaggag aaagttgctg
ctttgaaaga gaaggtcgcc gcacttaagg aaaaggtcgc
agccctgaaa gag

The amino acid sequence of the third polypeptide chain of gpA33 mAb 1×CD3 mAb 2×EphA2 mAb 2 is (SEQ ID NO:264):

QIQLVQSGPE LKKPGETVKI SCKASGFTFT NYGMNWVKQA
PGKGLKWMGW INTYIGEPTY ADDFKGRFVF SLETSASTAY
LQINNLKNED MATYFCAREL GPYYFDYWGQ GTTLTVSSAS
TKGPSVFPLA PSSKSTSGGT AALGCLVKDY FPEPVTVSWN
SGALTSGVHT FPAVLQSSGL YSLSSVVTVP SSSLGTQTYI
CNVNHKPSNT KVDKRVEPKS CDKTHTCPPC PAPEAAGGPS
VFLFPPKPKD TLMISRTPEV TCVVVDVSHE DPEVKFNWYV
DGVEVHNAKT KPREEQYNST YRVVSVLTVL HQDWLNGKEY
KCKVSNKALP APIEKTISKA KGQPREPQVY TLPPSREEMT
KNQVSLSCAV KGFYPSDIAV EWESNGQPEN NYKTTPPVLD
SDGSFFLVSK LTVDKSRWQQ GNVFSCSVMH EALHNRYTQK
SLSLSPGK

In SEQ ID NO:264, amino acid residues 1-118 correspond to the amino acid sequence of the VH Domain of EphA2 mAb 2 (SEQ ID NO:167), residues 119-216 correspond to a modified CH1 Domain (SEQ ID NO:208), residues 217-231 correspond to a linker (SEQ ID NO:209), and residues 232-448 correspond to the “hole-bearing” CH2-CH3 Domain (SEQ ID NO:53).

A polynucleotide that encodes SEQ ID NO:264 is (SEQ ID NO:265):

cagatccagt tggtgcagtc tggacctgag ctgaagaagc
ctggagagac agtcaagatc tcctgcaagg cttctgggtt
taccttcaca aactatggaa tgaactgggt gaagcaggct
ccaggaaagg gtttaaagtg gatgggctgg ataaacacct
atattggaga gccgacatat gctgatgact tcaagggacg
gtttgtcttc tctttggaaa cctctgccag cactgcctat
ttgcagatca acaacctcaa aaatgaggac atggccacat
atttctgtgc aagagaactg ggaccatact actttgacta
ctggggccaa ggcaccactc tcacagtctc ctccgcctcc
accaagggcc catcggtctt ccccctggca ccctcctcca
agagcacctc tgggggcaca gcggccctgg gctgcctggt
caaggactac ttccccgaac cggtgacggt gtcgtggaac
tcaggcgccc tgaccagcgg cgtgcacacc ttcccggctg
tcctacagtc ctcaggactc tactccctca gcagcgtggt
gaccgtgccc tccagcagct tgggcaccca gacctacatc
tgcaacgtga atcacaagcc cagcaacacc aaggtggaca
agagagttga gcccaaatct tgtgacaaaa ctcacacatg
cccaccgtgc ccagcacctg aagccgcggg gggaccgtca
gtcttcctct tccccccaaa acccaaggac accctcatga
tctcccggac ccctgaggtc acatgcgtgg tggtggacgt
gagccacgaa gaccctgagg tcaagttcaa ctggtacgtg
gacggcgtgg aggtgcataa tgccaagaca aagccgcggg
aggagcagta caacagcacg taccgtgtgg tcagcgtcct
caccgtcctg caccaggact ggctgaatgg caaggagtac
aagtgcaagg tctccaacaa agccctccca gcccccatcg
agaaaaccat ctccaaagcc aaagggcagc cccgagaacc
acaggtgtac accctgcccc catcccggga ggagatgacc
aagaaccagg tcagcctgag ttgcgcagtc aaaggcttct
atcccagcga catcgccgtg gagtgggaga gcaatgggca
gccggagaac aactacaaga ccacgcctcc cgtgctggac
tccgacggct ccttcttcct cgtcagcaag ctcaccgtgg
acaagagcag gtggcagcag gggaacgtct tctcatgctc
cgtgatgcat gaggctctgc acaaccgcta cacgcagaag
agcctctccc tgtctccggg taaa

The amino acid sequence of the fourth polypeptide chain of gpA33 mAb 1×CD3 mAb 2×EphA2 mAb 2 is (SEQ ID NO:266):

DVVMTQTPLS LPVSLGDQAS ISCRSSQSLV HSSGNTYLHW
YLQKPGQSPK LLIYKVSNRF SGVPDRFSGS GSGTDFTLKI
SRVEAEDLGV YFCSQSTHVP TFGSGTKLEI KRTVAAPSVF
IFPPSDEQLK SGTASVVCLL NNFYPREAKV QWKVDNALQS
GNSQESVTEQ DSKDSTYSLS STLTLSKADY EKHKVYACEV
THQGLSSPVT KSFNRGEC

In SEQ ID NO:266, amino acid residues 1-111 correspond to the amino acid sequence of the VL Domain of EphA2 mAb 1 (SEQ ID NO:163), and residues 112-218 correspond to the CL Kappa Domain (SEQ ID NO:210).

A polynucleotide that encodes SEQ ID NO:266 is (SEQ ID NO:267):

gatgttgtga tgacccaaac tccactctcc ctgcctgtca
gtcttggaga tcaagcctcc atctcttgca gatctagtca
gagccttgta cacagtagtg gaaacaccta tttacattgg
tacctgcaga agccaggcca gtctccaaag ctcctgatct
acaaagtttc caaccgattt tctggggtcc cagacaggtt
cagtggcagt ggatcaggga cagatttcac actcaagatc
agcagagtgg aggctgagga tctgggagtt tatttctgct
ctcaaagtac acatgttccc acgttcggct cggggacaaa
gttggaaata aaacgtacgg tggctgcacc atcggtcttc
atcttcccgc catctgatga gcagttgaaa tctggaactg
cctctgttgt gtgcctgctg aataacttct atcccagaga
ggccaaagta cagtggaagg tggataacgc cctccaatcg
ggtaactccc aggagagtgt cacagagcag gacagcaagg
acagcaccta cagcctcagc agcaccctga cgctgagcaa
agcagactac gagaaacaca aagtctacgc ctgcgaagtc
acccatcagg gcctgagctc gcccgtcaca aagagcttca
acaggggaga gtgt

### M. gpA33 mAb 1×CD3 mAb 2×EphA2 mAb 3

A Tri-Specific Binding Molecule composed of four polypeptide chains was constructed that comprises the VL and VH domains of gpA33 mAb 1, the VL and VH domains of antibody CD3 mAb 2 and the VL and VH domains of EphA2 mAb 3. The Tri-Specific Binding Molecule was accordingly designated “gpA33 mAb 1×CD3 mAb 2×EphA2 mAb 3.” The amino acid sequence of the first polypeptide chain of this Tri-Specific Binding Molecule is (SEQ ID NO:268):

DIQLTQSPSF LSASVGDRVT ITCSARSSIS FMYWYQQKPG
KAPKLLIYDT SNLASGVPSR FSGSGSGTEF TLTISSLEAE
DAATYYCQQW SSYPLTFGQG TKLEIKGGGS GGGGEVQLVE
SGGGLVQPGG SLRLSCAASG FTFSTYAMNW VRQAPGKGLE
WVGRIRSKYN NYATYYADSV KGRFTISRDD SKNSLYLQMN
SLKTEDTAVY YCVRHGNFGN SYVSWFAYWG QGTLVTVSSG
GCGGGEVAAL EKEVAALEKE VAALEKEVAA LEKGGGDKTH
TCPPCPAPEA AGGPSVFLFP PKPKDTLMIS RTPEVTCVVV
DVSHEDPEVK FNWYVDGVEV HNAKTKPREE QYNSTYRVVS
VLTVLHQDWL NGKEYKCKVS NKALPAPIEK TISKAKGQPR
EPQVYTLPPS REEMTKNQVS LWCLVKGFYP SDIAVEWESN
GQPENNYKTT PPVLDSDGSF FLYSKLTVDK SRWQQGNVFS
CSVMHEALHN HYTQKSLSLS PGK

In SEQ ID NO:268, amino acid residues 1-106 correspond to the amino acid sequence of the VL Domain of gpA33 mAb 1 (SEQ ID NO:181), residues 107-114 correspond to the intervening spacer peptide GGGSGGGG (Linker 1) (SEQ ID NO:33), residues 115-239 correspond to the amino acid sequence of the VH Domain of CD3 mAb 2 having the D65G substitution (SEQ ID NO:112), residues 240-245 correspond to the GGCGGG linker (SEQ ID NO:34), residues 246-273 correspond to an E-coil Domain (SEQ ID NO:39), residues 274-276 are the linker GGG, residues 277-286 are the linker DKTHTCPPCP (SEQ ID NO:48), and residues 287-503 are the “knob-bearing” CH2-CH3 Domain (SEQ ID NO:52).

A polynucleotide that encodes SEQ ID NO:268 is (SEQ ID NO:269):

gacattcagc tgactcagtc cccctctttt ctgtccgcat
ccgtcggaga tcgagtgact attacttgct ctgctaggtc
ctcaatcagc ttcatgtact ggtatcagca gaagcccggc
aaagcaccta agctgctgat ctacgacaca agcaacctgg
cctccggggt gccatctcgg ttctctggca gtgggtcagg
aactgagttt accctgacaa ttagctccct ggaggctgaa
gatgccgcta cctactattg ccagcagtgg agcagctatc
ctctgacctt cggacagggg actaaactgg aaatcaaggg
tggaggatcc ggcggcggag gcgaggtgca gctggtggag
tctgggggag gcttggtcca gcctggaggg tccctgagac
tctcctgtgc agcctctgga ttcaccttca gcacatacgc
tatgaattgg gtccgccagg ctccagggaa ggggctggag
tgggttggaa ggatcaggtc caagtacaac aattatgcaa
cctactatgc cgactctgtg aagggtagat tcaccatctc
aagagatgat tcaaagaact cactgtatct gcaaatgaac
agcctgaaaa ccgaggacac ggccgtgtat tactgtgtga
gacacggtaa cttcggcaat tcttacgtgt cttggtttgc
ttattgggga caggggacac tggtgactgt gtcttccgga
ggatgtggcg gtggagaagt ggccgcactg gagaaagagg
ttgctgcttt ggagaaggag gtcgctgcac ttgaaaagga
ggtcgcagcc ctggagaaag gcggcgggga caaaactcac
acatgcccac cgtgcccagc acctgaagcc gcggggggac
cgtcagtctt cctcttcccc ccaaaaccca aggacaccct
catgatctcc cggacccctg aggtcacatg cgtggtggtg
gacgtgagcc acgaagaccc tgaggtcaag ttcaactggt
acgtggacgg cgtggaggtg cataatgcca agacaaagcc
gcgggaggag cagtacaaca gcacgtaccg tgtggtcagc
gtcctcaccg tcctgcacca ggactggctg aatggcaagg
agtacaagtg caaggtctcc aacaaagccc tcccagcccc
catcgagaaa accatctcca aagccaaagg gcagccccga
gaaccacagg tgtacaccct gcccccatcc cgggaggaga
tgaccaagaa ccaggtcagc ctgtggtgcc tggtcaaagg
cttctatccc agcgacatcg ccgtggagtg ggagagcaat
gggcagccgg agaacaacta caagaccacg cctcccgtgc
tggactccga cggctccttc ttcctctaca gcaagctcac
cgtggacaag agcaggtggc agcaggggaa cgtcttctca
tgctccgtga tgcatgaggc tctgcacaac cactacacgc
agaagagcct ctccctgtct ccgggtaaa

The amino acid sequence of the second polypeptide chain of gpA33 mAb 1×CD3 mAb 2×EphA2 mAb 3 is (SEQ ID NO:270):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGQV
QLVQSGAEVK KPGASVKVSC KASGYTFTGS WMNWVRQAPG
QGLEWIGRIY PGDGETNYNG KFKDRVTITA DKSTSTAYME
LSSLRSEDTA VYYCARIYGN NVYFDVWGQG TTVTVSSGGC
GGGKVAALKE KVAALKEKVA ALKEKVAALK E

In SEQ ID NO:270, amino acid residues 1-110 correspond to the amino acid sequence of the VL Domain of CD3 mAb 2 (SEQ ID NO:104), residues 111-118 correspond to the intervening spacer peptide GGGSGGGG (Linker 1) (SEQ ID NO:33), residues 119-237 correspond to the amino acid sequence of the VH Domain of gpA33 mAb 1 (SEQ ID NO:186), residues 238-243 correspond to the linker GGCGGG (SEQ ID NO:34), and residues 244-271 are a K-coil Domain (SEQ ID NO:40).

A polynucleotide that encodes SEQ ID NO:270 is (SEQ ID NO:271):

caggctgtgg tgactcagga gccttcactg accgtgtccc
caggcggaac tgtgaccctg acatgcagat ccagcacagg
cgcagtgacc acatctaact acgccaattg ggtgcagcag
aagccaggac aggcaccaag gggcctgatc gggggtacaa
acaaaagggc tccctggacc cctgcacggt tttctggaag
tctgctgggc ggaaaggccg ctctgactat taccggggca
caggccgagg acgaagccga ttactattgt gctctgtggt
atagcaatct gtgggtgttc gggggtggca caaaactgac
tgtgctggga gggggtggat ccggcggagg tggacaggtc
cagctggtcc agagcggggc cgaagtcaaa aaacccggag
caagcgtgaa ggtctcctgc aaagcatcag gctatacatt
tacaggcagc tggatgaact gggtgaggca ggctccagga
cagggactgg agtggatcgg gcgcatctac cctggagacg
gcgaaactaa ctataatgga aagttcaaag accgagtgac
catcacagcc gataagtcta ctagtaccgc ctacatggag
ctgagctccc tgcggtctga agataccgcc gtctactatt
gcgctagaat ttacggaaac aatgtctatt ttgacgtgtg
ggggcaggga acaactgtga ctgtctcctc cggaggatgt
ggcggtggaa aagtggccgc actgaaggag aaagttgctg
ctttgaaaga gaaggtcgcc gcacttaagg aaaaggtcgc
agccctgaaa gag

The amino acid sequence of the third polypeptide chain of gpA33 mAb 1×CD3 mAb 2×EphA2 mAb 3 is (SEQ ID NO:272):

EVQLVESGGG SVKPGGSLKL SCAASGFTFT DHYMYWVRQT
PEKRLEWVAT ISDGGSFTSY PDSVKGRFTI SRDIAKNNLY
LQMSSLKSED TAMYYCTRDE SDRPFPYWGQ GTLVTVSSAS
TKGPSVFPLA PSSKSTSGGT AALGCLVKDY FPEPVTVSWN
SGALTSGVHT FPAVLQSSGL YSLSSVVTVP SSSLGTQTYI
CNVNHKPSNT KVDKRVEPKS CDKTHTCPPC PAPEAAGGPS
VFLFPPKPKD TLMISRTPEV TCVVVDVSHE DPEVKFNWYV
DGVEVHNAKT KPREEQYNST YRVVSVLTVL HQDWLNGKEY
KCKVSNKALP APIEKTISKA KGQPREPQVY TLPPSREEMT
KNQVSLSCAV KGFYPSDIAV EWESNGQPEN NYKTTPPVLD
SDGSFFLVSK LTVDKSRWQQ GNVFSCSVMH EALHNRYTQK
SLSLSPGK

In SEQ ID NO:272, amino acid residues 1-118 correspond to the amino acid sequence of the VH Domain of EphA2 mAb 3 (SEQ ID NO:177), residues 119-216 correspond to a modified CH1 Domain (SEQ ID NO:208), residues 217-231 correspond to a linker (SEQ ID NO:209), and residues 232-448 correspond to the “hole-bearing” CH2-CH3 Domain (SEQ ID NO:53).

A polynucleotide that encodes SEQ ID NO:272 is (SEQ ID NO:273):

gaagtgcagc tggtggagtc tgggggaggc tcagtgaagc
ctggagggtc cctgaaactc tcctgtgcag cctctggatt
cactttcact gaccattaca tgtattgggt tcgccagact
ccggaaaaga ggctggagtg ggtcgcaacc attagtgatg
gcggtagttt cacctcctat ccagacagtg tgaaggggcg
attcaccatc tccagagaca ttgccaagaa caacctgtac
ctccaaatga gcagtctgaa gtctgaggac acagccatgt
attactgtac aagagatgag agcgataggc cgtttcctta
ctggggccaa gggactctgg tcactgtctc ctccgcctcc
accaagggcc catcggtctt ccccctggca ccctcctcca
agagcacctc tgggggcaca gcggccctgg gctgcctggt
caaggactac ttccccgaac cggtgacggt gtcgtggaac
tcaggcgccc tgaccagcgg cgtgcacacc ttcccggctg
tcctacagtc ctcaggactc tactccctca gcagcgtggt
gaccgtgccc tccagcagct tgggcaccca gacctacatc
tgcaacgtga atcacaagcc cagcaacacc aaggtggaca
agagagttga gcccaaatct tgtgacaaaa ctcacacatg
cccaccgtgc ccagcacctg aagccgcggg gggaccgtca
gtcttcctct tccccccaaa acccaaggac accctcatga
tctcccggac ccctgaggtc acatgcgtgg tggtggacgt
gagccacgaa gaccctgagg tcaagttcaa ctggtacgtg
gacggcgtgg aggtgcataa tgccaagaca aagccgcggg
aggagcagta caacagcacg taccgtgtgg tcagcgtcct
caccgtcctg caccaggact ggctgaatgg caaggagtac
aagtgcaagg tctccaacaa agccctccca gcccccatcg
agaaaaccat ctccaaagcc aaagggcagc cccgagaacc
acaggtgtac accctgcccc catcccggga ggagatgacc
aagaaccagg tcagcctgag ttgcgcagtc aaaggcttct
atcccagcga catcgccgtg gagtgggaga gcaatgggca
gccggagaac aactacaaga ccacgcctcc cgtgctggac
tccgacggct ccttcttcct cgtcagcaag ctcaccgtgg
acaagagcag gtggcagcag gggaacgtct tctcatgctc
cgtgatgcat gaggctctgc acaaccgcta cacgcagaag
agcctctccc tgtctccggg taaa

The amino acid sequence of the fourth polypeptide chain of gpA33 mAb 1×CD3 mAb 2×EphA2 mAb 3 is (SEQ ID NO:274):

DIVLTQSHRS MSTSVGDRVN ITCKASQDVT TAVAWYQQKP
GQSPKLLIFW ASTRHAGVPD RFTGSGSGTD FTLTISSVQA
GDLALYYCQQ HYSTPYTFGG GTKLEIKRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC

In SEQ ID NO:274, amino acid residues 1-107 correspond to the amino acid sequence of the VL Domain of EphA2 mAb 3 (SEQ ID NO:172), and residues 108-214 correspond to the CL Kappa Domain (SEQ ID NO:210).

A polynucleotide that encodes SEQ ID NO:274 is (SEQ ID NO:275):

gacattgtgc tgacccagtc tcacagatcc atgtccacat
cagtaggaga cagggtcaac atcacctgca aggccagtca
ggatgtgact actgctgtag cctggtatca acaaaaacca
gggcaatctc ctaaattact gattttctgg gcatccaccc
ggcacgctgg agtccctgat cgcttcacag gcagtggatc
tgggacagat tttactctca ccatcagcag tgtgcaggct
ggagacctgg cactttatta ctgtcaacaa cattatagca
caccgtacac attcggaggg gggaccaagc tggaaataaa
acgtacggtg gctgcaccat cggtcttcat cttcccgcca
tctgatgagc agttgaaatc tggaactgcc tctgttgtgt
gcctgctgaa taacttctat cccagagagg ccaaagtaca
gtggaaggtg gataacgccc tccaatcggg taactcccag
gagagtgtca cagagcagga cagcaaggac agcacctaca
gcctcagcag caccctgacg ctgagcaaag cagactacga
gaaacacaaa gtctacgcct gcgaagtcac ccatcagggc
ctgagctcgc ccgtcacaaa gagcttcaac aggggagagt
gt

### N. EphA2 mAb 1×CD3 mAb 2×gpA33 mAb 1

An alternative EphA2/CD3/gpA33 Tri-Specific Binding Molecule was constructed. The molecule was composed of four polypeptide chains and comprises the VL and VH domains of EphA2 mAb 1, the VL and VH domains of antibody CD3 mAb 2 and the VL and VH domains of gpA33 mAb 1. The molecule was designated “EphA2 mAb 1×CD3 mAb 2×gpA33 mAb 1.” The amino acid sequence of the first polypetide chain of this Tri-Specific Binding Molecule is (SEQ ID NO:276):

DIQMTQTTSS LSASLGDRIT ISCRASQDIS NYLNWYQQKP
DGTVKLLIYY TSRLHSGVPS RFSGSGSGTD YSLTISNLEQ
EDIATYFCQQ GYTLYTFGGG TKLEIKGGGS GGGGEVQLVE
SGGGLVQPGG SLRLSCAASG FTFSTYAMNW VRQAPGKGLE
WVGRIRSKYN NYATYYADSV KGRFTISRDD SKNSLYLQMN
SLKTEDTAVY YCVRHGNFGN SYVSWFAYWG QGTLVTVSSG
GCGGGEVAAL EKEVAALEKE VAALEKEVAA LEKGGGDKTH
TCPPCPAPEA AGGPSVFLFP PKPKDTLMIS RTPEVTCVVV
DVSHEDPEVK FNWYVDGVEV HNAKTKPREE QYNSTYRVVS
VLTVLHQDWL NGKEYKCKVS NKALPAPIEK TISKAKGQPR
EPQVYTLPPS REEMTKNQVS LWCLVKGFYP SDIAVEWESN
GQPENNYKTT PPVLDSDGSF FLYSKLTVDK SRWQQGNVFS
CSVMHEALHN HYTQKSLSLS PGK

In SEQ ID NO:276, amino acid residues 1-106 correspond to the amino acid sequence of the VL Domain of EphA2 mAb 1 (SEQ ID NO:153), residues 107-114 correspond to the intervening spacer peptide GGGSGGGG (Linker 1) (SEQ ID NO:33), residues 115-239 correspond to the amino acid sequence of the VH Domain of CD3 mAb 2 having the D65G substitution (SEQ ID NO:112), residues 240-245 correspond to the GGCGGG linker (SEQ ID NO:34), residues 246-273 correspond to an E-coil Domain (SEQ ID NO:39), residues 274-276 are the linker GGG, residues 277-286 are the linker DKTHTCPPCP (SEQ ID NO:48), and residues 287-503 are the “knob-bearing” CH2-CH3 Domain (SEQ ID NO:52).

A polynucleotide that encodes SEQ ID NO:276 is (SEQ ID NO:277):

gatatccaga tgacacagac tacatcctcc ctgtctgcct
ctctgggaga cagaatcacc atcagttgca gggcaagtca
ggacattagc aattatttaa actggtatca gcagaaacca
gatggaactg ttaaactcct gatctactac acatcaagat
tacactcagg agtcccatca aggttcagtg gcagtgggtc
tggaacagat tattctctca ccattagcaa cctggagcaa
gaagatattg ccacttactt ttgccaacag ggttatacgc
tgtacacgtt cggagggggg accaagctgg aaataaaagg
tggaggatcc ggcggcggag gcgaggtgca gctggtggag
tctgggggag gcttggtcca gcctggaggg tccctgagac
tctcctgtgc agcctctgga ttcaccttca gcacatacgc
tatgaattgg gtccgccagg ctccagggaa ggggctggag
tgggttggaa ggatcaggtc caagtacaac aattatgcaa
cctactatgc cgactctgtg aagggtagat tcaccatctc
aagagatgat tcaaagaact cactgtatct gcaaatgaac
agcctgaaaa ccgaggacac ggccgtgtat tactgtgtga
gacacggtaa cttcggcaat tcttacgtgt cttggtttgc
ttattgggga caggggacac tggtgactgt gtcttccgga
ggatgtggcg gtggagaagt ggccgcactg gagaaagagg
ttgctgcttt ggagaaggag gtcgctgcac ttgaaaagga
ggtcgcagcc ctggagaaag gcggcgggga caaaactcac
acatgcccac cgtgcccagc acctgaagcc gcggggggac
cgtcagtctt cctcttcccc ccaaaaccca aggacaccct
catgatctcc cggacccctg aggtcacatg cgtggtggtg
gacgtgagcc acgaagaccc tgaggtcaag ttcaactggt
acgtggacgg cgtggaggtg cataatgcca agacaaagcc
gcgggaggag cagtacaaca gcacgtaccg tgtggtcagc
gtcctcaccg tcctgcacca ggactggctg aatggcaagg
agtacaagtg caaggtctcc aacaaagccc tcccagcccc
catcgagaaa accatctcca aagccaaagg gcagccccga
gaaccacagg tgtacaccct gcccccatcc cgggaggaga
tgaccaagaa ccaggtcagc ctgtggtgcc tggtcaaagg
cttctatccc agcgacatcg ccgtggagtg ggagagcaat
gggcagccgg agaacaacta caagaccacg cctcccgtgc
tggactccga cggctccttc ttcctctaca gcaagctcac
cgtggacaag agcaggtggc agcaggggaa cgtcttctca
tgctccgtga tgcatgaggc tctgcacaac cactacacgc
agaagagcct ctccctgtct ccgggtaaa

The amino acid sequence of the second polypeptide chain of EphA2 mAb 1×CD3 mAb 2×gpA33 mAb 1 is (SEQ ID NO:278):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGQV
QLKESGPGLV APSQSLSITC TVSGFSLSRY SVHWVRQPPG
KGLEWLGMIW GGGSTDYNSA LKSRLSISKD NSKSQVFLKM
NSLQTDDTAM YYCARKHGNY YTMDYWGQGT SVTVSSGGCG
GGKVAALKEK VAALKEKVAA LKEKVAALKE

In SEQ ID NO:278, amino acid residues 1-110 correspond to the amino acid sequence of the VL Domain of CD3 mAb 2 (SEQ ID NO:104), residues 111-118 correspond to the intervening spacer peptide GGGSGGGG (Linker 1) (SEQ ID NO:33), residues 119-236 correspond to the amino acid sequence of the VH Domain of EphA2 mAb 1 (SEQ ID NO:158), residues 237-242 correspond to the linker GGCGGG (SEQ ID NO:34), and residues 243-270 are a K-coil Domain (SEQ ID NO:40).

A polynucleotide that encodes SEQ ID NO:278 is (SEQ ID NO:279):

caggctgtgg tgactcagga gccttcactg accgtgtccc
caggcggaac tgtgaccctg acatgcagat ccagcacagg
cgcagtgacc acatctaact acgccaattg ggtgcagcag
aagccaggac aggcaccaag gggcctgatc gggggtacaa
acaaaagggc tccctggacc cctgcacggt tttctggaag
tctgctgggc ggaaaggccg ctctgactat taccggggca
caggccgagg acgaagccga ttactattgt gctctgtggt
atagcaatct gtgggtgttc gggggtggca caaaactgac
tgtgctggga gggggtggat ccggcggagg tggacaggtg
cagctgaagg agtcaggacc tggcctggtg gcaccctcac
agagcctgtc catcacatgc actgtctctg ggttctcatt
atccagatat agtgtacact gggttcgcca gcctccagga
aagggtctgg agtggctggg aatgatatgg ggtggtggaa
gcacagacta taattcagct ctcaaatcca gactgagtat
cagcaaggac aactccaaga gccaagtttt cttaaaaatg
aacagtctgc aaactgatga cacagccatg tactactgtg
ccagaaaaca tggtaactac tatactatgg actactgggg
tcaaggaacc tcagtcaccg tctcctccgg aggatgtggc
ggtggaaaag tggccgcact gaaggagaaa gttgctgctt
tgaaagagaa ggtcgccgca cttaaggaaa aggtcgcagc
cctgaaagag

The amino acid sequence of the third polypeptide chain of EphA2 mAb 1×CD3 mAb 2×gpA33 mAb 1 is (SEQ ID NO:280):

QVQLVQSGAE VKKPGASVKV SCKASGYTFT GSWMNWVRQA
PGQGLEWIGR IYPGDGETNY NGKFKDRVTI TADKSTSTAY
MELSSLRSED TAVYYCARIY GNNVYFDVWG QGTTVTVSSA
STKGPSVFPL APSSKSTSGG TAALGCLVKD YFPEPVTVSW
NSGALTSGVH TFPAVLQSSG LYSLSSVVTV PSSSLGTQTY
ICNVNHKPSN TKVDKRVEPK SCDKTHTCPP CPAPEAAGGP
SVFLFPPKPK DTLMISRTPE VTCVVVDVSH EDPEVKFNWY
VDGVEVHNAK TKPREEQYNS TYRVVSVLTV LHQDWLNGKE
YKCKVSNKAL PAPIEKTISK AKGQPREPQV YTLPPSREEM
TKNQVSLSCA VKGFYPSDIA VEWESNGQPE NNYKTTPPVL
DSDGSFFLVS KLTVDKSRWQ QGNVFSCSVM HEALHNRYTQ
KSLSLSPGK

In SEQ ID NO:280, amino acid residues 1-119 correspond to the amino acid sequence of the VH Domain of gpA33 mAb 1 (SEQ ID NO:186), residues 120-217 correspond to a modified CH1 Domain (SEQ ID NO:208), residues 218-232 correspond to a linker (SEQ ID NO:209), and residues 233-449 correspond to the “hole-bearing” CH2-CH3 Domain (SEQ ID NO:53).

A polynucleotide that encodes SEQ ID NO:280 is (SEQ ID NO:281):

caggtccagc tggtccagag cggggccgaa gtcaaaaaac
ccggagcaag cgtgaaggtc tcctgcaaag catcaggcta
tacatttaca ggcagctgga tgaactgggt gaggcaggct
ccaggacagg gactggagtg gatcgggcgc atctaccctg
gagacggcga aactaactat aatggaaagt tcaaagaccg
agtgaccatc acagccgata agtctactag taccgcctac
atggagctga gctccctgcg gtctgaagat accgccgtct
actattgcgc tagaatttac ggaaacaatg tctattttga
cgtgtggggg cagggaacaa ctgtgactgt ctcctccgcc
tccaccaagg gcccatcggt cttccccctg gcaccctcct
ccaagagcac ctctgggggc acagcggccc tgggctgcct
ggtcaaggac tacttccccg aaccggtgac ggtgtcgtgg
aactcaggcg ccctgaccag cggcgtgcac accttcccgg
ctgtcctaca gtcctcagga ctctactccc tcagcagcgt
ggtgaccgtg ccctccagca gcttgggcac ccagacctac
atctgcaacg tgaatcacaa gcccagcaac accaaggtgg
acaagagagt tgagcccaaa tcttgtgaca aaactcacac
atgcccaccg tgcccagcac ctgaagccgc ggggggaccg
tcagtcttcc tcttcccccc aaaacccaag gacaccctca
tgatctcccg gacccctgag gtcacatgcg tggtggtgga
cgtgagccac gaagaccctg aggtcaagtt caactggtac
gtggacggcg tggaggtgca taatgccaag acaaagccgc
gggaggagca gtacaacagc acgtaccgtg tggtcagcgt
cctcaccgtc ctgcaccagg actggctgaa tggcaaggag
tacaagtgca aggtctccaa caaagccctc ccagccccca
tcgagaaaac catctccaaa gccaaagggc agccccgaga
accacaggtg tacaccctgc ccccatcccg ggaggagatg
accaagaacc aggtcagcct gagttgcgca gtcaaaggct
tctatcccag cgacatcgcc gtggagtggg agagcaatgg
gcagccggag aacaactaca agaccacgcc tcccgtgctg
gactccgacg gctccttctt cctcgtcagc aagctcaccg
tggacaagag caggtggcag caggggaacg tcttctcatg
ctccgtgatg catgaggctc tgcacaaccg ctacacgcag
aagagcctct ccctgtctcc gggtaaa

The amino acid sequence of the fourth polypeptide chain of EphA2 mAb 1×CD3 mAb 2×gpA33 mAb 1 is (SEQ ID NO:282):

DIQLTQSPSF LSASVGDRVT ITCSARSSIS FMYWYQQKPG
KAPKLLIYDT SNLASGVPSR FSGSGSGTEF TLTISSLEAE
DAATYYCQQW SSYPLTFGQG TKLEIKRTVA APSVFIFPPS
DEQLKSGTAS VVCLLNNFYP REAKVQWKVD NALQSGNSQE
SVTEQDSKDS TYSLSSTLTL SKADYEKHKV YACEVTHQGL
SSPVTKSFNR GEC

In SEQ ID NO:282, amino acid residues 1-106 correspond to the amino acid sequence of the VL Domain of gpA33 mAb 1 (SEQ ID NO:181), and residues 107-213 correspond to the CL Kappa Domain (SEQ ID NO:210).

A polynucleotide that encodes SEQ ID NO:282 is (SEQ ID NO:283):

gacattcagc tgactcagtc cccctctttt ctgtccgcat
ccgtcggaga tcgagtgact attacttgct ctgctaggtc
ctcaatcagc ttcatgtact ggtatcagca gaagcccggc
aaagcaccta agctgctgat ctacgacaca agcaacctgg
cctccggggt gccatctcgg ttctctggca gtgggtcagg
aactgagttt accctgacaa ttagctccct ggaggctgaa
gatgccgcta cctactattg ccagcagtgg agcagctatc
ctctgacctt cggacagggg actaaactgg aaatcaagcg
tacggtggct gcaccatcgg tcttcatctt cccgccatct
gatgagcagt tgaaatctgg aactgcctct gttgtgtgcc
tgctgaataa cttctatccc agagaggcca aagtacagtg
gaaggtggat aacgccctcc aatcgggtaa ctcccaggag
agtgtcacag agcaggacag caaggacagc acctacagcc
tcagcagcac cctgacgctg agcaaagcag actacgagaa
acacaaagtc tacgcctgcg aagtcaccca tcagggcctg
agctcgcccg tcacaaagag cttcaacagg ggagagtgt

### O. EphA2 mAb 2×CD3 mAb 2×gpA33 mAb 1

A second alternative EphA2/CD3/gpA33 Tri-Specific Binding Molecule was constructed. The molecule was composed of four polypeptide chains and comprises the VL and VH domains of EphA2 mAb 2, the VL and VH domains of antibody CD3 mAb 2 and the VL and VH domains of gpA33 mAb 1. The molecule was designated “EphA2 mAb 2×CD3 mAb 2×gpA33 mAb 1.” The amino acid sequence of the first polypeptide chain of this Tri-Specific Binding Molecule is (SEQ ID NO:284):

DVVMTQTPLS LPVSLGDQAS ISCRSSQSLV HSSGNTYLHW
YLQKPGQSPK LLIYKVSNRF SGVPDRFSGS GSGTDFTLKI
SRVEAEDLGV YFCSQSTHVP TFGSGTKLEI KGGGSGGGGE
VQLVESGGGL VQPGGSLRLS CAASGFTFST YAMNWVRQAP
GKGLEWVGRI RSKYNNYATY YADSVKGRFT ISRDDSKNSL
YLQMNSLKTE DTAVYYCVRH GNFGNSYVSW FAYWGQGTLV
TVSSGGCGGG EVAALEKEVA ALEKEVAALE KEVAALEKGG
GDKTHTCPPC PAPEAAGGPS VFLFPPKPKD TLMISRTPEV
TCVVVDVSHE DPEVKFNWYV DGVEVHNAKT KPREEQYNST
YRVVSVLTVL HQDWLNGKEY KCKVSNKALP APIEKTISKA
KGQPREPQVY TLPPSREEMT KNQVSLWCLV KGFYPSDIAV
EWESNGQPEN NYKTTPPVLD SDGSFFLYSK LTVDKSRWQQ
GNVFSCSVMH EALHNHYTQK SLSLSPGK

In SEQ ID NO:284, amino acid residues 1-111 correspond to the amino acid sequence of the VL Domain of EphA2 mAb 2 (SEQ ID NO:163), residues 112-119 correspond to the intervening spacer peptide GGGSGGGG (Linker 1) (SEQ ID NO:33), residues 120-244 correspond to the amino acid sequence of the VH Domain of CD3 mAb 2 having the D65G substitution (SEQ ID NO:112), residues 245-250 correspond to the GGCGGG linker (SEQ ID NO:34), residues 251-278 correspond to an E-coil Domain (SEQ ID NO:39), residues 279-281 are the linker GGG, residues 282-291 are the linker DKTHTCPPCP (SEQ ID NO:48), and residues 292-508 are the “knob-bearing” CH2-CH3 Domain (SEQ ID NO:52).

A polynucleotide that encodes SEQ ID NO:284 is (SEQ ID NO:285):

gatgttgtga tgacccaaac tccactctcc ctgcctgtca
gtcttggaga tcaagcctcc atctcttgca gatctagtca
gagccttgta cacagtagtg gaaacaccta tttacattgg
tacctgcaga agccaggcca gtctccaaag ctcctgatct
acaaagtttc caaccgattt tctggggtcc cagacaggtt
cagtggcagt ggatcaggga cagatttcac actcaagatc
agcagagtgg aggctgagga tctgggagtt tatttctgct
ctcaaagtac acatgttccc acgttcggct cggggacaaa
gttggaaata aaaggtggag gatccggcgg cggaggcgag
gtgcagctgg tggagtctgg gggaggcttg gtccagcctg
gagggtccct gagactctcc tgtgcagcct ctggattcac
cttcagcaca tacgctatga attgggtccg ccaggctcca
gggaaggggc tggagtgggt tggaaggatc aggtccaagt
acaacaatta tgcaacctac tatgccgact ctgtgaaggg
tagattcacc atctcaagag atgattcaaa gaactcactg
tatctgcaaa tgaacagcct gaaaaccgag gacacggccg
tgtattactg tgtgagacac ggtaacttcg gcaattctta
cgtgtcttgg tttgcttatt ggggacaggg gacactggtg
actgtgtctt ccggaggatg tggcggtgga gaagtggccg
cactggagaa agaggttgct gctttggaga aggaggtcgc
tgcacttgaa aaggaggtcg cagccctgga gaaaggcggc
ggggacaaaa ctcacacatg cccaccgtgc ccagcacctg
aagccgcggg gggaccgtca gtcttcctct tccccccaaa
acccaaggac accctcatga tctcccggac ccctgaggtc
acatgcgtgg tggtggacgt gagccacgaa gaccctgagg
tcaagttcaa ctggtacgtg gacggcgtgg aggtgcataa
tgccaagaca aagccgcggg aggagcagta caacagcacg
taccgtgtgg tcagcgtcct caccgtcctg caccaggact
ggctgaatgg caaggagtac aagtgcaagg tctccaacaa
agccctccca gcccccatcg agaaaaccat ctccaaagcc
aaagggcagc cccgagaacc acaggtgtac accctgcccc
catcccggga ggagatgacc aagaaccagg tcagcctgtg
gtgcctggtc aaaggcttct atcccagcga catcgccgtg
gagtgggaga gcaatgggca gccggagaac aactacaaga
ccacgcctcc cgtgctggac tccgacggct ccttcttcct
ctacagcaag ctcaccgtgg acaagagcag gtggcagcag
gggaacgtct tctcatgctc cgtgatgcat gaggctctgc
acaaccacta cacgcagaag agcctctccc tgtctccggg
taaa

The amino acid sequence of the second polypeptide chain of EphA2 mAb 2×CD3 mAb 2×gpA33 mAb 1 is (SEQ ID NO:286):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGQI
QLVQSGPELK KPGETVKISC KASGFTFTNY GMNWVKQAPG
KGLKWMGWIN TYIGEPTYAD DFKGRFVFSL ETSASTAYLQ
INNLKNEDMA TYFCARELGP YYFDYWGQGT TLTVSSGGCG
GGKVAALKEK VAALKEKVAA LKEKVAALKE

In SEQ ID NO:286, amino acid residues 1-110 correspond to the amino acid sequence of the VL Domain of CD3 mAb 2 (SEQ ID NO:104), residues 111-118 correspond to the intervening spacer peptide GGGSGGGG (Linker 1) (SEQ ID NO:33), residues 119-236 correspond to the amino acid sequence of the VH Domain of EphA2 mAb 2 (SEQ ID NO:167), residues 237-242 correspond to the linker GGCGGG (SEQ ID NO:34), and residues 243-270 are a K-coil Domain (SEQ ID NO:40).

A polynucleotide that encodes SEQ ID NO:286 is (SEQ ID NO:287):

caggctgtgg tgactcagga gccttcactg accgtgtccc
caggcggaac tgtgaccctg acatgcagat ccagcacagg
cgcagtgacc acatctaact acgccaattg ggtgcagcag
aagccaggac aggcaccaag gggcctgatc gggggtacaa
acaaaagggc tccctggacc cctgcacggt tttctggaag
tctgctgggc ggaaaggccg ctctgactat taccggggca
caggccgagg acgaagccga ttactattgt gctctgtggt
atagcaatct gtgggtgttc gggggtggca caaaactgac
tgtgctggga gggggtggat ccggcggagg tggacagatc
cagttggtgc agtctggacc tgagctgaag aagcctggag
agacagtcaa gatctcctgc aaggcttctg ggtttacctt
cacaaactat ggaatgaact gggtgaagca ggctccagga
aagggtttaa agtggatggg ctggataaac acctatattg
gagagccgac atatgctgat gacttcaagg gacggtttgt
cttctctttg gaaacctctg ccagcactgc ctatttgcag
atcaacaacc tcaaaaatga ggacatggcc acatatttct
gtgcaagaga actgggacca tactactttg actactgggg
ccaaggcacc actctcacag tctcctccgg aggatgtggc
ggtggaaaag tggccgcact gaaggagaaa gttgctgctt
tgaaagagaa ggtcgccgca cttaaggaaa aggtcgcagc
cctgaaagag

The amino acid sequence of the third polypeptide chain of EphA2 mAb 2×CD3 mAb 2×gpA33 mAb 1 is (SEQ ID NO:288):

QVQLVQSGAE VKKPGASVKV SCKASGYTFT GSWMNWVRQA
PGQGLEWIGR IYPGDGETNY NGKFKDRVTI TADKSTSTAY
MELSSLRSED TAVYYCARIY GNNVYFDVWG QGTTVTVSSA
STKGPSVFPL APSSKSTSGG TAALGCLVKD YFPEPVTVSW
NSGALTSGVH TFPAVLQSSG LYSLSSVVTV PSSSLGTQTY
ICNVNHKPSN TKVDKRVEPK SCDKTHTCPP CPAPEAAGGP
SVFLFPPKPK DTLMISRTPE VTCVVVDVSH EDPEVKFNWY
VDGVEVHNAK TKPREEQYNS TYRVVSVLTV LHQDWLNGKE
YKCKVSNKAL PAPIEKTISK AKGQPREPQV YTLPPSREEM
TKNQVSLSCA VKGFYPSDIA VEWESNGQPE NNYKTTPPVL
DSDGSFFLVS KLTVDKSRWQ QGNVFSCSVM HEALHNRYTQ
KSLSLSPGK

In SEQ ID NO:288, amino acid residues 1-119 correspond to the amino acid sequence of the VH Domain of gpA33 mAb 1 (SEQ ID NO:186), residues 120-217 correspond to a modified CH1 Domain (SEQ ID NO:208), residues 218-232 correspond to a linker (SEQ ID NO:209), and residues 233-449 correspond to the “hole-bearing” CH2-CH3 Domain (SEQ ID NO:53).

A polynucleotide that encodes SEQ ID NO:288 is (SEQ ID NO:289):

caggtccagc tggtccagag cggggccgaa gtcaaaaaac
ccggagcaag cgtgaaggtc tcctgcaaag catcaggcta
tacatttaca ggcagctgga tgaactgggt gaggcaggct
ccaggacagg gactggagtg gatcgggcgc atctaccctg
gagacggcga aactaactat aatggaaagt tcaaagaccg
agtgaccatc acagccgata agtctactag taccgcctac
atggagctga gctccctgcg gtctgaagat accgccgtct
actattgcgc tagaatttac ggaaacaatg tctattttga
cgtgtggggg cagggaacaa ctgtgactgt ctcctccgcc
tccaccaagg gcccatcggt cttccccctg gcaccctcct
ccaagagcac ctctgggggc acagcggccc tgggctgcct
ggtcaaggac tacttccccg aaccggtgac ggtgtcgtgg
aactcaggcg ccctgaccag cggcgtgcac accttcccgg
ctgtcctaca gtcctcagga ctctactccc tcagcagcgt
ggtgaccgtg ccctccagca gcttgggcac ccagacctac
atctgcaacg tgaatcacaa gcccagcaac accaaggtgg
acaagagagt tgagcccaaa tcttgtgaca aaactcacac
atgcccaccg tgcccagcac ctgaagccgc ggggggaccg
tcagtcttcc tcttcccccc aaaacccaag gacaccctca
tgatctcccg gacccctgag gtcacatgcg tggtggtgga
cgtgagccac gaagaccctg aggtcaagtt caactggtac
gtggacggcg tggaggtgca taatgccaag acaaagccgc
gggaggagca gtacaacagc acgtaccgtg tggtcagcgt
cctcaccgtc ctgcaccagg actggctgaa tggcaaggag
tacaagtgca aggtctccaa caaagccctc ccagccccca
tcgagaaaac catctccaaa gccaaagggc agccccgaga
accacaggtg tacaccctgc ccccatcccg ggaggagatg
accaagaacc aggtcagcct gagttgcgca gtcaaaggct
tctatcccag cgacatcgcc gtggagtggg agagcaatgg
gcagccggag aacaactaca agaccacgcc tcccgtgctg
gactccgacg gctccttctt cctcgtcagc aagctcaccg
tggacaagag caggtggcag caggggaacg tcttctcatg
ctccgtgatg catgaggctc tgcacaaccg ctacacgcag
aagagcctct ccctgtctcc gggtaaa

The amino acid sequence of the fourth polypeptide chain of EphA2 mAb 2×CD3 mAb 2×gpA33 mAb 1 is (SEQ ID NO:290):

DIQLTQSPSF LSASVGDRVT ITCSARSSIS FMYWYQQKPG
KAPKLLIYDT SNLASGVPSR FSGSGSGTEF TLTISSLEAE
DAATYYCQQW SSYPLTFGQG TKLEIKRTVA APSVFIFPPS
DEQLKSGTAS VVCLLNNFYP REAKVQWKVD NALQSGNSQE
SVTEQDSKDS TYSLSSTLTL SKADYEKHKV YACEVTHQGL
SSPVTKSFNR GEC

In SEQ ID NO:290, amino acid residues 1-106 correspond to the amino acid sequence of the VL Domain of gpA33 mAb 1 (SEQ ID NO:181), and residues 107-213 correspond to the CL Kappa Domain (SEQ ID NO:210).

A polynucleotide that encodes SEQ ID NO:290 is (SEQ ID NO:291):

gacattcagc tgactcagtc cccctctttt ctgtccgcat
ccgtcggaga tcgagtgact attacttgct ctgctaggtc
ctcaatcagc ttcatgtact ggtatcagca gaagcccggc
aaagcaccta agctgctgat ctacgacaca agcaacctgg
cctccggggt gccatctcgg ttctctggca gtgggtcagg
aactgagttt accctgacaa ttagctccct ggaggctgaa
gatgccgcta cctactattg ccagcagtgg agcagctatc
ctctgacctt cggacagggg actaaactgg aaatcaagcg
tacggtggct gcaccatcgg tcttcatctt cccgccatct
gatgagcagt tgaaatctgg aactgcctct gttgtgtgcc
tgctgaataa cttctatccc agagaggcca aagtacagtg
gaaggtggat aacgccctcc aatcgggtaa ctcccaggag
agtgtcacag agcaggacag caaggacagc acctacagcc
tcagcagcac cctgacgctg agcaaagcag actacgagaa
acacaaagtc tacgcctgcg aagtcaccca tcagggcctg
agctcgcccg tcacaaagag cttcaacagg ggagagtgt

### P. EphA2 mAb 3×CD3 mAb 2×gpA33 mAb 1

A third alternative EphA2/CD3/gpA33 Tri-Specific Binding Molecule was constructed. The molecule was composed of four polypeptide chains and comprises the VL and VH domains of EphA2 mAb 3, the VL and VH domains of antibody CD3 mAb 2 and the VL and VH domains of gpA33 mAb 1. The molecule was designated “EphA2 mAb 3×CD3 mAb 2×gpA33 mAb 1.” The amino acid sequence of the first polypeptide chain of this Tri-Specific Binding Molecule is (SEQ ID NO:292):

DIVLTQSHRS MSTSVGDRVN ITCKASQDVT TAVAWYQQKP
GQSPKLLIFW ASTRHAGVPD RFTGSGSGTD FTLTISSVQA
GDLALYYCQQ HYSTPYTFGG GTKLEIKGGG SGGGGEVQLV
ESGGGLVQPG GSLRLSCAAS GFTFSTYAMN WVRQAPGKGL
EWVGRIRSKY NNYATYYADS VKGRFTISRD DSKNSLYLQM
NSLKTEDTAV YYCVRHGNFG NSYVSWFAYW GQGTLVTVSS
GGCGGGEVAA LEKEVAALEK EVAALEKEVA ALEKGGGDKT
HTCPPCPAPE AAGGPSVFLF PPKPKDTLMI SRTPEVTCVV
VDVSHEDPEV KFNWYVDGVE VHNAKTKPRE EQYNSTYRVV
SVLTVLHQDW LNGKEYKCKV SNKALPAPIE KTISKAKGQP
REPQVYTLPP SREEMTKNQV SLWCLVKGFY PSDIAVEWES
NGQPENNYKT TPPVLDSDGS FFLYSKLTVD KSRWQQGNVF
SCSVMHEALH NHYTQKSLSL SPGK

In SEQ ID NO:292, amino acid residues 1-107 correspond to the amino acid sequence of the VL Domain of EphA2 mAb 3 (SEQ ID NO:172), residues 108-115 correspond to the intervening spacer peptide GGGSGGGG (Linker 1) (SEQ ID NO:33), residues 116-240 correspond to the amino acid sequence of the VH Domain of CD3 mAb 2 having the D65G substitution (SEQ ID NO:112), residues 241-246 correspond to the GGCGGG linker (SEQ ID NO:34), residues 247-274 correspond to an E-coil Domain (SEQ ID NO:39), residues 275-277 are the linker GGG, residues 278-287 are the linker DKTHTCPPCP (SEQ ID NO:48), and residues 288-504 are the “knob-bearing” CH2-CH3 Domain (SEQ ID NO:52).

A polynucleotide that encodes SEQ ID NO:292 is (SEQ ID NO:293):

gacattgtgc tgacccagtc tcacagatcc atgtccacat
cagtaggaga cagggtcaac atcacctgca aggccagtca
ggatgtgact actgctgtag cctggtatca acaaaaacca
gggcaatctc ctaaattact gattttctgg gcatccaccc
ggcacgctgg agtccctgat cgcttcacag gcagtggatc
tgggacagat tttactctca ccatcagcag tgtgcaggct
ggagacctgg cactttatta ctgtcaacaa cattatagca
caccgtacac attcggaggg gggaccaagc tggaaataaa
aggtggagga tccggcggcg gaggcgaggt gcagctggtg
gagtctgggg gaggcttggt ccagcctgga gggtccctga
gactctcctg tgcagcctct ggattcacct tcagcacata
cgctatgaat tgggtccgcc aggctccagg gaaggggctg
gagtgggttg gaaggatcag gtccaagtac aacaattatg
caacctacta tgccgactct gtgaagggta gattcaccat
ctcaagagat gattcaaaga actcactgta tctgcaaatg
aacagcctga aaaccgagga cacggccgtg tattactgtg
tgagacacgg taacttcggc aattcttacg tgtcttggtt
tgcttattgg ggacagggga cactggtgac tgtgtcttcc
ggaggatgtg gcggtggaga agtggccgca ctggagaaag
aggttgctgc tttggagaag gaggtcgctg cacttgaaaa
ggaggtcgca gccctggaga aaggcggcgg ggacaaaact
cacacatgcc caccgtgccc agcacctgaa gccgcggggg
gaccgtcagt cttcctcttc cccccaaaac ccaaggacac
cctcatgatc tcccggaccc ctgaggtcac atgcgtggtg
gtggacgtga gccacgaaga ccctgaggtc aagttcaact
ggtacgtgga cggcgtggag gtgcataatg ccaagacaaa
gccgcgggag gagcagtaca acagcacgta ccgtgtggtc
agcgtcctca ccgtcctgca ccaggactgg ctgaatggca
aggagtacaa gtgcaaggtc tccaacaaag ccctcccagc
ccccatcgag aaaaccatct ccaaagccaa agggcagccc
cgagaaccac aggtgtacac cctgccccca tcccgggagg
agatgaccaa gaaccaggtc agcctgtggt gcctggtcaa
aggcttctat cccagcgaca tcgccgtgga gtgggagagc
aatgggcagc cggagaacaa ctacaagacc acgcctcccg
tgctggactc cgacggctcc ttcttcctct acagcaagct
caccgtggac aagagcaggt ggcagcaggg gaacgtcttc
tcatgctccg tgatgcatga ggctctgcac aaccactaca
cgcagaagag cctctccctg tctccgggta aa

The amino acid sequence of the second polypeptide chain of EphA2 mAb 3×CD3 mAb 2×gpA33 mAb 1 is (SEQ ID NO:294):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGEV
QLVESGGGSV KPGGSLKLSC AASGFTFTDH YMYWVRQTPE
KRLEWVATIS DGGSFTSYPD SVKGRFTISR DIAKNNLYLQ
MSSLKSEDTA MYYCTRDESD RPFPYWGQGT LVTVSSGGCG
GGKVAALKEK VAALKEKVAA LKEKVAALKE

In SEQ ID NO:294, amino acid residues 1-110 correspond to the amino acid sequence of the VL Domain of CD3 mAb 3 (SEQ ID NO:104), residues 111-118 correspond to the intervening spacer peptide GGGSGGGG (Linker 1) (SEQ ID NO:33), residues 119-236 correspond to the amino acid sequence of the VH Domain of EphA2 mAb 3 (SEQ ID NO:177), residues 237-242 correspond to the linker GGCGGG (SEQ ID NO:34), and residues 243-270 are a K-coil Domain (SEQ ID NO:40).

A polynucleotide that encodes SEQ ID NO:294 is (SEQ ID NO:295):

caggctgtgg tgactcagga gccttcactg accgtgtccc
caggcggaac tgtgaccctg acatgcagat ccagcacagg
cgcagtgacc acatctaact acgccaattg ggtgcagcag
aagccaggac aggcaccaag gggcctgatc gggggtacaa
acaaaagggc tccctggacc cctgcacggt tttctggaag
tctgctgggc ggaaaggccg ctctgactat taccggggca
caggccgagg acgaagccga ttactattgt gctctgtggt
atagcaatct gtgggtgttc gggggtggca caaaactgac
tgtgctggga gggggtggat ccggcggagg tggagaagtg
cagctggtgg agtctggggg aggctcagtg aagcctggag
ggtccctgaa actctcctgt gcagcctctg gattcacttt
cactgaccat tacatgtatt gggttcgcca gactccggaa
aagaggctgg agtgggtcgc aaccattagt gatggcggta
gtttcacctc ctatccagac agtgtgaagg ggcgattcac
catctccaga gacattgcca agaacaacct gtacctccaa
atgagcagtc tgaagtctga ggacacagcc atgtattact
gtacaagaga tgagagcgat aggccgtttc cttactgggg
ccaagggact ctggtcactg tctcctccgg aggatgtggc
ggtggaaaag tggccgcact gaaggagaaa gttgctgctt
tgaaagagaa ggtcgccgca cttaaggaaa aggtcgcagc
cctgaaagag

The amino acid sequence of the third polypeptide chain of EphA2 mAb 3×CD3 mAb 2×gpA33 mAb 1 is (SEQ ID NO:296):

QVQLVQSGAE VKKPGASVKV SCKASGYTFT GSWMNWVRQA
PGQGLEWIGR IYPGDGETNY NGKFKDRVTI TADKSTSTAY
MELSSLRSED TAVYYCARIY GNNVYFDVWG QGTTVTVSSA
STKGPSVFPL APSSKSTSGG TAALGCLVKD YFPEPVTVSW
NSGALTSGVH TFPAVLQSSG LYSLSSVVTV PSSSLGTQTY
ICNVNHKPSN TKVDKRVEPK SCDKTHTCPP CPAPEAAGGP
SVFLFPPKPK DTLMISRTPE VTCVVVDVSH EDPEVKFNWY
VDGVEVHNAK TKPREEQYNS TYRVVSVLTV LHQDWLNGKE
YKCKVSNKAL PAPIEKTISK AKGQPREPQV YTLPPSREEM
TKNQVSLSCA VKGFYPSDIA VEWESNGQPE NNYKTTPPVL
DSDGSFFLVS KLTVDKSRWQ QGNVFSCSVM HEALHNRYTQ
KSLSLSPGK

In SEQ ID NO:296, amino acid residues 1-119 correspond to the amino acid sequence of the VH Domain of gpA33 mAb 1 (SEQ ID NO:186), residues 120-217 correspond to a modified CH1 Domain (SEQ ID NO:208), residues 218-232 correspond to a linker (SEQ ID NO:209), and residues 233-449 correspond to the “hole-bearing” CH2-CH3 Domain (SEQ ID NO:53).

A polynucleotide that encodes SEQ ID NO:296 is (SEQ ID NO:297):

caggtccagc tggtccagag cggggccgaa gtcaaaaaac
ccggagcaag cgtgaaggtc tcctgcaaag catcaggcta
tacatttaca ggcagctgga tgaactgggt gaggcaggct
ccaggacagg gactggagtg gatcgggcgc atctaccctg
gagacggcga aactaactat aatggaaagt tcaaagaccg
agtgaccatc acagccgata agtctactag taccgcctac
atggagctga gctccctgcg gtctgaagat accgccgtct
actattgcgc tagaatttac ggaaacaatg tctattttga
cgtgtggggg cagggaacaa ctgtgactgt ctcctccgcc
tccaccaagg gcccatcggt cttccccctg gcaccctcct
ccaagagcac ctctgggggc acagcggccc tgggctgcct
ggtcaaggac tacttccccg aaccggtgac ggtgtcgtgg
aactcaggcg ccctgaccag cggcgtgcac accttcccgg
ctgtcctaca gtcctcagga ctctactccc tcagcagcgt
ggtgaccgtg ccctccagca gcttgggcac ccagacctac
atctgcaacg tgaatcacaa gcccagcaac accaaggtgg
acaagagagt tgagcccaaa tcttgtgaca aaactcacac
atgcccaccg tgcccagcac ctgaagccgc ggggggaccg
tcagtcttcc tcttcccccc aaaacccaag gacaccctca
tgatctcccg gacccctgag gtcacatgcg tggtggtgga
cgtgagccac gaagaccctg aggtcaagtt caactggtac
gtggacggcg tggaggtgca taatgccaag acaaagccgc
gggaggagca gtacaacagc acgtaccgtg tggtcagcgt
cctcaccgtc ctgcaccagg actggctgaa tggcaaggag
tacaagtgca aggtctccaa caaagccctc ccagccccca
tcgagaaaac catctccaaa gccaaagggc agccccgaga
accacaggtg tacaccctgc ccccatcccg ggaggagatg
accaagaacc aggtcagcct gagttgcgca gtcaaaggct
tctatcccag cgacatcgcc gtggagtggg agagcaatgg
gcagccggag aacaactaca agaccacgcc tcccgtgctg
gactccgacg gctccttctt cctcgtcagc aagctcaccg
tggacaagag caggtggcag caggggaacg tcttctcatg
ctccgtgatg catgaggctc tgcacaaccg ctacacgcag
aagagcctct ccctgtctcc gggtaaa

The amino acid sequence of the fourth polypeptide chain of EphA2 mAb 3×CD3 mAb 2×gpA33 mAb 1 is (SEQ ID NO:298):

DIQLTQSPSF LSASVGDRVT ITCSARSSIS FMYWYQQKPG
KAPKLLIYDT SNLASGVPSR FSGSGSGTEF TLTISSLEAE
DAATYYCQQW SSYPLTFGQG TKLEIKRTVA APSVFIFPPS
DEQLKSGTAS VVCLLNNFYP REAKVQWKVD NALQSGNSQE
SVTEQDSKDS TYSLSSTLTL SKADYEKHKV YACEVTHQGL
SSPVTKSFNR GEC

In SEQ ID NO:298, amino acid residues 1-106 correspond to the amino acid sequence of the VL Domain of gpA33 mAb 1 (SEQ ID NO:181), and residues 107-213 correspond to the CL Kappa Domain (SEQ ID NO:210).

A polynucleotide that encodes SEQ ID NO:298 is (SEQ ID NO:299):

gacattcagc tgactcagtc cccctctttt ctgtccgcat
ccgtcggaga tcgagtgact attacttgct ctgctaggtc
ctcaatcagc ttcatgtact ggtatcagca gaagcccggc
aaagcaccta agctgctgat ctacgacaca agcaacctgg
cctccggggt gccatctcgg ttctctggca gtgggtcagg
aactgagttt accctgacaa ttagctccct ggaggctgaa
gatgccgcta cctactattg ccagcagtgg agcagctatc
ctctgacctt cggacagggg actaaactgg aaatcaagcg
tacggtggct gcaccatcgg tcttcatctt cccgccatct
gatgagcagt tgaaatctgg aactgcctct gttgtgtgcc
tgctgaataa cttctatccc agagaggcca aagtacagtg
gaaggtggat aacgccctcc aatcgggtaa ctcccaggag
agtgtcacag agcaggacag caaggacagc acctacagcc
tcagcagcac cctgacgctg agcaaagcag actacgagaa
acacaaagtc tacgcctgcg aagtcaccca tcagggcctg
agctcgcccg tcacaaagag cttcaacagg ggagagtgt

### IV. Reference Antibodies and Diabodies

In order to assist in the characterization of the Tri-Specific Binding Molecules of the present invention, the following reference diabodies were constructed.

### Q. DR5 mAb 1×CD3 mAb 2 Diabody

An exemplary bi-specific diabody composed of two polypeptide chains was constructed having the VL and VH domains of anti-human DR5 antibody DR5 mAb 1 and the VL and VH domains of CD3 mAb 2. The diabody was designated “DR5 mAb 1×CD3 mAb 2 diabody.” The amino acid sequence of the first polypeptide chain of this diabody is (SEQ ID NO:140):

DIVLTQSPAS LAVSLGQRAT ISCRASKSVS SSGYSYMHWY
QQKPGQPPKV LIFLSSNLDS GVPARFSGSG SGTDFTLNIH
PVEDGDAATY YCQHSRDLPP TFGGGTKLEI KGGGSGGGGE
VQLVESGGGL VQPGGSLRLS CAASGFTFST YAMNWVRQAP
GKGLEWVGRI RSKYNNYATY YADSVKGRFT ISRDDSKNSL
YLQMNSLKTE DTAVYYCVRH GNFGNSYVSW FAYWGQGTLV
TVSSASTKGE VAACEKEVAA LEKEVAALEK EVAALEK

In SEQ ID NO:140, amino acid residues 1-111 correspond to the amino acid sequence of the VL Domain of DR5 mAb 1 (SEQ ID NO:3), residues 112-119 correspond to the intervening spacer peptide GGGSGGGG (Linker 1) (SEQ ID NO:33), residues 120-244 correspond to the amino acid sequence of the VH Domain of CD3 mAb 2 having the D65G substitution (SEQ ID NO:112), residues 245-249 correspond to the ASTKG linker (SEQ ID NO:47) and residues 250-277 correspond to a cysteine-containing E-coil Domain (SEQ ID NO:41). A polynucleotide that encodes SEQ ID NO:140 is SEQ ID NO:141:

gacattgtgc tgacacagtc tcctgcttcc ttagctgtat
ctctcgggca gagggccacc atctcatgca gggccagcaa
aagtgtcagt tcctctggct atagttatat gcactggtac
caacagaaac caggacagcc acccaaagtc ctcatctttc
tttcatccaa cctagattct ggggtccctg ccaggttcag
tggcagtggg tctgggacag acttcaccct caacatccat
cctgtggagg atggggatgc tgcaacctat tactgtcagc
acagtaggga tcttcctccg acgttcggtg gaggcaccaa
gctggaaatc aaaggaggcg gatccggcgg cggaggcgag
gtgcagctgg tggagtctgg gggaggcttg gtccagcctg
gagggtccct gagactctcc tgtgcagcct ctggattcac
cttcagcaca tacgctatga attgggtccg ccaggctcca
gggaaggggc tggagtgggt tggaaggatc aggtccaagt
acaacaatta tgcaacctac tatgccgact ctgtgaaggg
tagattcacc atctcaagag atgattcaaa gaactcactg
tatctgcaaa tgaacagcct gaaaaccgag gacacggccg
tgtattactg tgtgagacac ggtaacttcg gcaattctta
cgtgtcttgg tttgcttatt ggggacaggg gacactggtg
actgtgtctt ccgcctccac caagggcgaa gtggccgcat
gtgagaaaga ggttgctgct ttggagaagg aggtcgctgc
acttgaaaag gaggtcgcag ccctggagaa a

The amino acid sequence of the second polypeptide chain of the DR5 mAb 1×CD3 mAb 2 diabody is (SEQ ID NO:142):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGEV
KFLESGGGLV QPGGSLKLSC VASGFDFSRY WMSWVRQAPG
KGLEWIGEIN PDSNTINYTP SLKDKFIISR DNAKNTLYLQ
MTKVRSEDTA LYYCTRRAYY GNPAWFAYWG QGTLVTVSAA
STKGKVAACK EKVAALKEKV AALKEKVAAL KE

In SEQ ID NO:142, amino acid residues 1-110 correspond to the amino acid sequence of the VL Domain of CD3 mAb 2 (SEQ ID NO:104), residues 111-118 correspond to the intervening spacer peptide GGGSGGGG (Linker 1) (SEQ ID NO:33), residues 119-239 correspond to the amino acid sequence of the VH Domain of DR5 mAb 1 (SEQ ID NO:8), except that the C-terminal serine residue of SEQ ID NO:8 has been replaced with an alanine residue), residues 240-244 correspond to an ASTKG linker (SEQ ID NO:47), and residues 245-272 correspond to a cysteine-containing K-coil Domain (SEQ ID NO:42). A polynucleotide that encodes SEQ ID NO:142 is SEQ ID NO:143:

caggctgtgg tgactcagga gccttcactg accgtgtccc
caggcggaac tgtgaccctg acatgcagat ccagcacagg
cgcagtgacc acatctaact acgccaattg ggtgcagcag
aagccaggac aggcaccaag gggcctgatc gggggtacaa
acaaaagggc tccctggacc cctgcacggt tttctggaag
tctgctgggc ggaaaggccg ctctgactat taccggggca
caggccgagg acgaagccga ttactattgt gctctgtggt
atagcaatct gtgggtgttc gggggtggca caaaactgac
tgtgctggga ggtggtggat ccggcggcgg aggcgaggtg
aagtttctcg agtctggagg tggcctggtg cagcctggag
gatccctgaa actctcctgt gtagcctcag gattcgattt
tagtagatac tggatgagtt gggtccggca ggctccaggg
aaagggctag aatggattgg agaaattaat ccagatagca
atacgataaa ctatacgcca tctctaaagg ataaattcat
catctccaga gacaacgcca aaaatacgct gtatctgcaa
atgaccaaag tgagatctga ggacacagcc ctttattatt
gtacaagaag ggcctactat ggtaacccgg cctggtttgc
ttactggggc caagggactc tggtcactgt ctctgcagcc
tccaccaagg gcaaagtggc cgcatgtaag gagaaagttg
ctgctttgaa agagaaggtc gccgcactta aggaaaaggt
cgcagccctg aaagag

### R. DR5 mAb 2×CD3 mAb 2 Diabody

An exemplary bi-specific diabody composed of two polypeptide chains was constructed having the VL and VH domains of anti-human DR5 antibody DR5 mAb 2 and the VL and VH domains of CD3 mAb 2. The diabody was designated “DR5 mAb 2×CD3 mAb 2 diabody.” The amino acid sequence of the first polypeptide chain of this diabody is (SEQ ID NO:144):

DIVMTQSHKF MSTSVGDRVS ITCKASQDVN TAVAWYQQKP
GQSPKLLIYW ASTRHTGVPD RFTGSGSGTD YTLTIKSVQA
EDLTLYYCQQ HYITPWTFGG GTKLEIKGGG SGGGGEVQLV
ESGGGLVQPG GSLRLSCAAS GFTFSTYAMN WVRQAPGKGL
EWVGRIRSKY NNYATYYADS VKGRFTISRD DSKNSLYLQM
NSLKTEDTAV YYCVRHGNFG NSYVSWFAYW GQGTLVTVSS
ASTKGEVAAC EKEVAALEKE VAALEKEVAA LEK

In SEQ ID NO:144, amino acid residues 1-107 correspond to the amino acid sequence of the VL Domain of DR5 mAb 2 (SEQ ID NO:13), residues 108-115 correspond to intervening spacer peptide (Linker 1) (SEQ ID NO:33), residues 116-240 correspond to the amino acid sequence of the VH Domain of CD3 mAb 2 having the D65G substitution (SEQ ID NO:112), residues 241-245 correspond to an ASTKG linker (SEQ ID NO:47) and residues 246-273 correspond to a cysteine-containing E-coil Domain (SEQ ID NO:41). A polynucleotide that encodes SEQ ID NO:144 is SEQ ID NO:145:

gacattgtga tgacccagtc tcacaaattc atgtccactt
cagtaggaga cagggtcagc atcacctgca aggccagtca
ggatgtgaat actgctgtag cctggtatca acaaaaacca
gggcaatctc ctaaactact gatttactgg gcatccaccc
ggcacactgg agtccctgat cgcttcacag gcagtggatc
tgggacagat tatacactca ccatcaaaag tgtgcaggct
gaagacctga cactttatta ctgtcagcaa cactatatca
ctccgtggac gttcggtgga ggcaccaagc tggaaatcaa
aggaggcgga tccggcggcg gaggcgaggt gcagctggtg
gagtctgggg gaggcttggt ccagcctgga gggtccctga
gactctcctg tgcagcctct ggattcacct tcagcacata
cgctatgaat tgggtccgcc aggctccagg gaaggggctg
gagtgggttg gaaggatcag gtccaagtac aacaattatg
caacctacta tgccgactct gtgaagggta gattcaccat
ctcaagagat gattcaaaga actcactgta tctgcaaatg
aacagcctga aaaccgagga cacggccgtg tattactgtg
tgagacacgg taacttcggc aattcttacg tgtcttggtt
tgcttattgg ggacagggga cactggtgac tgtgtcttcc
gcctccacca agggcgaagt ggccgcatgt gagaaagagg
ttgctgcttt ggagaaggag gtcgctgcac ttgaaaagga
ggtcgcagcc ctggagaaa

The amino acid sequence of the second polypeptide chain of the DR5 mAb 2×CD3 mAb 2 diabody is (SEQ ID NO:146):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGKV
QLQQSGAELV KPGASVKLSC KASGYTFTEY ILHWVKQKSG
QGLEWIGWFY PGNNNIKYNE KFKDKATLTA DKSSSTVYME
LSRLTSEDSA VYFCARHEQG PGYFDYWGQG TTLTVSSAST
KGKVAACKEK VAALKEKVAA LKEKVAALKE

In SEQ ID NO:146, amino acid residues 1-110 correspond to the amino acid sequence of the VL Domain of CD3 mAb 2 (SEQ ID NO:104), residues 111-118 correspond to the intervening spacer peptide GGGSGGGG (Linker 1) (SEQ ID NO:33), residues 119-237 correspond to the amino acid sequence of the VH Domain of DR5 mAb 2 (SEQ ID NO:18), residues 238-242 correspond to an ASTKG linker (SEQ ID NO:47), and residues 243-270 correspond to a cysteine-containing K-coil Domain (SEQ ID NO:42). A polynucleotide that encodes SEQ ID NO:146 is SEQ ID NO:147:

caggctgtgg tgactcagga gccttcactg accgtgtccc
caggcggaac tgtgaccctg acatgcagat ccagcacagg
cgcagtgacc acatctaact acgccaattg ggtgcagcag
aagccaggac aggcaccaag gggcctgatc gggggtacaa
acaaaagggc tccctggacc cctgcacggt tttctggaag
tctgctgggc ggaaaggccg ctctgactat taccggggca
caggccgagg acgaagccga ttactattgt gctctgtggt
atagcaatct gtgggtgttc gggggtggca caaaactgac
tgtgctggga gggggtggat ccggcggcgg aggcaaggtc
cagctgcagc agtctggagc tgaactggtg aaacccgggg
catcagtgaa gctgtcctgc aaggcttctg ggtacacctt
cactgagtat attttacact gggtaaagca gaagtctgga
cagggtcttg agtggattgg gtggttttat cctggaaata
ataatataaa gtacaatgag aaattcaagg acaaggccac
actgactgcg gacaaatcct ccagcacagt ctatatggaa
cttagtagat tgacatctga agactctgcg gtctatttct
gtgcaagaca cgaacaagga ccaggttact ttgactactg
gggccaaggc accactctca cagtctcctc cgcctccacc
aagggcaaag tggccgcatg taaggagaaa gttgctgctt
tgaaagagaa ggtcgccgca cttaaggaaa aggtcgcagc
cctgaaagag

### S. DR5 mAb 3×CD3 mAb 2 Diabody

An exemplary bi-specific diabody composed of two polypeptide chains was constructed having the VL and VH domains of anti-human DR5 antibody DR5 mAb 3 and the VL and VH domains of CD3 mAb 2. The amino acid sequence of the first polypeptide chain of the diabody had the sequence (SEQ ID NO:148) (CDR residues are shown underlined):

SELTQDPAVS VALGQTVRIT CSGDSLRSYY ASWYQQKPGQ
APVLVIYGAN NRPSGIPDRF SGSSSGNTAS LTITGAQAED
EADYYCNSAD SSGNHVVFGG GTKLTVLGGG GSGGGGEVQL
VESGGGLVQP GGSLRLSCAA SGFTFSTYAM NWVRQAPGKG
LEWVGRIRSK YNNYATYYAD SVKGRFTISR DDSKNSLYLQ
MNSLKTEDTA VYYCVRHGNF GNSYVSWFAY WGQGTLVTVS
SASTKGEVAA CEKEVAALEK EVAALEKEVA ALEK

In SEQ ID NO:148, amino acid residues 1-108 correspond to the VL Domain of DR5 mAb 3 (SEQ ID NO:54), residues 109-116 correspond to the intervening spacer peptide GGGSGGGG (Linker 1) (SEQ ID NO:33), residues 117-241 correspond to the amino acid sequence of the VH Domain of CD3 mAb 2 having the D65G substitution (SEQ ID NO:112), residues 242-246 correspond to an ASTKG linker (SEQ ID NO:47), and residues 247-275 correspond to a cysteine-containing K-coil Domain (SEQ ID NO:42).

The amino acid sequence of the second polypeptide chain of the diabody had the sequence (SEQ ID NO:149) (CDR residues are shown underlined):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGEV
QLVQSGGGVE RPGGSLRLSC AASGFTFDDY AMSWVRQAPG
KGLEWVSGIN WQGGSTGYAD SVKGRVTISR DNAKNSLYLQ
MNSLRAEDTA VYYCAKILGA GRGWYFDYWG KGTTVTVSSA
STKGKVAACK EKVAALKEKV AALKEKVAAL KE

In SEQ ID NO:149, amino acid residues 1-110 correspond to the VL Domain of CD3 mAb 2 (SEQ ID NO:104), residues 111-118 correspond to the intervening spacer peptide GGGSGGGG (Linker 1) (SEQ ID NO:33), residues 119-239 correspond to the amino acid sequence of the VH Domain of DR5 mAb 3 (SEQ ID NO:58), residues 240-244 correspond to an ASTKG linker (SEQ ID NO:47), and residues 245-272 correspond to a cysteine-containing K-coil Domain (SEQ ID NO:42).

### T. DR5 mAb 4×CD3 mAb 2 Diabody

An exemplary bi-specific diabody composed of two polypeptide chains was constructed having the VL and VH domains of anti-human DR5 antibody DR5 mAb 4 and the VL and VH domains of CD3 mAb 2. The amino acid sequence of the first polypeptide chain of the diabody had the sequence (SEQ ID NO:150) (CDR residues are shown underlined):

EIVLTQSPGT LSLSPGERAT LSCRASQGIS RSYLAWYQQK
PGQAPSLLIY GASSRATGIP DRFSGSGSGT DFTLTISRLE
PEDFAVYYCQ QFGSSPWTFG QGTKVEIKGG GSGGGGEVQL
VESGGGLVQP GGSLRLSCAA SGFTFSTYAM NWVRQAPGKG
LEWVGRIRSK YNNYATYYAD SVKGRFTISR DDSKNSLYLQ
MNSLKTEDTA VYYCVRHGNF GNSYVSWFAY WGQGTLVTVS
SASTKGEVAA CEKEVAALEK EVAALEKEVA ALEK

In SEQ ID NO:150, amino acid residues 1-108 correspond to the VL Domain of DR5 mAb 4 (SEQ ID NO:62), residues 109-116 correspond to the intervening spacer peptide GGGSGGGG (Linker 1) (SEQ ID NO:33), residues 117-241 correspond to the amino acid sequence of the VH Domain of CD3 mAb 2 having the D65G substitution (SEQ ID NO:112), residues 242-246 correspond to an ASTKG linker (SEQ ID NO:47), and residues 247-275 correspond to a cysteine-containing E-coil Domain (SEQ ID NO:41).

The amino acid sequence of the second polypeptide chain of the diabody had the sequence (SEQ ID NO:151) (CDR residues are shown underlined):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGQV
QLQESGPGLV KPSQTLSLTC TVSGGSISSG DYFWSWIRQL
PGKGLEWIGH IHNSGTTYYN PSLKSRVTIS VDTSKKQFSL
RLSSVTAADT AVYYCARDRG GDYYYGMDVW GQGTTVTVSS
ASTKGKVAAC KEKVAALKEK VAALKEKVAA LKE

In SEQ ID NO:151, amino acid residues 1-110 correspond to the VL Domain of CD3 mAb 2 (SEQ ID NO:104), residues 111-118 correspond to the intervening spacer peptide GGGSGGGG (Linker 1) (SEQ ID NO:33), residues 119-240 correspond to the amino acid sequence of the VH Domain of DR5 mAb 4 (SEQ ID NO:66), residues 241-245 correspond to an ASTKG linker (SEQ ID NO:47), and residues 246-273 correspond to a cysteine-containing K-coil Domain (SEQ ID NO:42).

### U. Reference gpA33×CD3 mAb 2 Diabody

To further exemplify the bi-specific Tri-Specific Binding Molecules of the present invention, a diabody composed of two polypeptide chains was constructed using the VL and VH domains of gpA33 mAb 1 and CD3 mAb 2. The amino acid sequence of the first polypeptide chain of the diabody had the sequence (SEQ ID NO:316) (CDR residues are shown underlined):

DIQLTQSPSF LSASVGDRVT ITCSARSSIS FMYWYQQKPG
KAPKLLIYDT SNLASGVPSR FSGSGSGTEF TLTISSLEAE
DAATYYCQQW SSYPLTFGQG TKLEIKGGGS GGGGEVQLVE
SGGGLVQPGG SLRLSCAASG FTFSTYAMNW VRQAPGKGLE
WVGRIRSKYN NYATYYADSV KGRFTISRDD SKNSLYLQMN
SLKTEDTAVY YCVRHGNFGN SYVSWFAYWG QGTLVTVSSA
STKGEVAACE KEVAALEKEV AALEKEVAAL EK

In SEQ ID NO:316, amino acid residues 1-106 correspond to the VL Domain of gpA33 mAb 1 (SEQ ID NO:181), residues 107-114 correspond to the intervening spacer peptide GGGSGGGG (Linker 1) (SEQ ID NO:33), residues 115-239 correspond to the amino acid sequence of the VH Domain of CD3 mAb 2 having the D65G substitution (SEQ ID NO:112), residues 240-244 correspond to an ASTKG linker (SEQ ID NO:47), and residues 245-272 correspond to a cysteine-containing E-coil Domain (SEQ ID NO:41).

The amino acid sequence of the second polypeptide chain of the diabody had the sequence (SEQ ID NO:317) (CDR residues are shown underlined):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGQV
QLVQSGAEVK KPGASVKVSC KASGYTFTGS WMNWVRQAPG
QGLEWIGRIY PGDGETNYNG KFKDRVTITA DKSTSTAYME
LSSLRSEDTA VYYCARIYGN NVYFDVWGQG TTVTVSSAST
KGKVAACKEK VAALKEKVAA LKEKVAALKE

In SEQ ID NO:317, amino acid residues 1-110 correspond to the VL Domain of CD3 mAb 2 (SEQ ID NO:104), residues 111-118 correspond to the intervening spacer peptide GGGSGGGG (Linker 1) (SEQ ID NO:33), residues 119-237 correspond to the amino acid sequence of the VH Domain of gpA33 mAb 1 (SEQ ID NO:186), residues 238-242 correspond to an ASTKG linker (SEQ ID NO:47), and residues 243-270 correspond to a cysteine-containing K-coil Domain (SEQ ID NO:42).

### V. Reference Anti-Fluorescein Antibody

The anti-fluorescein antibody 4-4-20 (Gruber, M. et al. (1994) “Efficient Tumor Cell Lysis Mediated By A Bi-specific Single Chain Antibody Expressed In Escherichia coli,” J. Immunol. 152(11):5368-5374; Bedzyk, W. D. et al. (1989) “Comparison Of Variable Region Primary Structures Within An Anti-Fluorescein Idiotype Family,” J. Biol. Chem. 264(3): 1565-1569) was used in control diabodies. The amino acid sequences of the variable light and variable heavy Domains of anti-fluorescein antibody 4-4-20 are as follows:

Amino Acid Sequence Of The Variable Light Chain Domain Of Anti-Fluorescein Antibody 4-4-20 (SEQ ID NO:138) (CDR residues are underlined):

DVVMTQTPFS LPVSLGDQAS ISCRSSQSLV HSNGNTYLRW
YLQKPGQSPK VLIYKVSNRF SGVPDRFSGS GSGTDFTLKI
SRVEAEDLGV YFCSQSTHVP WTFGGGIKLE IK

Amino Acid Sequence Of The Variable Heavy Chain Domain Of Anti-Fluorescein Antibody 4-4-20 (SEQ ID NO:139) (CDR residues are underlined):

EVKLDETGGG LVQPGRPMKL SCVASGFTFS DYWMNWVRQS
PEKGLEWVAQ IRNKPYNYET YYSDSVKGRF TISRDDSKSS
VYLQMNNLRV EDMGIYYCTG SYYGMDYWGQ GTSVTVSS

### V. Methods of Production

The Tri-Specific Binding Molecules of the present invention can be created from the polynucleotides and/or sequences of antibodies that are immunospecific for DR5, a desired Cancer Antigen, and a desired Effector Cell by methods known in the art, for example, synthetically or recombinantly. One method of producing such peptide agonists, antagonists and modulators involves chemical synthesis of the polypeptide, followed by treatment under oxidizing conditions appropriate to obtain the native conformation, that is, the correct disulfide bond linkages. This can be accomplished using methodologies well-known to those skilled in the art (see, e.g., Kelley, R. F. et al. (1990) In: GENETIC ENGINEERING PRINCIPLES AND METHODS, Setlow, J. K. Ed., Plenum Press, N.Y., vol. 12, pp 1-19; Stewart, J. M et al. (1984) SOLID PHASE PEPTIDE SYNTHESIS, Pierce Chemical Co., Rockford, TL; see also U.S. Pat. Nos. 4,105,603; 3,972,859; 3,842,067; and 3,862,925).

Polypeptides of the invention may be conveniently prepared using solid phase peptide synthesis (Merrifield, B. (1986) “Solid Phase Synthesis,” Science 232(4748):341-347; Houghten, R. A. (1985) “General Method For The Rapid Solid-Phase Synthesis Of Large Numbers Of Peptides: Specificity Of Antigen-Antibody Interaction At The Level Of Individual Amino Acids,” Proc. Natl. Acad. Sci. (U.S.A.) 82(15):5131-5135; Ganesan, A. (2006) “Solid-Phase Synthesis In The Twenty-First Century,” Mini Rev. Med. Chem. 6(1):3-10).

In yet another alternative, suitable antibodies having one or more of the CDRs of a desired anti-DR5 antibody, anti-Cancer Antigen antibody or anti-Effector Cell antibody may be obtained through the use of commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are XENOMOUSE™ (Abgenix, Inc., Fremont, CA) and HUMAB-MOUSE® and TC MOUSE™ (both from Medarex, Inc., Princeton, NJ).

In an alternative, antibodies may be made recombinantly and expressed using any method known in the art. Antibodies may be made recombinantly by first isolating the antibodies made from host animals, obtaining the gene sequence, and using the gene sequence to express the antibody recombinantly in host cells (e.g., CHO cells). Another method that may be employed is to express the antibody sequence in plants {e.g., tobacco) or transgenic milk. Suitable methods for expressing antibodies recombinantly in plants or milk have been disclosed (see, for example, Peeters et al. (2001) “Production Of Antibodies And Antibody Fragments In Plants,” Vaccine 19:2756; Lonberg, N. et al. (1995) “Human Antibodies From Transgenic Mice,” Int. Rev. Immunol 13:65-93; and Pollock et al. (1999) “Transgenic Milk As A Method For The Production Of Recombinant Antibodies,” J. Immunol Methods 231:147-157). Suitable methods for making derivatives of antibodies, e.g., humanized, single-chain, etc. are known in the art. In another alternative, antibodies may be made recombinantly by phage display technology (see, for example, U.S. Pat. Nos. 5,565,332; 5,580,717; 5,733,743; 6,265,150; and Winter, G. et al. (1994) “Making Antibodies By Phage Display Technology,” Annu. Rev. Immunol. 12.433-455).

The antibodies or protein of interest may be subjected to sequencing by Edman degradation, which is well-known to those of skill in the art. The peptide information generated from mass spectrometry or Edman degradation can be used to design probes or primers that are used to clone the protein of interest.

An alternative method of cloning the protein of interest is by “panning” using purified DR5, and/or a desired Cancer Antigen, and/or a molecule expressed on the surface of a desired Effector Cell (or portions of any such molecules), for cells expressing an antibody or protein of interest that possesses one or more CDRs so as to be capable of binding to DR5, or such desired Cancer Antigen or Effector Cell molecule. The “panning” procedure may be conducted by obtaining a cDNA library from tissues or cells that express DR5, overexpressing the cDNAs in a second cell type, and screening the transfected cells of the second cell type for a specific binding to DR5 in the presence or absence of a known antibody that is capable of binding to such molecule (e.g., DR5 mAb 1 or DR5 mAb 2 in the case of panning for new anti-DR5 antibodies, etc.). Detailed descriptions of the methods used in cloning mammalian genes coding for cell surface proteins by “panning” can be found in the art (see, for example, Aruffo, A. et al. (1987) “Molecular Cloning Of A CD28 cDNA By A High-Efficiency COS Cell Expression System,” Proc. Natl. Acad. Sci. (U.S.A.) 84:8573-8577 and Stephan, J. et al. (1999) “Selective Cloning Of Cell Surface Proteins Involved In Organ Development: Epithelial Glycoprotein Is Involved In Normal Epithelial Differentiation,” Endocrinol. 140:5841-5854).

Vectors containing polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

Any host cells capable of overexpressing heterologous DNAs can be used for the purpose of isolating the genes encoding the antibody, polypeptide or protein of interest. Non-limiting examples of suitable mammalian host cells include but are not limited to COS, HeLa, and CHO cells. Preferably, the host cells express the cDNAs at a level of about 5-fold higher, more preferably 10-fold higher, even more preferably 20-fold higher than that of the corresponding endogenous antibody or protein of interest, if present, in the host cells. Screening the host cells for a specific binding to DR5 is effected by an immunoassay or FACS. A cell overexpressing the antibody or protein of interest can be identified.

The invention includes polypeptides comprising an amino acid sequence of the antibodies of this invention. The polypeptides of this invention can be made by procedures known in the art. The polypeptides can be produced by proteolytic or other degradation of the antibodies, by recombinant methods (i.e., single or fusion polypeptides) as described above or by chemical synthesis. Polypeptides of the antibodies, especially shorter polypeptides up to about 50 amino acids, are conveniently made by chemical synthesis. Methods of chemical synthesis are known in the art and are commercially available. For example, an anti-DR5 polypeptide could be produced by an automated polypeptide synthesizer employing the solid phase method.

The invention includes modifications to any such antibodies (or to any of their polypeptide fragments that bind to DR5, the Cancer Antigen or the effector cell, as the case may be) and the agonists, antagonists, and modulators of such molecules, including functionally equivalent antibodies and fusion polypeptides that do not significantly affect the properties of such molecules as well as variants that have enhanced or decreased activity. Modification of polypeptides is routine practice in the art and need not be described in detail herein. Examples of modified polypeptides include polypeptides with conservative substitutions of amino acid residues, one or more deletions or additions of amino acids which do not significantly deleteriously change the functional activity, or use of chemical analogs. Amino acid residues which can be conservatively substituted for one another include but are not limited to: glycine/alanine; serine/threonine; valine/isoleucine/leucine; asparagine/glutamine; aspartic acid/glutamic acid; lysine/arginine; and phenylalanine/tyrosine. These polypeptides also include glycosylated and non-glycosylated polypeptides, as well as polypeptides with other post-translational modifications, such as, for example, glycosylation with different sugars, acetylation, and phosphorylation. Preferably, the amino acid substitutions would be conservative, i.e., the substituted amino acid would possess similar chemical properties as that of the original amino acid. Such conservative substitutions are known in the art, and examples have been provided above. Amino acid modifications can range from changing or modifying one or more amino acids to complete redesign of a region, such as the variable region. Changes in the variable region can alter binding affinity and/or specificity. Other methods of modification include using coupling techniques known in the art, including, but not limited to, enzymatic means, oxidative substitution and chelation. Modifications can be used, for example, for attachment of labels for immunoassay, such as the attachment of radioactive moieties for radioimmunoassay. Modified polypeptides are made using established procedures in the art and can be screened using standard assays known in the art.

The invention encompasses fusion proteins comprising one or more of the polypeptides of this invention. In one embodiment, a fusion polypeptide is provided that comprises a light chain, a heavy chain or both a light and heavy chain. In another embodiment, the fusion polypeptide contains a heterologous immunoglobulin constant region. In another embodiment, the fusion polypeptide contains a light chain variable region and a heavy chain variable region of an antibody produced from a publicly-deposited hybridoma. For purposes of this invention, an antibody fusion protein contains one or more polypeptide domains that specifically bind to DR5, a Cancer Antigen, or an effector cell (as the case may be) and another amino acid sequence to which it is not attached in the native molecule, for example, a heterologous sequence or a homologous sequence from another region.

### VI. Uses of the Trispecific Binding Molecules of the Present Invention

The Tri-Specific Binding Molecules of the present invention provide a general therapy for cancer. The cancers that may be treated by such molecules include cancers characterized by the presence of a cancer cell selected from the group consisting of a cell of: an adrenal gland tumor, an AIDS-associated cancer, an alveolar soft part sarcoma, an astrocytic tumor, bladder cancer, bone cancer, a brain and spinal cord cancer, a metastatic brain tumor, a breast cancer, a carotid body tumors, a cervical cancer, a chondrosarcoma, a chordoma, a chromophobe renal cell carcinoma, a clear cell carcinoma, a colon cancer, a colorectal cancer, a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, gastric cancer, a gestational trophoblastic disease, a germ cell tumor, a head and neck cancer, hepatocellular carcinoma, an islet cell tumor, a Kaposi's Sarcoma, a kidney cancer, a leukemia, a lipoma/benign lipomatous tumor, a liposarcoma/malignant lipomatous tumor, a liver cancer, a lymphoma, a lung cancer, a medulloblastoma, a melanoma, a meningioma, a multiple endocrine neoplasia, a multiple myeloma, a myelodysplastic syndrome, a neuroblastoma, a neuroendocrine tumors, an ovarian cancer, a pancreatic cancer, a papillary thyroid carcinoma, a parathyroid tumor, a pediatric cancer, a peripheral nerve sheath tumor, a phaeochromocytoma, a pituitary tumor, a prostate cancer, a posterious uveal melanoma, a rare hematologic disorder, a renal metastatic cancer, a rhabdoid tumor, a rhabdomysarcoma, a sarcoma, a skin cancer, a soft-tissue sarcoma, a squamous cell cancer, a stomach cancer, a synovial sarcoma, a testicular cancer, a thymic carcinoma, a thymoma, a thyroid metastatic cancer, and a uterine cancer. The Tri-Specific Binding Molecules of the present invention may be used in the treatment of colorectal cancer, hepatocellular carcinoma, glioma, kidney cancer, breast cancer, multiple myeloma, bladder cancer, neuroblastoma; sarcoma, non-Hodgkin's lymphoma, non-small cell lung cancer, ovarian cancer, pancreatic cancer and rectal cancer.

The Tri-Specific Binding Molecules of the present invention augment the cancer therapy provided by an antibody directed to a Cancer Antigen that is characteristic of cells of a target tumor by being additionally able to bind to DR5 molecules arrayed on the surface of such tumor cells. The utility of the invention is particularly seen in circumstances in which the density of the Cancer Antigen is low, or when the binding kinetics of the anti-Cancer Antigen antibody is suboptimal (or insufficient) to promote a clinically sufficient therapeutic response. In such cases, the ability of the molecules of the present invention to bind both the Cancer Antigen and DR5 of the tumor cells provides enhanced binding (via avidity) that is sufficient to promote a clinically sufficient therapeutic response. Additionally, by also possessing a Binding Domain capable of binding to a molecule on the surface of an immune system effector cell, the Tri-Specific Binding Molecules of the present invention permit the co-localization of such immune system cells to the tumor cells, thereby promoting a cytotoxic response against the tumor cells via redirected killing.

As shown in Table 2, Tri-Specific Binding Molecules of the present invention that possess particular combinations of Cancer Antigen-Binding Domains have preferred utility in the treatment of specific cancers.

TABLE 2

| Cancer Antigen-Binding | | |
|---|---|---|
| Domains | | Preferred Utility |
| gpA33 | DR5 | Treatment Of Colorectal Cancer |
| gpA33 | EphA2 | |
| gpA33 | B7-H3 | |
| gpA33 | BST2 | |
| 5T4 | EphA2 | Broadly Applicable To Treatment |
| 5T4 | CEACAM5 | Of Many Types Of Cancers |
| 5T4 | B7-H3 | |
| 5T4 | DR5 | |
| B7-H3 | CEACAM5 | |
| B7-H3 | CEACAM6 | |
| B7-H3 | IL1Rα2 | Glioblastoma, Melanoma |
| EphA2 | IL1Rα2 | |
| EphA2 | DR5 | Broadly Applicable To Treatment |
| EphA2 | CEACAM5 | Of Many Types Of Cancers |
| EphA2 | CEACAM6 | |
| ITGB6 | B7-H3 | |
| ITGB6 | DR5 | |
| ITGB6 | BST2 | |
| BST2 | CEACAM5 | |
| BST2 | EGFR | |

In addition to their utility in therapy, the Tri-Specific Binding Molecules of the present invention may be detectably labeled and used in the diagnosis of cancer or in the imaging of tumors and tumor cells.

### VII. Pharmaceutical Compositions

The compositions of the invention include bulk drug compositions useful in the manufacture of pharmaceutical compositions (e.g., impure or non-sterile compositions) and pharmaceutical compositions (i.e., compositions that are suitable for administration to a subject or patient) which can be used in the preparation of unit dosage forms. Such compositions comprise a prophylactically or therapeutically effective amount of the Tri-Specific Binding Molecules of the present invention, or a combination of such agents and a pharmaceutically acceptable carrier. Preferably, compositions of the invention comprise a prophylactically or therapeutically effective amount of the Tri-Specific Binding Molecules of the present invention and a pharmaceutically acceptable carrier. The invention particularly encompasses such pharmaceutical compositions in which the Tri-Specific Binding Molecule has a DR5-Binding Domain of:
(1) a DR5 mAb 1 antibody;
(2) a DR5 mAb 2 antibody;
(3) a DR5 mAb 3 antibody;
(4) a DR5 mAb 4 antibody;
(5) a DR5 mAb 5 antibody;
(6) a DR5 mAb 6 antibody;
(7) a DR5 mAb 7 antibody; or
(8) a DR5 mAb 8 antibody
   (or a humanized derivative of any such antibodies).

The invention further particularly encompasses such pharmaceutical compositions in which the Tri-Specific Binding Molecule has a Cancer Antigen-Binding Domain that:
(A) binds to an epitope of EphA2, especially wherein the Tri-Specific Binding Molecule has a Cancer Antigen-Binding Domain of EphA2 mAb 1, EphA2 mAb 2 or EphA2 mAb 3, or a humanized or chimeric variant thereof; or
(B) binds to an epitope of gpA33, especially wherein the Tri-Specific Binding Molecule has a Cancer Antigen-Binding Domain of gpA33 mAb 1, or a humanized or chimeric variant thereof; or
(C) binds to an epitope of Her2, especially wherein the Tri-Specific Binding Molecule has a Cancer Antigen-Binding Domain of Her2 mAb 1 or trastuzumab, or a humanized or chimeric variant thereof; or (D) binds to an epitope of B7-H3, especially wherein the Tri-Specific Binding Molecule has a Cancer Antigen-Binding Domain of B7-H3 mAb 1, B7-H3 mAb 2, or B7-H3 mAb 3, or a humanized or chimeric variant thereof.

The invention further particularly encompasses such pharmaceutical compositions in which the Tri-Specific Binding Molecule has an Effector Cell-Binding Domain that binds to CD2, CD3, CD17, CD20, CD22, CD32B, CD64, BCR/CD79, the T cell Receptor or the NKG2D Receptor. The invention further particularly encompasses such pharmaceutical compositions in which the Tri-Specific Binding Molecule has an Effector Cell-Binding Domain of antibody: Lo-CD2a, CD3 mAb 2, OKT3, 3G8, A9, HD37, rituximab, epratuzumab, CD32B mAb 1, CD64 mAb 1, CD79 mAb 1, BMA 031, KYK-1.0, or KYK-2.0.

The invention specifically contemplates Tri-Specific Binding Molecules, pharmaceutical compositions that comprise such binding molecule and uses of such Tri-Specific Binding Molecules, in which:
(1) the DR5 Binding Domain is a DR5 binding domain of any anti-DR5 antibody;
(2) the Cancer Binding Domain is any of the Cancer Antigens disclosed herein;
and
(3) the Effector Cell-Binding Domains binds to any of CD2, CD3, CD17, CD20, CD22, CD32B, CD64, BCR/CD79, the T cell Receptor or the NKG2D Receptor.

The invention further specifically contemplates Tri-Specific Binding Molecules, pharmaceutical compositions that comprise such binding molecule and uses of such Tri-Specific Binding Molecules, in which:
(1) the DR5 Binding Domain is a DR5 binding domain of any anti-DR5 antibody;
(2) the Cancer Binding Domain is any of: EphA1, gpA33, Her2, or B7-H3;
and
(3) the Effector Cell-Binding Domains binds to any of CD2, CD3, CD17, CD20, CD22, CD32B, CD64, BCR/CD79, the T cell Receptor or the NKG2D Receptor.

The invention particularly contemplates each of the Tri-Specific Binding Molecules, as well as pharmaceutical compositions that comprise such binding molecule and uses of such Tri-Specific Binding Molecules, in which:
(1) the DR5 Binding Domain is a DR5 binding domain of any of: a DR5 mAb 1 antibody, a DR5 mAb 2 antibody, a DR5 mAb 3 antibody, a DR5 mAb 4 antibody, a DR5 mAb 5 antibody, a DR5 mAb 6 antibody, a DR5 mAb 7 antibody, or a DR5 mAb 8 antibody;
(2) the Cancer Antigen-Binding Domain is a binding domain of any of: EphA2 mAb 1, EphA2 mAb 2, EphA2 mAb 3, gpA33 mAb 1, Her2 mAb 1, trastuzumab, B7-H3 mAb 1, B7-H3 mAb 2, or B7-H3 mAb 3;
and
(3) the Effector Cell-Binding Domain is a binding domain of any of: Lo-CD2a, CD3 mAb 2, OKT3, 3G8, A9, HD37, rituximab, epratuzumab, CD32B mAb 1, CD64 mAb 1, CD79 mAb 1, BMA 031, KYK-1.0, or KYK-2.0.

As 8 anti-DR5 Binding Domain antibodies, 9 anti-Cancer Antigen-Binding Domain antibodies and 14 anti-Effector Cell-Binding Domain antibodies are listed, such specific contemplation encompasses all (8×9×14=) 1,008 combinations of such binding domains.

The invention also encompasses such pharmaceutical compositions that additionally include a second therapeutic antibody (e.g., tumor-specific monoclonal antibody) that is specific for a particular cancer antigen, and a pharmaceutically acceptable carrier.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include, but are not limited to those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The invention also provides a pharmaceutical pack or kit comprising one or more containers filled with a Tri-Specific Binding Molecule of the present invention (and more preferably, any of the specific binding molecules discussed or exemplified above). Additionally, one or more other prophylactic or therapeutic agents useful for the treatment of a disease can also be included in the pharmaceutical pack or kit. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The present invention provides kits that can be used in the above methods. A kit can comprise any of the Tri-Specific Binding Molecules of the present invention. The kit can further comprise one or more other prophylactic and/or therapeutic agents useful for the treatment of cancer, in one or more containers; and/or the kit can further comprise one or more cytotoxic antibodies that bind one or more cancer antigens associated with cancer. In certain embodiments, the other prophylactic or therapeutic agent is a chemotherapeutic. In other embodiments, the prophylactic or therapeutic agent is a biological or hormonal therapeutic.

### VIII. Methods of Administration

The compositions of the present invention may be provided for the treatment, prophylaxis, and amelioration of one or more symptoms associated with a disease, disorder or infection by administering to a subject an effective amount of a fusion protein or a conjugated molecule of the invention, or a pharmaceutical composition comprising a fusion protein or a conjugated molecule of the invention. In a preferred aspect, such compositions are substantially purified (i.e., substantially free from substances that limit its effect or produce undesired side effects). In a specific embodiment, the subject is an animal, preferably a mammal such as non-primate (e.g., bovine, equine, feline, canine, rodent, etc.) or a primate (e.g., monkey such as, a cynomolgus monkey, human, etc.). In a preferred embodiment, the subject is a human.

Various delivery systems are known and can be used to administer the compositions of the invention, e.g., encapsulation in liposomes, microparticles, microcapsules, recombinant cells capable of expressing the antibody or fusion protein, receptor-mediated endocytosis (See, e.g., Wu et al. (1987) “Receptor-Mediated In Vitro Gene Transformation By A Soluble DNA Carrier System,” J. Biol. Chem. 262:4429-4432), construction of a nucleic acid as part of a retroviral or other vector, etc.

Methods of administering a molecule of the invention include, but are not limited to, parenteral administration (e.g., intradermal, intramuscular, intraperitoneal, intravenous and subcutaneous), epidural, and mucosal (e.g., intranasal and oral routes). In a specific embodiment, the Tri-Specific Binding Molecules of the present invention are administered intramuscularly, intravenously, or subcutaneously. The compositions may be administered by any convenient route, for example, by infusion or bolus injection, by absorption through epithelial or mucocutaneous linings (e.g., oral mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent. See, e.g., U.S. Pat. Nos. 6,019,968; 5,985,320; 5,985,309; 5,934,272; 5,874,064; 5,855,913; 5,290,540; and 4,880,078; and PCT Publication Nos. WO 92/19244; WO 97/32572; WO 97/44013; WO 98/31346; and WO 99/66903, each of which is incorporated herein by reference in its entirety.

The invention also provides that the Tri-Specific Binding Molecules of the present invention are packaged in a hermetically sealed container such as an ampoule or sachette indicating the quantity of the molecule. In one embodiment, such molecules are supplied as a dry sterilized lyophilized powder or water free concentrate in a hermetically sealed container and can be reconstituted, e.g., with water or saline to the appropriate concentration for administration to a subject. Preferably, the Tri-Specific Binding Molecules of the present invention are supplied as a dry sterile lyophilized powder in a hermetically sealed container at a unit dosage of at least 5 μg, more preferably at least 10 μg, at least 15 μg, at least 25 μg, at least 50 μg, at least 100 μg, or at least 200 μg.

The lyophilized Tri-Specific Binding Molecules of the present invention should be stored at between 2 and 8° C. in their original container and the molecules should be administered within 12 hours, preferably within 6 hours, within 5 hours, within 3 hours, or within 1 hour after being reconstituted. In an alternative embodiment, such molecules are supplied in liquid form in a hermetically sealed container indicating the quantity and concentration of the molecule, fusion protein, or conjugated molecule. Preferably, such Tri-Specific Binding Molecules when provided in liquid form are supplied in a hermetically sealed container in which the molecules are present at a concentration of least 1 μg/ml, more preferably at least 2.5 μg/ml, at least 5 μg/ml, at least 10 μg/ml, at least 50 μg/ml, or at least 100 μg/ml.

The amount of the composition of the invention which will be effective in the treatment, prevention or amelioration of one or more symptoms associated with a disorder can be determined by standard clinical techniques. The precise dose to be employed in the formulation will also depend on the route of administration, and the seriousness of the condition, and should be decided according to the judgment of the practitioner and each patient's circumstances. Effective doses may be extrapolated from dose-response curves derived from in vitro or animal model test systems.

For the Tri-Specific Binding Molecules monovalent diabodies encompassed by the invention, the dosage administered to a patient is preferably determined based upon the body weight (kg) of the recipient subject. The dosage administered is typically from at least about 0.3 ng/kg per day to about 0.9 ng/kg per day, from at least about 1 ng/kg per day to about 3 ng/kg per day, from at least about 3 ng/kg per day to about 9 ng/kg per day, from at least about 10 ng/kg per day to about 30 ng/kg per day, from at least about 30 ng/kg per day to about 90 ng/kg per day, from at least about 100 ng/kg per day to about 300 ng/kg per day, from at least about 200 ng/kg per day to about 600 ng/kg per day, from at least about 300 ng/kg per day to about 900 ng/kg per day, from at least about 400 ng/kg per day to about 800 ng/kg per day, from at least about 500 ng/kg per day to about 1000 ng/kg per day, from at least about 600 ng/kg per day to about 1000 ng/kg per day, from at least about 700 ng/kg per day to about 1000 ng/kg per day, from at least about 800 ng/kg per day to about 1000 ng/kg per day, from at least about 900 ng/kg per day to about 1000 ng/kg per day, or at least about 1,000 ng/kg per day.

In another embodiment, the patient is administered a treatment regimen comprising one or more doses of such prophylactically or therapeutically effective amount of a Tri-Specific Binding Molecule of the present invention, wherein the treatment regimen is administered over 2 days, 3 days, 4 days, 5 days, 6 days or 7 days. In certain embodiments, the treatment regimen comprises intermittently administering doses of the prophylactically or therapeutically effective amount of the Tri-Specific Binding Molecules of the present invention (for example, administering a dose on day 1, day 2, day 3 and day 4 of a given week and not administering doses of the prophylactically or therapeutically effective amount of the Tri-Specific Binding Molecule

Especially encompassed is the administration of such Tri-Specific Binding Molecules that comprise any of the specific combinations of DR5 Binding Domains, Cancer Antigen-Binding Domains and Effector Cell-Binding Domains discussed above, on day 5, day 6 and day 7 of the same week). Typically, there are 1, 2, 3, 4, 5 or more courses of treatment. Each course may be the same regimen or a different regimen.

In another embodiment, the administered dose escalates over the first quarter, first half or first two-thirds or three-quarters of the regimen(s) (e.g., over the first, second, or third regimens of a 4 course treatment) until the daily prophylactically or therapeutically effective amount of the Tri-Specific Binding Molecule is achieved. Table 3 provides 5 examples of different dosing regimens described above for a typical course of treatment.

TABLE 3

| | | Diabody Dosage (ng diabody per kg | | | | |
|---|---|---|---|---|---|---|
| Regimen | Day | subject weight per day) | | | | |
| 1 | 1, 2, 3, 4 | 100 | 100 | 100 | 100 | 100 |
| | 5, 6, 7 | none | none | none | none | none |
| 2 | 1, 2, 3, 4 | 300 | 500 | 700 | 900 | 1,000 |
| | 5, 6, 7 | none | none | none | none | none |
| 3 | 1, 2, 3, 4 | 300 | 500 | 700 | 900 | 1,000 |
| | 5, 6, 7 | none | none | none | none | none |
| 4 | 1, 2, 3, 4 | 300 | 500 | 700 | 900 | 1,000 |
| | 5, 6, 7 | none | none | none | none | none |

The dosage and frequency of administration of a Tri-Specific Binding Molecule of the present invention may be reduced or altered by enhancing uptake and tissue penetration of the molecule by modifications such as, for example, lipidation.

The dosage of a Tri-Specific Binding Molecule of the invention administered to a patient may be calculated for use as a single agent therapy. Alternatively, the molecule may be used in combination with other therapeutic compositions and the dosage administered to a patient are lower than when said molecules are used as a single agent therapy.

The pharmaceutical compositions of the invention may be administered locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers. Preferably, when administering a molecule of the invention, care must be taken to use materials to which the molecule does not absorb.

The compositions of the invention can be delivered in a vesicle, in particular a liposome (See Langer (1990) “New Methods Of Drug Delivery,” Science 249:1527-1533); Treat et al., in LIPOSOMES IN THE THERAPY OF INFECTIOUS DISEASE AND CANCER, Lopez-Berestein and Fidler (eds.), Liss, New York, pp. 353-365 (1989); Lopez-Berestein, ibid., pp. 3 17-327).

The compositions of the invention can be delivered in a controlled-release or sustained-release system. Any technique known to one of skill in the art can be used to produce sustained-release formulations comprising one or more of the Tri-Specific Binding Molecule(s) of the invention. See, e.g., U.S. Pat. No. 4,526,938; PCT publication WO 91/05548; PCT publication WO 96/20698; Ning et al. (1996) “Intratumoral Radioimmunotherapy Of A Human Colon Cancer Xenograft Using A Sustained-Release Gel,” Radiotherapy & Oncology 39:179-189, Song et al. (1995) “Antibody Mediated Lung Targeting Of Long-Circulating Emulsions,” PDA Journal of Pharmaceutical Science & Technology 50:372-397; Cleek et al. (1997) “Biodegradable Polymeric Carriers For A bFGF Antibody For Cardiovascular Application,” Pro. Int'l. Symp. Control. Rel. Bioact. Mater. 24:853-854; and Lam et al. (1997) “Microencapsulation Of Recombinant Humanized Monoclonal Antibody For Local Delivery,” Proc. Int'l. Symp. Control Rel. Bioact. Mater. 24:759-760, each of which is incorporated herein by reference in its entirety. In one embodiment, a pump may be used in a controlled-release system (See Langer, supra; Sefton, (1987) “Implantable Pumps,” CRC Crit. Rev. Biomed. Eng. 14:201-240; Buchwald et al. (1980) “Long-Term, Continuous Intravenous Heparin Administration By An Implantable Infusion Pump In Ambulatory Patients With Recurrent Venous Thrombosis,” Surgery 88:507-516; and Saudek et al. (1989) “A Preliminary Trial Of The Programmable Implantable Medication System For Insulin Delivery,” N. Engl. J. Med. 321:574-579). In another embodiment, polymeric materials can be used to achieve controlled-release of the molecules (see e.g., MEDICAL APPLICATIONS OF CONTROLLED RELEASE, Langer and Wise (eds.), CRC Pres., Boca Raton, Florida (1974); CONTROLLED DRUG BIOAVAILABILITY, DRUG PRODUCT DESIGN AND PERFORMANCE, Smolen and Ball (eds.), Wiley, New York (1984); Levy et al. (1985) “Inhibition Of Calcification Of Bioprosthetic Heart Valves By Local Controlled-Release Diphosphonate,” Science 228:190-192; During et al. (1989) “Controlled Release Of Dopamine From A Polymeric Brain Implant: In Vivo Characterization,” Ann. Neurol. 25:351-356; Howard et al. (1989) “Intracerebral Drug Delivery In Rats With Lesion-Induced Memory Deficits,” J. Neurosurg. 7(1):105-112); U.S. Pat. Nos. 5,679,377; 5,916,597; 5,912,015; 5,989,463; 5,128,326; PCT Publication No. WO 99/15154; and PCT Publication No. WO 99/20253). Examples of polymers used in sustained-release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. A controlled-release system can be placed in proximity of the therapeutic target (e.g., the lungs), thus requiring only a fraction of the systemic dose (see, e.g., Goodson, in MEDICAL APPLICATIONS OF CONTROLLED RELEASE, supra, vol. 2, pp. 115-138 (1984)). Polymeric compositions useful as controlled-release implants can be used according to Dunn et al. (See U.S. Pat. No. 5,945,155). This particular method is based upon the therapeutic effect of the in situ controlled-release of the bioactive material from the polymer system. The implantation can generally occur anywhere within the body of the patient in need of therapeutic treatment. A non-polymeric sustained delivery system can be used, whereby a non-polymeric implant in the body of the subject is used as a drug delivery system. Upon implantation in the body, the organic solvent of the implant will dissipate, disperse, or leach from the composition into surrounding tissue fluid, and the non-polymeric material will gradually coagulate or precipitate to form a solid, microporous matrix (See U.S. Pat. No. 5,888,533).

Controlled-release systems are discussed in the review by Langer (1990, “New Methods Of Drug Delivery,” Science 249:1527-1533). Any technique known to one of skill in the art can be used to produce sustained-release formulations comprising one or more therapeutic agents of the invention. See, e.g., U.S. Pat. No. 4,526,938; International Publication Nos. WO 91/05548 and WO 96/20698; Ning et al. (1996) “Intratumoral Radioimmunotherapy Of A Human Colon Cancer Xenograft Using A Sustained-Release Gel,” Radiotherapy & Oncology 39:179-189, Song et al. (1995) “Antibody Mediated Lung Targeting Of Long-Circulating Emulsions,” PDA Journal of Pharmaceutical Science & Technology 50:372-397; Cleek et al. (1997) “Biodegradable Polymeric Carriers For A bFGF Antibody For Cardiovascular Application,” Pro. Int'l. Symp. Control. Rel. Bioact. Mater. 24:853-854; and Lam et al. (1997) “Microencapsulation Of Recombinant Humanized Monoclonal Antibody For Local Delivery,” Proc. Int'l. Symp. Control Rel. Bioact. Mater. 24:759-760, each of which is incorporated herein by reference in its entirety.

Where the composition of the invention is a nucleic acid encoding a Tri-Specific Binding Molecule of the present invention, the nucleic acid can be administered in vivo to promote expression of its encoded Tri-Specific Binding Molecule by constructing it as part of an appropriate nucleic acid expression vector and administering it so that it becomes intracellular, e.g., by use of a retroviral vector (See U.S. Pat. No. 4,980,286), or by direct injection, or by use of microparticle bombardment (e.g., a gene gun; Biolistic, Dupont), or coating with lipids or cell surface receptors or transfecting agents, or by administering it in linkage to a homeobox-like peptide which is known to enter the nucleus (See e.g., Joliot et al. (1991) “Antennapedia Homeobox Peptide Regulates Neural Morphogenesis,” Proc. Natl. Acad. Sci. (U.S.A.) 88:1864-1868), etc. Alternatively, a nucleic acid can be introduced intracellularly and incorporated within host cell DNA for expression by homologous recombination.

Treatment of a subject with a therapeutically or prophylactically effective amount of a Tri-Specific Binding Molecule of the present invention can include a single treatment or, preferably, can include a series of treatments. In a preferred example, a subject is treated with such a diabody one time per week for between about 1 to 10 weeks, preferably between 2 to 8 weeks, more preferably between about 3 to 7 weeks, and even more preferably for about 4, 5, or 6 weeks. The pharmaceutical compositions of the invention can be administered once a day, twice a day, or three times a day. Alternatively, the pharmaceutical compositions can be administered once a week, twice a week, once every two weeks, once a month, once every six weeks, once every two months, twice a year or once per year. It will also be appreciated that the effective dosage of the molecules used for treatment may increase or decrease over the course of a particular treatment.

### EXAMPLES

Having now generally described the invention, the same will be more readily understood through reference to the following examples, which are provided by way of illustration and are not intended to be limiting of the present invention unless specified. It will be apparent to those skilled in the art that many modifications, both to materials and methods, can be practiced without departing from the scope of the present disclosure.

### W. Example 1: Characterization of Anti-Human DR5 Monoclonal Antibodies DR5 mAb 1 and DR5 mAb 2

Two monoclonal antibodies were isolated as being capable of immunospecifically binding to human DR5, and accorded the designations “DR5 mAb 1” and “DR5 mAb 2”. As discussed above, the CDRs of these antibodies were found to differ. In order to determine whether the antibodies bound to different DR5 epitopes, a human DR5-Fc fusion protein was prepared and was coated to an immobilized surface. DR5 mAb 1 (1 μg/mL) was biotinylated and incubated with either a control IgG or with DR5 mAb 2 (10 μg/mL), and the ability of the IgG or DR5 mAb 2 antibody to compete for binding (to human DR5-Fc fusion protein) with DR5 mAb 1 was assessed by measuring the amount of immobilized biotinylated antibody. Additionally, the ability of the IgG or DR5 mAb 1 antibody to compete for binding with biotinylated DR5 mAb 2 was assessed. The results of this experiment are shown in Table 4.

TABLE 4

| | | 10 μg/mL Competitor mAb | | |
|---|---|---|---|---|
| 1 μg/mL DR5-Fc | | | DR5 | DR5 |
| Fusion coat | | mIgG | mAb 1 | mAb 2 |
| 1 μg/mL | DR5 mAb 1 | 2.162 | self | 0.826 |
| biotinylated | DR5 mAb 2 | 2.102 | 2.377 | self |
| DR5 mAb | | | | |

The results of this experiment indicate that the biotinylated antibody was capable of binding to the DR5 protein even in the presence of excess amounts of the non-biotinylated antibody. Thus, the results show that DR5 mAb 1 and DR5 mAb 2 bind to different epitopes of DR5.

In order to further characterize the DR5 mAb 1 and DR mAb 2 antibodies, their ability to block binding between DR5 and the TRAIL ligand as assessed. Thus, biotinylated DR5 mAb 1, biotinylated DR5 mAb 2 or biotinylated DR5-Fc fusion (each at 2 μg/mL) were separately incubated with immobilized DR5-Fc fusion (1 μg/mL) in the presence of either buffer or histidine tagged TRAIL (20 μg/mL). The amount of immobilized biotinylated antibody was assessed. The results of this experiment are shown in Table 5.

TABLE 5

| 2 μg/mL | 1 μg/mL DR5-Fc fusion coat | | |
|---|---|---|---|
| Biotinylated | 20 μg/mL | | 1 μg/mL TRAIL- |
| DR5 mAb | TRAIL-His | Buffer | His coat |
| DR5 mAb 1 | 1.939 | 2.118 | 0.007 |
| DR5 mAb 2 | 2.052 | 2.052 | 0.008 |
| DR5-Fc fusion | — | — | 0.288 |

The results show that the amount of DR5 mAb 1 or DR5 mAb 2 bound to the immobilized DR5-Fc was not affected by the presence of the histidine tagged TRAIL, thus indicating that neither DR5 mAb 1 nor DR5 mAb 2 block the TRAIL ligand binding site of DR5. Additionally, neither antibody was capable of binding to the histidine tagged TRAIL ligand.

### X. Example 2: Species Specificity of Anti-Human DR5 Monoclonal Antibodies DR5 mAb 1 and DR5 mAb 2

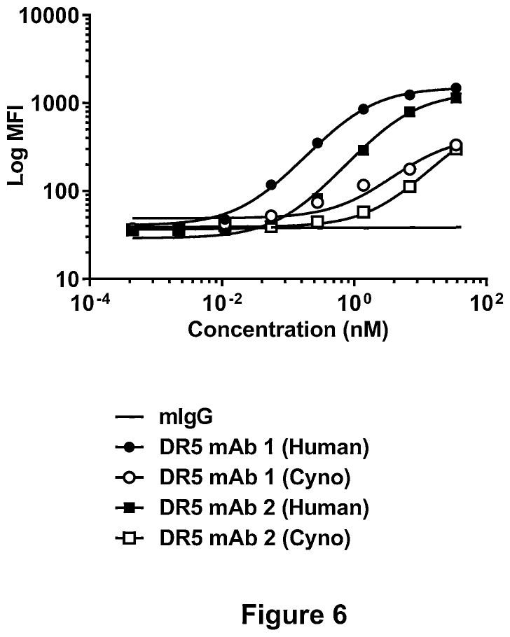

In order to assess the species specificity of anti-human DR5 monoclonal antibodies DR5 mAb 1 and DR5 mAb 2, the ability of the antibodies to bind to human DR5 was compared with their ability to bind cynomolgus monkey (Macaca fascicularis) DR5. The results of this experiment are shown in FIG. 6. The results show that both antibodies are capable of binding to cynomolgus monkey DR5, but that they each exhibit higher binding affinity for human DR5.

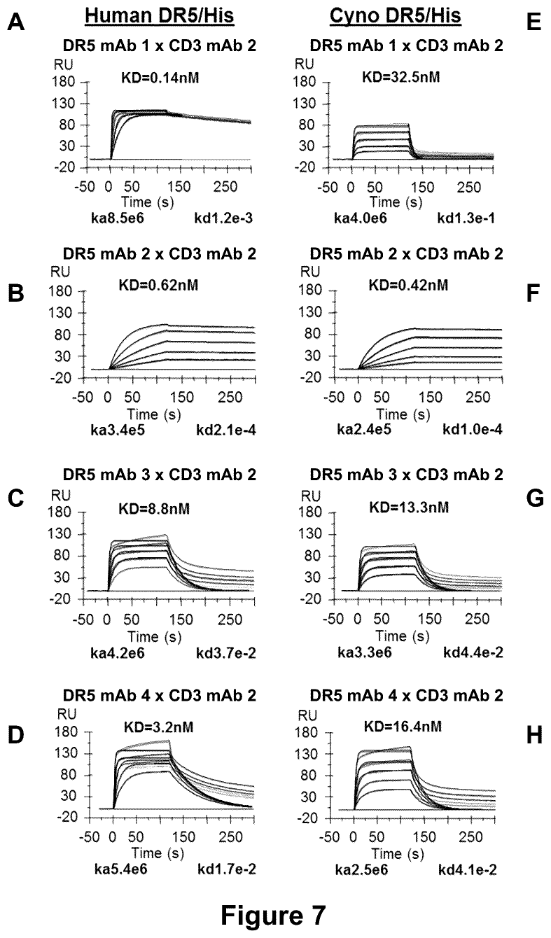

The kinetics of binding was investigated using Biacore Analysis, as shown in FIG. 7. Bi-specific DR5×CD3 diabodies were incubated with His-tagged DR5 and the kinetics of binding was determined via Biacore analysis. The diabodies employed were DR5 mAb 1×CD3 mAb 2 (FIG. 7, Panels A and E), DR5 mAb 2×CD3 mAb 2 (FIG. 7, Panels B and F), DR5 mAb 3×CD3 mAb 2 (FIG. 7, Panels C and G), and DR5 mAb 4×CD3 mAb 2 (FIG. 7, Panels D and H). FIG. 7, Panels A-D show the results for human DR5. FIG. 7, Panels E-H show the results for cynomolgus monkey DR5. The calculated ka, kd and KD are presented in Table 6.

TABLE 6

| | Human | | | Cynomolgus Monkey | | |
|---|---|---|---|---|---|---|
| Anti-DR | | | KD | | | KD |
| Antibody | ka | kd | (nM) | ka | kd | (nM) |
| DR mAb 1 | 8.5 × 10⁶ | 1.2 × 10⁻³ | 0.14 | 4.0 × 10⁶ | 1.3 × 10⁻¹ | 32.5 |
| DR mAb 2 | 3.4 × 10⁵ | 2.1 × 10⁻⁴ | 0.62 | 2.4 × 10⁵ | 1.0 × 10⁻⁴ | 0.42 |
| DR mAb 3 | 4.2 × 10⁶ | 3.7 × 10⁻² | 8.8 | 3.3 × 10⁶ | 4.4 × 10⁻² | 13.3 |
| DR mAb 4 | 5.4 × 10⁶ | 1.7 × 10⁻² | 3.2 | 2.5 × 10⁶ | 4.1 × 10⁻² | 16.4 |

The results demonstrate that DR5 mAb 1 and DR5 mAb 2 exhibit altered kinetics of binding relative to reference antibodies DR5 mAb 3 and DR5 mAb 4.

### Y. Example 3: Unexpected Superiority of DR5 mAb 1 and DR5 mAb 2

The ability of DR5-binding molecules DR5 mAb 1 and DR5 mAb 2 of the present invention to mediate cytotoxicity was compared with that of the reference anti-DR5 antibodies: DR5 mAb 3 and DR5 mAb 4. In order to make such a comparison, a bi-specific DR5×CD3 diabody containing the VL and VH Domains of these antibodies and the VL and VH Domains of CD3 mAb 2 were prepared. The prepared diabodies were: DR5 mAb 1×CD3 mAb 2; DR5 mAb 2×CD3 mAb 2; DR5 mAb 3×CD3 mAb 2; and DR5 mAb 4×CD3 mAb 2.

The employed control diabody contained the VL and VH domains of anti-fluorescein antibody 4-4-20 (respectively, SEQ ID NOs:138 and 139) and the VL and VH domains of CD3 mAb 2 (respectively, SEQ ID NOs:102 and 108), and was designated as the anti-fluorescein×anti-CD3 control diabody “4-4-20×CD3 mAb 2.” The diabody was composed of two polypeptide chains. The first polypeptide chain of the diabody had the amino acid sequence (SEQ ID NO:300) (CDRs are shown in underline):

DVVMTQTPFS LPVSLGDQAS ISCRSSQSLV HSNGNTYLRW
YLQKPGQSPK VLIYKVSNRF SGVPDRFSGS GSGTDFTLKI
SRVEAEDLGV YFCSQSTHVP WTFGGGTKLE IKGGGSGGGG
EVQLVESGGG LVQPGGSLRL SCAASGFTFN TYAMNWVRQA
PGKGLEWVAR IRSKYNNYAT YYADSVKDRF TISRDDSKNS
LYLQMNSLKT EDTAVYYCVR HGNFGNSYVS WFAYWGQGTL
VTVSSGGCGG GEVAALEKEV AALEKEVAAL EKEVAALEK

In SEQ ID NO:300, amino acid residues 1-112 correspond to the VL Domain of anti-fluorescein antibody 4-4-20 (SEQ ID NO:138), residues 113-120 correspond to the intervening spacer peptide GGGSGGGG (Linker 1) (SEQ ID NO:33), residues 121-245 correspond to the VH Domain of CD3 mAb 2 (SEQ ID NO:108), residues 246-251 are a cysteine-containing spacer peptide (GGCGGG) (SEQ ID NO:34), and residues 252-280 correspond to an E-coil Domain (SEQ ID NO:39).

The second polypeptide chain of the diabody had the amino acid sequence (SEQ ID NO:301) (CDRs are shown in underline):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGEV
KLDETGGGLV QPGRPMKLSC VASGFTFSDY WMNWVRQSPE
KGLEWVAQIR NKPYNYETYY SDSVKGRFTI SRDDSKSSVY
LQMNNLRVED MGIYYCTGSY YGMDYWGQGT SVTVSSGGCG
GGKVAALKEK VAALKEKVAA LKEKVAALKE

In SEQ ID NO:301, amino acid residues 1-110 correspond to the VL Domain of CD3 mAb 2 (SEQ ID NO:104), residues 111-118 correspond to the intervening spacer peptide GGGSGGGG (Linker 1) (SEQ ID NO:33), residues 119-236 correspond to the VH Domain of anti-fluorescein antibody 4-4-20 (SEQ ID NO:139), residues 237-242 are a cysteine-containing spacer peptide (GGCGGG) (SEQ ID NO:34), and residues 243-270 correspond to a K-coil Domain (SEQ ID NO:40).

Target tumor cells were incubated with one of these diabodies or with the control diabody (4-4-20×CD3 mAb 2) in the presence of peripheral blood mononuclear cells (PBMC) and A549 adenocarcinomic human alveolar basal epithelial cells for 24 hours at an effector to target cell ratio of 20:1. The percentage cytotoxicity was determined by measuring the release of lactate dehydrogenase (LDH) into the media by damaged cells.

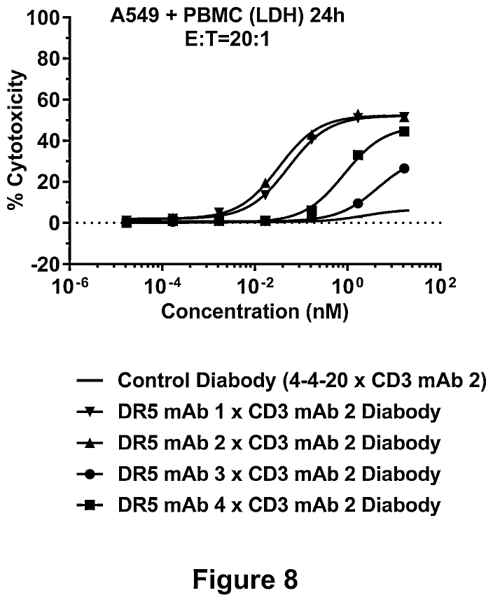

The results of this investigation are shown in FIG. 8. Similar results were obtained using SKMES human lung cancer cells, DU145 human prostate cancer cells, A375 human malignant melanoma cells, SKBR3 human HER2-overexpressing breast carcinoma cells, and JIMT human breast carcinoma cells. The results indicate that the VL and VH domains of DR5 mAb 1 and DR5 mAb 2 are significantly and unexpectedly more potent in inducing cytotoxicity than those of the reference DR5 mAbs.

### Z. Example 4: The Trispecific Binding Molecules Mediate Coordinated and Simultaneous Binding to Target Cells

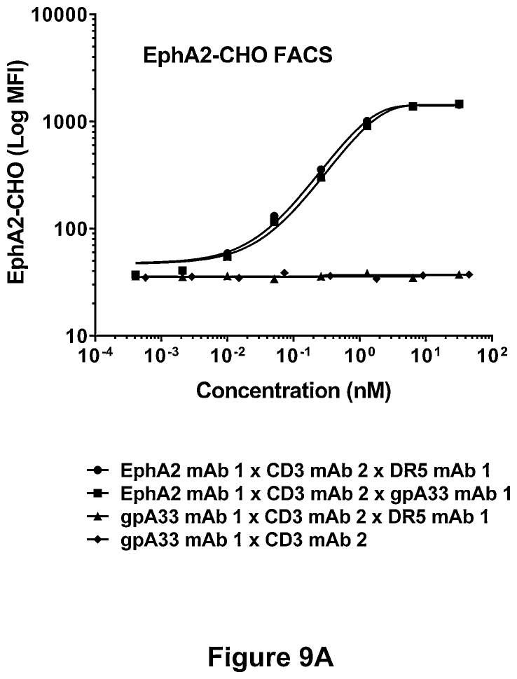

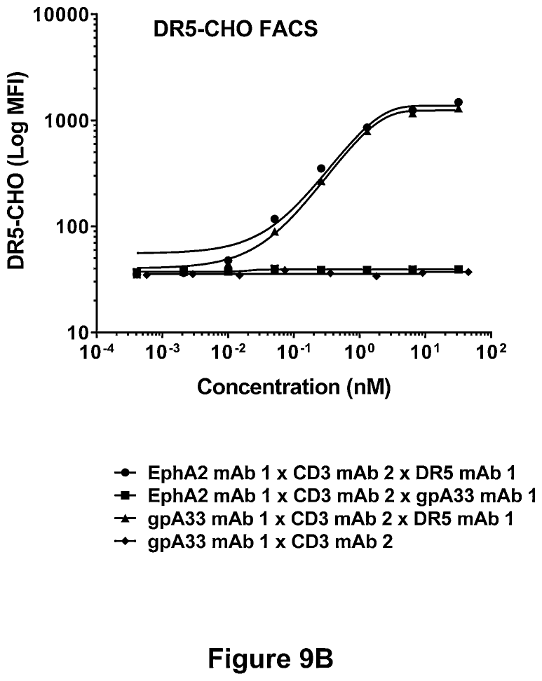

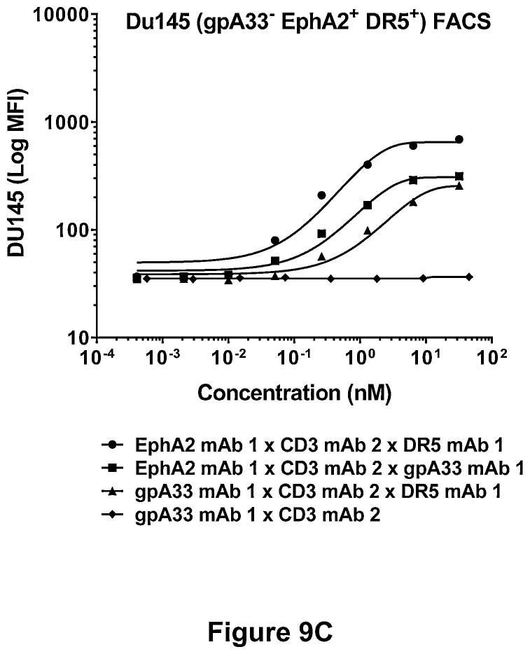

The ability of Tri-Specific Binding Molecules of the present invention to bind to target cells was investigated. The employed trispecific molecules were: EphA2 mAb 1×CD3 mAb 2×DR5 mAb 1; EphA2 mAb 1×CD3 mAb 2×gpA33 mAb 1; and gpA33 mAb 1×CD3 mAb 2×DR5 mAb 1. As shown in FIG. 9A, those Tri-Specific Binding Molecules that comprise an EphA2 Cancer Antigen-Binding Domain were found to be capable of binding to EphA2-expressing CHO target cells. As shown in FIG. 9B, those Tri-Specific Binding Molecules that comprise a DR5 Cancer Antigen-Binding Domain were found to be capable of binding to DR5-expressing CHO target cells. As shown in FIG. 9C, those Tri-Specific Binding Molecules that comprise an EphA2 Cancer Antigen-Binding or a DR5 Cancer Binding Domain were found to be capable of binding to DU145 cells. DU145 cells are a human prostate cell line that express both EphA2 and DR5, but not gpA33. The above-described reference gpA33 mAb 1×CD3 mAb 2 diabody was used as a control.

Significantly, the data show that when both of the two Cancer Antigen-Binding Domains of a Tri-Specific Binding Molecule of the present invention are able to bind to a target cell, such dual binding is associated with a synergistic (e.g., a 5-25 fold) enhancement in target binding.

### AA. Example 5: The Trispecific Binding Molecules Mediate Cytotoxicity of Bound Target Cells

The ability of Tri-Specific Binding Molecules of the present invention to mediate the cytotoxicity of bound target cells in the presence of cytotoxic lymphocytes was investigated. The employed trispecific molecules were: EphA2 mAb 1×CD3 mAb 2×DR5 mAb 1; EphA2 mAb 1×CD3 mAb 2×gpA33 mAb 1; and gpA33 mAb 1×CD3 mAb 2×DR5 mAb 1. The above-described reference gpA33 mAb 1×CD3 mAb 2 diabody and the 4-4-20×CD3 mAb 2 diabody were used as controls.

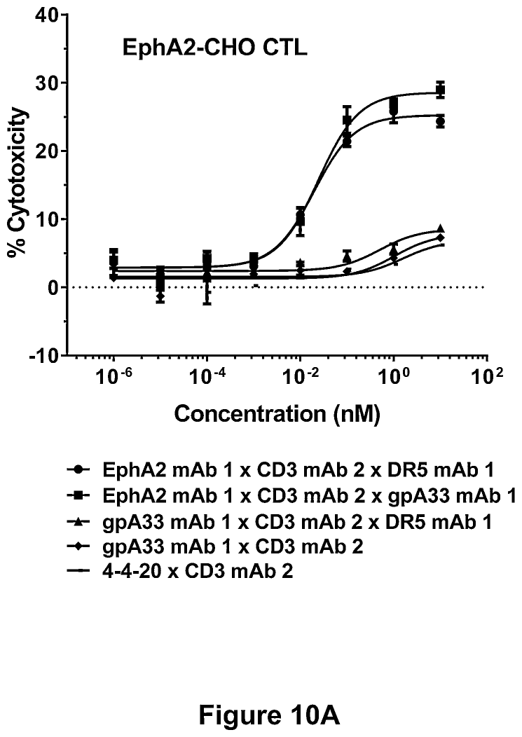

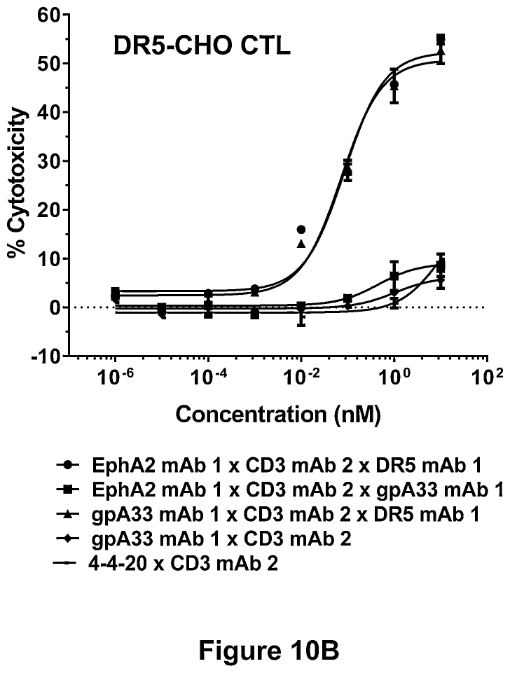

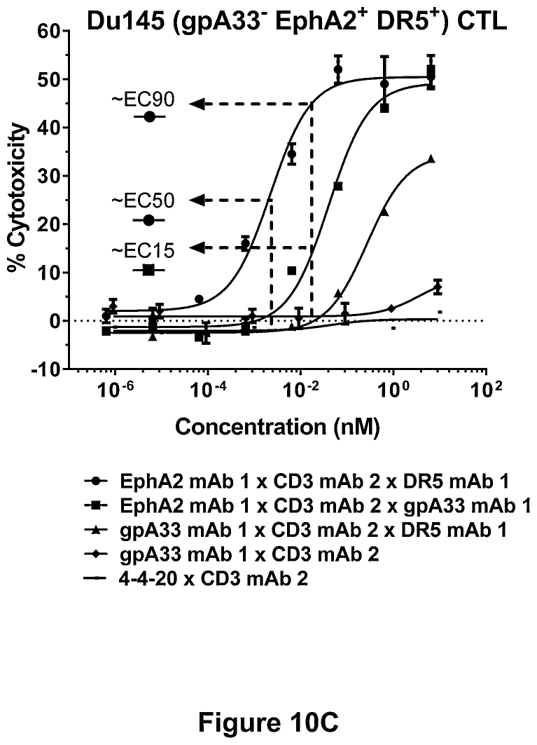

As shown in FIG. 10A, those Tri-Specific Binding Molecules that comprise an EphA2 Cancer Antigen-Binding Domain, and thus were able to bind to bind to EphA2-expressing CHO cells, were able to mediate the cytotoxicity of such cells in the presence of the cytotoxic lymphocytes. As shown in FIG. 10B, those Tri-Specific Binding Molecules that comprise a DR5 Cancer Antigen-Binding Domain, and thus were able to bind to bind to DR5-expressing CHO cells, were found to be capable of mediating cytotoxicity of DR5-expressing CHO target cells in the presence of the cytotoxic lymphocytes.

As shown in FIG. 10C, those Tri-Specific Binding Molecules that comprise an EphA2 Cancer Antigen-Binding or a DR5 Cancer Binding Domain, and thus are capable of binding to DU145 cells, were able to mediate the cytotoxicity of such cells in the presence of the cytotoxic lymphocytes. Significantly, the data show that when both of the two Cancer Antigen-Binding Domains of a Tri-Specific Binding Molecule of the present invention are able to bind to a target cell, such dual binding is associated with a synergistic enhancement in target binding. Thus, EphA2 mAb 1×CD3 mAb 2×DR5 mAb 1, which is capable of binding to both EphA2 and DR5, mediated substantially greater cytotoxicity than EphA2 mAb 1×CD3 mAb 2×gpA33 mAb 1 or gpA33 mAb 1×CD3 mAb 2×DR5 mAb 1, which were capable of binding to only EphA2 or DR5 molecules of the DU146 cells (since such cells lack gpA33).

In this regard, at approximately the EC50 of EphA2 mAb 1×CD3 mAb 2×DR5 mAb 1, no cytotoxic lymphocyte response is seen for either EphA2 mAb 1×CD3 mAb 2×gpA33 mAb 1 or gpA33 mAb 1×CD3 mAb 2×DR5 mAb 1. At approximately the EC90 of EphA2 mAb 1×CD3 mAb 2×DR5 mAb 1, EphA2 mAb 1×CD3 mAb 2×gpA33 mAb 1 shows only an EC15, and gpA33 mAb 1×CD3 mAb 2×DR5 mAb 1 shows no cytotoxic lymphocyte response at all.

All publications and patents mentioned in this specification are herein incorporated by reference to the same extent as if each individual publication or patent application was specifically and individually indicated to be incorporated by reference in its entirety. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

1-20. (canceled)

21. A method for treating cancer comprising administering to a subject in need thereof a therapeutically effective amount of a Tri-Specific Binding Molecule that immunospecifically binds to three different epitopes, comprising:
(I) four different polypeptide chains covalently complexed together;
(II) an Antigen-Binding Domain I that immunospecifically binds to an Epitope I present on a first antigen, an Antigen-Binding Domain II that immunospecifically binds to an Epitope II present on a second antigen, and an Antigen-Binding Domain III that immunospecifically binds to an Epitope III present on a third antigen; and
(III) a Fc Domain, wherein:
(A) one of Epitope I, Epitope II or Epitope III is an epitope of an Effector Cell Antigen, a second of Epitope I, Epitope II or Epitope III is an epitope of a first Cancer Antigen, and a third of Epitope I, Epitope II or Epitope III is an epitope of a second Cancer Antigen;
(B) a first polypeptide chain comprises, from the N-terminus to the C-terminus:
(VLI Domain)-(Linker 1)-(VHII Domain)-(Linker 2)-(Heterodimer-Promoting Domain)-(Linker 3)-(CH2-CH3 Domain);
(C) a second polypeptide chain comprises, from the N-terminus to the C-terminus:
(VLII Domain)-(Linker 1)-(VHI Domain)-(Linker 2)-(Heterodimer-Promoting Domain);
(D) a third polypeptide chain comprises, from the N-terminus to the C-terminus:
(VHIII Domain)-(Cysteine-Containing Domain)-(CH2-CH3 Domain);
(E) a fourth polypeptide chain comprises, from the N-terminus to the C-terminus:
(VLIII Domain)-(Cysteine-Containing Domain);
(F) the VLI Domain is a Light Chain Variable Domain of an immunoglobulin that binds to Epitope I, the VHI Domain is a Heavy Chain Variable Domain of an immunoglobulin that binds to Epitope I, the VLII Domain is a Light Chain Variable Domain of an immunoglobulin that binds to Epitope II, the VHII Domain is a Heavy Chain Variable Domain of an immunoglobulin that binds to Epitope II, the VLIII Domain is a Light Chain Variable Domain of an immunoglobulin that binds to Epitope III, and the VHIII Domain is a Heavy Chain Variable Domain of an immunoglobulin that binds to Epitope III;
(G) the VLI Domain and the VHI Domain associate to form the Antigen-Binding Domain I, the VLII Domain and the VHII Domain associate to form the Antigen-Binding Domain II, the VLIII Domain and the VHIII Domain associate to form the Antigen-Binding Domain III, the CH2-CH3 Domain of the first polypeptide chain and the CH2-CH3 Domain of the third polypeptide chain associate to form the Fc Domain, the Antigen-Binding Domain I and the Antigen-Binding Domain II are Diabody-Type Binding Domains, and the Antigen-Binding Domain III is a Non-Diabody-Type Binding Domain; and
(H) the Linker 1 comprises the sequence of SEQ ID NO: 33;
the Linker 2 comprises the sequence of SEQ ID NO: 34 or 47;
the Heterodimer-Promoting Domain on the first polypeptide chain is an E-coil Domain and the Heterodimer-Promoting Domain on the second polypeptide chain is a K-coil Domain, or the Heterodimer-Promoting Domain on the first polypeptide chain is a K-coil Domain and the Heterodimer-Promoting Domain on the second polypeptide chain is an E-coil Domain, the E-coil Domain independently comprises the sequence of SEQ ID NO: 39 or 41, and the K-coil Domain independently comprises the sequence of SEQ ID NO: 40 or 42;
the Linker 3 comprises the sequence of SEQ ID NO: 46, 47, 48, 49, 50, 51, 152, or GCG or GGG; and
the Cysteine-Containing Domain independently comprises the sequence of SEQ ID NO: 34, 36, 38, 48, 210 or 211.

22. The method of claim 21, wherein (i) the first polypeptide chain and the second polypeptide chain are covalently bonded to one another; (ii) the first polypeptide chain and the third polypeptide chain are covalently bonded to one another; and (iii) the third polypeptide chain and the fourth polypeptide chain are covalently bonded to one another.

23. The method of claim 21, wherein the CH2-CH3 Domain of the first polypeptide chain and the third polypeptide chain comprise at least one amino acid substitution, relative to the sequence of SEQ ID NO:1, and the Fc Domain formed from their association exhibits altered FcγR-mediated effector function.

24. The method of claim 21, wherein:
(A) the Epitope I, Epitope II and Epitope III are, respectively, an epitope of the first Cancer Antigen, an epitope of the second Cancer Antigen and an epitope of the Effector Cell Antigen;
(B) the Epitope I, Epitope II and Epitope III are, respectively, an epitope of the first Cancer Antigen, an epitope of the Effector Cell Antigen and an epitope of the second Cancer Antigen;
(C) the Epitope I, Epitope II and Epitope III are, respectively, an epitope of second Cancer Antigen, an epitope of the first Cancer Antigen, and an epitope of the Effector Cell Antigen;
(D) the Epitope I, Epitope II and Epitope III are, respectively, an epitope of the second Cancer Antigen, an epitope of the Effector Cell Antigen and an epitope of the first Cancer Antigen;
(E) the Epitope I, Epitope II and Epitope III are, respectively, an epitope of the Effector Cell Antigen, an epitope of the first Cancer Antigen, and an epitope of the second Cancer Antigen; or
(F) the Epitope I, Epitope II and Epitope III are, respectively, an epitope of the Effector Cell Antigen, an epitope of second Cancer Antigen, and an epitope of the first Cancer Antigen.

25. The method of claim 21, wherein the Effector Cell Antigen is CD2, CD3, CD16, CD19, CD20, CD22, CD32B, CD64, B cell Receptor (BCR), T cell Receptor (TCR), or NKG2D Receptor.

26. The method of claim 21, wherein the first Cancer Antigen and the second Cancer Antigen are independently chosen from: colon cancer antigen 19.9; a gastric cancer mucin; antigen 4.2; glycoprotein A33 (gpA33); ADAM-9; gastric cancer antigen AH6; ALCAM; malignant human lymphocyte antigen APO-1; cancer antigen B1; B7-H3; beta-catenin; blood group ALeb/Ley; Burkitt's lymphoma antigen-38.13, colonic adenocarcinoma antigen C14; ovarian carcinoma antigen CA125; Carboxypeptidase M; CD5; CD19; CD20; CD22; CD23; CD25; CD27; CD28; CD30; CD33; CD36; CD45; CD46; CD52; CD79a/CD79b; CD103; CD317; CDK4; carcinoembryonic antigen (CEA); CEACAM5; CEACAM6; CO17-1A; CO-43 (blood group Leb); CO-514 (blood group Lea); CTA-1; CTLA4; Cytokeratin 8; antigen D1.1; antigen D156-22; DR5; E1 series (blood group B); EGFR (Epidermal Growth Factor Receptor); Ephrin receptor A2 (EphA2); ErbB1; ErbB3; ErbB4; GAGE-1; GAGE-2; GD2/GD3/GM2; lung adenocarcinoma antigen F3; antigen FC10.2; G49, ganglioside GD2; ganglioside GD3; ganglioside GM2; ganglioside GM3; GD2; GD3; GICA 19-9; GM2; gp100; human leukemia T cell antigen Gp37; melanoma antigen gp75; gpA33; HER2 antigen (p185HER2); human milk fat globule antigen (HMFG); human papillomavirus-E6/human papillomavirus-E7; high molecular weight melanoma antigen (HMW-MAA); I antigen (differentiation antigen) I(Ma); Integrin Alpha-V-Beta-6 Integrinβ6 (ITGB6); Interleukin-13; Receptor α2 (IL13Rα2); JAM-3; KID3; KID31; KS 1/4 pan-carcinoma antigen; human lung carcinoma antigens L6 and L20; LEA; LUCA-2; M1:22:25:8; M18; M39; MAGE-1; MAGE-3; MART; MUC-1; MUM-1; Myl; N-acetylglucosaminyltransferase; neoglycoprotein; NS-10; OFA-1; OFA-2; Oncostatin M; p15; melanoma-associated antigen p97; polymorphic epithelial mucin (PEM); polymorphic epithelial mucin antigen (PEMA); PIPA; prostate-specific antigen (PSA); prostate-specific membrane antigen (PSMA); prostatic acid phosphate; R24, ROR1; sphingolipids; SSEA-1; SSEA-3; SSEA-4; sTn; T cell receptor derived peptide; T5A7; TAG-72; TL5 (blood group A); TNF-α receptor; TNF-β receptor; TNF-γ receptor; TRA-1-85 (blood group H); Transferrin Receptor; tumor-specific transplantation antigen (TSTA), oncofetal antigen-alpha-fetoprotein (AFP); VEGF, VEGFR; VEP8; VEP9; VIM-D5; Y hapten, Ley and 5T4.

27. The method of claim 26, wherein one of Epitope I, Epitope II or Epitope III is an epitope of a Cancer Antigen and the Antigen-Binding Domain that immunospecifically binds to the epitope comprises the six CDRs of SEQ ID NO: 3 and 8, 13 and 18, 23 and 31, 25 and 31, 27 and 31, 29 and 31, 54 and 58, 62 and 66, 70 and 74, 78 and 82, 86 and 90, 94 and 98, 153 and 158, 163 and 167, 172 and 177, 181 and 186, 191 and 192, 193 and 194, 195 and 196, 197 and 198, 199 and 200, 201 and 202, 203 and 204, 205 and 206, 302 and 303, 304 and 305, 306 and 307, 308 and 309, 310 and 311, 312 and 313, 314 and 315, or 321 and 322.

28. The method of claim 21, wherein:
the CH2-CH3 Domain of the first polypeptide chain is a knob-bearing CH2-CH3 Domain and the CH2-CH3 Domain of the third polypeptide chain is a hole-bearing CH2-CH3 Domain; or
the CH2-CH3 Domain of the third polypeptide chain is a knob-bearing CH2-CH3 Domain and the CH2-CH3 Domain of the first polypeptide chain is a hole-bearing CH2-CH3 Domain.

29. The method of claim 28, wherein:
the knob-bearing CH2-CH3 Domain comprises the sequence of SEQ ID NO: 52, and
the hole-bearing CH2-CH3 Domain comprises the sequence of SEQ ID NO: 53.

30. The method of claim 21, wherein:
(A) the epitope of an Effector Cell Antigen is a CD2 epitope recognized by antibody Lo-CD2a;
(B) the epitope of an Effector Cell Antigen is a CD3 epitope recognized by antibody OKT3, M291, YTH12.5, Anti-CD3 mAb 1 or Anti-CD3 mAb 2;
(C) the epitope of an Effector Cell Antigen is a CD16 epitope recognized by antibody 3G8 or A9;
(D) the epitope of an Effector Cell Antigen is a CD19 epitope recognized by antibody MD1342, MEDI-551, blinatumomab or HD37;
(E) the epitope of an Effector Cell Antigen is a CD20 epitope recognized by antibody rituximab, ibritumomab, ofatumumab, and tositumomab;
(F) the epitope of an Effector Cell Antigen is a CD22 epitope recognized by antibody epratuzumab;
(G) the epitope of an Effector Cell Antigen is a CD32B epitope recognized by antibody CD32B mAb 1;
(H) the epitope of an Effector Cell Antigen is a CD64 epitope recognized by antibody CD64 mAb 1;
(I) the epitope of an Effector Cell Antigen is a BCR/CD79 epitope recognized by antibody CD79 mAb 1;
(J) the epitope of an Effector Cell Antigen is a TCR epitope recognized by antibody BMA 031;
(K) the epitope of an Effector Cell Antigen is a NKG2D Receptor epitope recognized by antibody KYK-2.0; or
(L) one of Epitope I, Epitope II or Epitope III is an epitope of an Effector Cell Antigen and the Antigen-Binding Domain that immunospecifically binds to the epitope comprises the six CDRs of SEQ ID NO:102 and 103; 104 and 108; 104 and 112; 114 and 115; 116 and 117; 118 and 119; 120 and 121; 122 and 123; 124 and 125; 126 and 127; 128 and 129; 130 and 131; 132 and 133; 134 and 135; or 136 and 137.

31. The method of claim 21, wherein:
the Linker 2 comprises a cysteine residue;
the E-coil Domain and the K-coil Domain adjacent to the Linker 2 each comprise a cysteine residue; or
the Linker 2 comprises a cysteine residue and the E-coil Domain or the K-coil Domain adjacent to the Linker 2 comprises a cysteine residue.

32. The method of claim 31, wherein:
the E-coil Domain of SEQ ID NO: 41 or the K-coil Domain of SEQ ID NO: 42 is adjacent to the Linker 2 when the Linker 2 comprises the sequence of SEQ ID NO: 47;
the E-coil Domain of SEQ ID NO: 39 or the K-coil Domain of SEQ ID NO: 40 is adjacent to the Linker 2 when the Linker 2 comprises the sequence of SEQ ID NO: 34; or
the E-coil Domain of SEQ ID NO: 41 or the K-coil Domain of SEQ ID NO: 42 is adjacent to the Linker 2 when the Linker 2 comprises the sequence of SEQ ID NO: 34.

33. The method of claim 21, wherein the CH2-CH3 Domain of the first polypeptide chain and the third polypeptide chain comprise:
(A) one substitution selected from the group consisting of:
F243L, R292P, Y300L, V305I, and P396L;
(B) two substitutions selected from the group consisting of:
(1) F243L and P396L;
(2) F243L and R292P; and
(3) R292P and V305I;
(C) three substitutions selected from the group consisting of:
(1) F243L, R292P and Y300L;
(2) F243L, R292P and V305I;
(3) F243L, R292P and P396L; and
(4) R292P, V305I and P396L;
(D) four substitutions selected from the group consisting of:
(1) F243L, R292P, Y300L and P396L; and
(2) F243L, R292P, V305I and P396L; or
(E) five substitutions selected from the group consisting of:
(1) F243L, R292P, Y300L, V305I and P396L; and
(2) L235V, F243L, R292P, Y300L and P396L.

34. The method of claim 21, wherein the third polypeptide chain comprises a CH1 Domain.

35. The method of claim 34, wherein the CH1 Domain comprises the sequence of SEQ ID NO: 207 or 208.

36. The method of claim 34, wherein the fourth polypeptide chain comprises a CL Domain.

37. The method of claim 36, wherein the CL Domain comprises the sequence of SEQ ID NO: 210 or 211.
